(19) United States
(12) Patent Application Publication
(10) Pub. No.: US 2021/0305519 A1
(43) Pub. Date: Sep. 30, 2021

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young Jung, Gyeonggi-do (KR); Su-Hyun LEE, Gyeonggi-do (KR); Jin-Ri HONG, Gyeonggi-do (KR); Ji-Song JUN, Gyeonggi-do (KR); Sang-Hee CHO, Gyeonggi-do (KR)

(21) Appl. No.: 17/203,629

(22) Filed: Mar. 16, 2021

(30) Foreign Application Priority Data
Mar. 17, 2020 (KR) 10-2020-0032582

(51) Int. Cl. H01L 51/00

(52) U.S. Cl. CPC H01L 51/0072; H01L 51/0054; H01L 51/0056; H01L 51/0074; H01L 51/006; H01L 51/0073; H01L 51/0067

(57) ABSTRACT

The present disclosure relates to a plurality of host materials comprising a first host material comprising a compound represented by formula 1 and a second host material comprising a compound represented by formula 2, and an organic electroluminescent device comprising the same. By comprising the specific combination of the compound as host materials, an organic electroluminescent device having low driving voltage and/or high luminous efficiency and/or long lifespan can be provided compared with a conventional organic electroluminescent device.

### TECHNICAL FIELD

The present disclosure relates to a plurality of host materials and organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an OLED have been rapidly commercialized.

The most important factor determining luminous efficiency in an OLED is light-emitting materials. Until now, Iridium(III) complexes have been widely known as phosphorescent light-emitting materials, including bis(2-(2′-benzothienyl)-pyridinato-N,C-3′)iridium(acetylacetonate) [(acac)Ir(btp)₂], tris(2-phenylpyridine)iridium [Ir(ppy),] and bis(4,6-difluorophenylpyridinato-N,C2)picolinato iridium (Firpic) as red-, green-, and blue-emitting materials, respectively.

However, although the conventional materials have an advantage in terms of luminous characteristics, when they are used in OLEDs, they are not satisfactory in terms of operational lifespan, and luminous efficiency is also inferior, and there is still a need for improvement of efficient luminous materials for OLEDs. In particular, recently, OLEDs having high luminous efficiency and/or long lifespan are required for long-time use and high resolution of displays.

### DISCLOSURE OF THE INVENTION

### Technical Problem

The object of the present disclosure is firstly, to provide a plurality of host material which is able to produce an organic electroluminescent device having low driving voltage and/or high luminous efficiency and/or long lifespan, and secondly, to provide an organic electroluminescent device comprising the host materials.

### Solution to Problem

As a result of intensive studies to solve the technical problem above, the present inventors found that the aforementioned objective can be achieved by using a compound represented by the following formula 1 having a structure in which an aryl is linked to a heteroaryl moiety as an electron-host material; and a compound represented by the following formula 2 having a structure in which the residues of the 8-membered ring are multi-fused as a hole-host material. As a result, the formation of excitons in the light-emitting layer is improved and at the same time the highest occupied molecular orbital (HOMO) barrier between the hole transport layer and the light-emitting layer is lowered, and then completed the present invention.

HA-(L₁-Ar₁)ₐ (1)

wherein

HAr represents a substituted or unsubstituted nitrogen-containing (3- to 10-membered)heteroaryl;

L₁ represents a single bond or a substituted or unsubstituted (C6-C30)arylene;

Ar₁ represents a substituted or unsubstituted (C6-C30)aryl; and

a represents an integer of 1 to 3, when a is an integer of 2 or more, each of (L₁-Ar₁) may be the same or different;

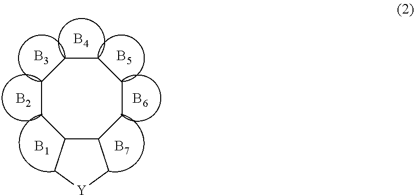

wherein

B₁ to B₇ each independently are absent, or each independently represent a substituted or unsubstituted (C5-C20) ring, in which the carbon atom of the ring may be replaced with one or more heteroatom(s) selected from nitrogen, oxygen, and sulfur, with the proviso that at least five of B₁ to B₇ are present, and the immediately adjacent rings of B₁ to B₇ may be fused with each other;

Y represents —N-L₂-(Ar₂)ₙ, —O—, —S—, or —CR₁R₂;

L₂ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene:

Ar₂ represents a substituted or unsubstituted (C6-C30)aryl, a substituted or unsubstituted (3- to 30-membered)heteroaryl, or —NR₃R₄;

R₁ to R₄ each independently represent hydrogen, deuterium, halogen, cyano, a substituted or unsubstituted (C1-C30)alkyl, a substituted or unsubstituted (C2-C30)alkenyl, a substituted or unsubstituted fused ring of (C3-C30)aliphatic ring and (C6-C30)aromatic ring, a substituted or unsubstituted (C6-C30)aryl, a substituted or unsubstituted (3- to 30-membered)heteroaryl, or a substituted or unsubstituted (C3-C30)cycloalkyl; or may be linked to the adjacent substituent to form a ring(s); and

n represents an integer of 1 or 2, when n is 2, each of Ar₂ may be the same or different.

### Advantageous Effects of Invention

By comprising the specific combination of the compound as host materials according to the present disclosure, an organic electroluminescent device having low driving voltage and/or high luminous efficiency and/or long lifespan can be provided as compared with a conventional organic electroluminescent device, and a display device or a lighting device using the same can be manufactured.

### MODE FOR THE INVENTION

Hereinafter, the present disclosure will be described in detail. However, the following description is intended to explain the invention, and is not meant in any way to restrict the scope of the invention.

The present disclosure relates to a plurality of host materials comprising at least one first host material represented by formula 1 and at least one second host material represented by formula 2 and an organic electroluminescent device comprising the host materials.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The two or more compounds comprised in the plurality of host materials of the present disclosure may be included in one light-emitting layer or may be respectively included in different light-emitting layers. When the at least two host materials are comprised in one layer, the at least two host materials may be mixture-evaporated to form a layer, or simultaneously may be co-evaporated individually to form a layer.

The term “(C1-C30)alkyl(ene)” in the present disclosure is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, and tert-butyl, etc. Herein, the term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. Herein, the term “(C3-C30)cycloalkyl(ene)” is a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. Herein, the term “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and comprise a spiro structure. Examples of the aryl specifically include phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-fluorene]yl, azulenyl, etc. More specifically, the aryl may be phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yI, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-dipheyl-3-fluorenyl, 9,9-diphenyl-4-fluorenyl, etc. Herein, the term “(3- to 30-membered)heteroaryl(ene)” is an aryl having 3 to 30 ring backbone atoms, in which the number of ring backbone atoms is preferably 3 to 30, more preferably 5 to 20, including at least one, preferably 1 to 4 heteroatoms selected from the group consisting of B, N, O, S, Si, P, and Ge. The above heteroaryl or heteroarylene may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s) and may include a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, benzonaphthofuranyl, dibenzothiophenyl, benzonaphthothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, naphthiridinyl, carbazolyl, benzocarbazolyl, phenoxazinyl, phenanthridinyl, phenanthrooxazolyl, benzodioxolyl, etc. More specifically, the heteroaryl may be 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acrydinyl, 2-acrydinyl, 3-acrydinyl, 4-acrydinyl, 9-acrydinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. Herein, the term “fused ring of an (C3-C30) aliphatic ring and an (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of an (C3-C30) aliphatic ring and an (C6-C30) aromatic ring may be replaced with at least one hetero atoms selected from B, N, O, S, Si and P, preferably at least one hetero atoms selected from N, O and S. Herein, the term “halogen” includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (5- to 25-membered) mono- or polycyclic, alicyclic, aromatic ring, or a combination thereof. Further, the formed ring may be included at least one heteroatom selected from the group consisting of B, N, O, S, Si and P, preferably at least one heteroatom selected from the group consisting of N, O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” described in of the present disclosure means that a hydrogen atom in a certain functional group is replaced with another atom or functional group, i.e., a substituent. The substituents of the substituted (C1-C30)alkyl(ene), the substituted (C2-C30)alkenyl, the substituted (C3-C30)cycloalkyl(ene), the fused ring of the substituted (C3-C30) aliphatic ring and the (C6-C30) aromatic ring, the substituted (C6-C30)aryl(ene), the substituted nitrogen-containing (3- to 10-membered)heteroaryl, and the substituted (3- to 30-membered)hetroaryl(ene) in formulas of the present disclosure, each independently represent at least one selected from the group consisting of deuterium, halogen, cyano, carboxyl, nitro, hydroxy, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, a (5- to 30-membered)heteroaryl unsubstituted or substituted with (C6-C30)aryl, a (C6-C30)aryl unsubstituted or substituted with (5- to 30-membered)heteroaryl, tri(C1-C30)alkylsilyl, tri(C6-C30)arylsilyl, di(C1-C30)alkyl(C6-C30)arylsilyl, (C1-C30)alkyldi(C6-C30)arylsilyl, a fused ring of an (C3-C30) aliphatic ring and an (C6-C30) aromatic ring, amino, a mono- or di-(C1-C30)alkylamino, a mono- or di-(C2-C30)alkenylamino, (C1-C30)alkyl(C2-C30)alkenylamino, a substituted or unsubstituted mono- or di-(C6-C30)arylamino, (C1-C30)alkyl(C6-C30)arylamino, a mono- or di-(3- to 30-membered)heteroarylamino, (C1-C30)alkyl(3- to 30-membered)heteroarylamino, (C2-C30)alkeyl(C6-C30)arylamino, (C2-C30)alkenyl(3- to 30-membered)heteroarylamino, (C6-C30)aryl(3- to 30-membered)heteroarylamino, (C1-C30)alkylcarbonyl, (C1-C30)alkoxycarbonyl, (C6-C30)arylcarbonyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl (C6-C30)aryl. For example, the substituents may be deuterium; methyl; phenyl unsubstituted or substituted with deuterium or naphthyl; biphenyl; naphthyl; dimethylfluorenyl; dimethylbenzofluorenyl; pyridyl unsubstituted or substituted with phenyl; dibenzofuranyl, dibenzothiophenyl; or a substituted or unsubstituted carbazolyl, etc.

In the formulas of the present disclosure, heteroaryl(ene) each independently may contain at least one heteroatom selected from the group consisting of B, N, O, S, Si, and P, preferably at least one heteroatom selected from the group consisting of N, O and S. Further, the above heteroatom may be linked with at least one substituent selected from the group consisting of hydrogen, deuterium, halogen, cyano, a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the host materials according to one embodiment will be described.

The host materials according to one embodiment comprise at least one first host compound represented by the above formula 1 and at least one second host compound represented by the above formula 2; and the host materials may be contained in the light-emitting layer of an organic electroluminescent device according to one embodiment.

The first host compound as the host material according to one embodiment may be represented by the following formula 1.

HAr-(L₁-Ar₁)ₐ (1)

in formula 1,

HAr represents a substituted or unsubstituted nitrogen-containing (3- to 10-membered)heteroaryl;

L₁ represents a single bond or a substituted or unsubstituted (C6-C30)arylene;

Ar represents a substituted or unsubstituted (C6-C30)aryl; and

a represents an integer of 1 to 3, when a is an integer of 2 or more, each of (L₁-Ar₁) may be the same or different.

In one embodiment, HAr may be a substituted or unsubstituted nitrogen-containing (5- to 10-membered)heteroaryl, preferably an unsubstituted nitrogen-containing (6- to 10-membered)heteroaryl. Specifically. HAr may be a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthiridinyl, a substituted or unsubstituted triazanaphthyl, or a substituted or unsubstituted benzothienopyrimidinyl. For example, HAr may be triazinyl, pyrimidinyl, quinolinyl, quinoxalinyl, or quinazolinyl.

In one embodiment, L₁ may be a single bond, a substituted or unsubstituted (C6-C25)arylene, or a substituted or unsubstituted (5- to 25-membered)heteroarylene, preferably a single bond, unsubstituted (C6-C20)arylene, or a substituted or unsubstituted (5- to 18-membered)heteroarylene. For example, L₁, may be a single bond, phenylene unsubstituted or substituted with naphthyl, a substituted or unsubstituted m-biphenylene, a substituted or unsubstituted p-biphenylene, or a substituted or unsubstituted naphthylene.

In one embodiment, Ar₁ may be a substituted or unsubstituted (C6-C25)aryl or a substituted or unsubstituted (5- to 25-membered)heteroaryl, preferably (C6-C18)aryl or a substituted or unsubstituted (5- to 18-membered)heteroaryl. For example, An may be phenyl unsubstituted or substituted with phenyl, naphthyl, or fluorenyl, a substituted or unsubstituted m-biphenyl, a substituted or unsubstituted p-biphenyl, naphthyl unsubstituted or substituted with phenyl, a substituted or unsubstituted m-terphenyl, a substituted or unsubstituted p-terphenyl, a substituted or unsubstituted triphenylenyl, a substituted or unsubstituted phenanthrenyl, fluorenyl substituted with at least one phenyl or methyl, or benzofluorenyl unsubstituted or substituted with at least one methyl or phenyl.

In one embodiment, a may be an integer of 2 or 3, in which each of (L₁-Ar₁) may be the same or different.

According to one embodiment, the host material represented by the above formula 1 may be represented by the following formula 1-1 or 1-2.

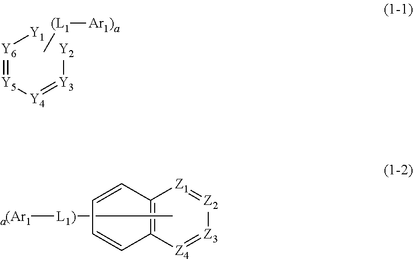

in formulas 1-1 and 1-2,

Y₁ to Y₆ and Z₁ to Z₄ each independently represent CRₐ or N, provided that at least one of Y₁ to Y₆ represents N, and at least one of Z₁ to Z₄ represents N;

Rₐ each independently represent hydrogen, a substituted or unsubstituted (C1-C30)alkyl, a substituted or unsubstituted (C2-C30)alkenyl, or a substituted or unsubstituted (C8-C30)aryl; or may be linked to the adjacent substituent to form a ring(s); and

L₁, Ar₁, and a are as defined in formula 1.

In one embodiment, at least one of Y₁ to Y₆ in formula 1-1 represents N, preferably at least two of Y₁ to Y₆ may be N, more preferably at least three of Y₁ to Y₆ may be N. For example, the compound represented by formula 1-1 may be pyrimidine or triazine in which (L₁-Ar₁)ₐ is substituted.

In one embodiment, at least one of Z₁ to Z₄ in formula 1-2 represents N, preferably at least two of Z₁ to Z₄ may be N. For example, the compound represented by formula 1-2 may be quinoline, quinoxaline, or quinazoline in which (L₁-Ar₁)ₐ is substituted.

In one embodiment, all of Rₐ may be hydrogen.

According to one embodiment, the first host compound represented by the above formula 1 may be more specifically illustrated by the following compounds, but is not limited thereto.

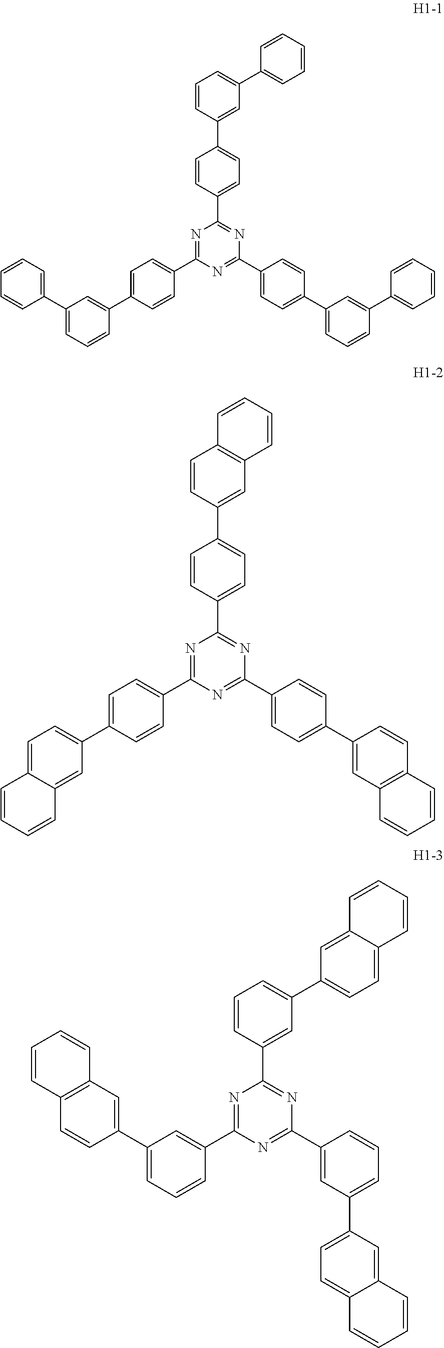 [SMILES: c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2nc(-c3cccc(-c4ccc5ccccc5c4)c3)nc(-c3cccc(-c4ccc5ccccc5c4)c3)n2)c1]

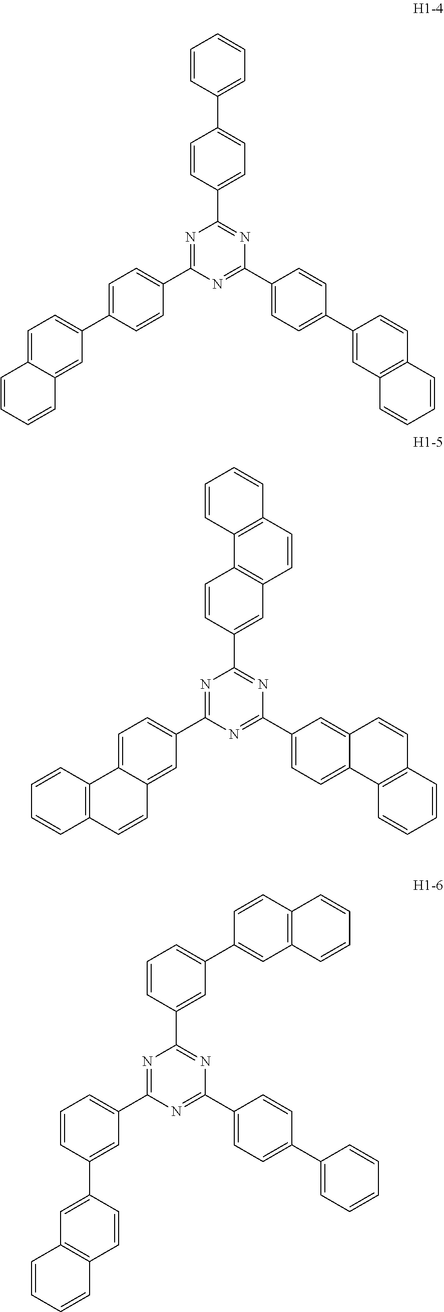 [SMILES: c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

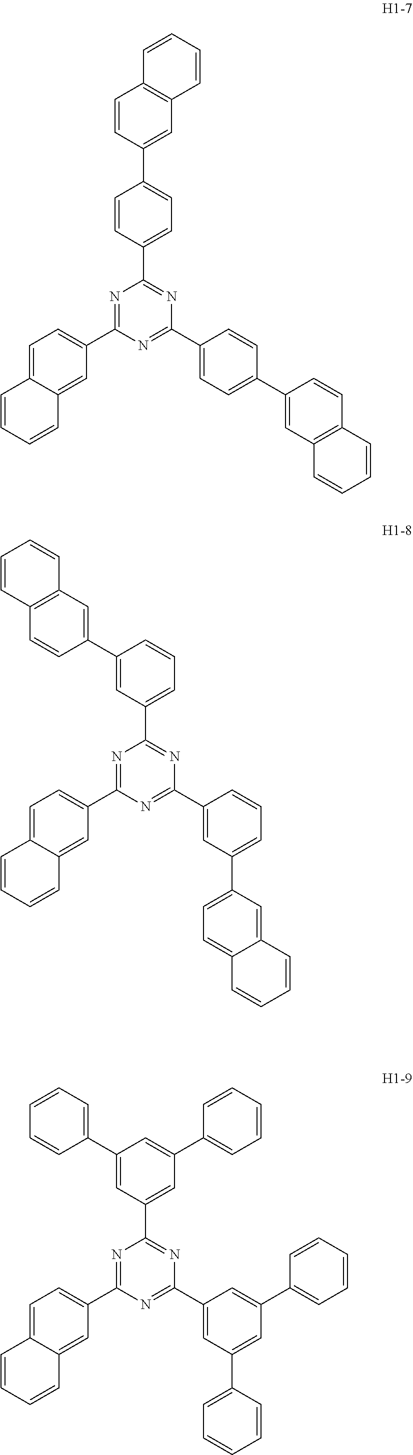 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1 ; c1ccc(-c2cc(-c3ccccc3)cc(-c3nc(-c4cc(-c5ccccc5)cc(-c5ccccc5)c4)nc(-c4ccc5ccccc5c4)n3)c2)cc1]

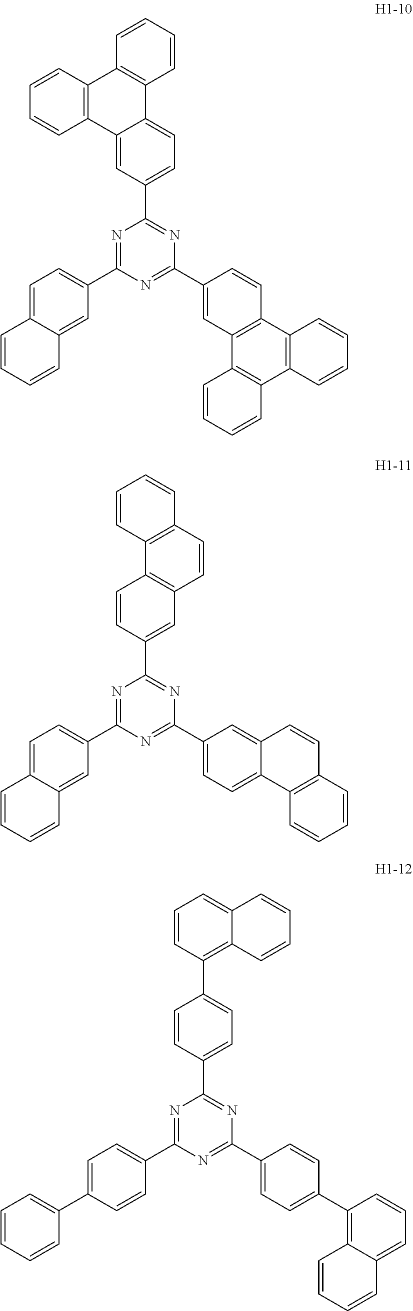 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1 ; c1ccc(-c2ccc(-c3nc(-c4ccc(-c5cccc6ccccc56)cc4)nc(-c4ccc(-c5cccc6ccccc56)cc4)n3)cc2)cc1]

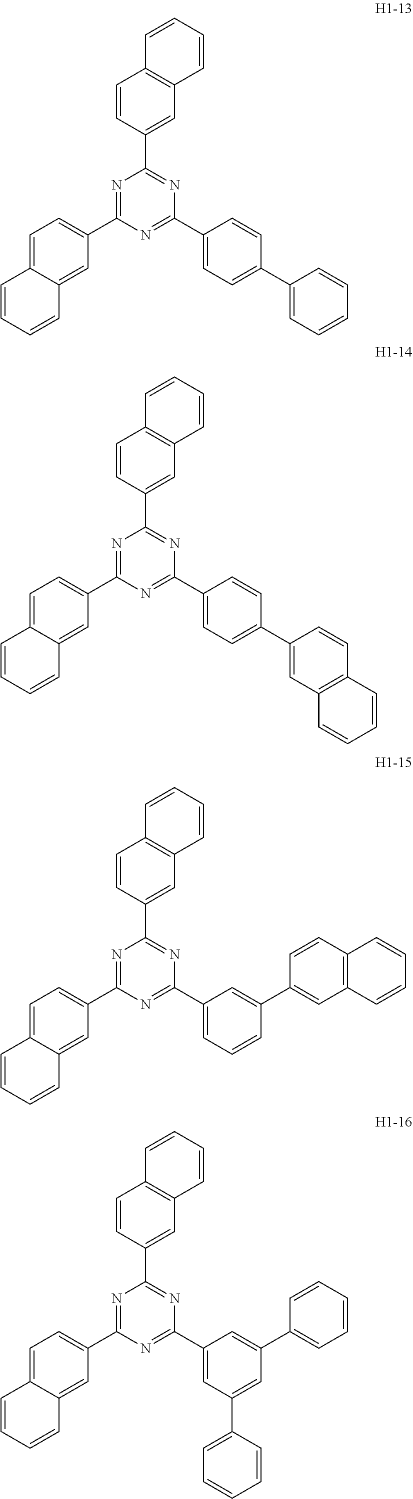 [SMILES: c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1 ; c1cc(-c2ccc3ccccc3c2)cc(-c2nc(-c3ccc4ccccc4c3)nc(-c3ccc4ccccc4c3)n2)c1 ; c1ccc(-c2cc(-c3ccccc3)cc(-c3nc(-c4ccc5ccccc5c4)nc(-c4ccc5ccccc5c4)n3)c2)cc1]

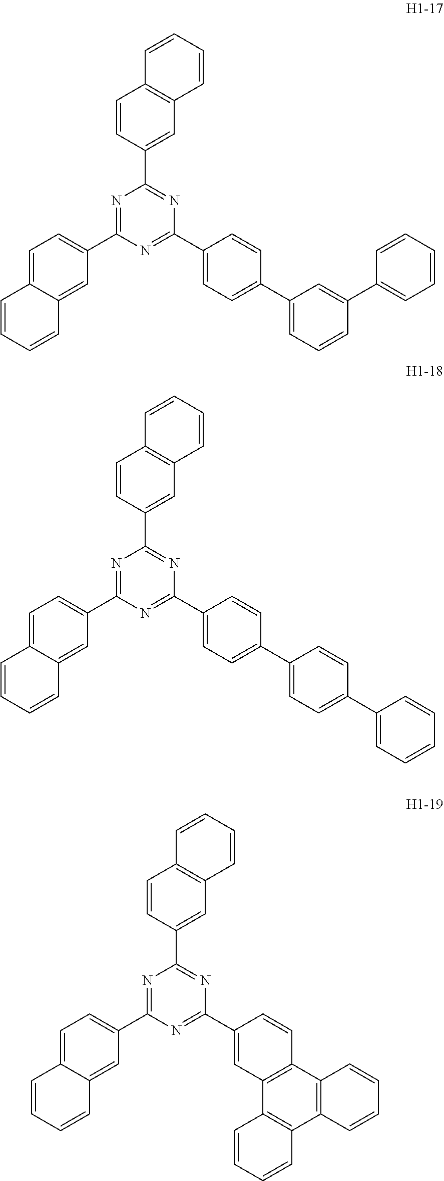 [SMILES: c1ccc(-c2cccc(-c3ccc(-c4nc(-c5ccc6ccccc6c5)nc(-c5ccc6ccccc6c5)n4)cc3)c2)cc1 ; c1ccc(-c2ccc(-c3ccc(-c4nc(-c5ccc6ccccc6c5)nc(-c5ccc6ccccc6c5)n4)cc3)cc2)cc1 ; c1ccc2cc(-c3nc(-c4ccc5ccccc5c4)nc(-c4ccc5c6ccccc6c6ccccc6c5c4)n3)ccc2c1]

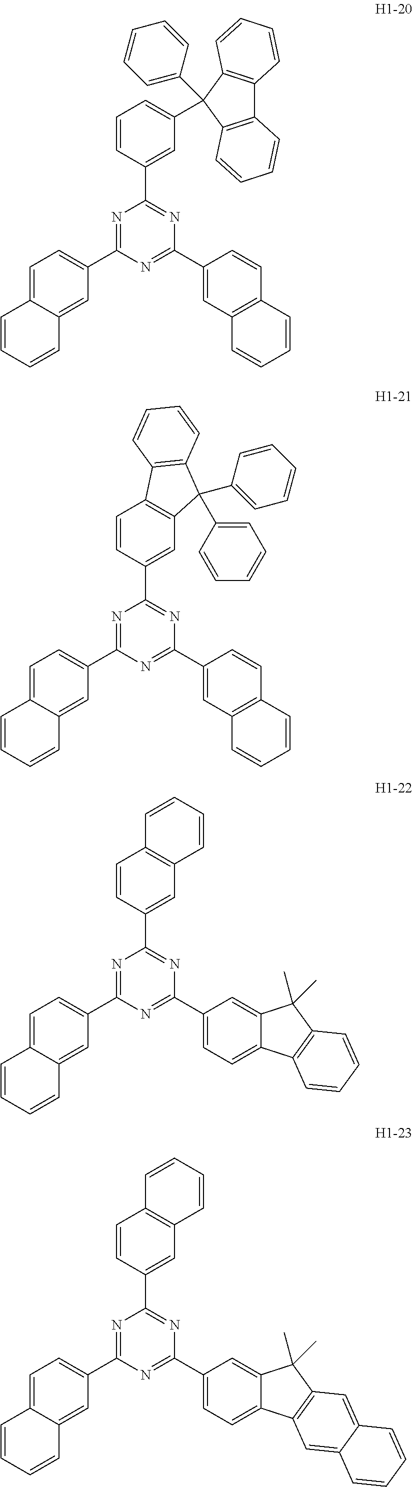 [SMILES: c1ccc(C2(c3cccc(-c4nc(-c5ccc6ccccc6c5)nc(-c5ccc6ccccc6c5)n4)c3)c3ccccc3-c3ccccc32)cc1 ; c1ccc(C2(c3ccccc3)c3ccccc3-c3ccc(-c4nc(-c5ccc6ccccc6c5)nc(-c5ccc6ccccc6c5)n4)cc32)cc1 ; CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21]

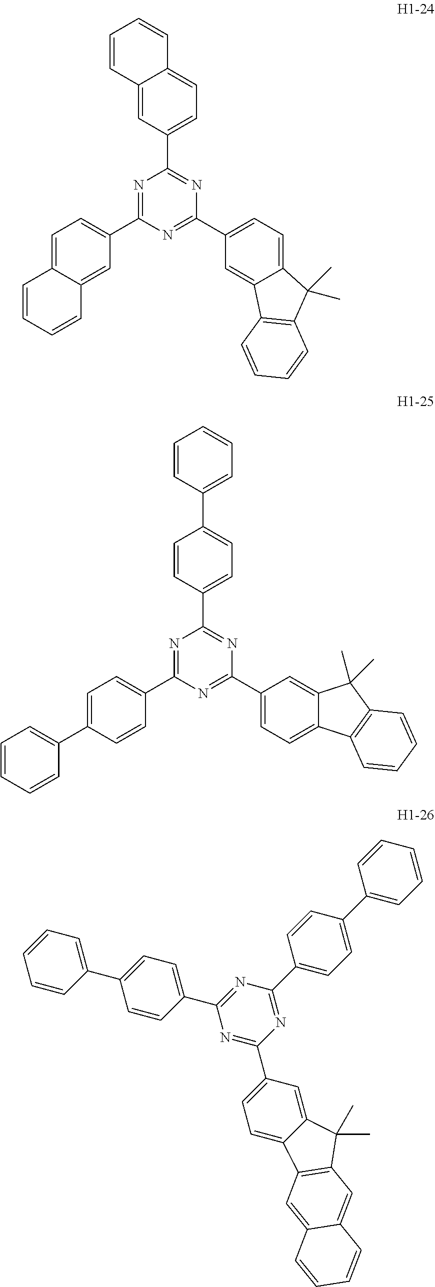 [SMILES: CC1(C)c2ccccc2-c2cc(-c3nc(-c4ccc5ccccc5c4)nc(-c4ccc5ccccc5c4)n3)ccc21 ; CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21]

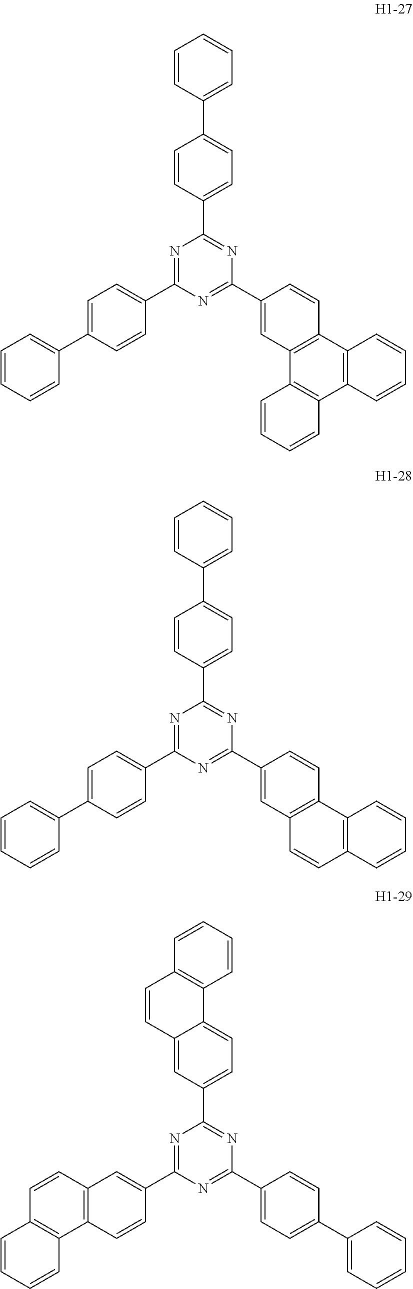 [SMILES: c1ccc(-c2ccc(-c3nc(-c4ccc(-c5ccccc5)cc4)nc(-c4ccc5c6ccccc6c6ccccc6c5c4)n3)cc2)cc1 ; c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1]

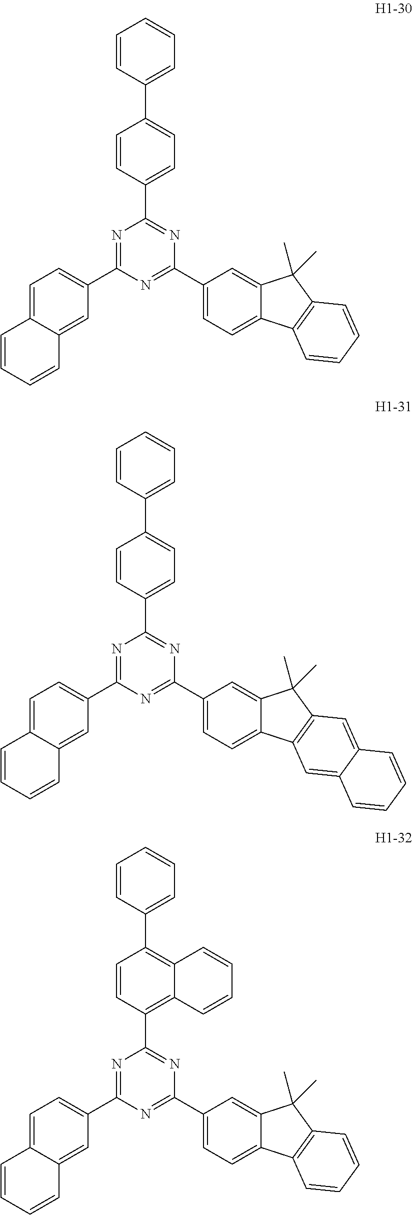 [SMILES: CC1(C)c2ccccc2-c2ccc(-c3nc(-c4ccc(-c5ccccc5)cc4)nc(-c4ccc5ccccc5c4)n3)cc21 ; CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21]

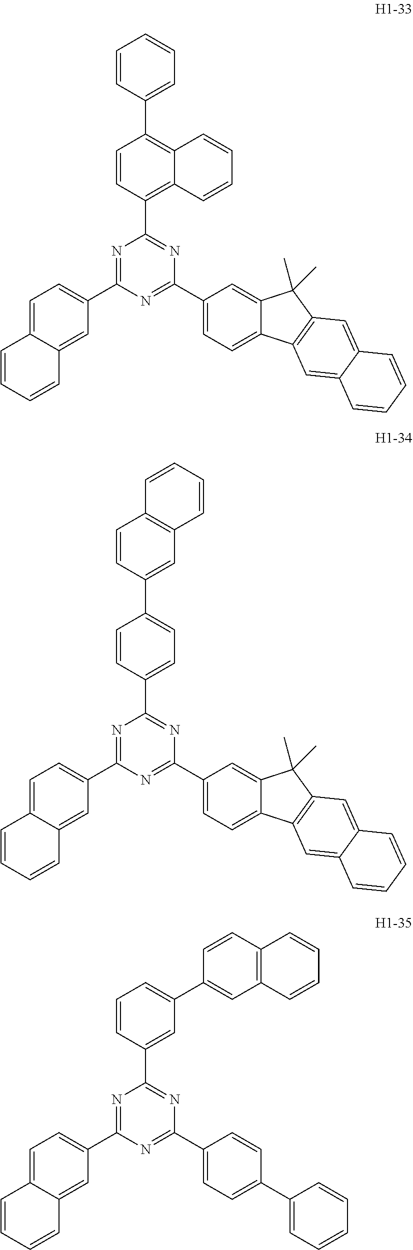 [SMILES: CC1(C)c2cc(-c3nc(-c4ccc5ccccc5c4)nc(-c4ccc(-c5ccccc5)c5ccccc45)n3)ccc2-c2cc3ccccc3cc21 ; CC1(C)c2cc(-c3nc(-c4ccc(-c5ccc6ccccc6c5)cc4)nc(-c4ccc5ccccc5c4)n3)ccc2-c2cc3ccccc3cc21 ; c1ccc(-c2ccc(-c3nc(-c4cccc(-c5ccc6ccccc6c5)c4)nc(-c4ccc5ccccc5c4)n3)cc2)cc1]

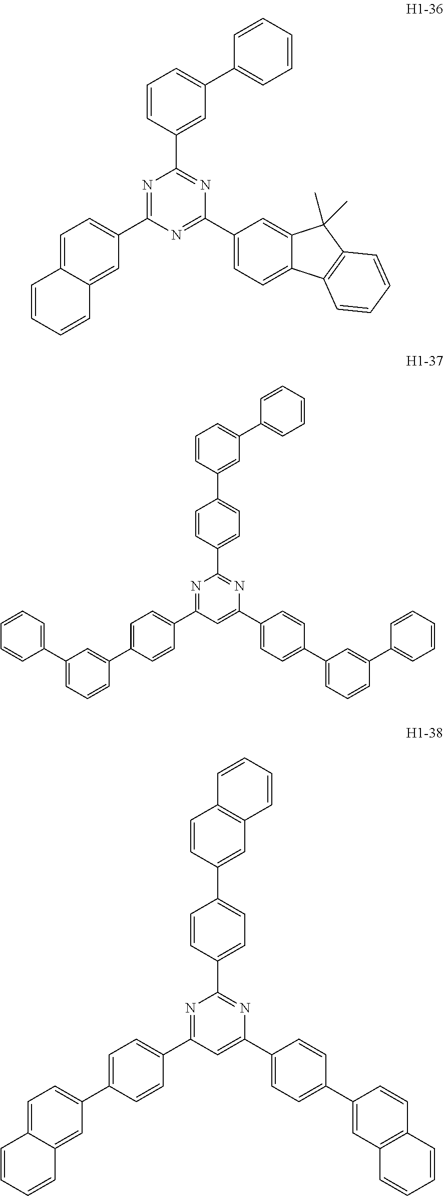 [SMILES: CC1(C)c2ccccc2-c2ccc(-c3nc(-c4cccc(-c5ccccc5)c4)nc(-c4ccc5ccccc5c4)n3)cc21 ; c1ccc(-c2cccc(-c3ccc(-c4cc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4cc(-c5ccc(-c6ccc7ccccc7c6)cc5)nc(-c5ccc(-c6ccc7ccccc7c6)cc5)n4)cc3)ccc2c1]

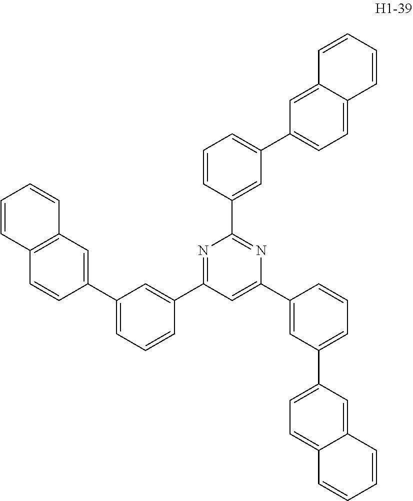 [SMILES: c1cc(-c2ccc3ccccc3c2)cc(-c2cc(-c3cccc(-c4ccc5ccccc5c4)c3)nc(-c3cccc(-c4ccc5ccccc5c4)c3)n2)c1]

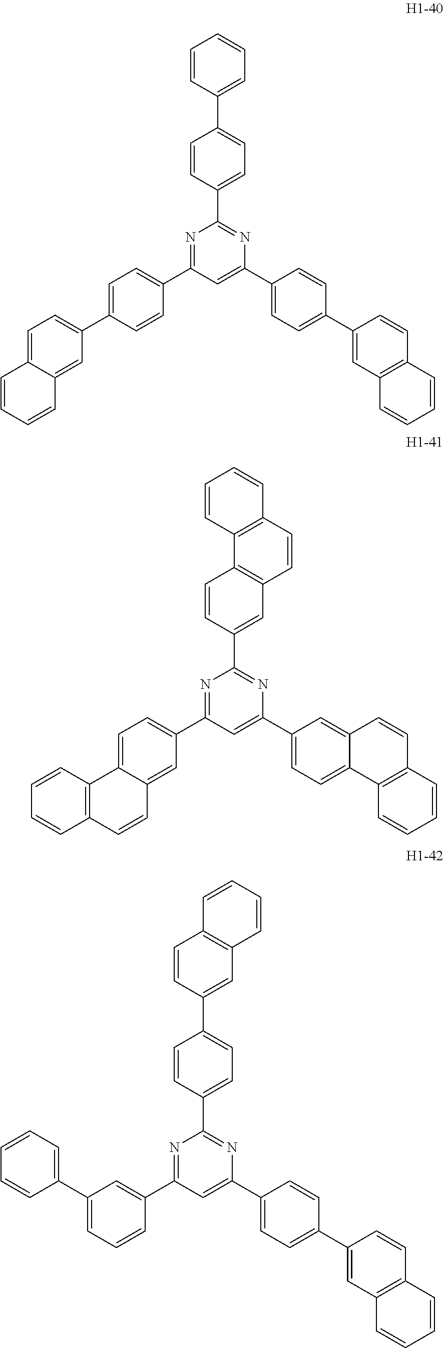 [SMILES: c1ccc(-c2ccc(-c3nc(-c4ccc(-c5ccc6ccccc6c5)cc4)cc(-c4ccc(-c5ccc6ccccc6c5)cc4)n3)cc2)cc1 ; c1ccc2c(c1)ccc1cc(-c3cc(-c4ccc5c(ccc6ccccc65)c4)nc(-c4ccc5c(ccc6ccccc65)c4)n3)ccc12 ; c1ccc(-c2cccc(-c3cc(-c4ccc(-c5ccc6ccccc6c5)cc4)nc(-c4ccc(-c5ccc6ccccc6c5)cc4)n3)c2)cc1]

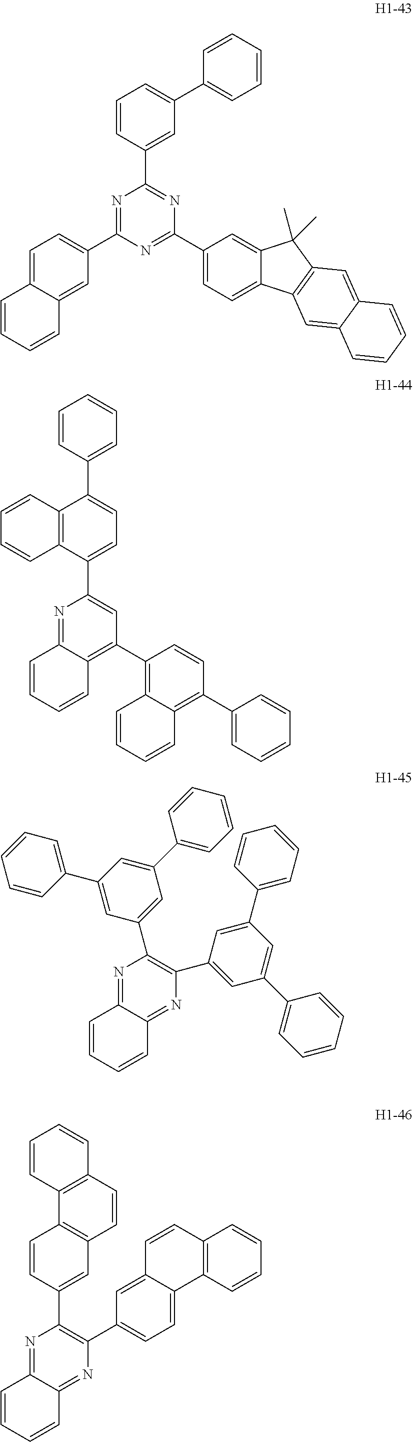

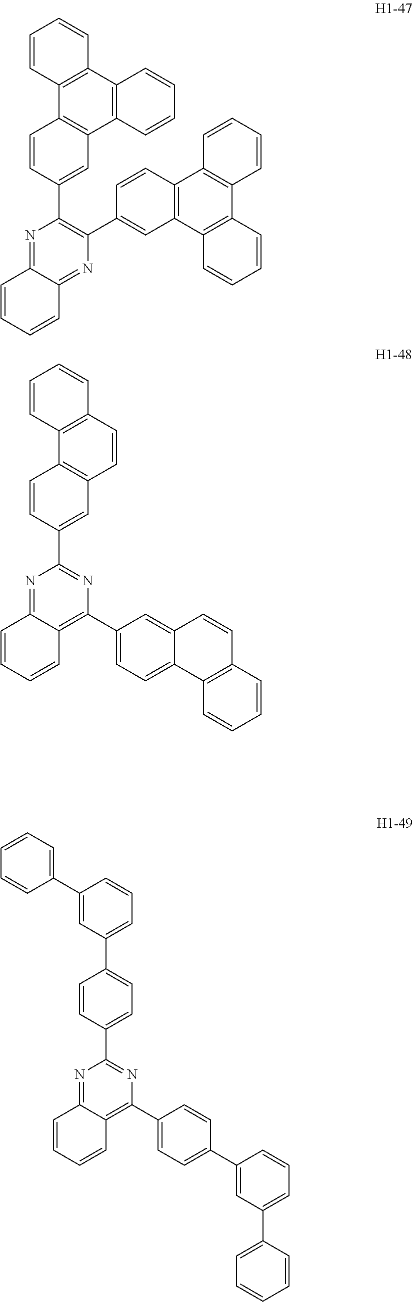

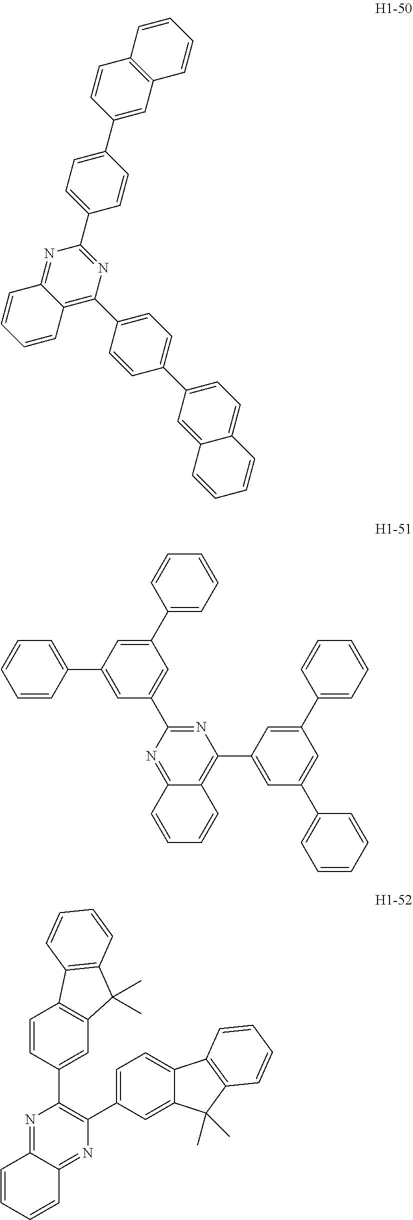 [SMILES: c1ccc2cc(-c3ccc(-c4nc(-c5ccc(-c6ccc7ccccc7c6)cc5)c5ccccc5n4)cc3)ccc2c1 ; c1ccc(-c2cc(-c3ccccc3)cc(-c3nc(-c4cc(-c5ccccc5)cc(-c5ccccc5)c4)c4ccccc4n3)c2)cc1 ; CC1(C)c2ccccc2-c2ccc(-c3nc4ccccc4nc3-c3ccc4c(c3)C(C)(C)c3ccccc3-4)cc21]

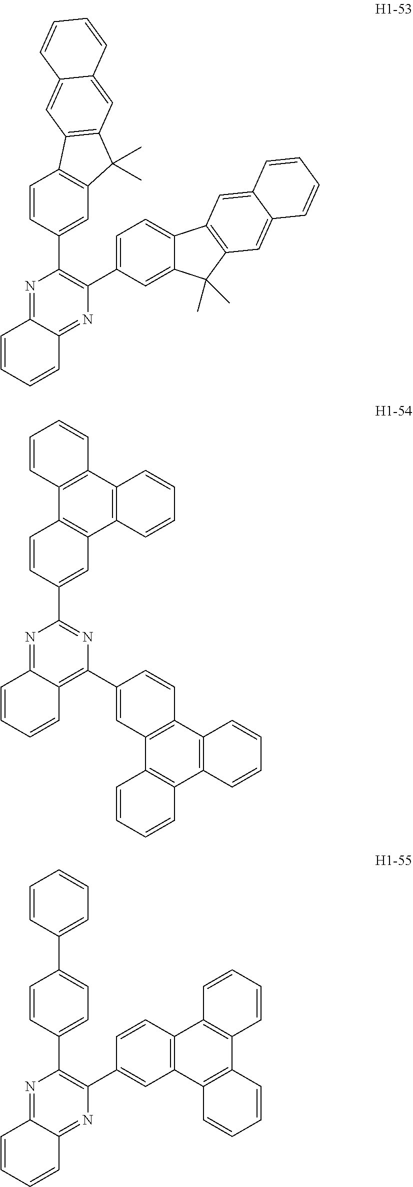

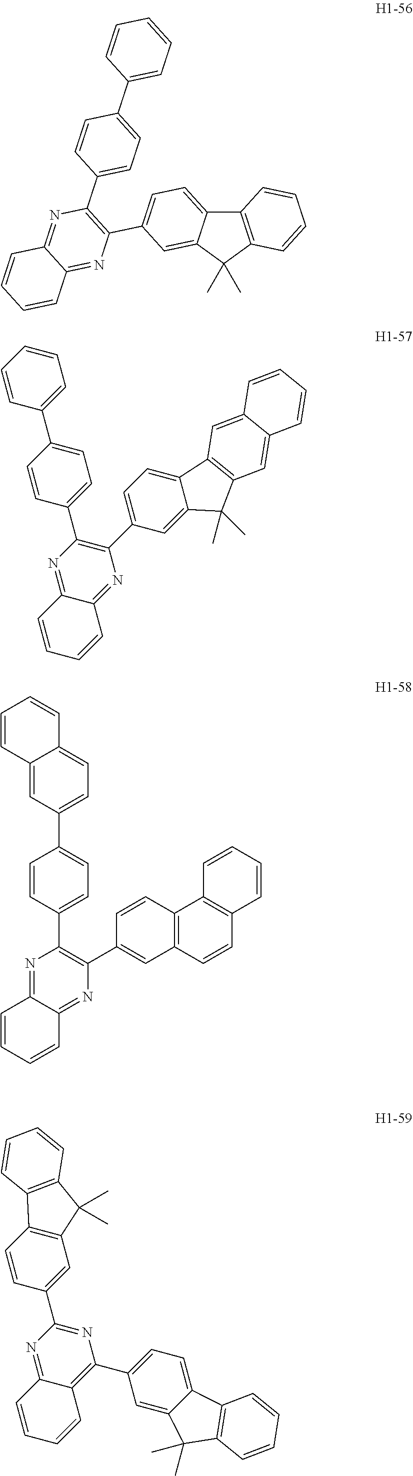

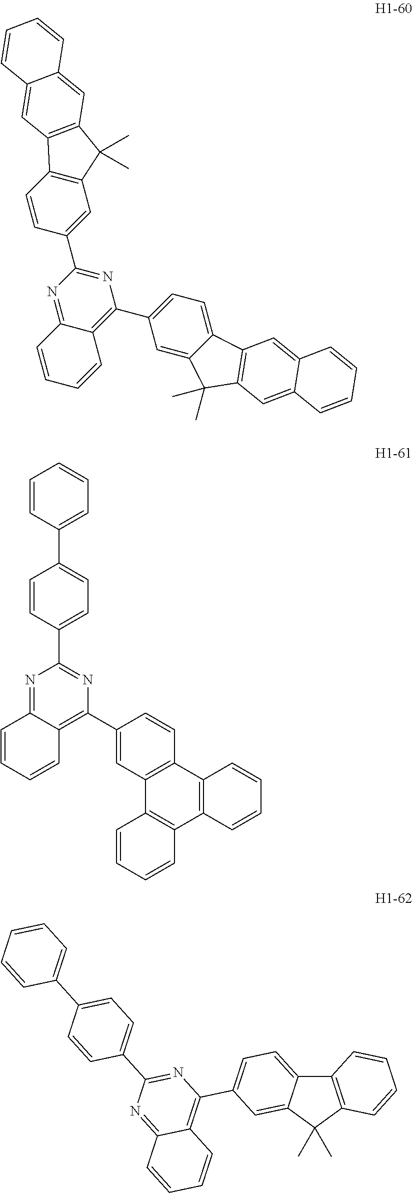 [SMILES: CC1(C)c2cc(-c3nc(-c4ccc5c(c4)C(C)(C)c4cc6ccccc6cc4-5)c4ccccc4n3)ccc2-c2cc3ccccc3cc21 ; c1ccc(-c2ccc(-c3nc(-c4ccc5c6ccccc6c6ccccc6c5c4)c4ccccc4n3)cc2)cc1 ; CC1(C)c2ccccc2-c2ccc(-c3nc(-c4ccc(-c5ccccc5)cc4)nc4ccccc34)cc21]

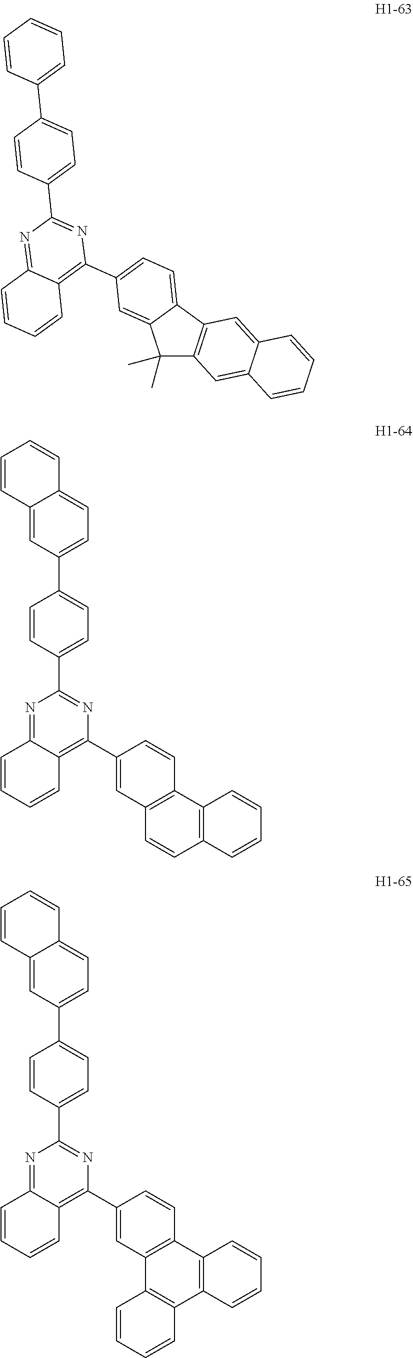 [SMILES: CC1(C)c2cc(-c3nc(-c4ccc(-c5ccccc5)cc4)nc4ccccc34)ccc2-c2cc3ccccc3cc21 ; c1ccc2cc(-c3ccc(-c4nc(-c5ccc6c(ccc7ccccc76)c5)c5ccccc5n4)cc3)ccc2c1 ; c1ccc2cc(-c3ccc(-c4nc(-c5ccc6c7ccccc7c7ccccc7c6c5)c5ccccc5n4)cc3)ccc2c1]

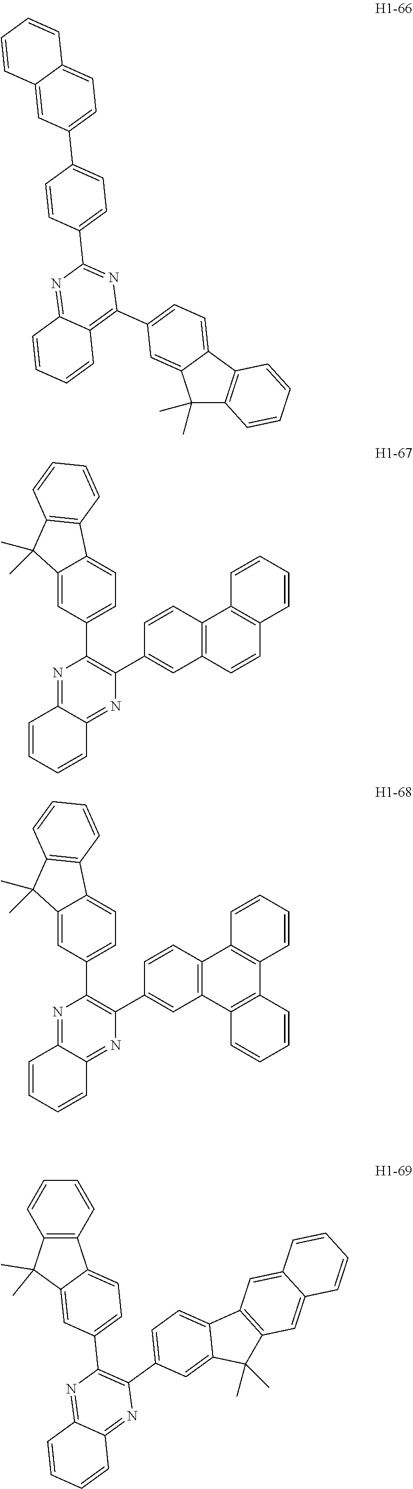

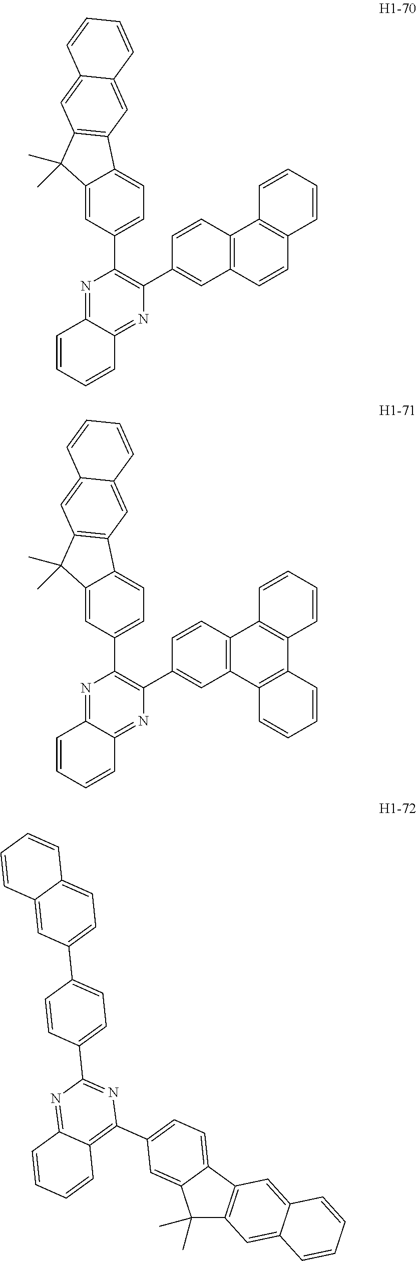

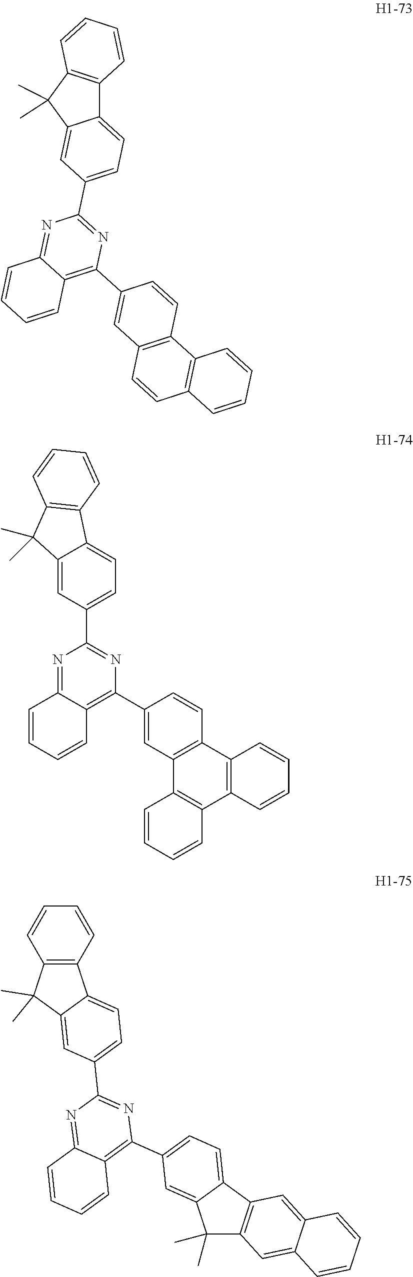 [SMILES: CC1(C)c2ccccc2-c2ccc(-c3nc(-c4ccc5c(ccc6ccccc65)c4)c4ccccc4n3)cc21 ; CC1(C)c2ccccc2-c2ccc(-c3nc(-c4ccc5c6ccccc6c6ccccc6c5c4)c4ccccc4n3)cc21 ; CC1(C)c2ccccc2-c2ccc(-c3nc(-c4ccc5c(c4)C(C)(C)c4cc6ccccc6cc4-5)c4ccccc4n3)cc21]

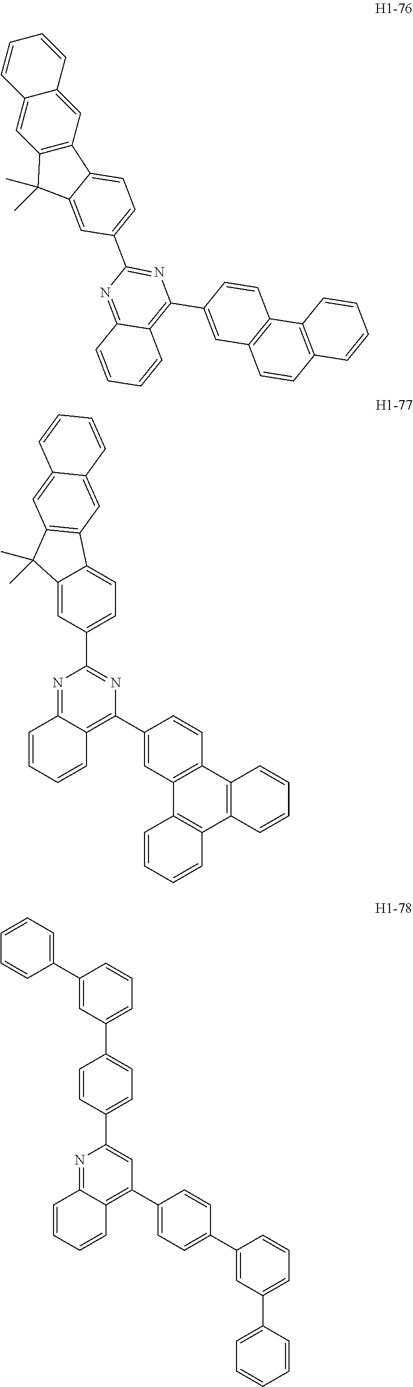

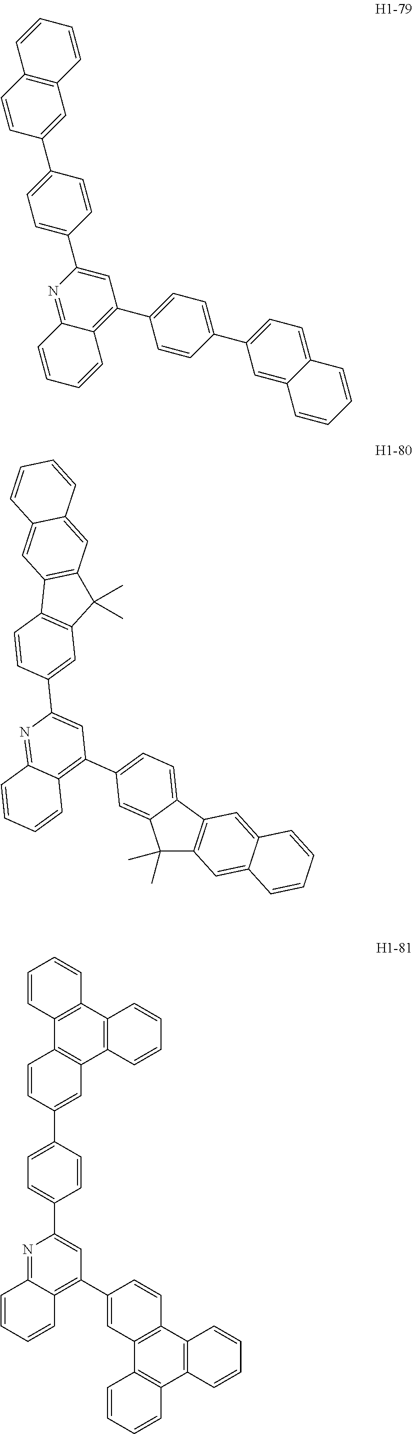 [SMILES: c1ccc2cc(-c3ccc(-c4cc(-c5ccc(-c6ccc7ccccc7c6)cc5)c5ccccc5n4)cc3)ccc2c1 ; CC1(C)c2cc(-c3cc(-c4ccc5c(c4)C(C)(C)c4cc6ccccc6cc4-5)c4ccccc4n3)ccc2-c2cc3ccccc3cc21 ; c1ccc2c(-c3ccc4c5ccccc5c5ccccc5c4c3)cc(-c3ccc(-c4ccc5c6ccccc6c6ccccc6c5c4)cc3)nc2c1]

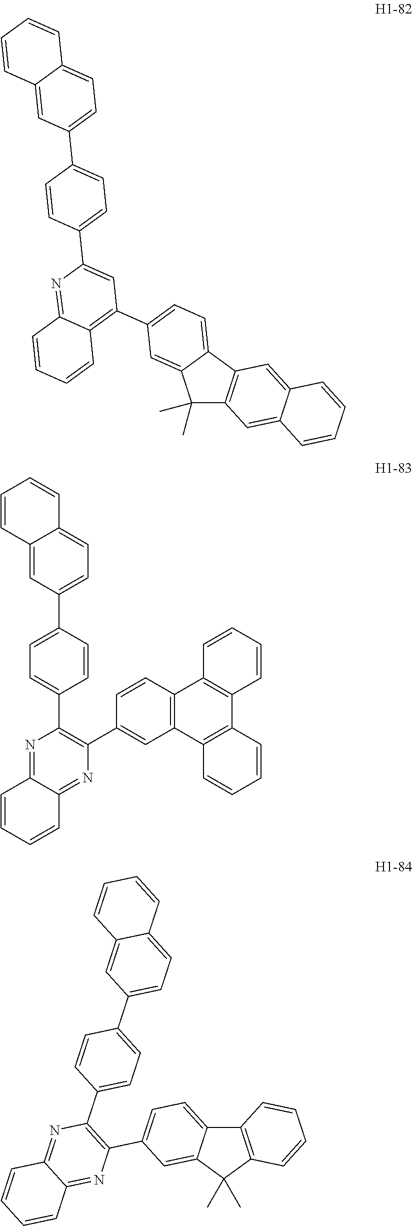 [SMILES: CC1(C)c2cc(-c3cc(-c4ccc(-c5ccc6ccccc6c5)cc4)nc4ccccc34)ccc2-c2cc3ccccc3cc21 ; c1ccc2cc(-c3ccc(-c4nc5ccccc5nc4-c4ccc5c6ccccc6c6ccccc6c5c4)cc3)ccc2c1 ; CC1(C)c2ccccc2-c2ccc(-c3nc4ccccc4nc3-c3ccc(-c4ccc5ccccc5c4)cc3)cc21]

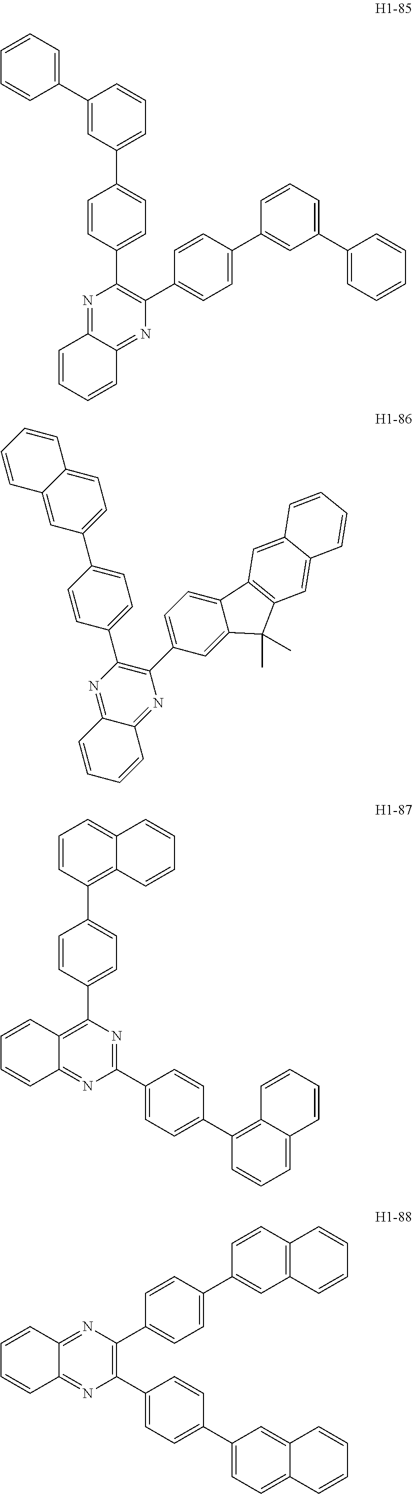

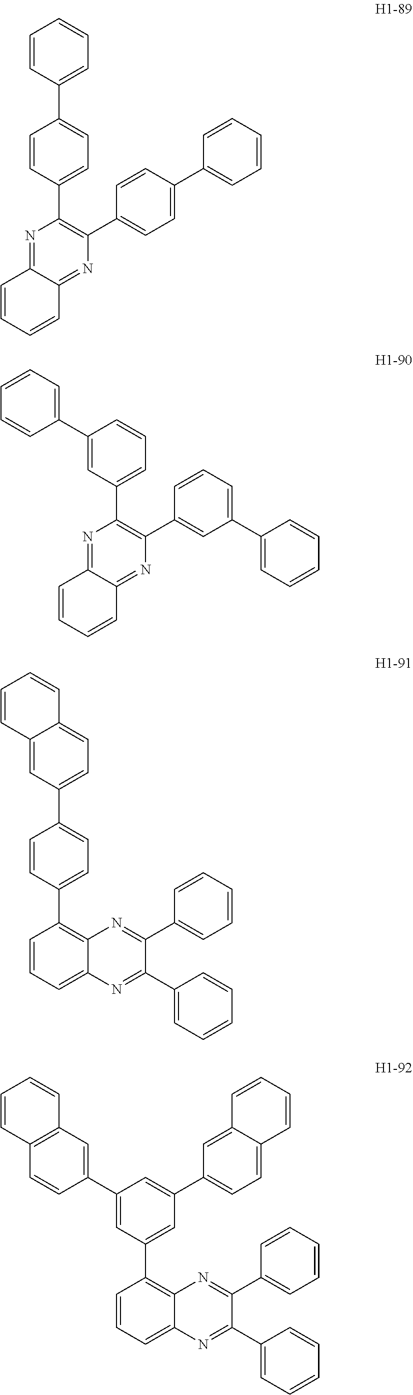

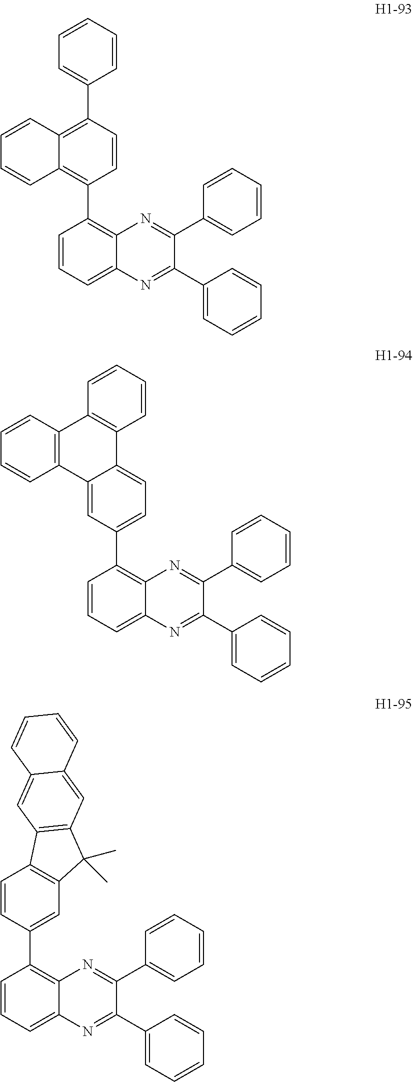 [SMILES: c1ccc(-c2nc3cccc(-c4ccc(-c5ccccc5)c5ccccc45)c3nc2-c2ccccc2)cc1 ; c1ccc(-c2nc3cccc(-c4ccc5c6ccccc6c6ccccc6c5c4)c3nc2-c2ccccc2)cc1 ; CC1(C)c2cc(-c3cccc4nc(-c5ccccc5)c(-c5ccccc5)nc34)ccc2-c2cc3ccccc3cc21]

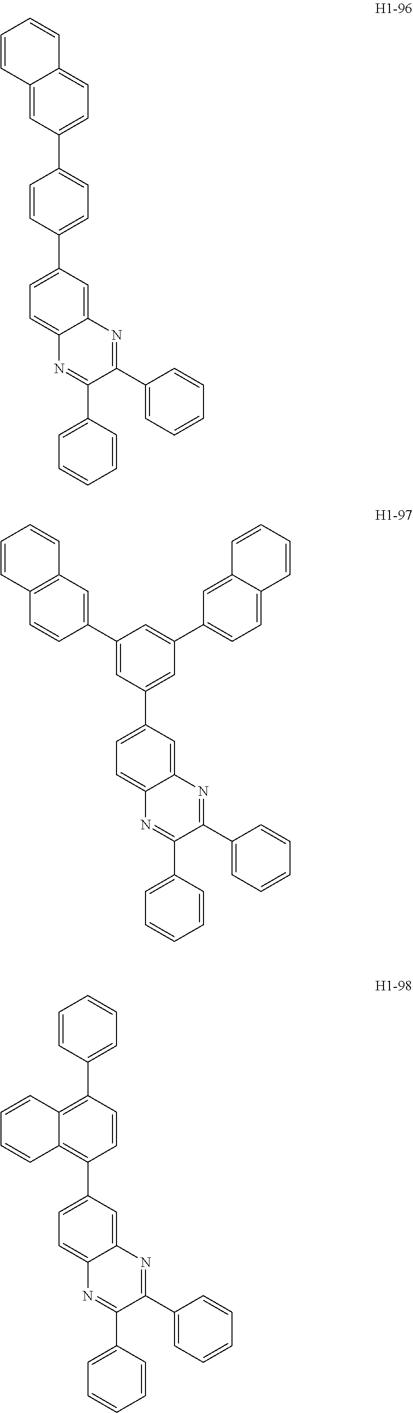 [SMILES: c1ccc(-c2nc3ccc(-c4ccc(-c5ccc6ccccc6c5)cc4)cc3nc2-c2ccccc2)cc1 ; c1ccc(-c2nc3ccc(-c4cc(-c5ccc6ccccc6c5)cc(-c5ccc6ccccc6c5)c4)cc3nc2-c2ccccc2)cc1 ; c1ccc(-c2nc3ccc(-c4ccc(-c5ccccc5)c5ccccc45)cc3nc2-c2ccccc2)cc1]

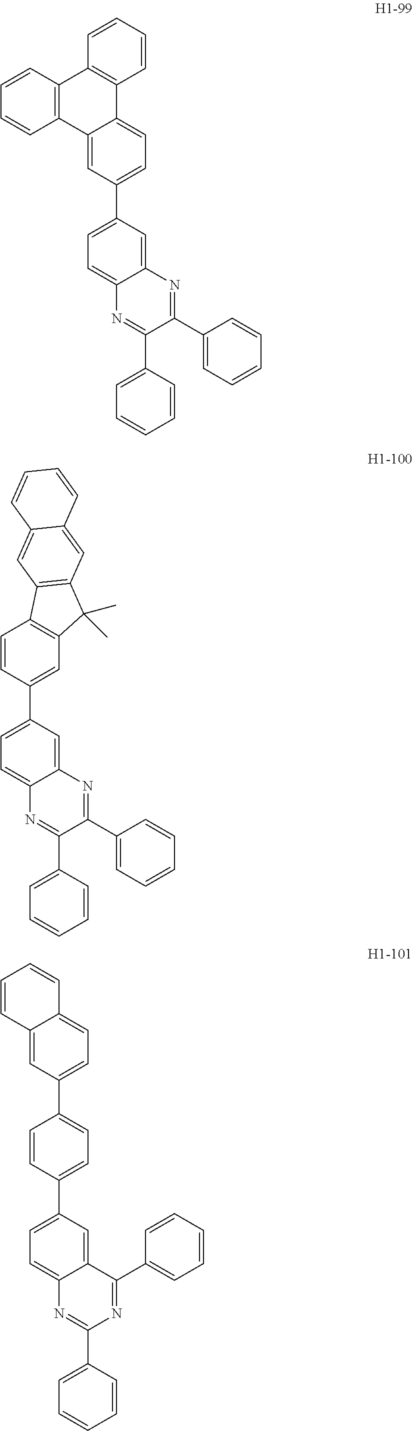

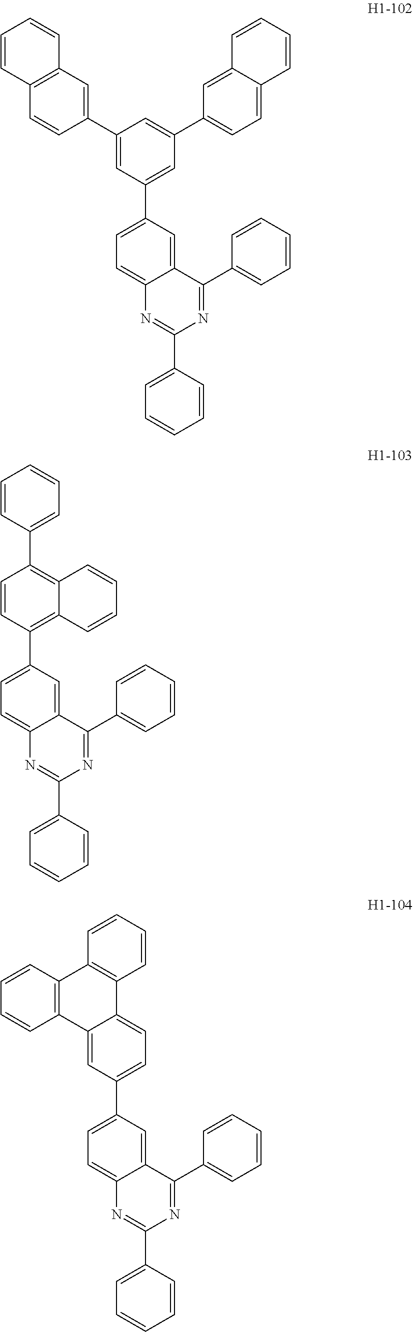 [SMILES: c1ccc(-c2nc(-c3ccccc3)c3cc(-c4cc(-c5ccc6ccccc6c5)cc(-c5ccc6ccccc6c5)c4)ccc3n2)cc1 ; c1ccc(-c2nc(-c3ccccc3)c3cc(-c4ccc(-c5ccccc5)c5ccccc45)ccc3n2)cc1 ; c1ccc(-c2nc(-c3ccccc3)c3cc(-c4ccc5c6ccccc6c6ccccc6c5c4)ccc3n2)cc1]

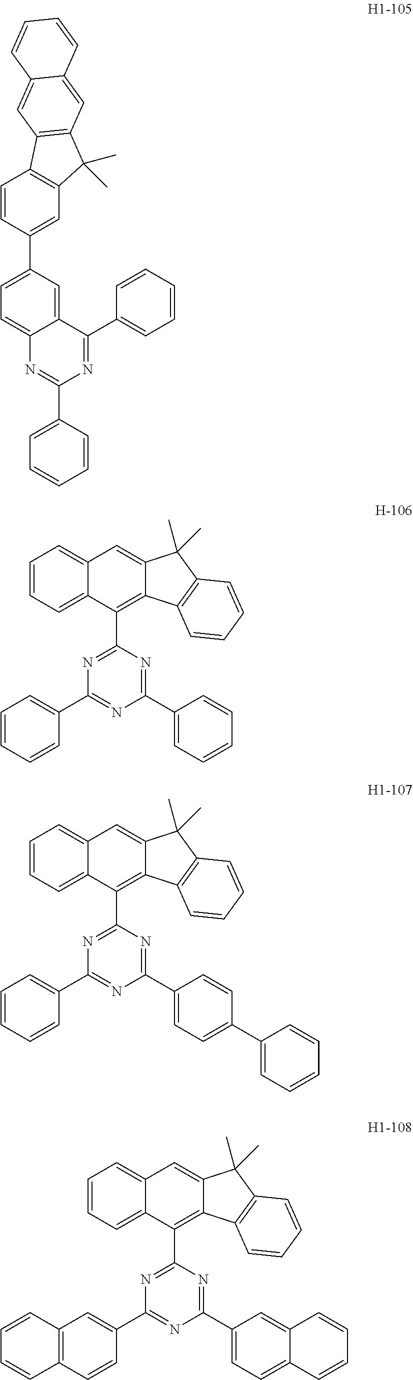

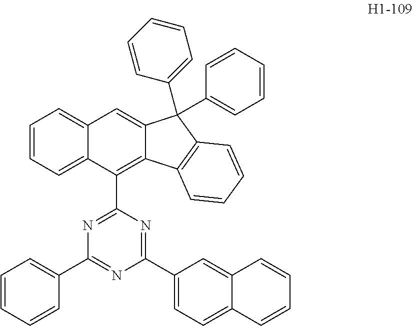 [SMILES: c1ccc(-c2nc(-c3ccc4ccccc4c3)nc(-c3c4c(cc5ccccc35)C(c3ccccc3)(c3ccccc3)c3ccccc3-4)n2)cc1]

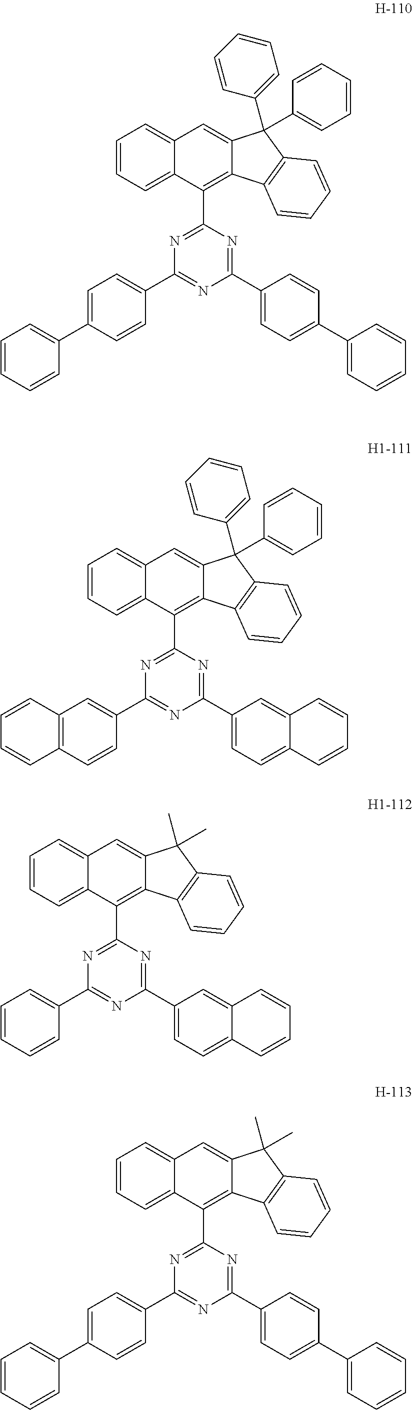 [SMILES: c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1 ; CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1]

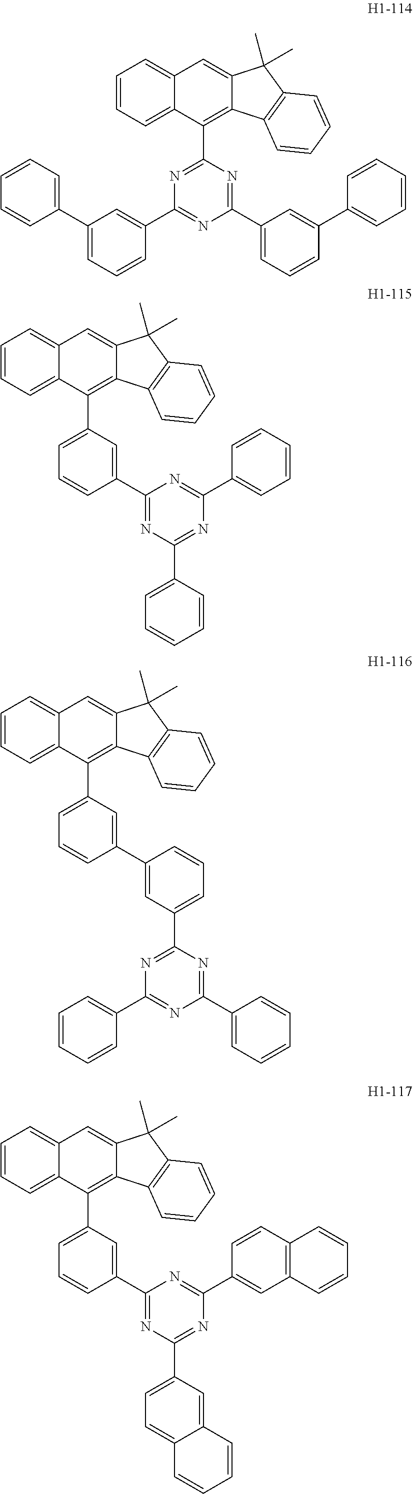

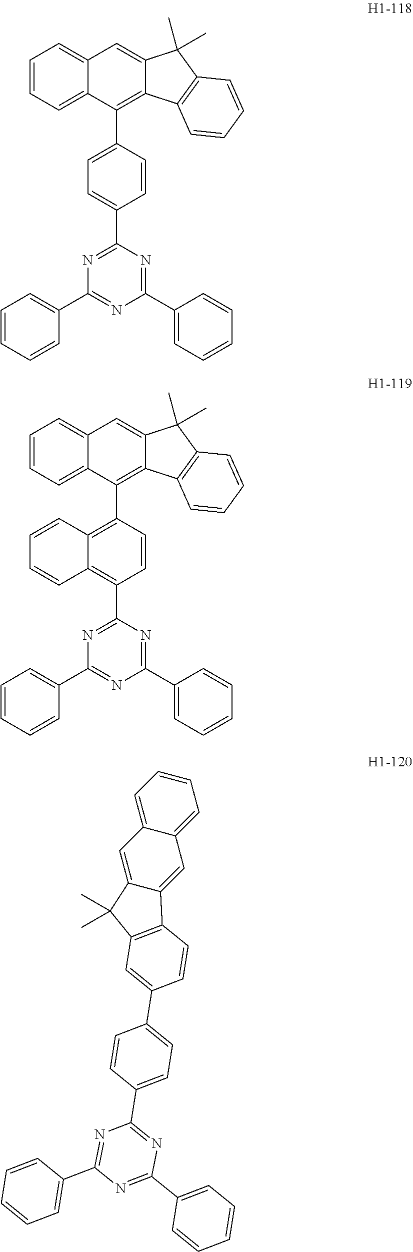

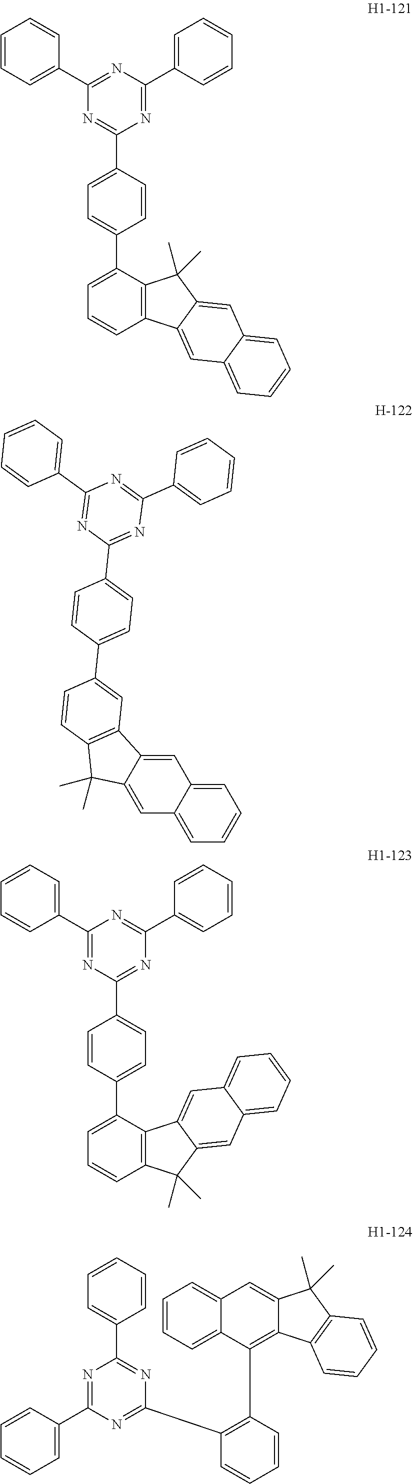

The compound represented by formula 1 of the present disclosure may be produced by a synthetic method known to a person skilled in the art, for example, the compound represented by formula 1-1 or 1-2 may be synthesized by referring to the following reaction scheme 1 or 2, but is not limited thereto:

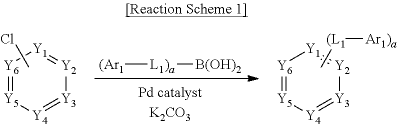

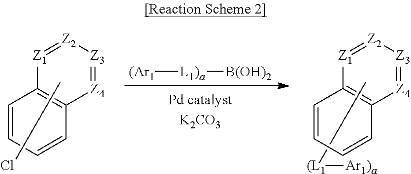

In the reaction schemes 1 and 2, the definition of the substituents is as defined in formulas 1-1 and 1-2.

As described above, exemplary synthesis examples of the compounds represented by formula 1-1 or 1-2 according to one embodiment are described, but they are based on Buchwald-Hartwig cross coupling reaction, N-arylation reaction, H-mont-mediated etherification reaction, Miyaura borylation reaction, Suzuki cross-coupling reaction, Intramolecular acid-induced cyclization reaction, Pd(II)-catalyzed oxidative cyclization reaction, Grignard reaction, Heck reaction, Cyclic Dehydration reaction, SN₁ substitution reaction, SN₂ substitution reaction, and Phosphine-mediated reductive cyclization reaction, etc. It will be understood by one skilled in the art that the above reaction proceeds even if other substituents defined in formula 1-1 or 1-2 other than the substituents described in the specific synthesis examples are bonded.

The second host compound as another host material according to one embodiment may be represented by the following formula 2.

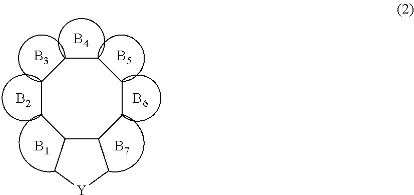

in formula 2,

B₁ to B₇ each independently are absent, or each independently represent a substituted or unsubstituted (C5-C20) ring, in which the carbon atom of the ring may be replaced with one or more heteroatom(s) selected from nitrogen, oxygen, and sulfur, with the proviso that at least five of B₁ to B₇ are present, and the immediately adjacent rings of B₁ to B₇ may be fused with each other;

Y represents —N-L₂-(Ar₂)ₙ, —O—, —S—, or —CR₁R₂;

L₂ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₂ represents a substituted or unsubstituted (C6-C30)aryl, a substituted or unsubstituted (3- to 30-membered)heteroaryl, or —NR₃R₄;

R₁ to R₄ each independently represent hydrogen, deuterium, halogen, cyano, a substituted or unsubstituted (C1-C30)alkyl, a substituted or unsubstituted (C2-C30)alkenyl, a substituted or unsubstituted fused ring of (C3-C30)aliphatic ring and (C6-C30)aromatic ring, a substituted or unsubstituted (C6-C30)aryl, a substituted or unsubstituted (3- to 30-membered)heteroaryl, or a substituted or unsubstituted (C3-C30)cycloalkyl; or may be linked to the adjacent substituent to form a ring(s); and

n represents an integer of 1 or 2, when n is 2, each of Ar₂ may be the same or different.

In one embodiment, B₁ to B₇ each independently are absent, or each independently represent a substituted or unsubstituted (C5-C20) ring, preferably a substituted or unsubstituted (C5-C13) ring, in which the carbon atom of the ring may be replaced with one or more heteroatom(s) selected from nitrogen, oxygen, and sulfur, with the proviso that at least five of B₁ to B₇ are present, and the immediately adjacent rings of B₁ to B₇ may be fused with each other. Herein, “fusing the immediately adjacent rings of the B₁ to B₇ with each other” means that B₁ ring and B₂ ring, B₂ ring and B₃ ring, B₃ ring and B₄ ring, B₄ ring and B₅ ring, B₅ ring and B₆ ring, or B₆ ring and B₇ ring are fused with each other.

According to one embodiment of the present disclosure, when any one of B₁ to B₇ represents a (C6-C20) ring, the immediately adjacent rings may be absent, or a C5 ring, in which the carbon atom of the ring may be replaced with one or more heteroatom(s) selected from nitrogen, oxygen, and sulfur.

According to another embodiment of the present disclosure, B₁ to B₇ each independently are absent, or each independently may be a substituted or unsubstituted benzene ring, a substituted or unsubstituted naphthalene ring, a substituted or unsubstituted pyrrole ring, a substituted or unsubstituted furan ring, a substituted or unsubstituted thiophene ring, a substituted or unsubstituted cyclopentadiene ring, a substituted or unsubstituted fluorene ring, a substituted or unsubstituted pyridine ring, or a substituted or unsubstituted dibenzofuran ring. For example, B₁ to B₇ each independently are absent, or each independently may be benzene ring unsubstituted or substituted with phenyl, naphthyl and/or diphenyltriazinyl; naphthalene ring; cyclopentadiene ring unsubstituted or substituted with at least one methyl; fluorene ring substituted with at least one methyl; pyrrole ring substituted with unsubstituted phenyl, phenyl substituted with at least one deuterium, biphenyl, and/or pyridyl; furan ring; thiophene ring; pyridine ring; or dibenzofuran ring unsubstituted or substituted with diphenyltriazinyl.

In one embodiment, Y may be —N-L₂-(Ar₂)ₙ or —O—, preferably, —N-L₂-(Ar₂)ₙ.

In one embodiment, Ar₂ may be a substituted or unsubstituted (C6-C30)aryl, a substituted or unsubstituted (5- to 30-membered)heteroaryl, or —NR₃R₄, preferably, a substituted or unsubstituted (C6-C25)aryl, a substituted or unsubstituted (5- to 25-membered)heteroaryl, or —NR₃R₄, more preferably, (C6-C25)aryl unsubstituted or substituted with at least one of deuterium, (C1-C6)alkyl and (3- to 30-membered)heteroaryl; (5- to 25-membered)heteroaryl unsubstituted or substituted with at least one of deuterium, (C6-C18)aryl, and (3- to 30-membered)heteroaryl; or —NR₃R₄. For example. Ar₂ may be phenyl unsubstituted or substituted with deuterium; a substituted or unsubstituted naphthyl; a substituted or unsubstituted m-biphenyl; a substituted or unsubstituted p-biphenyl; a substituted or unsubstituted o-terphenyl; a substituted or unsubstituted p-terphenyl; a substituted or unsubstituted m-terphenyl; a substituted or unsubstituted triphenylenyl; pyridyl unsubstituted or substituted with phenyl; pyrimidinyl unsubstituted or substituted with phenyl; a substituted or unsubstituted dibenzothiophenyl; a substituted or unsubstituted dibenzofuranyl; quioxaline unsubstituted or substituted with at least one of phenyl, m-biphenyl, p-biphenyl, dibenzofuranyl, and dibenzothiophenyl; benzoquioxaline unsubstituted or substituted with phenyl; quinazoline unsubstituted or substituted with at least one of phenyl, m-biphenyl, p-biphenyl, dibenzofuranyl, and dibenzothiophenyl; benzofuropyrimidinyl unsubstituted or substituted with phenyl; benzothienopyrimidinyl unsubstituted or substituted with phenyl; triazinyl unsubstituted or substituted with at least one of phenyl unsubstituted or substituted with deuterium or 26-membered heteroaryl, naphthyl, pyridyl unsubstituted or substituted with phenyl, m-biphenyl, p-biphenyl, m-terphenyl, fluorenyl unsubstituted or substituted with methyl, dibenzofuranyl, and dibenzothiophenyl; or —NR₃R₄.

In one embodiment, L₂ may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene. For example, L₂ may be a single bond, phenylene, m-biphenylene, naphthylene, pyridylene, triazinylene, dibenzofuranylene, quinoxalinylene, benzoquinoxalinylene, quinazolinylene, benzofuropyrimidinylene, or benzothienopyrimidinylene.

According to one embodiment, the second host material represented by formula 2 above may be represented by any one of the following formulas 2-1 to 2-5.

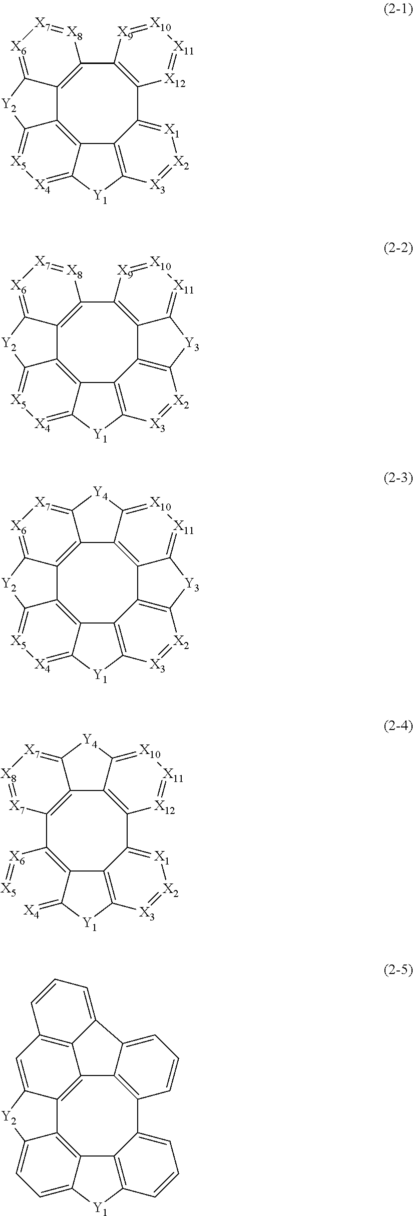

in formulas 2-1 to 2-5,

Y₁, Y₂, Y₃, and Y₄ each independently are as defined as Y in formula 2, and where if a plurality of Ar₂ is present, each of Ar₂ may be the same or different;

X₁ to X₁₂ each independently represent —N═ or —C(R_{b})═;

R_{b} represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or the adjacent R_{b}'s may be linked to each other to form a ring(s), and where if a plurality of R_{b} is present, each of R_{b} may be the same or different.

In one embodiment, R_{b} may be hydrogen, a substituted or unsubstituted (C6-C30)aryl, or a substituted or unsubstituted (5- to 30-membered)heteroaryl; or the adjacent R_{b}'s may be linked to each other to form a ring(s), preferably, hydrogen, a substituted or unsubstituted (C6-C25)aryl, or a substituted or unsubstituted (5- to 25-membered)heteroaryl; or the adjacent R_{b}'s may be linked to each other to form a substituted or unsubstituted (5- to 30-membered) monocylic or polycyclic alicyclic, aromatic ring, or a combination thereof, more preferably, hydrogen, a substituted or unsubstituted (C6-C18)aryl, or a substituted or unsubstituted (5- to 18-membered)heteroaryl; or the adjacent R_{b}'s may be linked to each other to form a substituted or unsubstituted (5- to 25-membered) monocylic or polycyclic aromatic ring. For example, R_{b} may be phenyl, naphthyl, or triazinyl substituted with phenyl; or the adjacent R_{b}'s may be linked to each other to form benzene ring, indene ring substituted with at least one methyl, or benzofuran ring unsubstituted or substituted with diphenyltriazinyl.

According to one embodiment, Ar₂ and R_{b} each independently may be selected from any one of the substituents listed in the following Group 1.

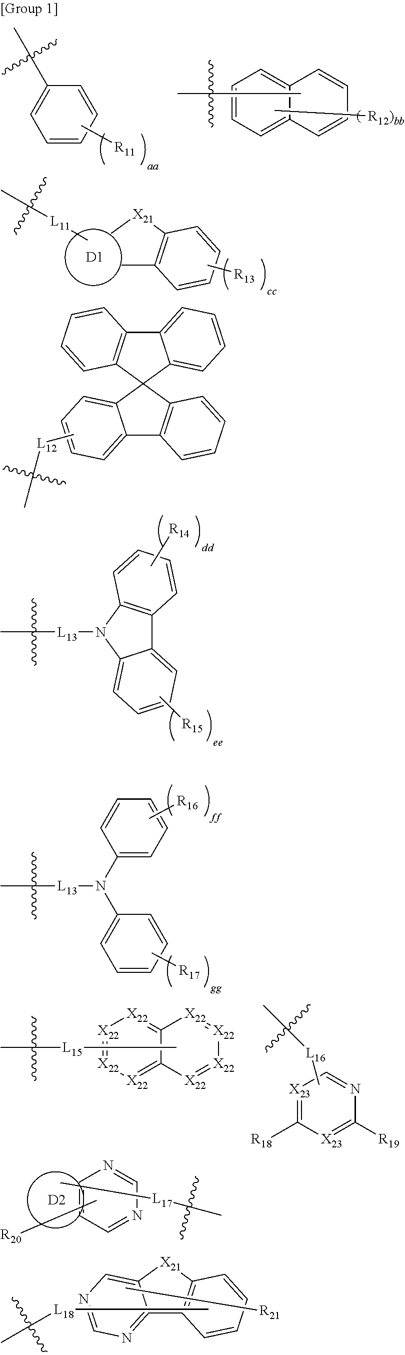

in the Group 1,

D1 and D2 each independently represent a benzene ring or a naphthalene ring;

X₂₁ represents O, S, NR₅, or CR₆R₇;

X₂₂ each independently represents CR₈ or N; provided that at least one of X₂₂ represent(s) N;

X₂₃ each independently represent CR₉ or N;

L₁₁ to L₁₈ each independently represent a single bond, a substituted or unsubstituted (C6-C30)arylene, or a substituted or unsubstituted (3- to 30-membered)heteroarylene;

R₁₁ to R₂₁ and R₅ to R₉ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to the adjacent substituent to form a ring(s); and

aa, ff, and gg each independently represent an integer of 1 to 5, bb represents an integer of 1 to 7, and cc, dd, and ee each independently represent an integer of 1 to 4.

In one embodiment, D1 and D2 may be a benzene ring; X₂₁ may be O, S, or CR₆R₇; L₁₁ to L₁₈ each independently may be a single bond; R₁₁ to R₂₁ and R₅ to R₉ each independently may be hydrogen, deuterium, a substituted or unsubstituted (C1-C20)alkyl, a substituted or unsubstituted (C6-C25)aryl, or a substituted or unsubstituted (5- to 25-membered)heteroaryl, or the adjacent substituents may be linked to each other to form a ring(s); aa, bb, ff, and gg each independently may be an integer of 1 to 5; and cc, dd and ee each independently may be an integer of 1 to 4. For example, R₁₁ may be hydrogen, deuterium, phenyl, biphenyl or 26-membered heteroaryl; R₁₂ may be hydrogen, or the adjacent R₁₂'s may be linked to each other to form a benzene ring; R₁₃, R₁₆ and R₁₇ may be hydrogen; R₁₈ and R₁₉ may be hydrogen or phenyl; R₂₁ may be phenyl; R₆ and R₇ may be methyl; R₈ may be hydrogen, phenyl, biphenyl, dibenzofuranyl, or dibenzothiophenyl, or the adjacent R₈'s may be linked to each other to form a benzene ring; R₉ may be hydrogen, unsubstituted phenyl, phenyl substituted with at least one deuterium, phenyl substituted with 26-membered heteroaryl, naphthyl, biphenyl, dimethylfluorenyl, terphenyl, pyridyl substituted with phenyl, dibenzofuranyl, or dibenzothiophenyl; as may be an integer of 1 or 5; bb may be an integer of 1 or 4; and cc may be 1.

According to another embodiment, Ar₂ and R_{b} each independently may be selected from any one of the substituents listed in the following Group 2.

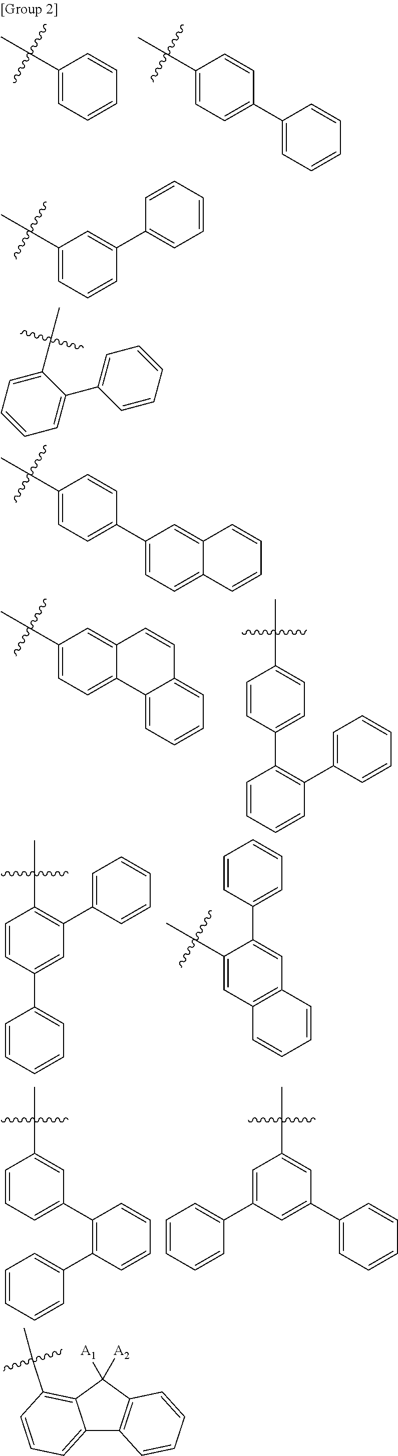

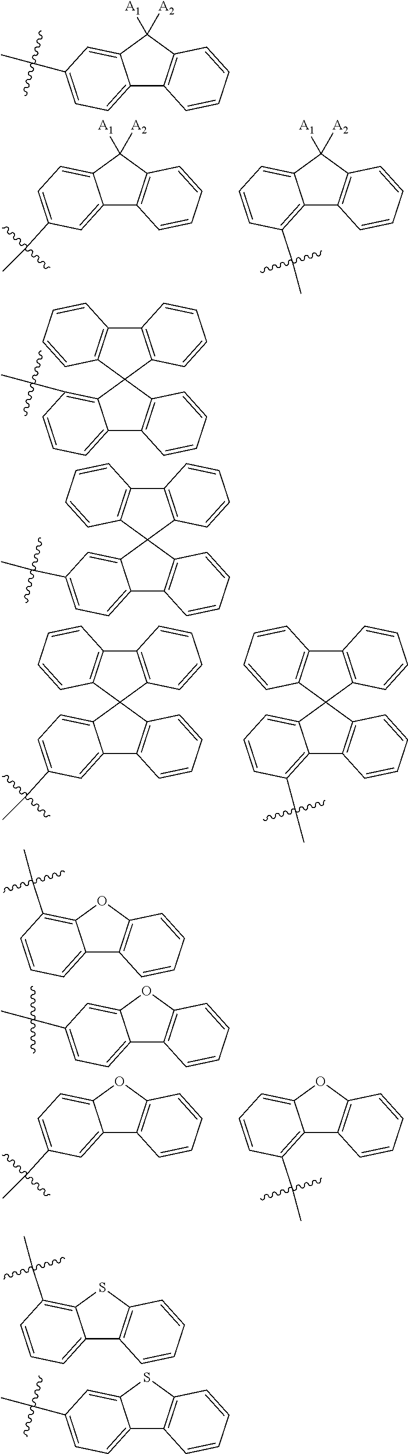

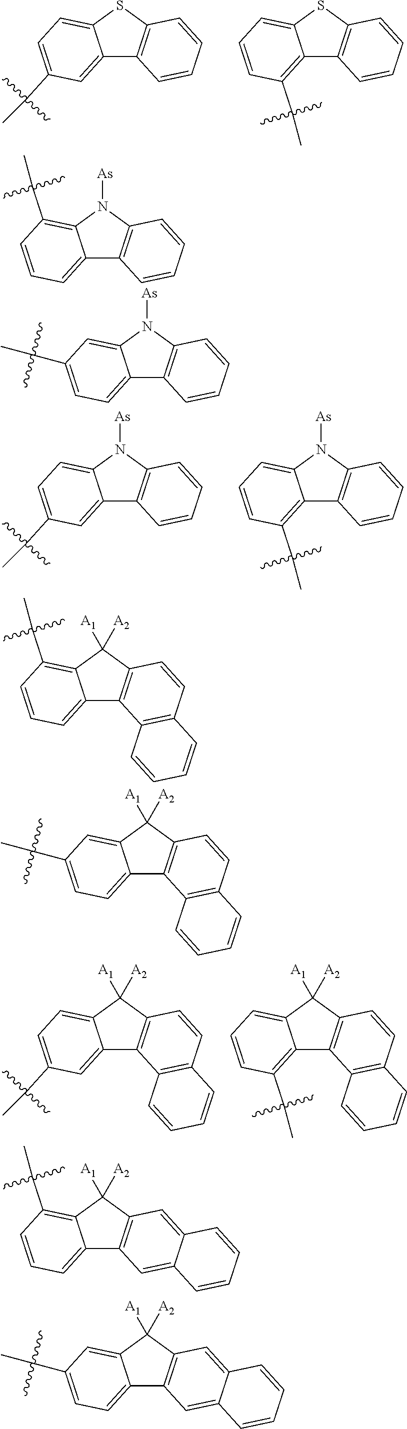

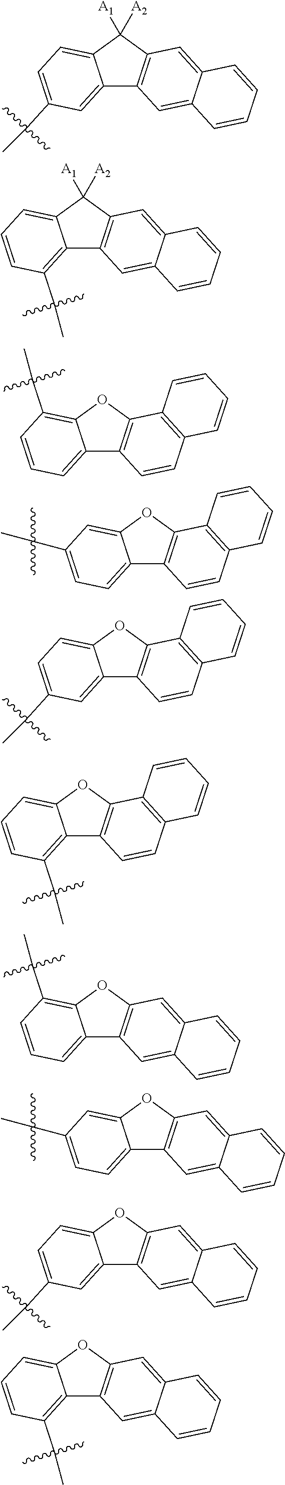

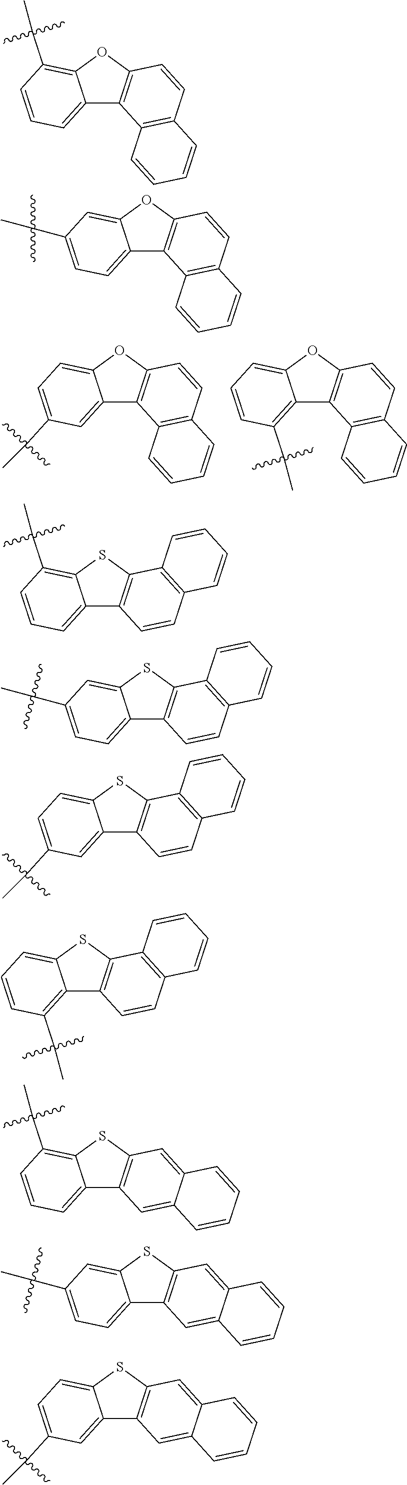

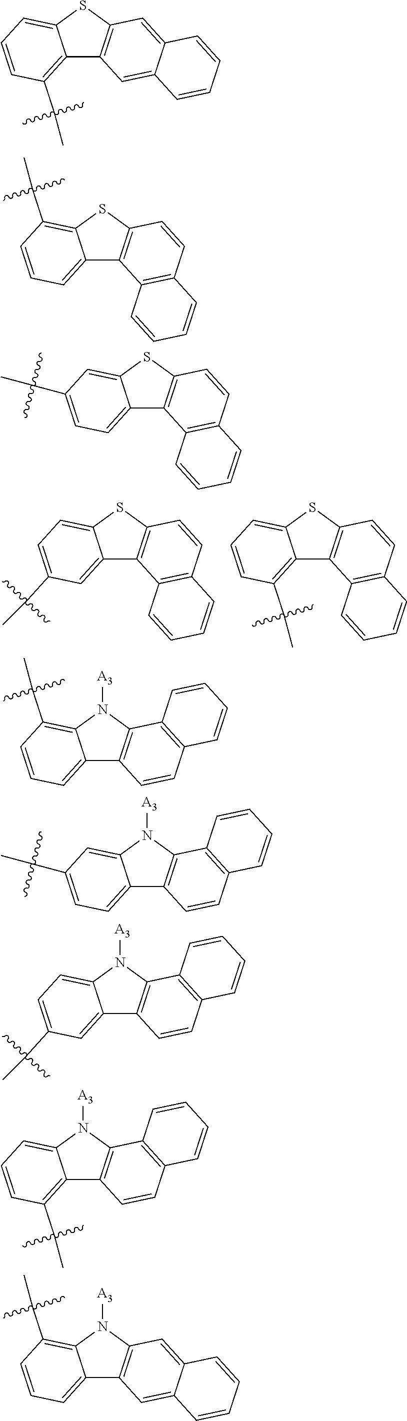

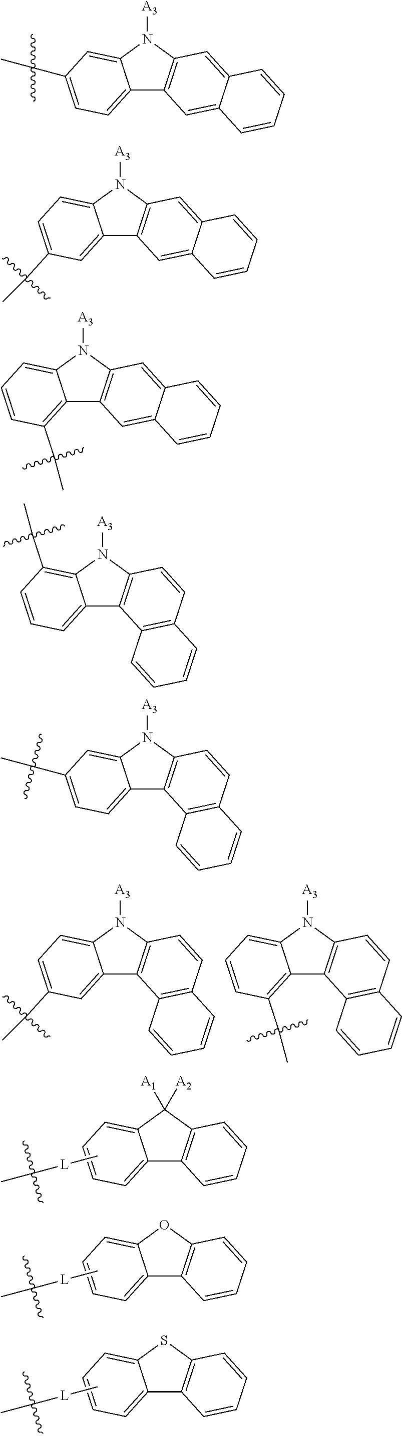

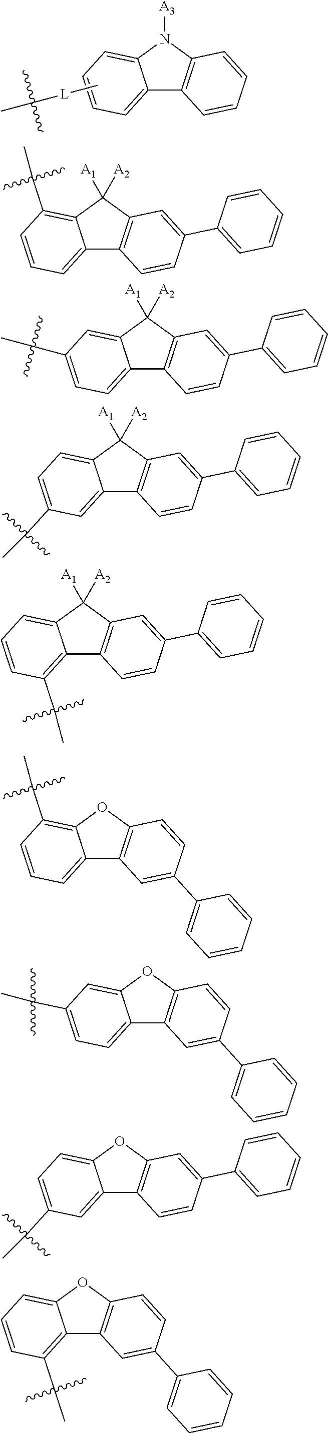

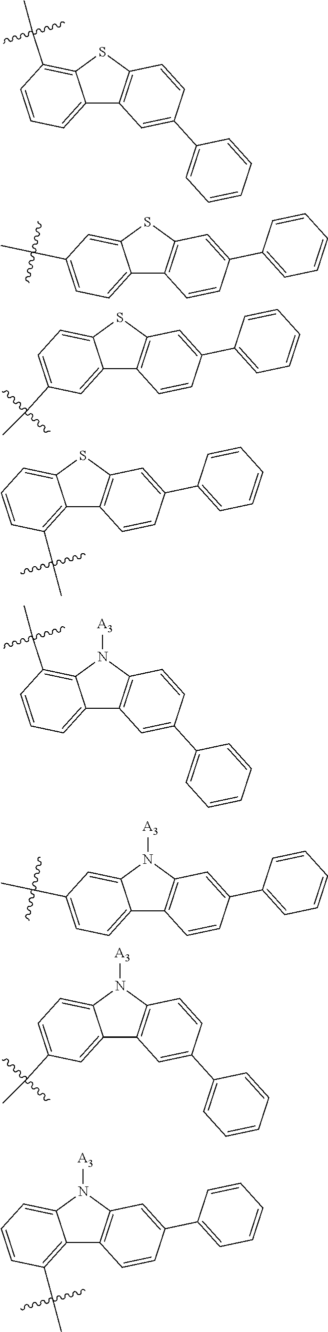

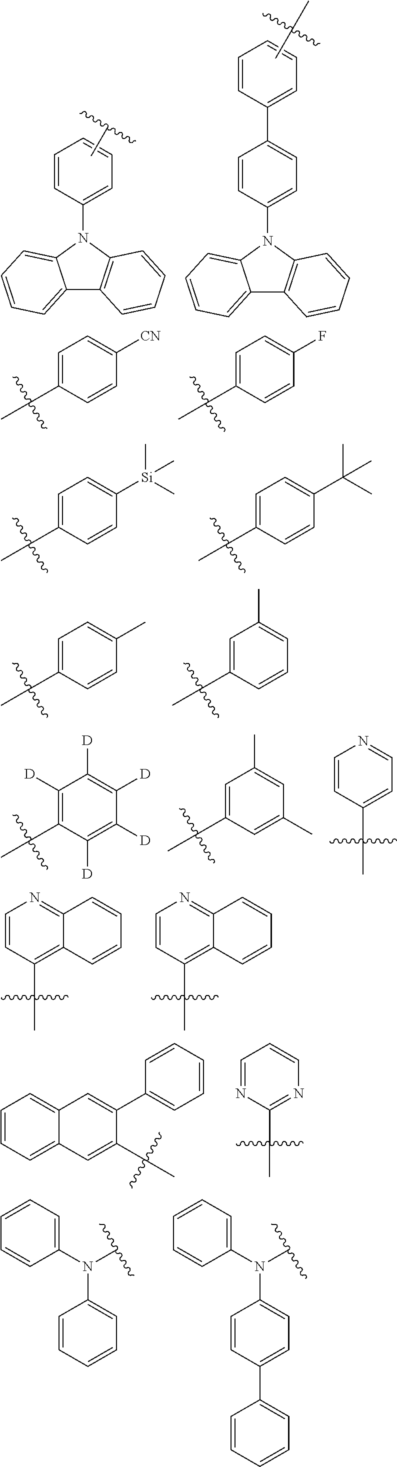

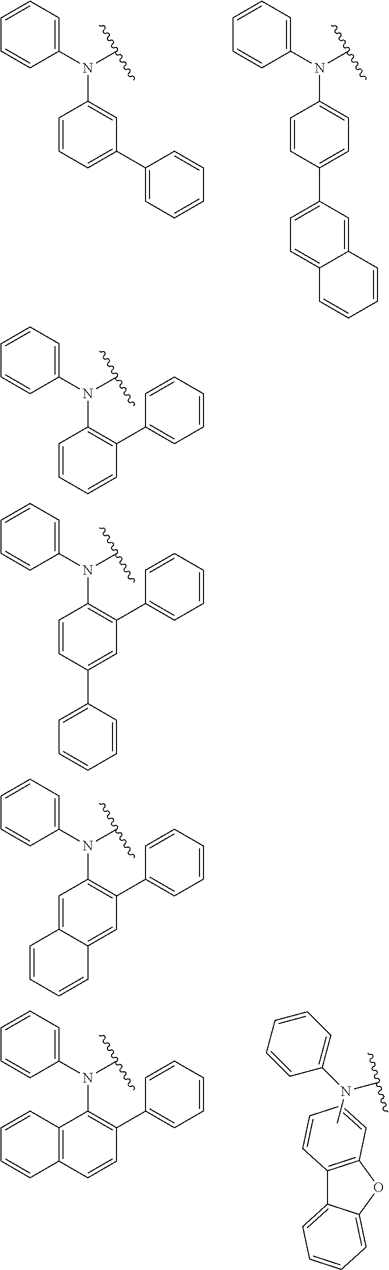

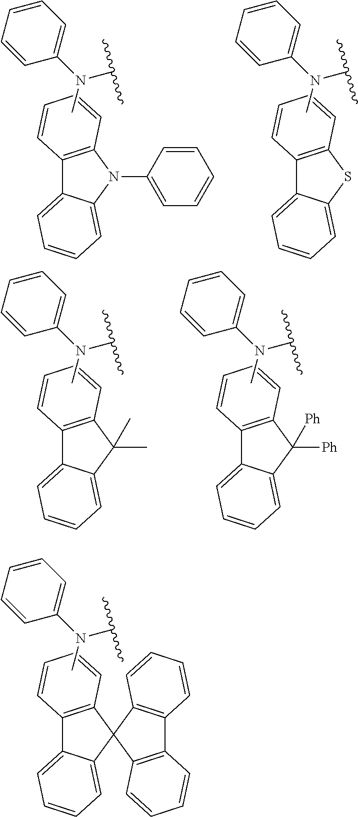

in the Group 2,

L represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; and

A₁ to A₃ each independently represent a substituted or unsubstituted (C1-C30)alkyl or a substituted or unsubstituted (C6-C30)aryl.

According to the other embodiment, Ar₂ and R_{b} each independently may be selected from any one of the substituents listed in the following Group 3.

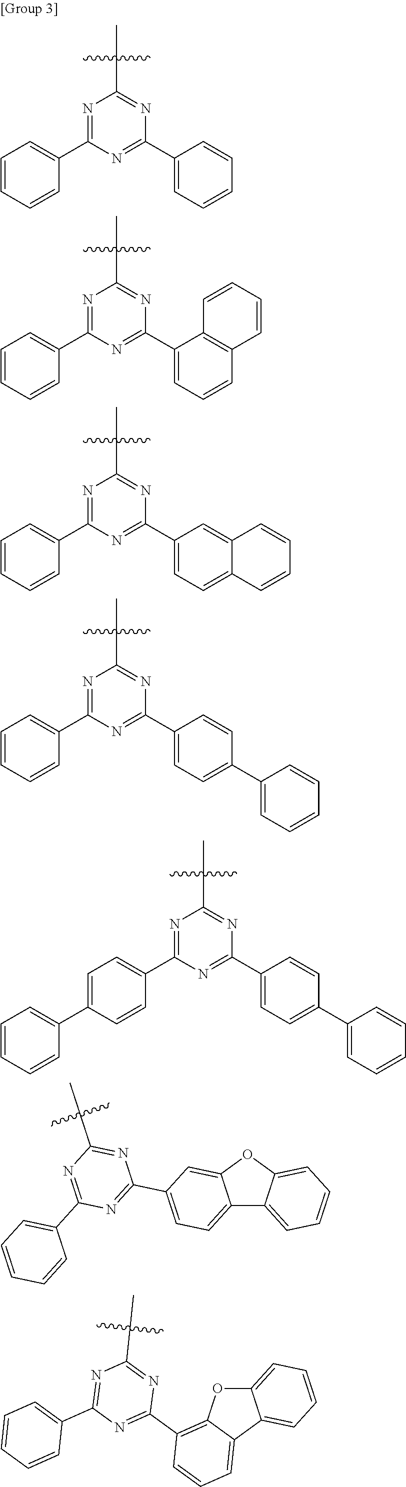

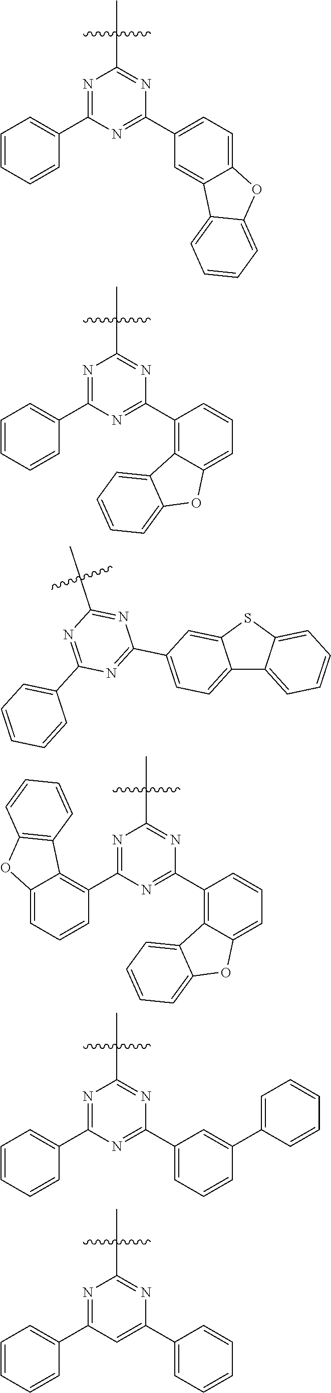

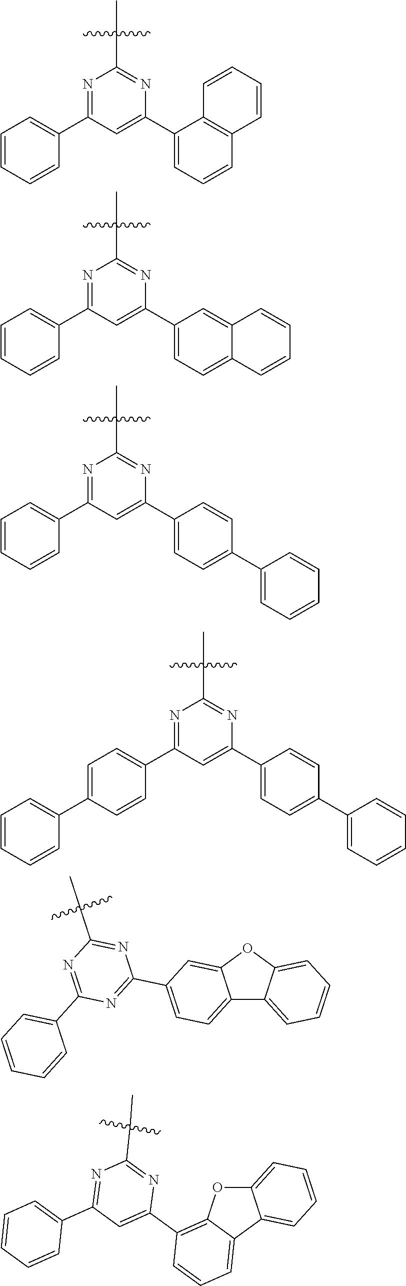 [SMILES: *c1nc(-c2ccccc2)cc(-c2cccc3ccccc23)n1 ; *c1nc(-c2ccccc2)cc(-c2ccc3ccccc3c2)n1 ; *c1nc(-c2ccccc2)cc(-c2ccc(-c3ccccc3)cc2)n1 ; *c1nc(-c2ccc(-c3ccccc3)cc2)cc(-c2ccc(-c3ccccc3)cc2)n1 ; *c1nc(-c2ccccc2)nc(-c2ccc3c(c2)oc2ccccc23)n1 ; *c1nc(-c2ccccc2)cc(-c2cccc3c2oc2ccccc23)n1]

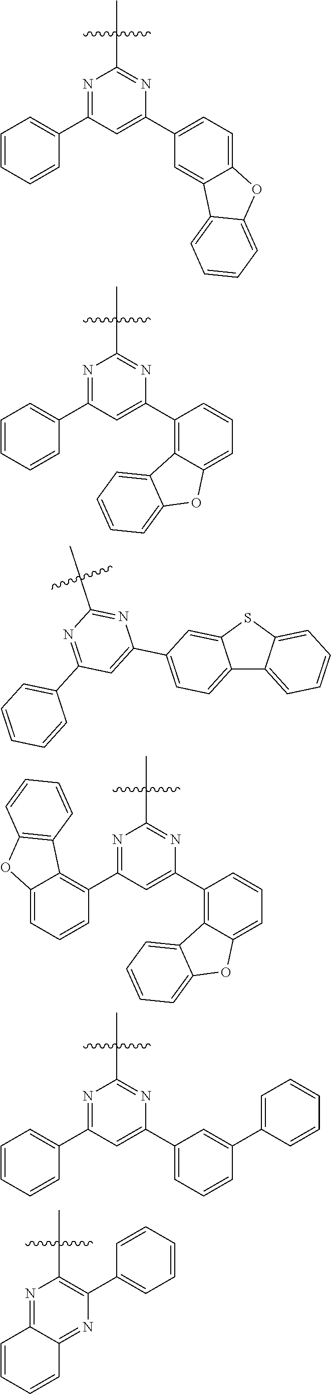

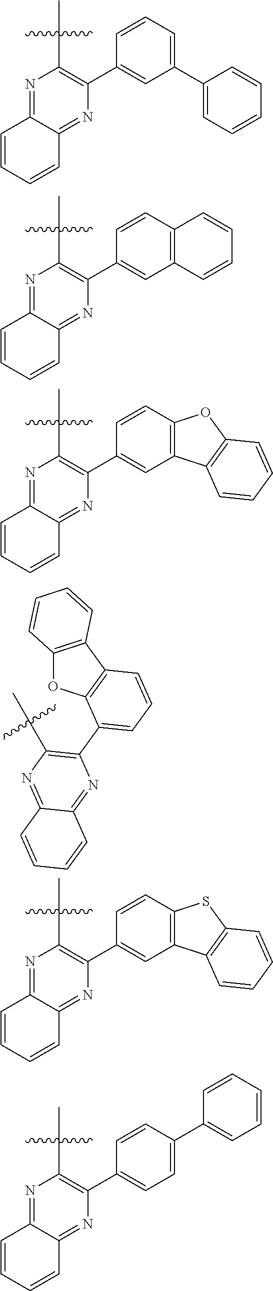

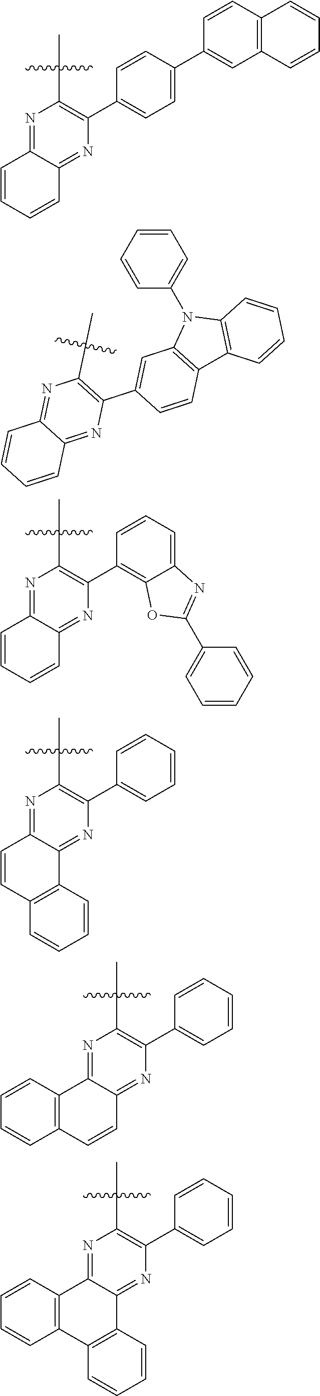 [SMILES: *c1nc2ccccc2nc1-c1ccc(-c2ccc3ccccc3c2)cc1 ; *c1nc2ccccc2nc1-c1ccc2c3ccccc3n(-c3ccccc3)c2c1 ; *c1nc2ccccc2nc1-c1cccc2nc(-c3ccccc3)oc12 ; *c1nc2ccc3ccccc3c2nc1-c1ccccc1 ; *c1nc2c(ccc3ccccc32)nc1-c1ccccc1 ; *c1nc2c3ccccc3c3ccccc3c2nc1-c1ccccc1]

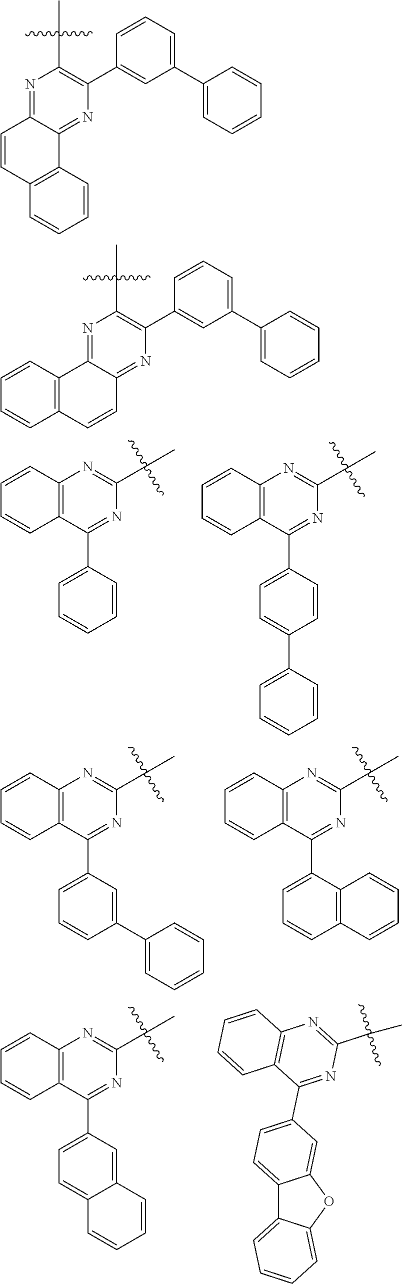

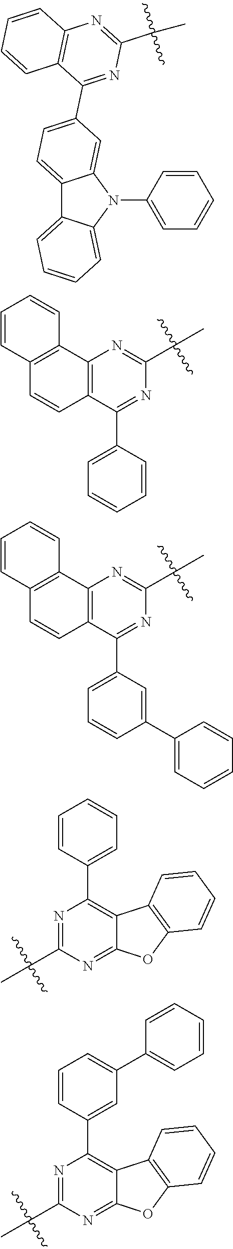

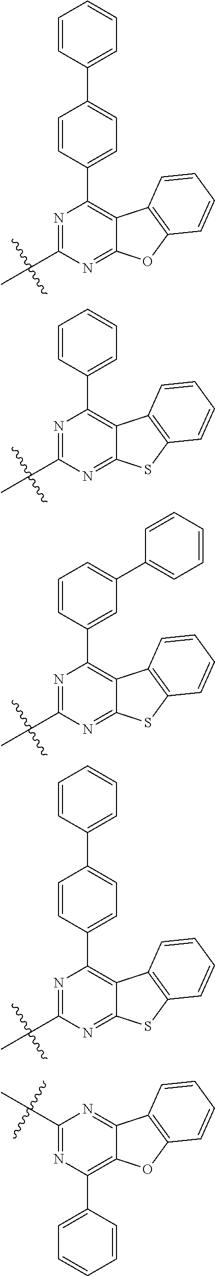

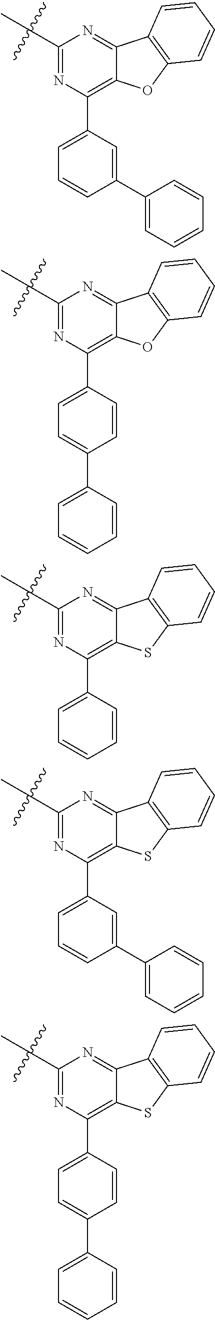

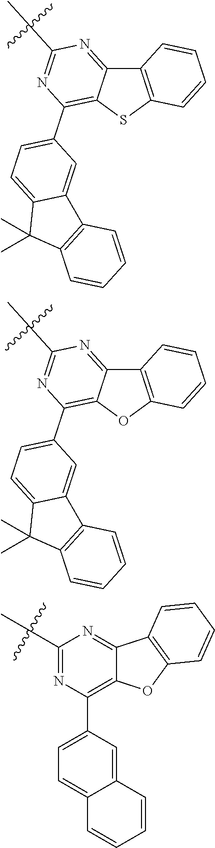

According to one embodiment, the second host material represented by formula 2 above may be more specifically illustrated by the following compounds, but is not limited thereto.

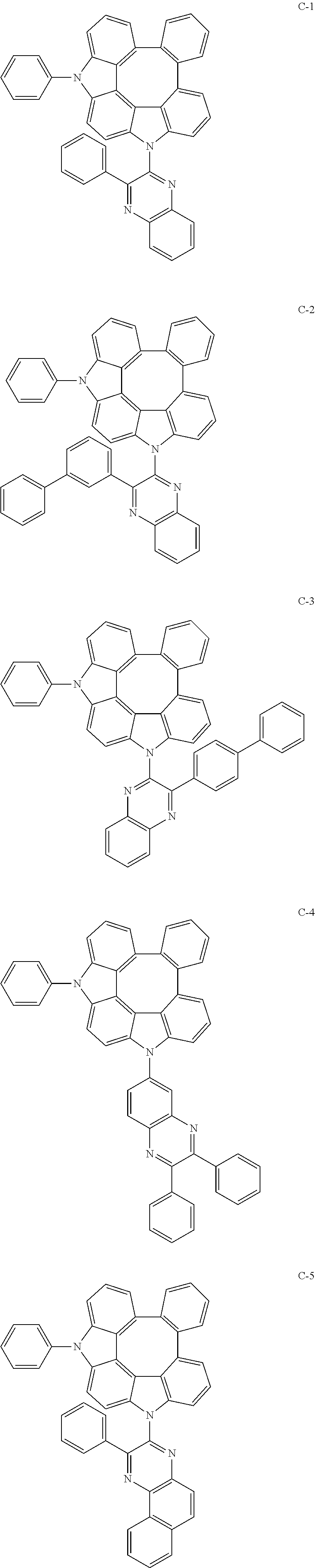 [SMILES: c1ccc(-c2nc3ccccc3nc2-n2c3cccc4c5ccccc5c5cccc6c5c5c(c43)c2ccc5n6-c2ccccc2)cc1 ; c1ccc(-c2cccc(-c3nc4ccccc4nc3-n3c4cccc5c6ccccc6c6cccc7c6c6c(c54)c3ccc6n7-c3ccccc3)c2)cc1 ; c1ccc(-c2ccc(-c3nc4ccccc4nc3-n3c4cccc5c6ccccc6c6cccc7c6c6c(c54)c3ccc6n7-c3ccccc3)cc2)cc1 ; c1ccc(-c2nc3ccc(-n4c5cccc6c7ccccc7c7cccc8c7c7c(c65)c4ccc7n8-c4ccccc4)cc3nc2-c2ccccc2)cc1 ; c1ccc(-c2nc3c(ccc4ccccc43)nc2-n2c3cccc4c5ccccc5c5cccc6c5c5c(c43)c2ccc5n6-c2ccccc2)cc1]

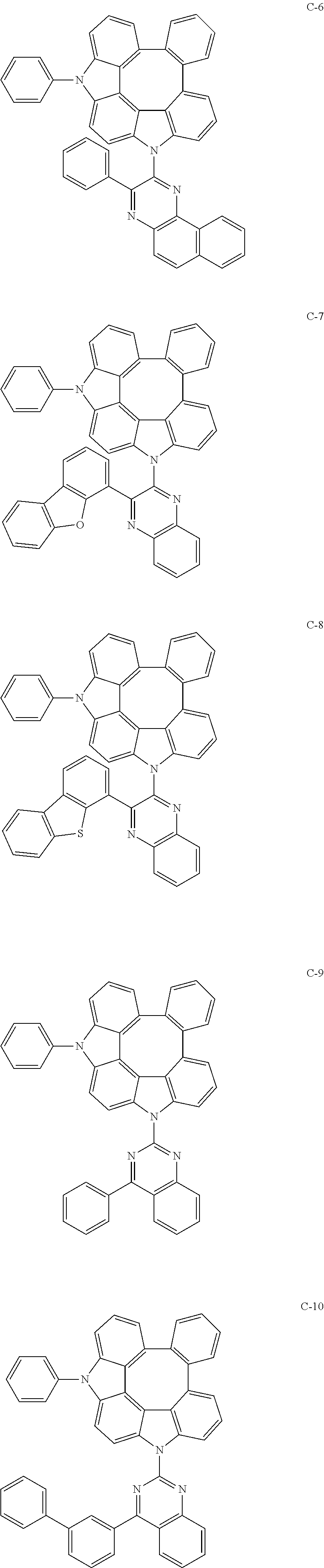 [SMILES: c1ccc(-c2nc3ccc4ccccc4c3nc2-n2c3cccc4c5ccccc5c5cccc6c5c5c(c43)c2ccc5n6-c2ccccc2)cc1 ; c1ccc(-n2c3cccc4c5ccccc5c5cccc6c5c5c(c43)c2ccc5n6-c2nc3ccccc3nc2-c2cccc3c2oc2ccccc23)cc1 ; c1ccc(-n2c3cccc4c5ccccc5c5cccc6c5c5c(c43)c2ccc5n6-c2nc3ccccc3nc2-c2cccc3c2sc2ccccc23)cc1 ; c1ccc(-c2nc(-n3c4cccc5c6ccccc6c6cccc7c6c6c(c54)c3ccc6n7-c3ccccc3)nc3ccccc23)cc1 ; c1ccc(-c2cccc(-c3nc(-n4c5cccc6c7ccccc7c7cccc8c7c7c(c65)c4ccc7n8-c4ccccc4)nc4ccccc34)c2)cc1]

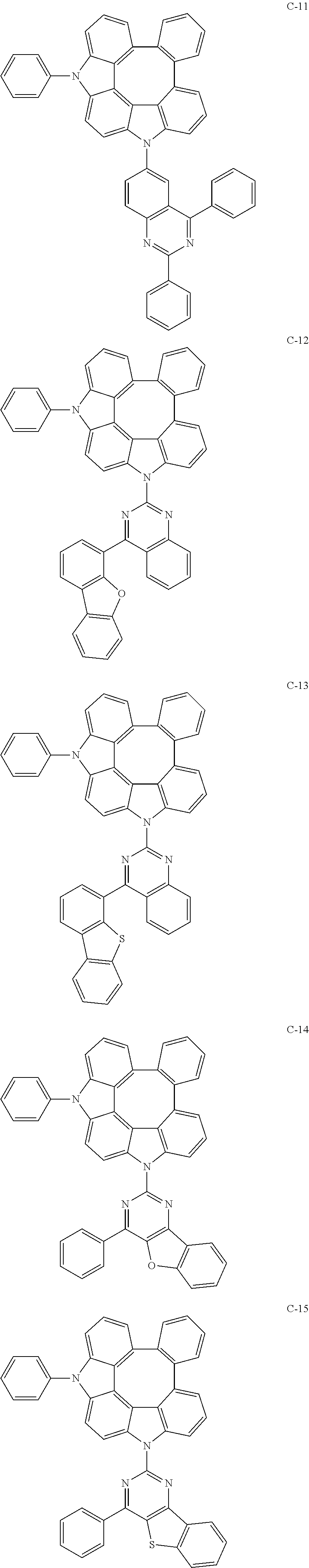

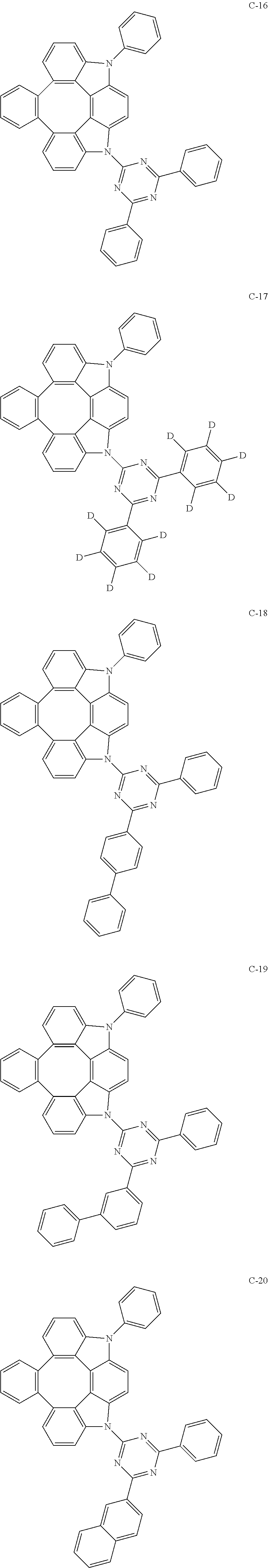

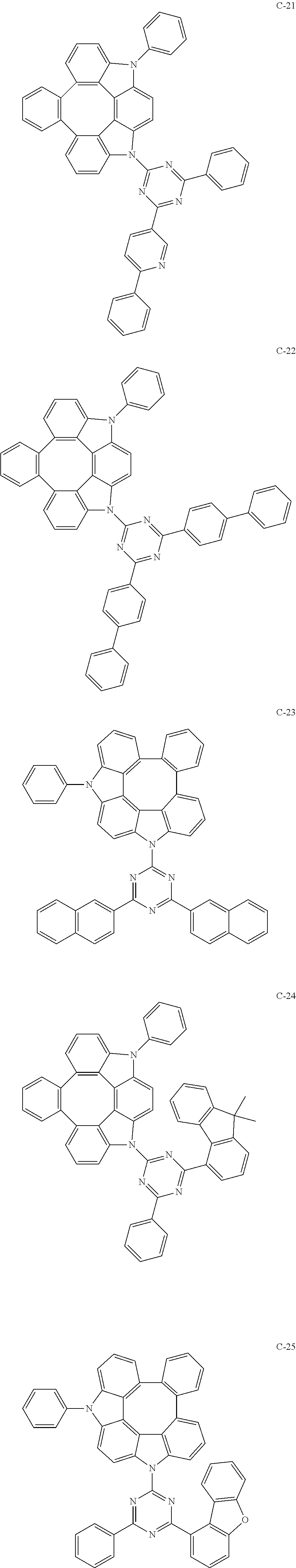

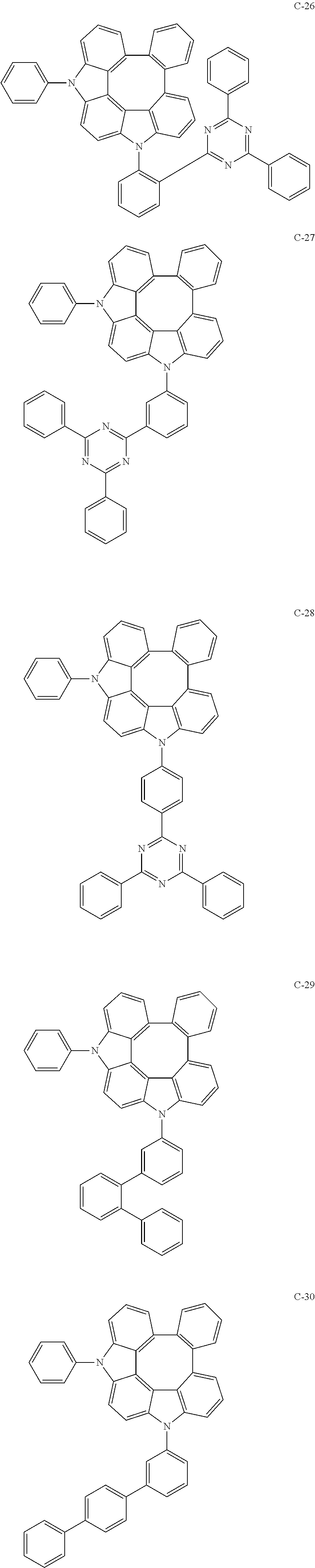

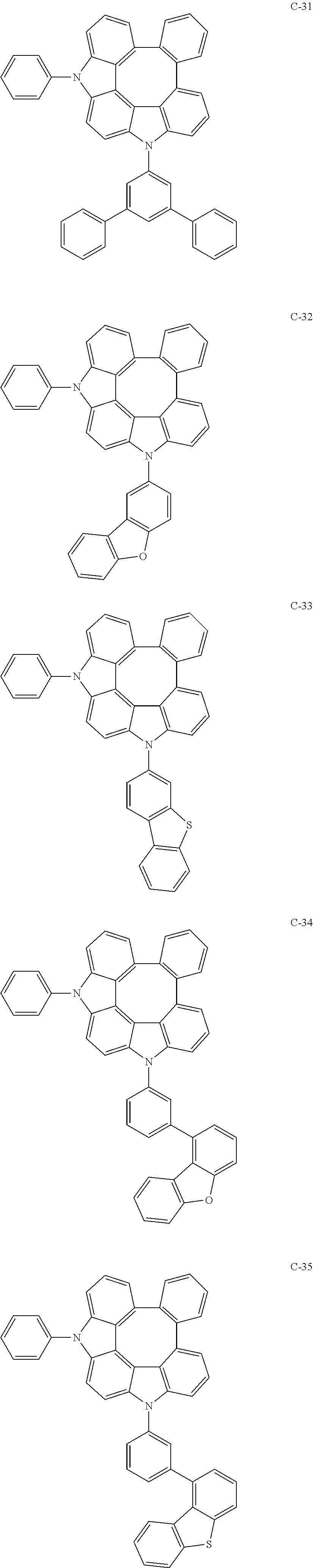

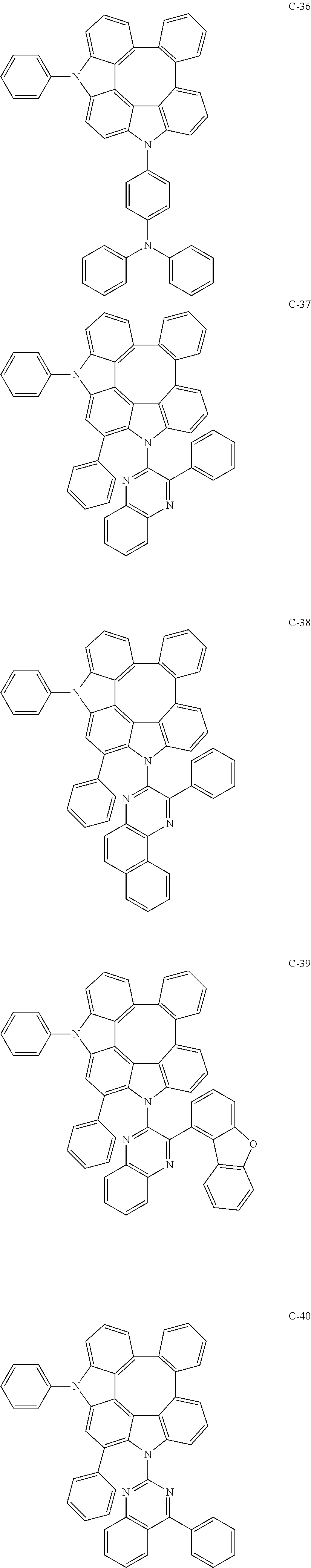

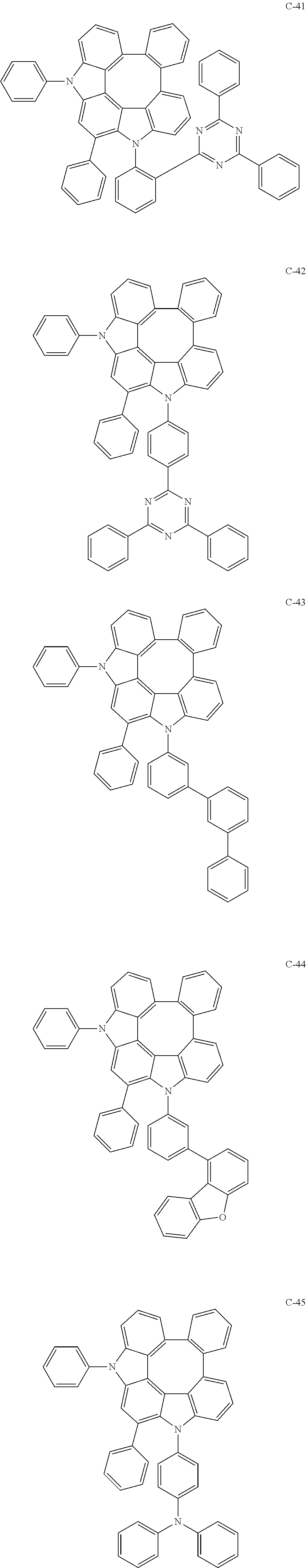

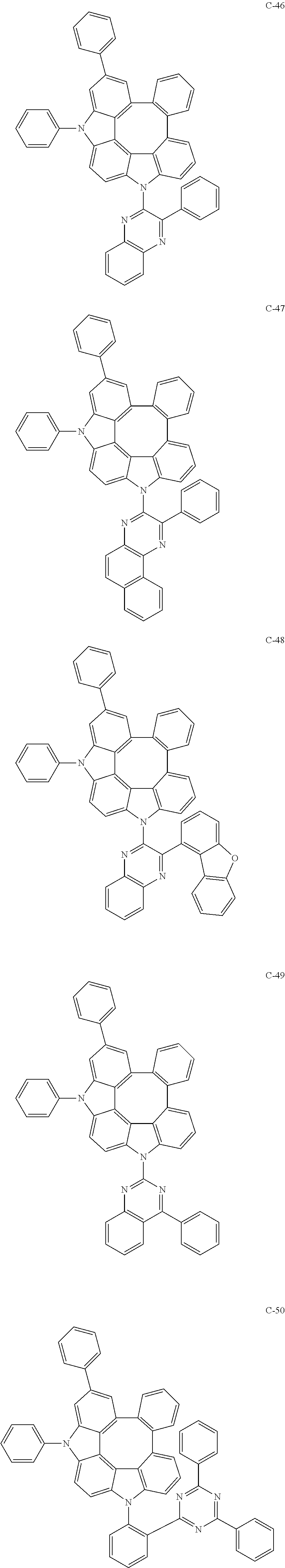 [SMILES: c1ccc(-c2cc3c4ccccc4c4cccc5c4c4c6c3c(c2)n(-c2ccccc2)c6ccc4n5-c2nc3ccccc3nc2-c2ccccc2)cc1 ; c1ccc(-c2cc3c4ccccc4c4cccc5c4c4c6c3c(c2)n(-c2ccccc2)c6ccc4n5-c2nc3ccc4ccccc4c3nc2-c2ccccc2)cc1 ; c1ccc(-c2cc3c4ccccc4c4cccc5c4c4c6c3c(c2)n(-c2ccccc2)c6ccc4n5-c2nc3ccccc3nc2-c2cccc3oc4ccccc4c23)cc1 ; c1ccc(-c2cc3c4ccccc4c4cccc5c4c4c6c3c(c2)n(-c2ccccc2)c6ccc4n5-c2nc(-c3ccccc3)c3ccccc3n2)cc1 ; c1ccc(-c2cc3c4ccccc4c4cccc5c4c4c6c3c(c2)n(-c2ccccc2)c6ccc4n5-c2ccccc2-c2nc(-c3ccccc3)nc(-c3ccccc3)n2)cc1]

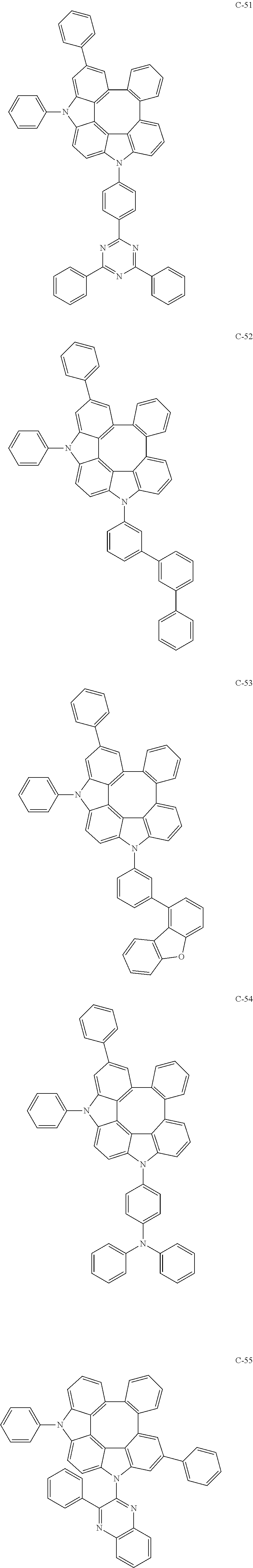 [SMILES: c1ccc(-c2cc3c4ccccc4c4cccc5c4c4c6c3c(c2)n(-c2ccccc2)c6ccc4n5-c2ccc(-c3nc(-c4ccccc4)nc(-c4ccccc4)n3)cc2)cc1 ; c1ccc(-c2cccc(-c3cccc(-n4c5cccc6c7ccccc7c7cc(-c8ccccc8)cc8c7c7c(c65)c4ccc7n8-c4ccccc4)c3)c2)cc1 ; c1ccc(-c2cc3c4ccccc4c4cccc5c4c4c6c3c(c2)n(-c2ccccc2)c6ccc4n5-c2cccc(-c3cccc4oc5ccccc5c34)c2)cc1 ; c1ccc(-c2cc3c4ccccc4c4cccc5c4c4c6c3c(c2)n(-c2ccccc2)c6ccc4n5-c2ccc(N(c3ccccc3)c3ccccc3)cc2)cc1 ; c1ccc(-c2cc3c4ccccc4c4cccc5c4c4c6c3c(c2)n(-c2nc3ccccc3nc2-c2ccccc2)c6ccc4n5-c2ccccc2)cc1]

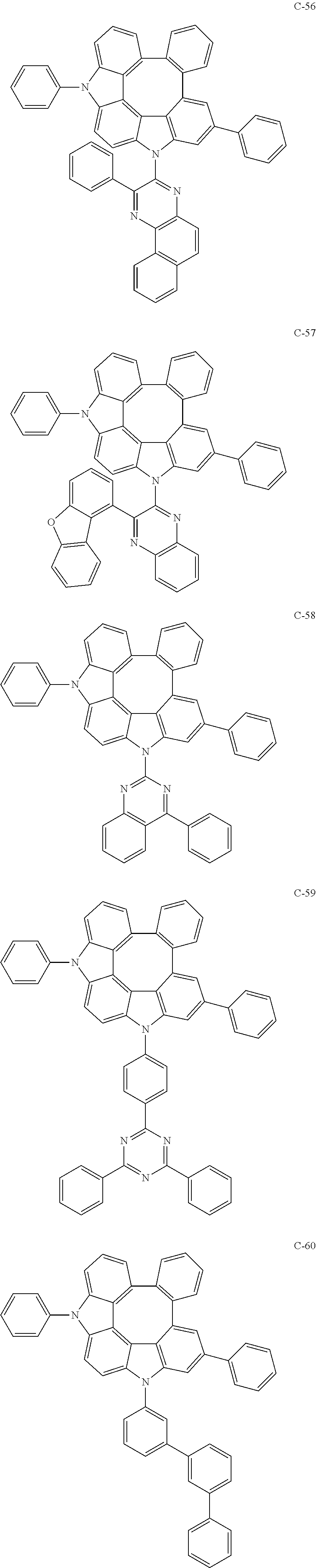

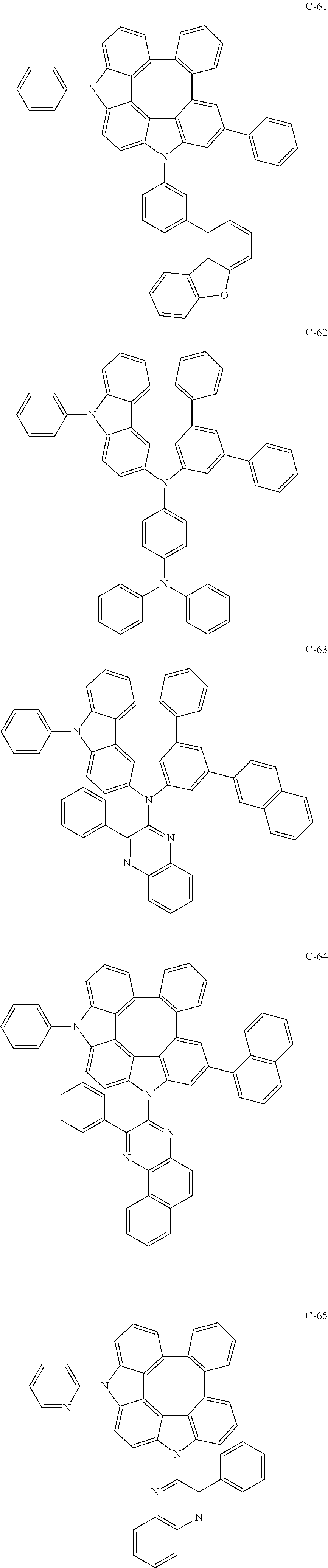 [SMILES: c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1 ; c1ccc(-c2nc3ccccc3nc2-n2c3cc(-c4ccc5ccccc5c4)cc4c5ccccc5c5cccc6c5c5c(c43)c2ccc5n6-c2ccccc2)cc1 ; c1ccc(-c2nc3c(ccc4ccccc43)nc2-n2c3cc(-c4cccc5ccccc45)cc4c5ccccc5c5cccc6c5c5c(c43)c2ccc5n6-c2ccccc2)cc1 ; c1ccc(-c2nc3ccccc3nc2-n2c3cccc4c5ccccc5c5cccc6c5c5c(c43)c2ccc5n6-c2ccccn2)cc1]

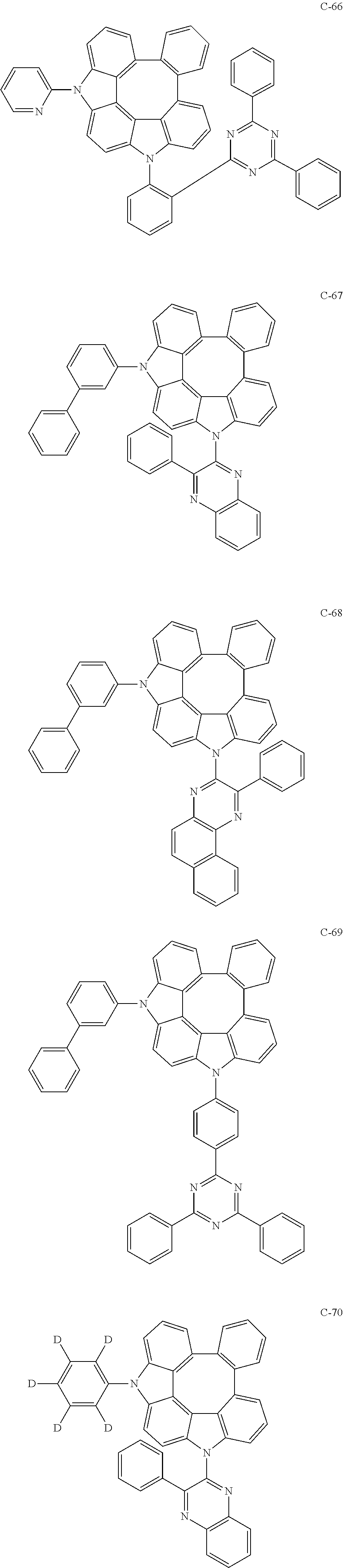 [SMILES: c1ccc(-c2nc(-c3ccccc3)nc(-c3ccccc3-n3c4cccc5c6ccccc6c6cccc7c6c6c(c54)c3ccc6n7-c3ccccn3)n2)cc1 ; c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3nc4ccc5ccccc5c4nc3-c3ccccc3)c2)cc1 ; c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1 ; [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H]]

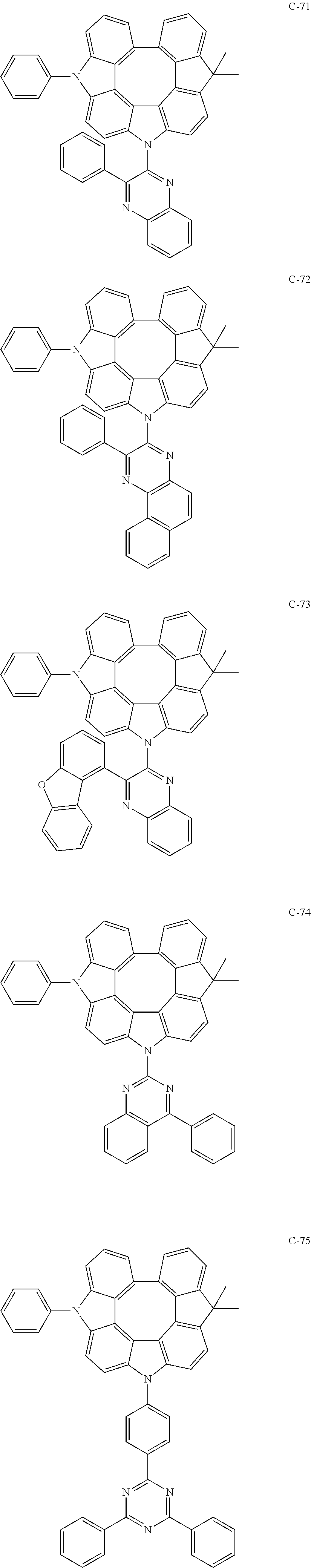

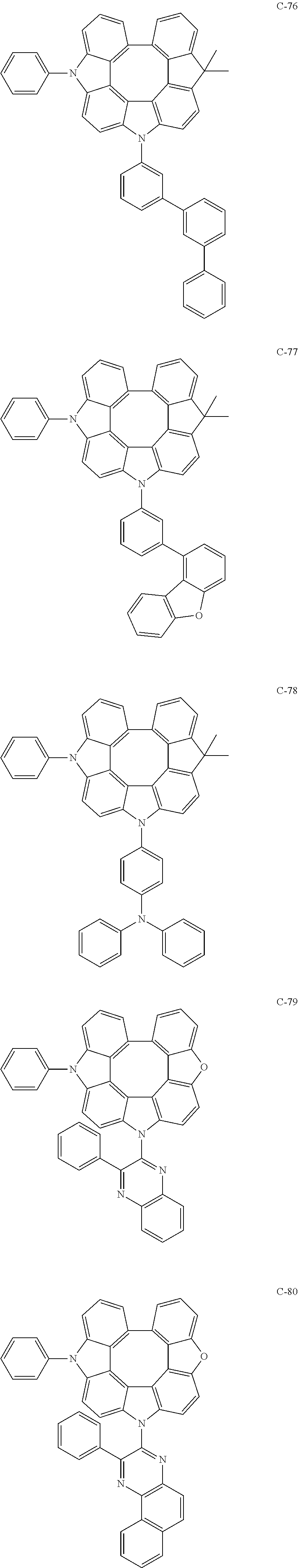 [SMILES: CC1(C)c2cccc3c4cccc5c4c4c6c7c(c1ccc7n(-c1cccc(-c7cccc(-c8ccccc8)c7)c1)c6ccc4n5-c1ccccc1)c23 ; CC1(C)c2cccc3c4cccc5c4c4c6c7c(c1ccc7n(-c1cccc(-c7cccc8oc9ccccc9c78)c1)c6ccc4n5-c1ccccc1)c23 ; CC1(C)c2cccc3c4cccc5c4c4c6c7c(c1ccc7n(-c1ccc(N(c7ccccc7)c7ccccc7)cc1)c6ccc4n5-c1ccccc1)c23 ; c1ccc(-c2nc3ccccc3nc2-n2c3ccc4oc5cccc6c7cccc8c7c7c(c3c4c56)c2ccc7n8-c2ccccc2)cc1 ; c1ccc(-c2nc3c(ccc4ccccc43)nc2-n2c3ccc4oc5cccc6c7cccc8c7c7c(c3c4c56)c2ccc7n8-c2ccccc2)cc1]

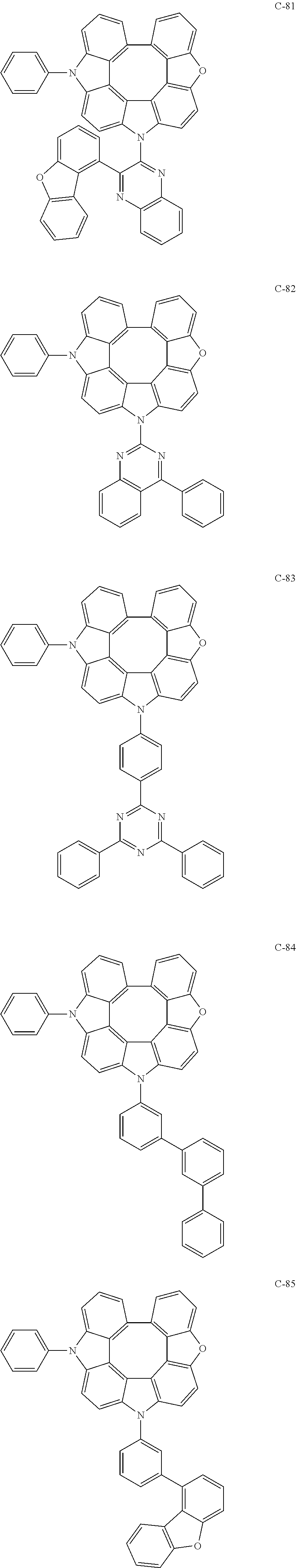 [SMILES: c1ccc(-n2c3cccc4c5cccc6oc7ccc8c(c7c65)c5c(c43)c2ccc5n8-c2nc3ccccc3nc2-c2cccc3oc4ccccc4c23)cc1 ; c1ccc(-c2nc(-n3c4ccc5oc6cccc7c8cccc9c8c8c(c4c5c67)c3ccc8n9-c3ccccc3)nc3ccccc23)cc1 ; c1ccc(-c2nc(-c3ccccc3)nc(-c3ccc(-n4c5ccc6oc7cccc8c9cccc%10c9c9c(c5c6c78)c4ccc9n%10-c4ccccc4)cc3)n2)cc1 ; c1ccc(-c2cccc(-c3cccc(-n4c5ccc6oc7cccc8c9cccc%10c9c9c(c5c6c78)c4ccc9n%10-c4ccccc4)c3)c2)cc1 ; c1ccc(-n2c3cccc4c5cccc6oc7ccc8c(c7c65)c5c(c43)c2ccc5n8-c2cccc(-c3cccc4oc5ccccc5c34)c2)cc1]

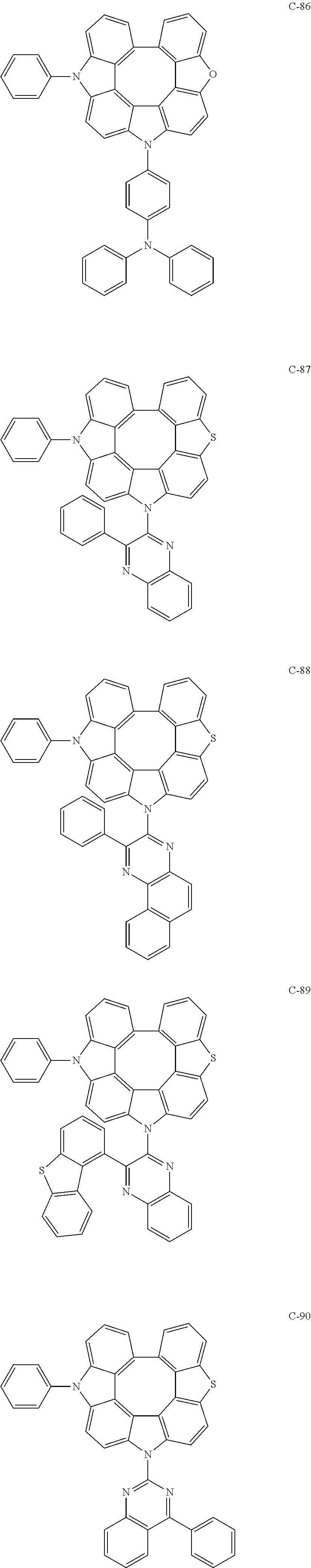 [SMILES: c1ccc(N(c2ccccc2)c2ccc(-n3c4ccc5oc6cccc7c8cccc9c8c8c(c4c5c67)c3ccc8n9-c3ccccc3)cc2)cc1 ; c1ccc(-c2nc3ccccc3nc2-n2c3ccc4sc5cccc6c7cccc8c7c7c(c3c4c56)c2ccc7n8-c2ccccc2)cc1 ; c1ccc(-c2nc3c(ccc4ccccc43)nc2-n2c3ccc4sc5cccc6c7cccc8c7c7c(c3c4c56)c2ccc7n8-c2ccccc2)cc1 ; c1ccc(-n2c3cccc4c5cccc6sc7ccc8c(c7c65)c5c(c43)c2ccc5n8-c2nc3ccccc3nc2-c2cccc3sc4ccccc4c23)cc1 ; c1ccc(-c2nc(-n3c4ccc5sc6cccc7c8cccc9c8c8c(c4c5c67)c3ccc8n9-c3ccccc3)nc3ccccc23)cc1]

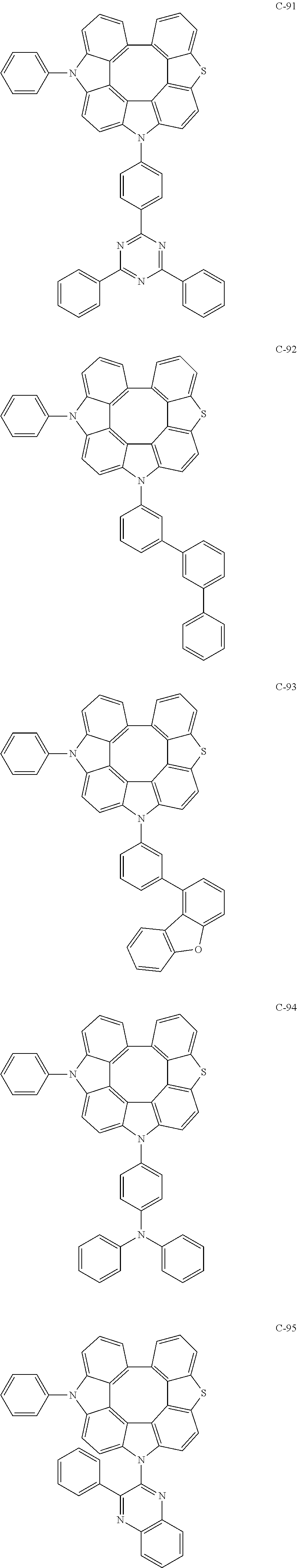 [SMILES: c1ccc(-c2nc(-c3ccccc3)nc(-c3ccc(-n4c5ccc6sc7cccc8c9cccc%10c9c9c(c5c6c78)c4ccc9n%10-c4ccccc4)cc3)n2)cc1 ; c1ccc(-c2cccc(-c3cccc(-n4c5ccc6sc7cccc8c9cccc%10c9c9c(c5c6c78)c4ccc9n%10-c4ccccc4)c3)c2)cc1 ; c1ccc(-n2c3cccc4c5cccc6sc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5sc6cccc7c8cccc9c8c8c(c4c5c67)c3ccc8n9-c3ccccc3)cc2)cc1 ; c1ccc(-c2nc3ccccc3nc2-n2c3ccc4sc5cccc6c7cccc8c7c7c(c3c4c56)c2ccc7n8-c2ccccc2)cc1]

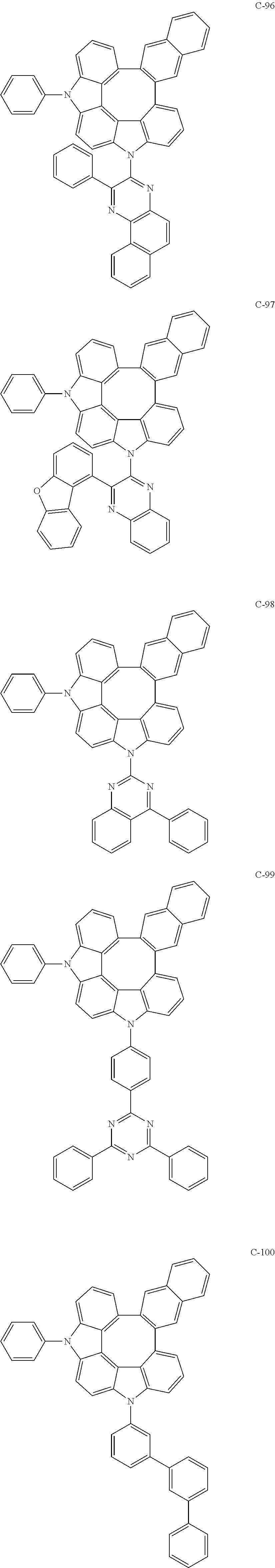

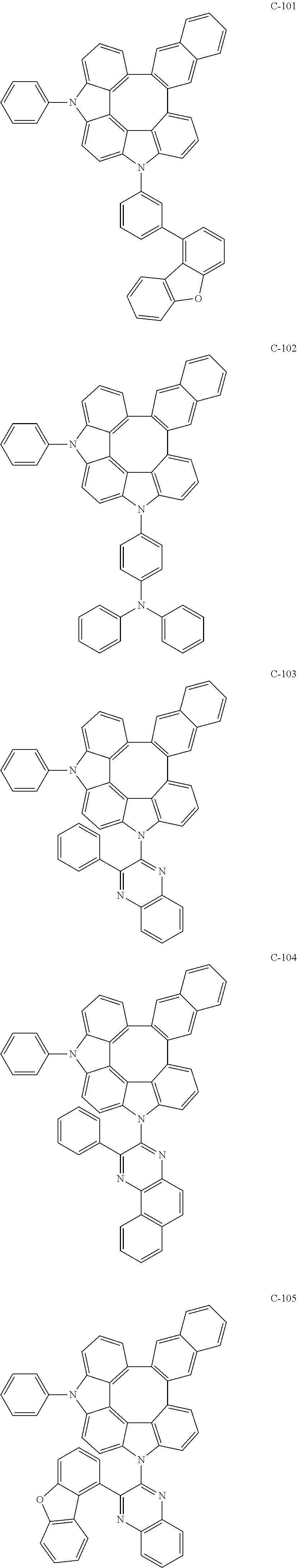 [SMILES: c1ccc(-n2c3cccc4c5cc6ccccc6cc5c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cc7ccccc7cc6c6cccc7c6c6c(c54)c3ccc6n7-c3ccccc3)cc2)cc1 ; c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

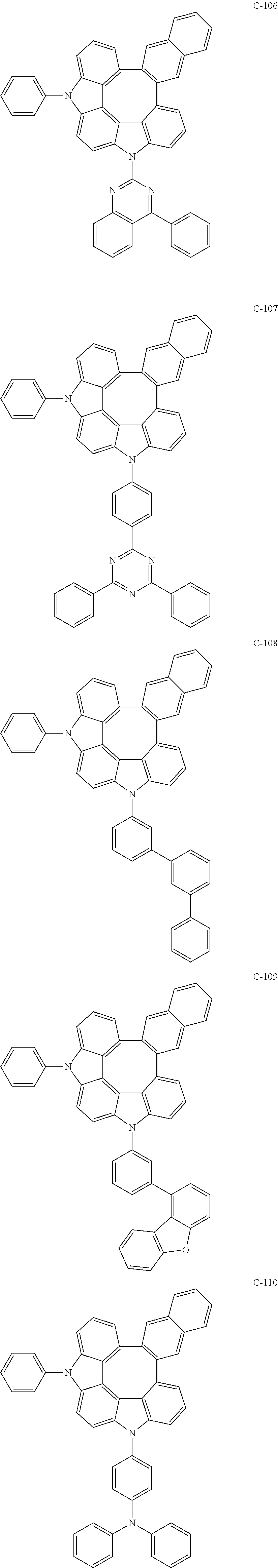

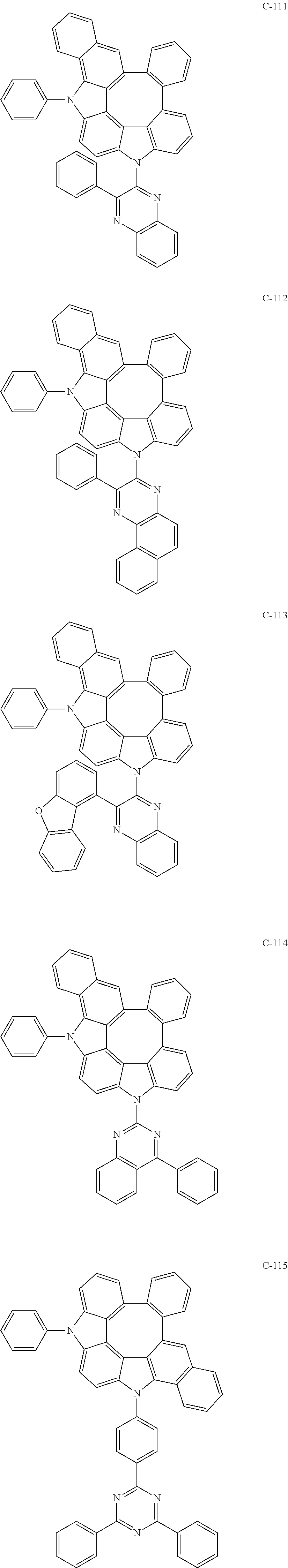 [SMILES: c1ccc(-c2nc3ccccc3nc2-n2c3cccc4c5ccccc5c5cc6ccccc6c6c5c5c(c43)c2ccc5n6-c2ccccc2)cc1 ; c1ccc(-c2nc3c(ccc4ccccc43)nc2-n2c3cccc4c5ccccc5c5cc6ccccc6c6c5c5c(c43)c2ccc5n6-c2ccccc2)cc1 ; c1ccc(-n2c3ccc4c5c6c(cccc6n4-c4nc6ccccc6nc4-c4cccc6oc7ccccc7c46)c4ccccc4c4cc6ccccc6c2c4c53)cc1 ; c1ccc(-c2nc(-n3c4cccc5c6ccccc6c6cc7ccccc7c7c6c6c(c54)c3ccc6n7-c3ccccc3)nc3ccccc23)cc1 ; c1ccc(-c2nc(-c3ccccc3)nc(-c3ccc(-n4c5ccc6c7c8c(cccc8n6-c6ccccc6)c6ccccc6c6cc8ccccc8c4c6c75)cc3)n2)cc1]

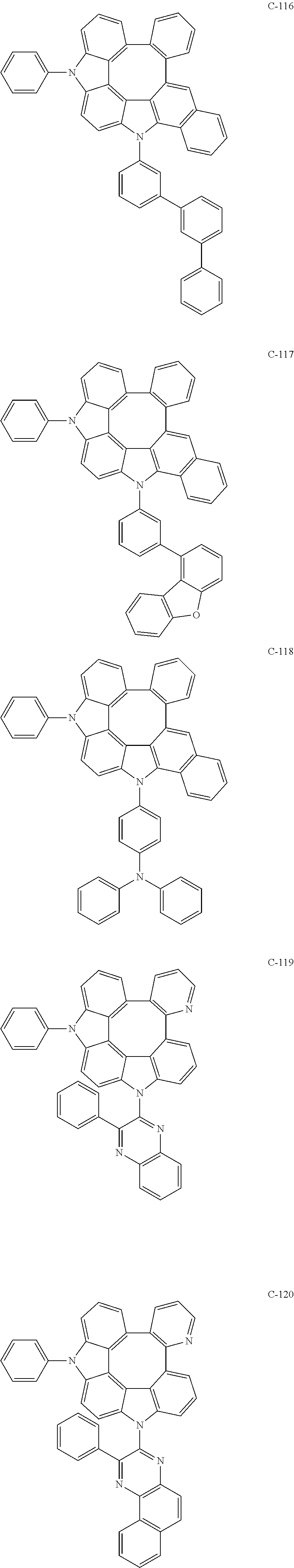

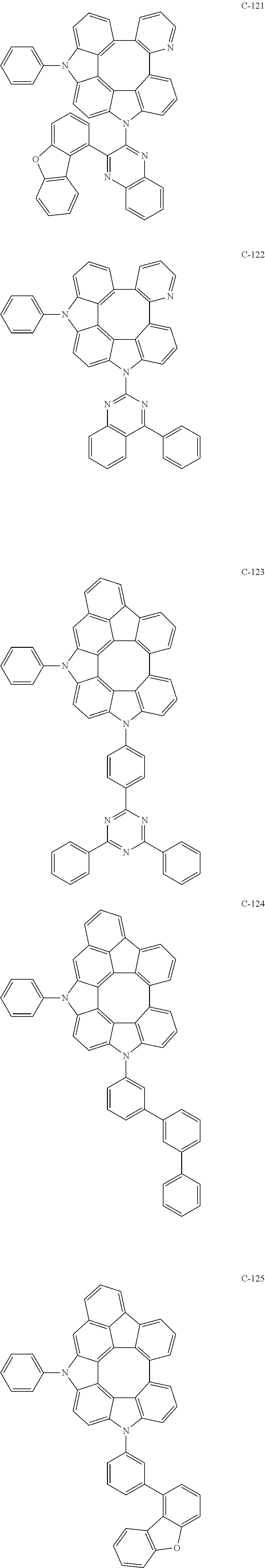 [SMILES: c1ccc(-n2c3cccc4c5cccnc5c5cccc6c5c5c(c43)c2ccc5n6-c2nc3ccccc3nc2-c2cccc3oc4ccccc4c23)cc1 ; c1ccc(-c2nc(-n3c4cccc5c6ncccc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cccc8c7c7c9c-8cccc9cc8c7c7c(c65)c4ccc7n8-c4ccccc4)cc3)n2)cc1 ; c1ccc(-c2cccc(-c3cccc(-n4c5cccc6c7cccc8c7c7c9c-8cccc9cc8c7c7c(c65)c4ccc7n8-c4ccccc4)c3)c2)cc1 ; c1ccc(-n2c3cc4cccc5c4c4c6c-5cccc6c5cccc6c5c5c(c43)c2ccc5n6-c2cccc(-c3cccc4oc5ccccc5c34)c2)cc1]

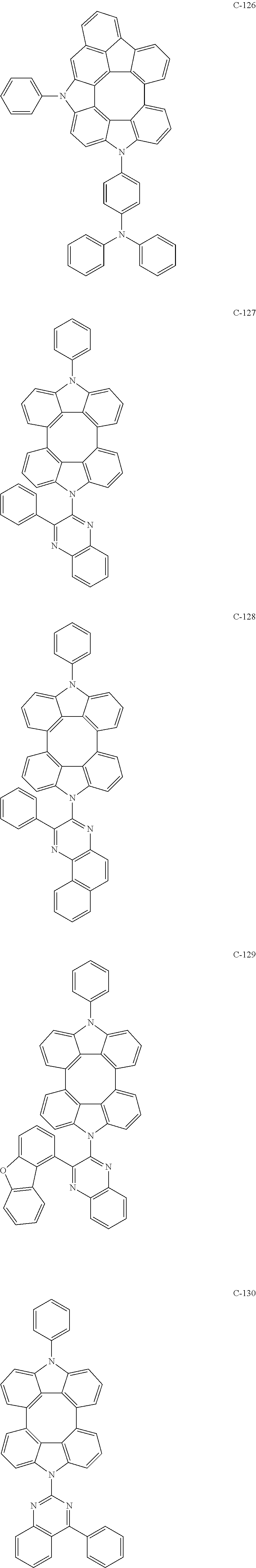 [SMILES: c1ccc(N(c2ccccc2)c2ccc(-n3c4cccc5c6cccc7c6c6c8c-7cccc8cc7c6c6c(c54)c3ccc6n7-c3ccccc3)cc2)cc1 ; c1ccc(-c2nc3ccccc3nc2-n2c3cccc4c5cccc6c5c5c(cccc5n6-c5ccccc5)c5cccc2c5c43)cc1 ; c1ccc(-c2nc3c(ccc4ccccc43)nc2-n2c3cccc4c5cccc6c5c5c(cccc5n6-c5ccccc5)c5cccc2c5c43)cc1 ; c1ccc(-n2c3cccc4c5cccc6c5c5c(cccc5n6-c5nc6ccccc6nc5-c5cccc6oc7ccccc7c56)c5cccc2c5c43)cc1 ; c1ccc(-c2nc(-n3c4cccc5c6cccc7c6c6c(cccc6n7-c6ccccc6)c6cccc3c6c54)nc3ccccc23)cc1]

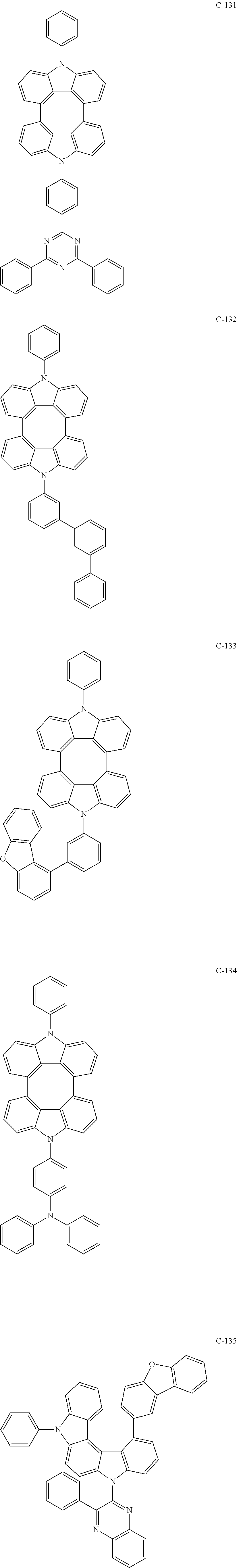 [SMILES: c1ccc(-c2nc(-c3ccccc3)nc(-c3ccc(-n4c5cccc6c7cccc8c7c7c(cccc7n8-c7ccccc7)c7cccc4c7c65)cc3)n2)cc1 ; c1ccc(-c2cccc(-c3cccc(-n4c5cccc6c7cccc8c7c7c(cccc7n8-c7ccccc7)c7cccc4c7c65)c3)c2)cc1 ; c1ccc(-n2c3cccc4c5cccc6c5c5c(cccc5n6-c5cccc(-c6cccc7oc8ccccc8c67)c5)c5cccc2c5c43)cc1 ; c1ccc(N(c2ccccc2)c2ccc(-n3c4cccc5c6cccc7c6c6c(cccc6n7-c6ccccc6)c6cccc3c6c54)cc2)cc1 ; c1ccc(-c2nc3ccccc3nc2-n2c3cccc4c5cc6c(cc5c5cccc7c5c5c(c43)c2ccc5n7-c2ccccc2)oc2ccccc26)cc1]

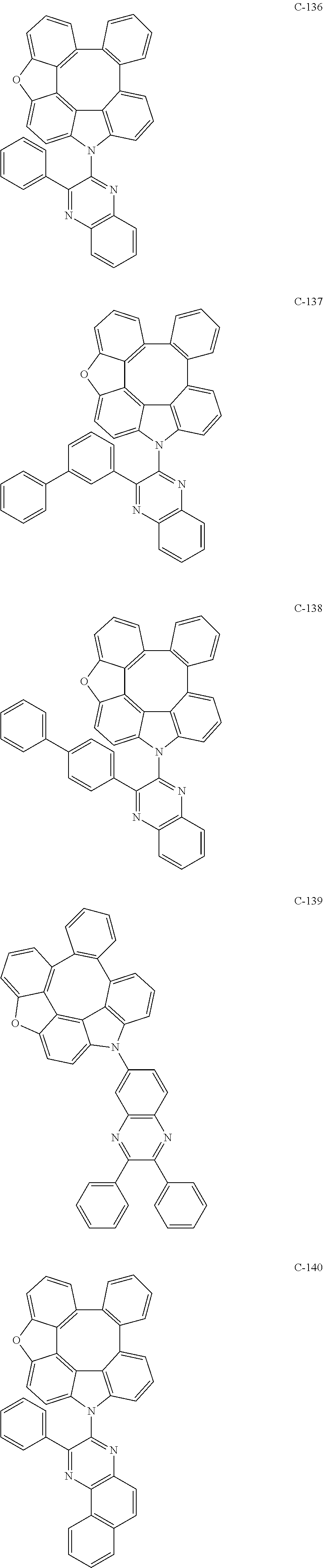 [SMILES: c1ccc(-c2nc3ccccc3nc2-n2c3cccc4c5ccccc5c5cccc6oc7ccc2c(c7c65)c43)cc1 ; c1ccc(-c2cccc(-c3nc4ccccc4nc3-n3c4cccc5c6ccccc6c6cccc7oc8ccc3c(c8c76)c54)c2)cc1 ; c1ccc(-c2ccc(-c3nc4ccccc4nc3-n3c4cccc5c6ccccc6c6cccc7oc8ccc3c(c8c76)c54)cc2)cc1 ; c1ccc(-c2nc3ccc(-n4c5cccc6c7ccccc7c7cccc8oc9ccc4c(c9c87)c65)cc3nc2-c2ccccc2)cc1 ; c1ccc(-c2nc3c(ccc4ccccc43)nc2-n2c3cccc4c5ccccc5c5cccc6oc7ccc2c(c7c65)c43)cc1]

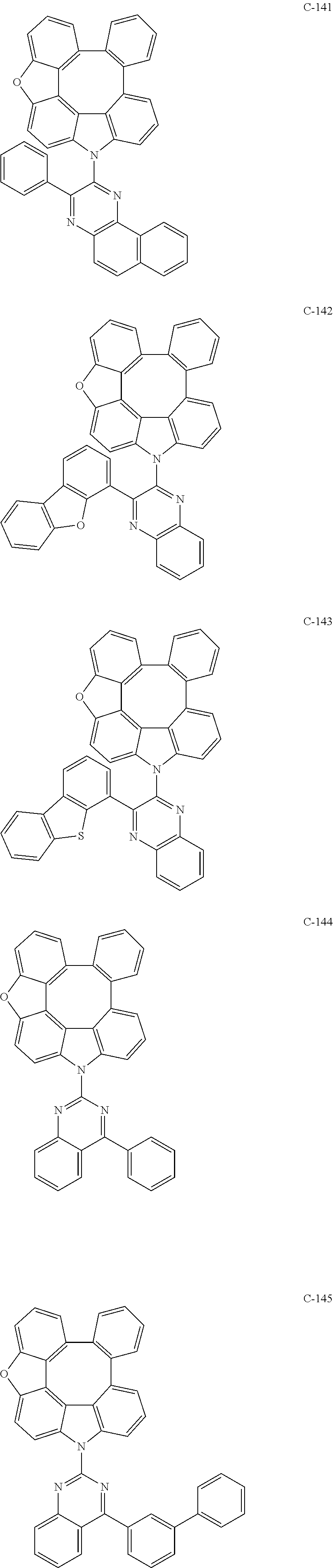 [SMILES: c1ccc(-c2nc3ccc4ccccc4c3nc2-n2c3cccc4c5ccccc5c5cccc6oc7ccc2c(c7c65)c43)cc1 ; c1ccc2nc(-n3c4cccc5c6ccccc6c6cccc7oc8ccc3c(c8c76)c54)c(-c3cccc4c3oc3ccccc34)nc2c1 ; c1ccc2nc(-n3c4cccc5c6ccccc6c6cccc7oc8ccc3c(c8c76)c54)c(-c3cccc4c3sc3ccccc34)nc2c1 ; c1ccc(-c2nc(-n3c4cccc5c6ccccc6c6cccc7oc8ccc3c(c8c76)c54)nc3ccccc23)cc1 ; c1ccc(-c2cccc(-c3nc(-n4c5cccc6c7ccccc7c7cccc8oc9ccc4c(c9c87)c65)nc4ccccc34)c2)cc1]

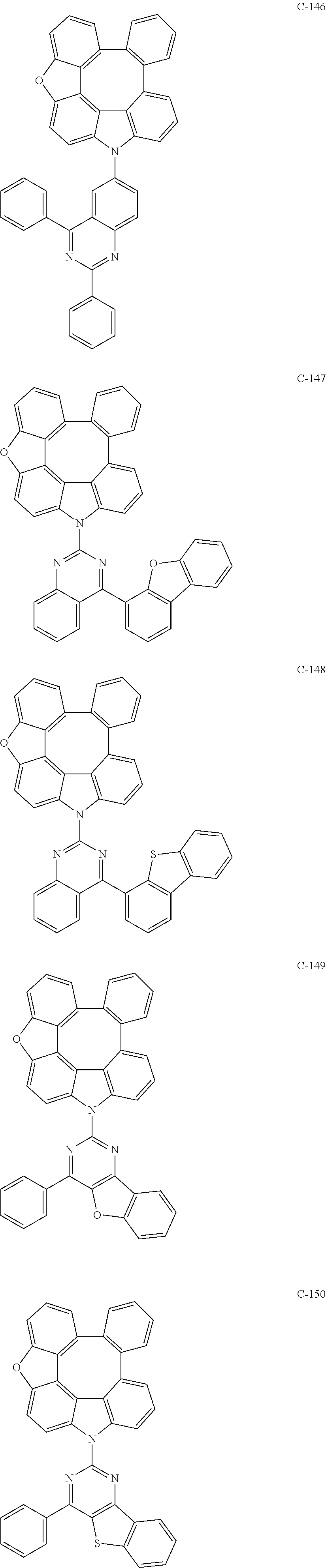 [SMILES: c1ccc(-c2nc(-c3ccccc3)c3cc(-n4c5cccc6c7ccccc7c7cccc8oc9ccc4c(c9c87)c65)ccc3n2)cc1 ; c1ccc2c(-c3cccc4c3oc3ccccc34)nc(-n3c4cccc5c6ccccc6c6cccc7oc8ccc3c(c8c76)c54)nc2c1 ; c1ccc2c(-c3cccc4c3sc3ccccc34)nc(-n3c4cccc5c6ccccc6c6cccc7oc8ccc3c(c8c76)c54)nc2c1 ; c1ccc(-c2nc(-n3c4cccc5c6ccccc6c6cccc7oc8ccc3c(c8c76)c54)nc3c2oc2ccccc23)cc1 ; c1ccc(-c2nc(-n3c4cccc5c6ccccc6c6cccc7oc8ccc3c(c8c76)c54)nc3c2sc2ccccc23)cc1]

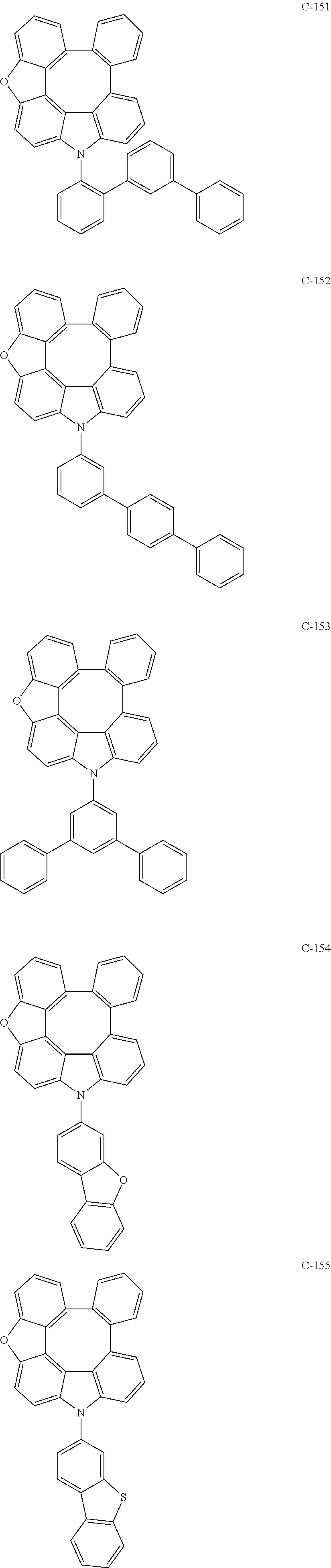

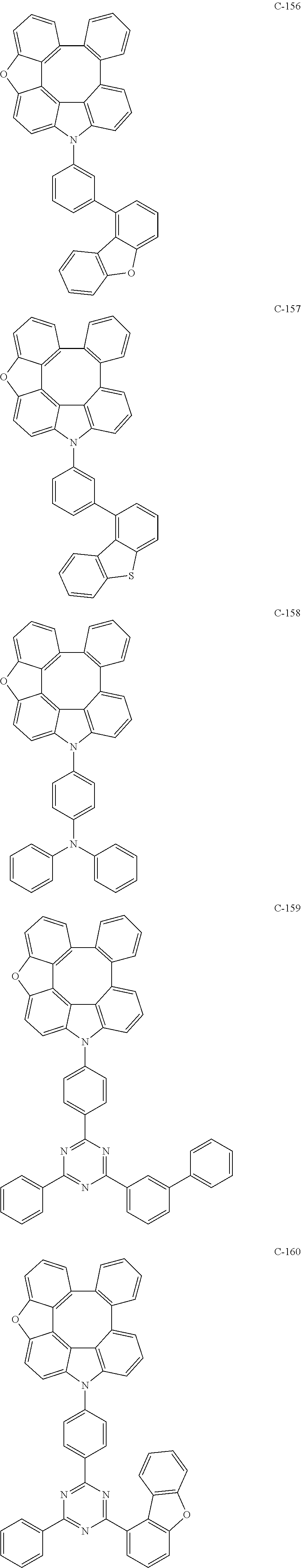

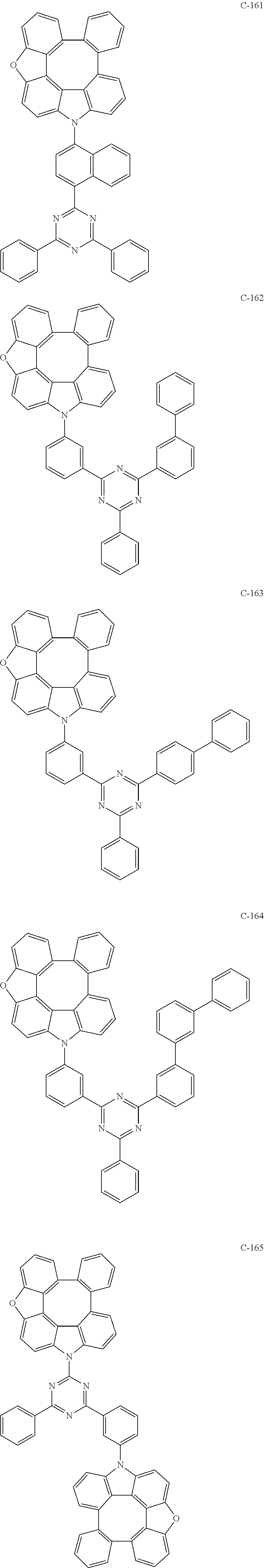

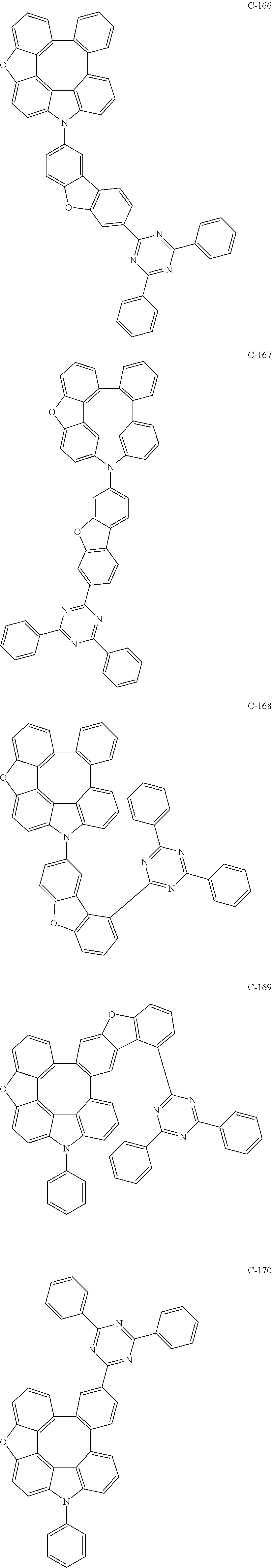

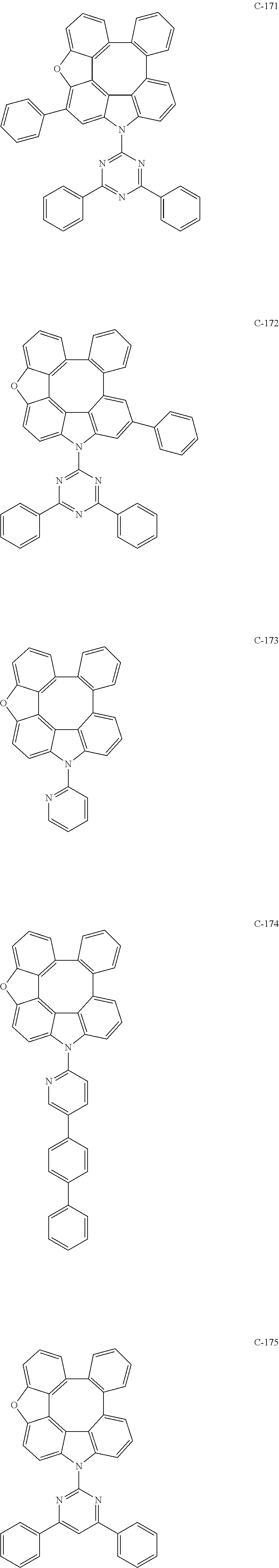 [SMILES: c1ccc(-c2nc(-c3ccccc3)nc(-n3c4cccc5c6ccccc6c6cccc7oc8c(-c9ccccc9)cc3c(c8c76)c54)n2)cc1 ; c1ccc(-c2cc3c4ccccc4c4cccc5oc6ccc7c(c6c54)c3c(c2)n7-c2nc(-c3ccccc3)nc(-c3ccccc3)n2)cc1 ; c1ccc(-n2c3cccc4c5ccccc5c5cccc6oc7ccc2c(c7c65)c43)nc1 ; c1ccc(-c2ccc(-c3ccc(-n4c5cccc6c7ccccc7c7cccc8oc9ccc4c(c9c87)c65)nc3)cc2)cc1 ; c1ccc(-c2cc(-c3ccccc3)nc(-n3c4cccc5c6ccccc6c6cccc7oc8ccc3c(c8c76)c54)n2)cc1]

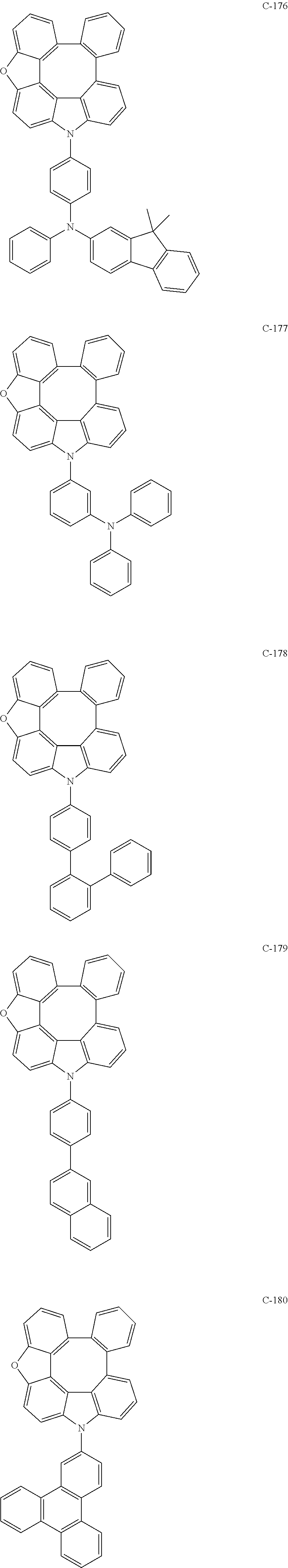 [SMILES: CC1(C)c2ccccc2-c2ccc(N(c3ccccc3)c3ccc(-n4c5cccc6c7ccccc7c7cccc8oc9ccc4c(c9c87)c65)cc3)cc21 ; c1ccc(N(c2ccccc2)c2cccc(-n3c4cccc5c6ccccc6c6cccc7oc8ccc3c(c8c76)c54)c2)cc1 ; c1ccc(-c2ccccc2-c2ccc(-n3c4cccc5c6ccccc6c6cccc7oc8ccc3c(c8c76)c54)cc2)cc1 ; c1ccc2cc(-c3ccc(-n4c5cccc6c7ccccc7c7cccc8oc9ccc4c(c9c87)c65)cc3)ccc2c1 ; c1ccc2c(c1)c1ccccc1c1cc(-n3c4cccc5c6ccccc6c6cccc7oc8ccc3c(c8c76)c54)ccc21]

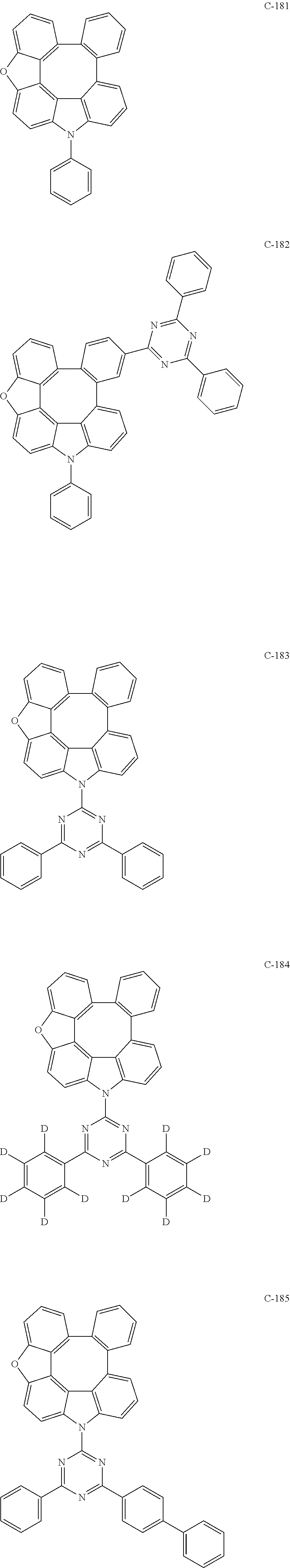 [SMILES: c1ccc(-n2c3cccc4c5ccccc5c5cccc6oc7ccc2c(c7c65)c43)cc1 ; c1ccc(-c2nc(-c3ccccc3)nc(-c3ccc4c(c3)c3cccc5c3c3c6c(ccc3n5-c3ccccc3)oc3cccc4c36)n2)cc1 ; c1ccc(-c2nc(-c3ccccc3)nc(-n3c4cccc5c6ccccc6c6cccc7oc8ccc3c(c8c76)c54)n2)cc1 ; [2H]c1c([2H])c([2H])c(-c2nc(-c3c([2H])c([2H])c([2H])c([2H])c3[2H])nc(-n3c4cccc5c6ccccc6c6cccc7oc8ccc3c(c8c76)c54)n2)c([2H])c1[2H] ; c1ccc(-c2ccc(-c3nc(-c4ccccc4)nc(-n4c5cccc6c7ccccc7c7cccc8oc9ccc4c(c9c87)c65)n3)cc2)cc1]

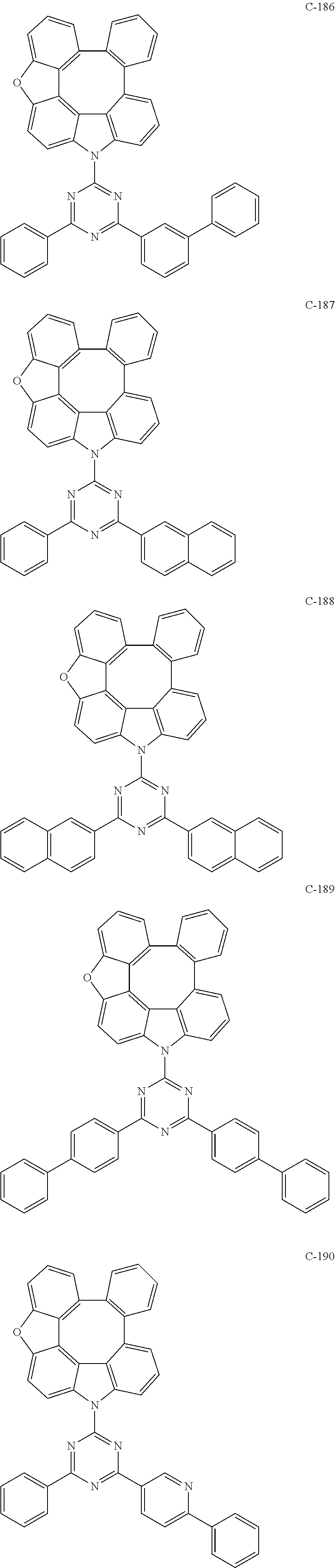 [SMILES: c1ccc(-c2cccc(-c3nc(-c4ccccc4)nc(-n4c5cccc6c7ccccc7c7cccc8oc9ccc4c(c9c87)c65)n3)c2)cc1 ; c1ccc(-c2nc(-c3ccc4ccccc4c3)nc(-n3c4cccc5c6ccccc6c6cccc7oc8ccc3c(c8c76)c54)n2)cc1 ; c1ccc2cc(-c3nc(-c4ccc5ccccc5c4)nc(-n4c5cccc6c7ccccc7c7cccc8oc9ccc4c(c9c87)c65)n3)ccc2c1 ; c1ccc(-c2ccc(-c3nc(-c4ccc(-c5ccccc5)cc4)nc(-n4c5cccc6c7ccccc7c7cccc8oc9ccc4c(c9c87)c65)n3)cc2)cc1 ; c1ccc(-c2ccc(-c3nc(-c4ccccc4)nc(-n4c5cccc6c7ccccc7c7cccc8oc9ccc4c(c9c87)c65)n3)cn2)cc1]

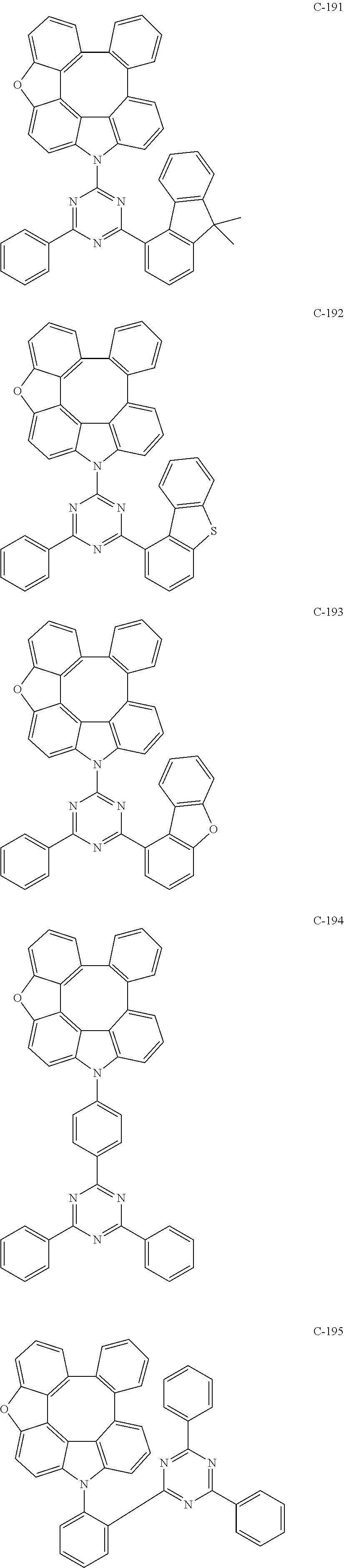

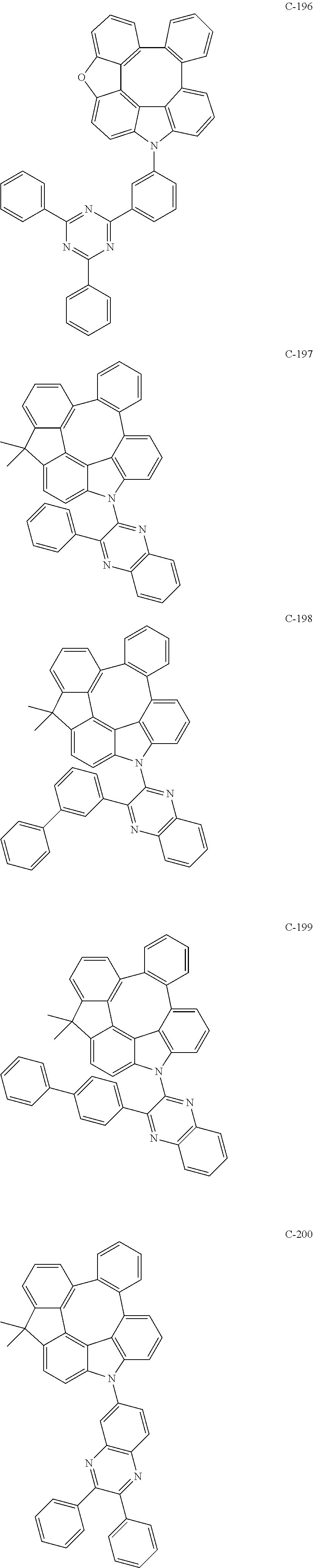

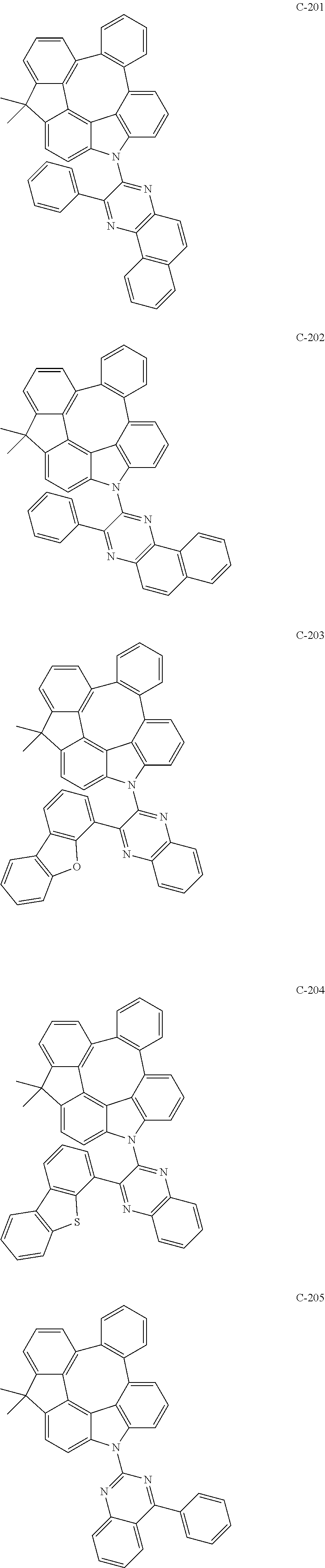 [SMILES: CC1(C)c2cccc3c4ccccc4c4cccc5c4c4c(c1ccc4n5-c1nc4ccc5ccccc5c4nc1-c1ccccc1)c23 ; CC1(C)c2cccc3c4ccccc4c4cccc5c4c4c(c1ccc4n5-c1nc4c(ccc5ccccc54)nc1-c1ccccc1)c23 ; CC1(C)c2cccc3c4ccccc4c4cccc5c4c4c(c1ccc4n5-c1nc4ccccc4nc1-c1cccc4c1oc1ccccc14)c23 ; CC1(C)c2cccc3c4ccccc4c4cccc5c4c4c(c1ccc4n5-c1nc4ccccc4nc1-c1cccc4c1sc1ccccc14)c23 ; CC1(C)c2cccc3c4ccccc4c4cccc5c4c4c(c1ccc4n5-c1nc(-c4ccccc4)c4ccccc4n1)c23]

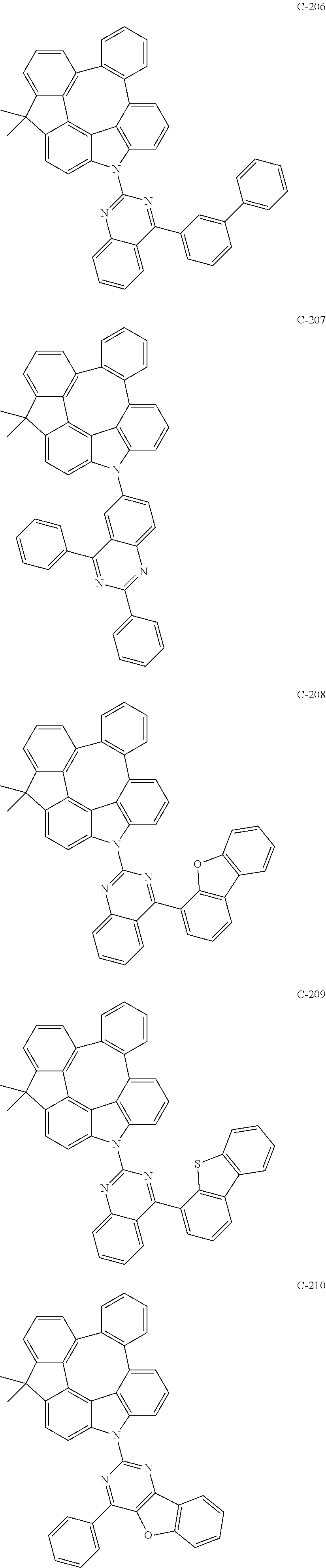

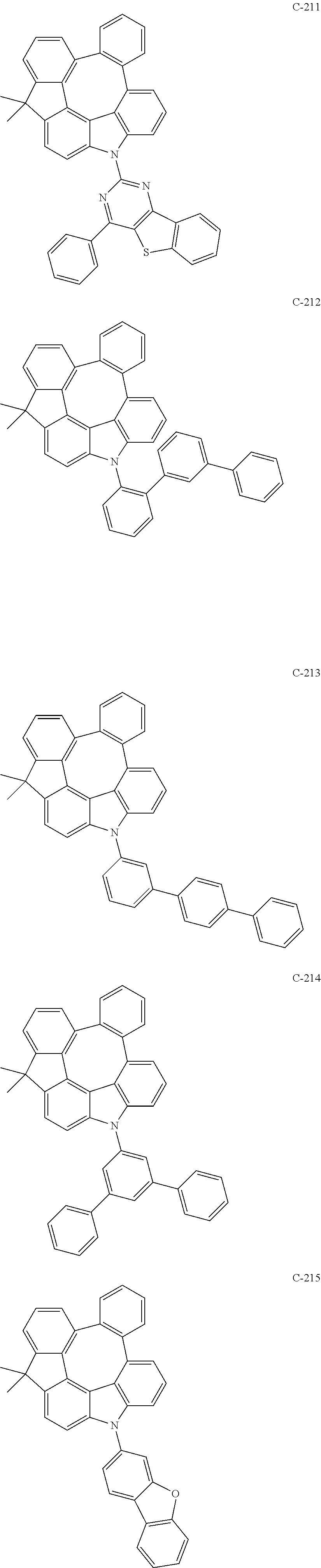 [SMILES: CC1(C)c2cccc3c4ccccc4c4cccc5c4c4c(c1ccc4n5-c1nc(-c4ccccc4)c4sc5ccccc5c4n1)c23 ; CC1(C)c2cccc3c4ccccc4c4cccc5c4c4c(c1ccc4n5-c1ccccc1-c1cccc(-c4ccccc4)c1)c23 ; CC1(C)c2cccc3c4ccccc4c4cccc5c4c4c(c1ccc4n5-c1cccc(-c4ccc(-c5ccccc5)cc4)c1)c23 ; CC1(C)c2cccc3c4ccccc4c4cccc5c4c4c(c1ccc4n5-c1cc(-c4ccccc4)cc(-c4ccccc4)c1)c23 ; CC1(C)c2cccc3c4ccccc4c4cccc5c4c4c(c1ccc4n5-c1ccc4c(c1)oc1ccccc14)c23]

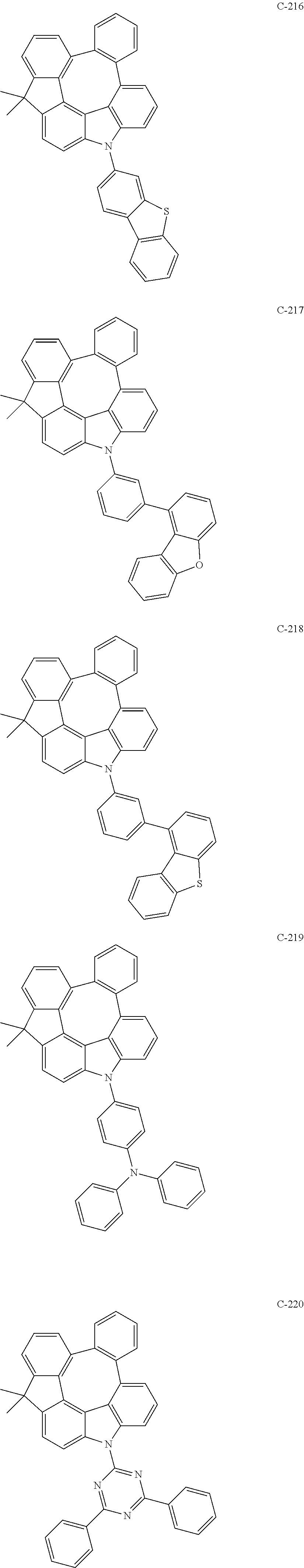 [SMILES: CC1(C)c2cccc3c4ccccc4c4cccc5c4c4c(c1ccc4n5-c1ccc4c(c1)sc1ccccc14)c23 ; CC1(C)c2cccc3c4ccccc4c4cccc5c4c4c(c1ccc4n5-c1cccc(-c4cccc5oc6ccccc6c45)c1)c23 ; CC1(C)c2cccc3c4ccccc4c4cccc5c4c4c(c1ccc4n5-c1cccc(-c4cccc5sc6ccccc6c45)c1)c23 ; CC1(C)c2cccc3c4ccccc4c4cccc5c4c4c(c1ccc4n5-c1ccc(N(c4ccccc4)c4ccccc4)cc1)c23 ; CC1(C)c2cccc3c4ccccc4c4cccc5c4c4c(c1ccc4n5-c1nc(-c4ccccc4)nc(-c4ccccc4)n1)c23]

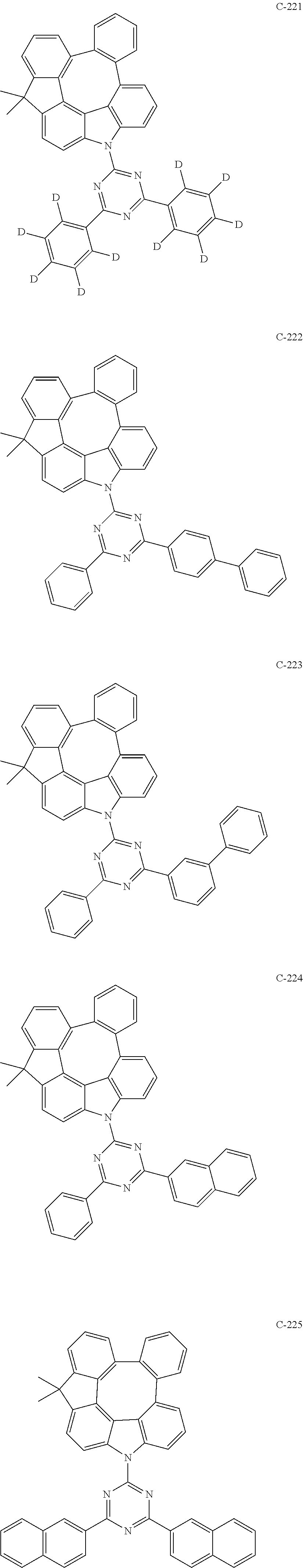 [SMILES: [2H]c1c([2H])c([2H])c(-c2nc(-c3c([2H])c([2H])c([2H])c([2H])c3[2H])nc(-n3c4cccc5c6ccccc6c6cccc7c6c6c(ccc3c6c54)C7(C)C)n2)c([2H])c1[2H] ; CC1(C)c2cccc3c4ccccc4c4cccc5c4c4c(c1ccc4n5-c1nc(-c4ccccc4)nc(-c4ccc(-c5ccccc5)cc4)n1)c23 ; CC1(C)c2cccc3c4ccccc4c4cccc5c4c4c(c1ccc4n5-c1nc(-c4ccccc4)nc(-c4cccc(-c5ccccc5)c4)n1)c23 ; CC1(C)c2cccc3c4ccccc4c4cccc5c4c4c(c1ccc4n5-c1nc(-c4ccccc4)nc(-c4ccc5ccccc5c4)n1)c23 ; CC1(C)c2cccc3c4ccccc4c4cccc5c4c4c(c1ccc4n5-c1nc(-c4ccc5ccccc5c4)nc(-c4ccc5ccccc5c4)n1)c23]

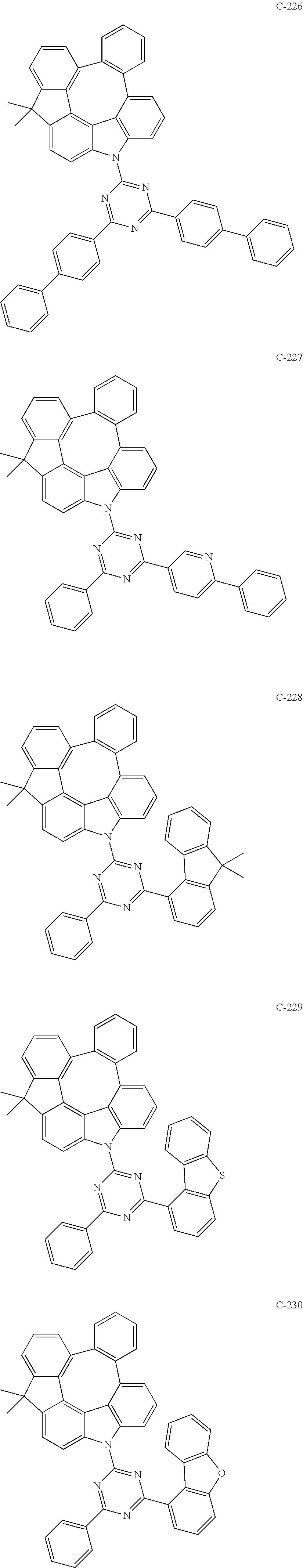

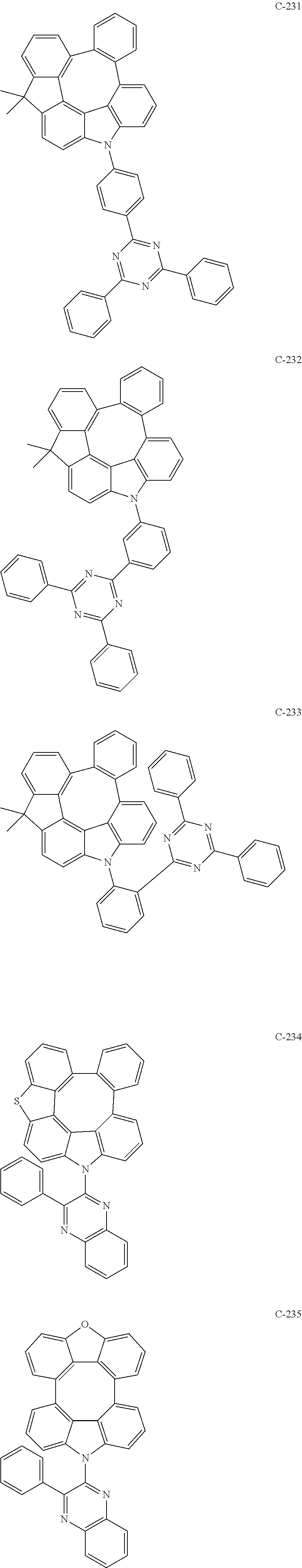 [SMILES: CC1(C)c2cccc3c4ccccc4c4cccc5c4c4c(c1ccc4n5-c1ccc(-c4nc(-c5ccccc5)nc(-c5ccccc5)n4)cc1)c23 ; CC1(C)c2cccc3c4ccccc4c4cccc5c4c4c(c1ccc4n5-c1cccc(-c4nc(-c5ccccc5)nc(-c5ccccc5)n4)c1)c23 ; CC1(C)c2cccc3c4ccccc4c4cccc5c4c4c(c1ccc4n5-c1ccccc1-c1nc(-c4ccccc4)nc(-c4ccccc4)n1)c23 ; c1ccc(-c2nc3ccccc3nc2-n2c3cccc4c5ccccc5c5cccc6sc7ccc2c(c7c65)c43)cc1 ; c1ccc(-c2nc3ccccc3nc2-n2c3cccc4c5cccc6oc7cccc(c8cccc2c8c43)c7c65)cc1]

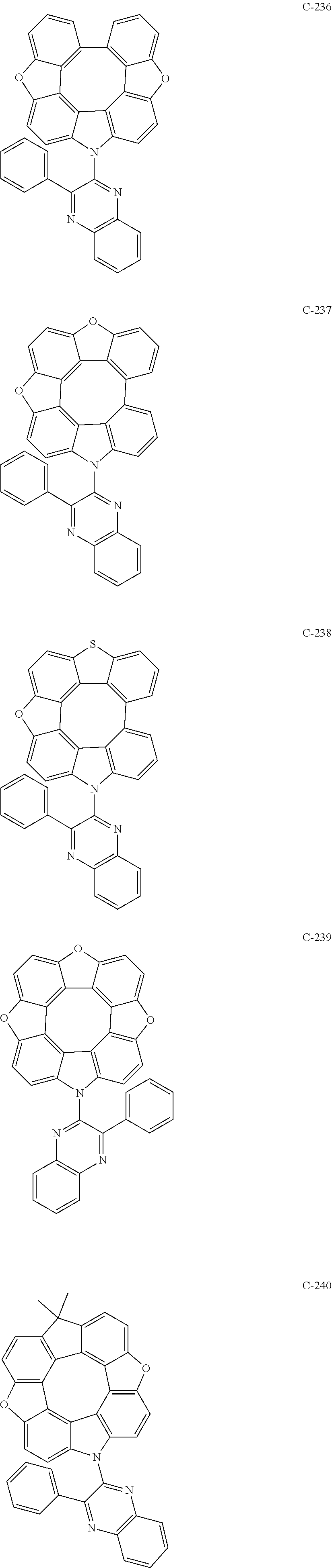 [SMILES: c1ccc(-c2nc3ccccc3nc2-n2c3ccc4oc5cccc6c7cccc8oc9ccc2c(c9c87)c3c4c56)cc1 ; c1ccc(-c2nc3ccccc3nc2-n2c3cccc4c5cccc6oc7ccc8oc9ccc2c(c9c8c7c65)c43)cc1 ; c1ccc(-c2nc3ccccc3nc2-n2c3cccc4c5cccc6sc7ccc8oc9ccc2c(c9c8c7c65)c43)cc1 ; c1ccc(-c2nc3ccccc3nc2-n2c3ccc4oc5ccc6oc7ccc8oc9ccc2c2c9c8c7c6c5c4c23)cc1 ; CC1(C)c2ccc3oc4ccc5c6c4c3c2c2c1ccc1oc3ccc(c6c3c12)n5-c1nc2ccccc2nc1-c1ccccc1]

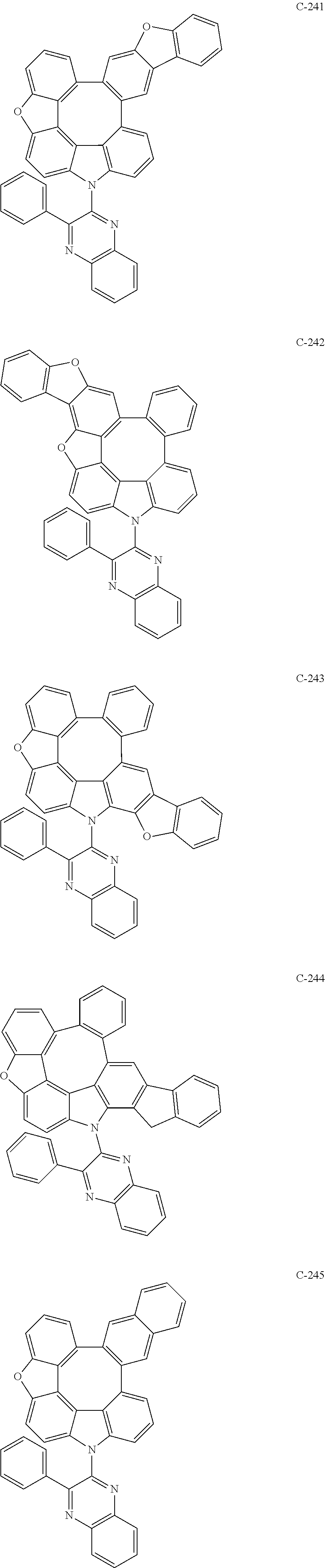 [SMILES: c1ccc(-c2nc3ccccc3nc2-n2c3cccc4c5cc6c(cc5c5cccc7oc8ccc2c(c8c75)c43)oc2ccccc26)cc1 ; c1ccc(-c2nc3ccccc3nc2-n2c3cccc4c5ccccc5c5cc6oc7ccccc7c6c6oc7ccc2c(c7c56)c43)cc1 ; c1ccc(-c2nc3ccccc3nc2-n2c3ccc4oc5cccc6c7ccccc7c7cc8c9ccccc9oc8c2c7c3c4c56)cc1 ; c1ccc(-c2nc3ccccc3nc2-n2c3ccc4oc5cccc6c7ccccc7c7cc8c(c2c7c3c4c56)Cc2ccccc2-8)cc1 ; c1ccc(-c2nc3ccccc3nc2-n2c3cccc4c5cc6ccccc6cc5c5cccc6oc7ccc2c(c7c65)c43)cc1]

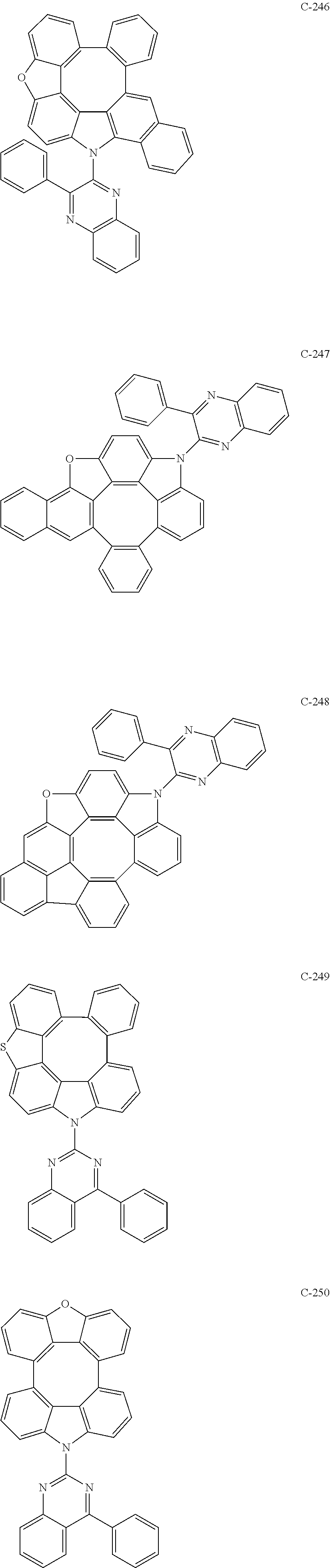 [SMILES: c1ccc(-c2nc3ccccc3nc2-n2c3ccc4oc5cccc6c7ccccc7c7cc8ccccc8c2c7c3c4c56)cc1 ; c1ccc(-c2nc3ccccc3nc2-n2c3cccc4c5ccccc5c5cc6ccccc6c6oc7ccc2c(c7c56)c43)cc1 ; c1ccc(-c2nc3ccccc3nc2-n2c3cccc4c5cccc6c5c5c7c-6cccc7cc6oc7ccc2c(c7c65)c43)cc1 ; c1ccc(-c2nc(-n3c4cccc5c6ccccc6c6cccc7sc8ccc3c(c8c76)c54)nc3ccccc23)cc1 ; c1ccc(-c2nc(-n3c4cccc5c6cccc7oc8cccc(c9cccc3c9c54)c8c76)nc3ccccc23)cc1]

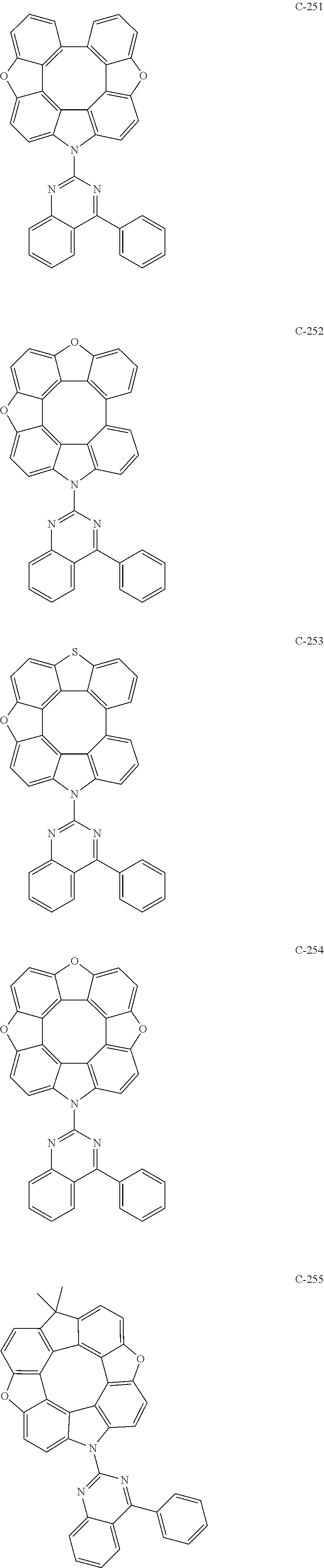 [SMILES: c1ccc(-c2nc(-n3c4ccc5oc6cccc7c8cccc9oc%10ccc3c(c%10c98)c4c5c67)nc3ccccc23)cc1 ; c1ccc(-c2nc(-n3c4cccc5c6cccc7oc8ccc9oc%10ccc3c(c%10c9c8c76)c54)nc3ccccc23)cc1 ; c1ccc(-c2nc(-n3c4cccc5c6cccc7sc8ccc9oc%10ccc3c(c%10c9c8c76)c54)nc3ccccc23)cc1 ; c1ccc(-c2nc(-n3c4ccc5oc6ccc7oc8ccc9oc%10ccc3c3c%10c9c8c7c6c5c34)nc3ccccc23)cc1 ; CC1(C)c2ccc3oc4ccc5c6c4c3c2c2c1ccc1oc3ccc(c6c3c12)n5-c1nc(-c2ccccc2)c2ccccc2n1]

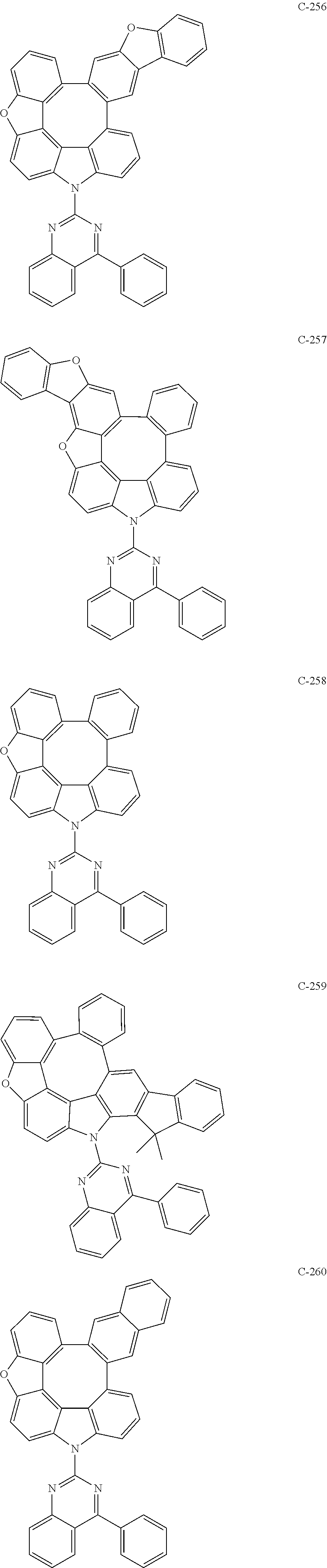 [SMILES: c1ccc(-c2nc(-n3c4cccc5c6cc7c(cc6c6cccc8oc9ccc3c(c9c86)c54)oc3ccccc37)nc3ccccc23)cc1 ; c1ccc(-c2nc(-n3c4cccc5c6ccccc6c6cc7oc8ccccc8c7c7oc8ccc3c(c8c67)c54)nc3ccccc23)cc1 ; c1ccc(-c2nc(-n3c4cccc5c6ccccc6c6cccc7oc8ccc3c(c8c76)c54)nc3ccccc23)cc1 ; CC1(C)c2ccccc2-c2cc3c4ccccc4c4cccc5oc6ccc7c(c6c54)c3c(c21)n7-c1nc(-c2ccccc2)c2ccccc2n1 ; c1ccc(-c2nc(-n3c4cccc5c6cc7ccccc7cc6c6cccc7oc8ccc3c(c8c76)c54)nc3ccccc23)cc1]

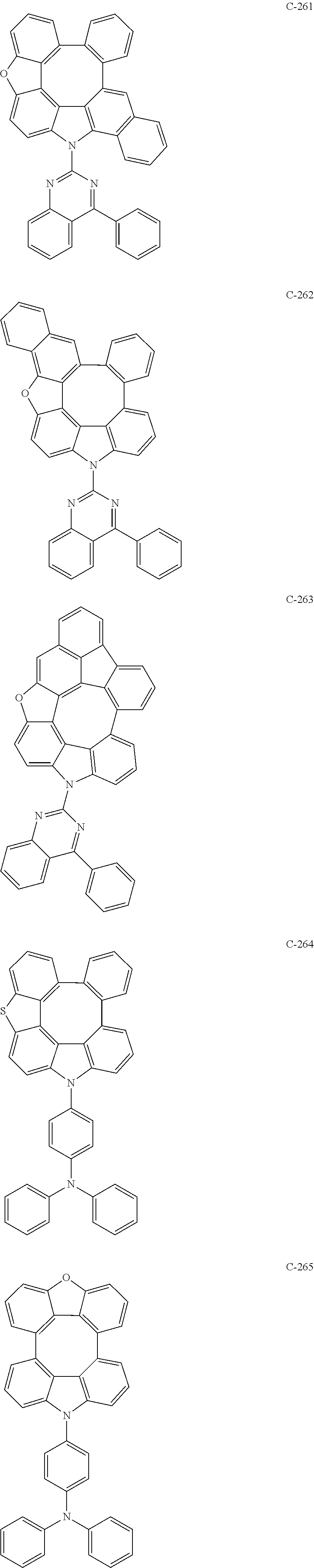

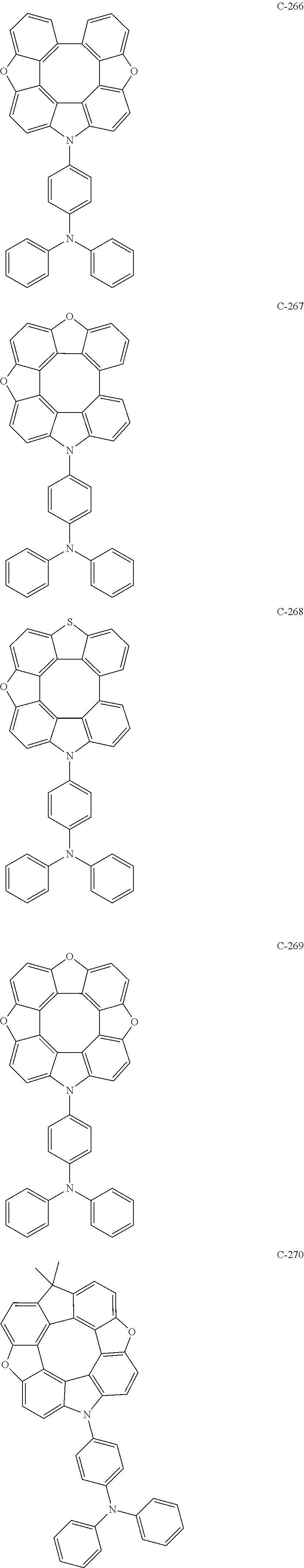

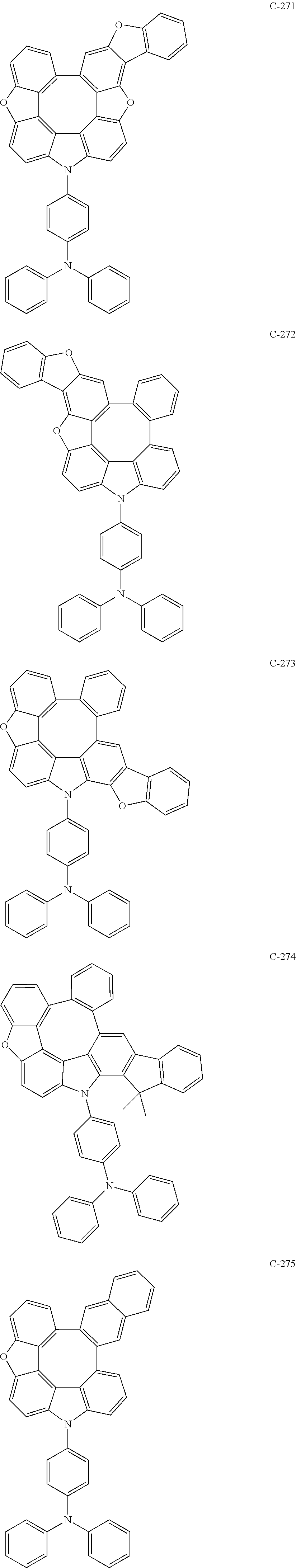

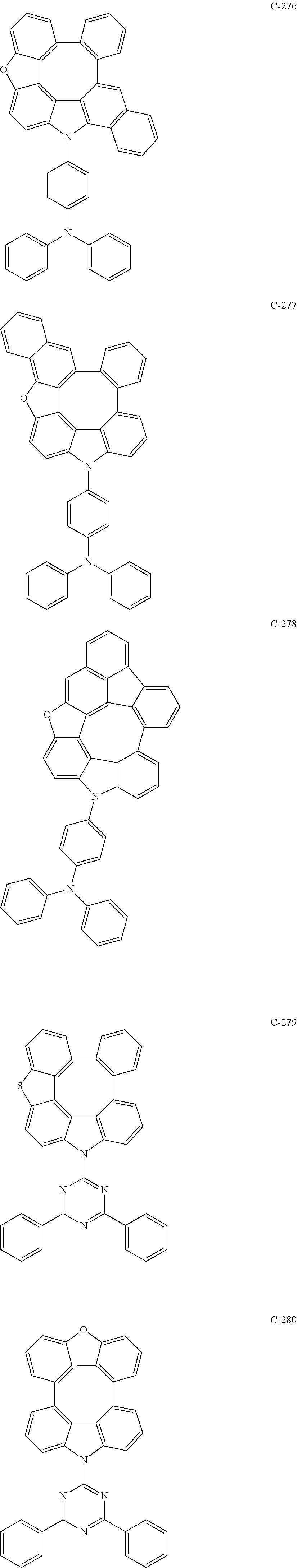

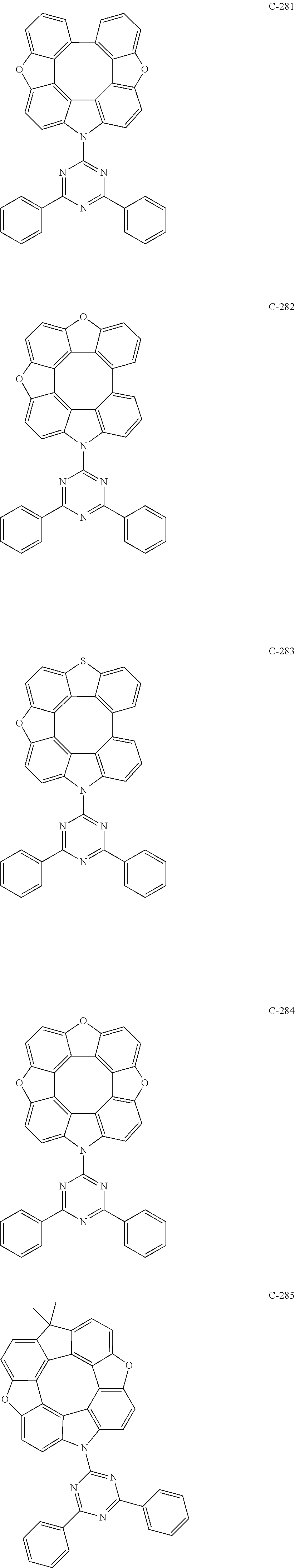 [SMILES: c1ccc(-c2nc(-c3ccccc3)nc(-n3c4ccc5oc6cccc7c8cccc9oc%10ccc3c(c%10c98)c4c5c67)n2)cc1 ; c1ccc(-c2nc(-c3ccccc3)nc(-n3c4cccc5c6cccc7oc8ccc9oc%10ccc3c(c%10c9c8c76)c54)n2)cc1 ; c1ccc(-c2nc(-c3ccccc3)nc(-n3c4cccc5c6cccc7sc8ccc9oc%10ccc3c(c%10c9c8c76)c54)n2)cc1 ; c1ccc(-c2nc(-c3ccccc3)nc(-n3c4ccc5oc6ccc7oc8ccc9oc%10ccc3c3c%10c9c8c7c6c5c34)n2)cc1 ; CC1(C)c2ccc3oc4ccc5c6c4c3c2c2c1ccc1oc3ccc(c6c3c12)n5-c1nc(-c2ccccc2)nc(-c2ccccc2)n1]

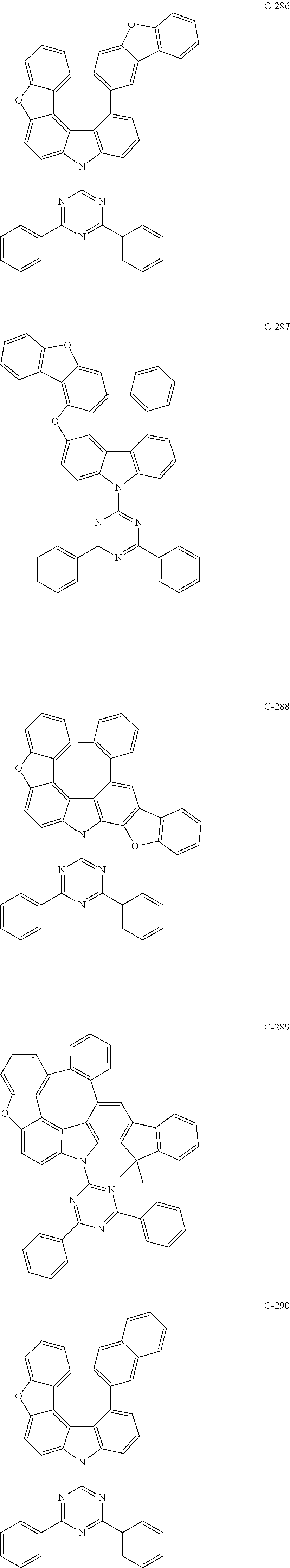 [SMILES: c1ccc(-c2nc(-c3ccccc3)nc(-n3c4cccc5c6cc7c(cc6c6cccc8oc9ccc3c(c9c86)c54)oc3ccccc37)n2)cc1 ; c1ccc(-c2nc(-c3ccccc3)nc(-n3c4cccc5c6ccccc6c6cc7oc8ccccc8c7c7oc8ccc3c(c8c67)c54)n2)cc1 ; c1ccc(-c2nc(-c3ccccc3)nc(-n3c4ccc5oc6cccc7c8ccccc8c8cc9c%10ccccc%10oc9c3c8c4c5c67)n2)cc1 ; CC1(C)c2ccccc2-c2cc3c4ccccc4c4cccc5oc6ccc7c(c6c54)c3c(c21)n7-c1nc(-c2ccccc2)nc(-c2ccccc2)n1 ; c1ccc(-c2nc(-c3ccccc3)nc(-n3c4cccc5c6cc7ccccc7cc6c6cccc7oc8ccc3c(c8c76)c54)n2)cc1]

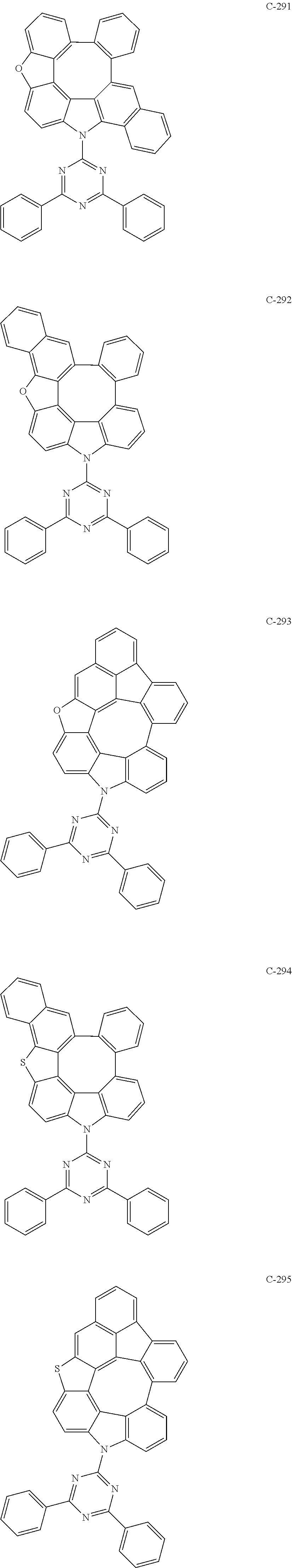 [SMILES: c1ccc(-c2nc(-c3ccccc3)nc(-n3c4ccc5oc6cccc7c8ccccc8c8cc9ccccc9c3c8c4c5c67)n2)cc1 ; c1ccc(-c2nc(-c3ccccc3)nc(-n3c4cccc5c6ccccc6c6cc7ccccc7c7oc8ccc3c(c8c67)c54)n2)cc1 ; c1ccc(-c2nc(-c3ccccc3)nc(-n3c4cccc5c6cccc7c6c6c8c-7cccc8cc7oc8ccc3c(c8c76)c54)n2)cc1 ; c1ccc(-c2nc(-c3ccccc3)nc(-n3c4cccc5c6ccccc6c6cc7ccccc7c7sc8ccc3c(c8c67)c54)n2)cc1 ; c1ccc(-c2nc(-c3ccccc3)nc(-n3c4cccc5c6cccc7c6c6c8c-7cccc8cc7sc8ccc3c(c8c76)c54)n2)cc1]

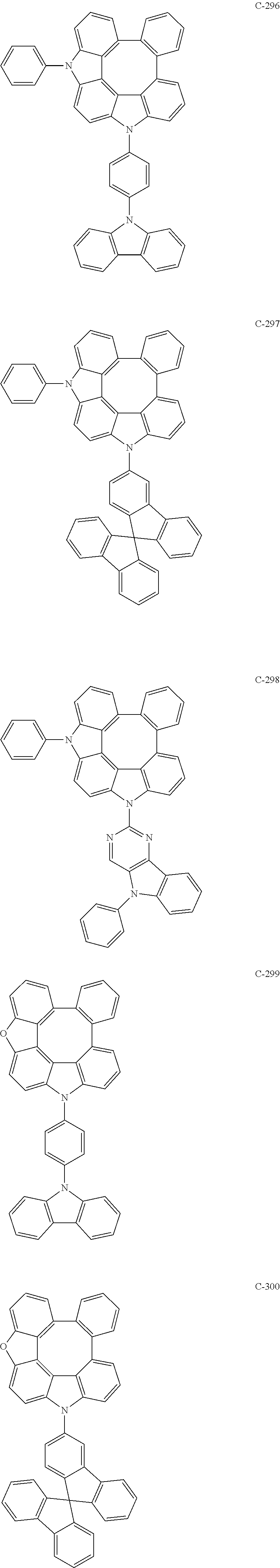 [SMILES: c1ccc(-n2c3cccc4c5ccccc5c5cccc6c5c5c(c43)c2ccc5n6-c2ccc(-n3c4ccccc4c4ccccc43)cc2)cc1 ; c1ccc(-n2c3cccc4c5ccccc5c5cccc6c5c5c(c43)c2ccc5n6-c2ccc3c(c2)-c2ccccc2C32c3ccccc3-c3ccccc32)cc1 ; c1ccc(-n2c3ccccc3c3nc(-n4c5cccc6c7ccccc7c7cccc8c7c7c(c65)c4ccc7n8-c4ccccc4)ncc32)cc1 ; c1ccc2c(c1)c1cccc3oc4ccc5c(c4c31)c1c2cccc1n5-c1ccc(-n2c3ccccc3c3ccccc32)cc1 ; c1ccc2c(c1)-c1ccccc1C21c2ccccc2-c2cc(-n3c4cccc5c6ccccc6c6cccc7oc8ccc3c(c8c76)c54)ccc21]

The compound represented by formula 2 according to the present disclosure may be prepared by a synthetic method known to a person skilled in the art. For example, the compound represented by formula 2 may be prepared by referring to the following reaction schemes 3 to 6, but is not limited thereto:

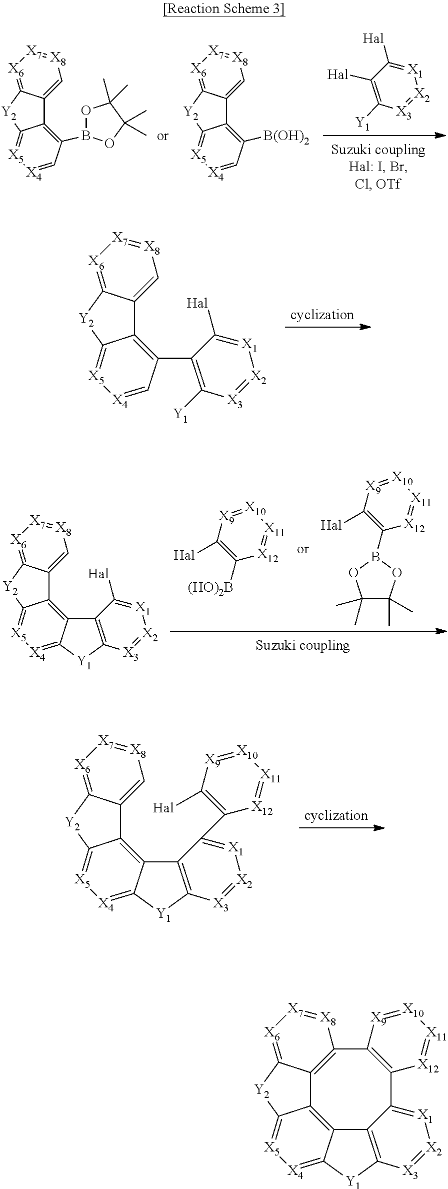

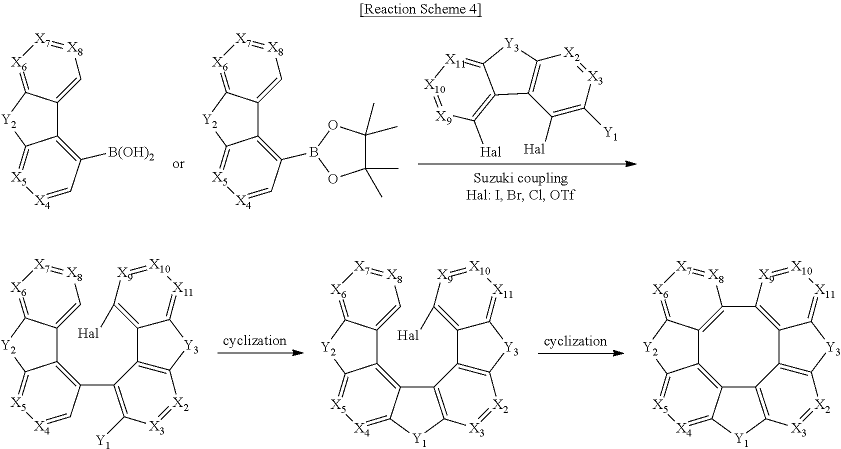

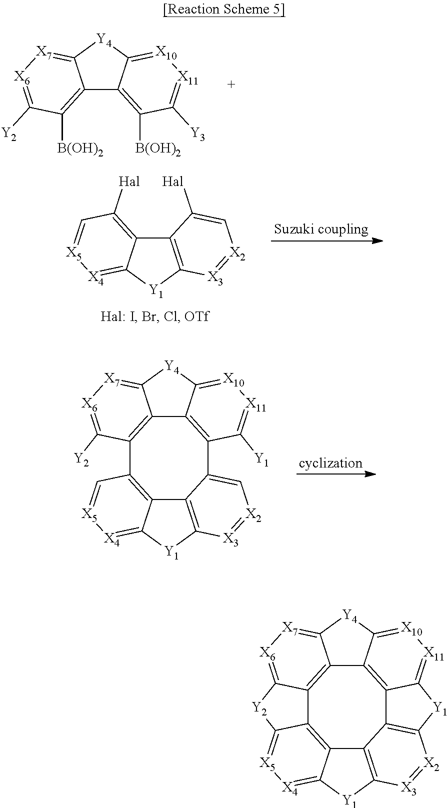

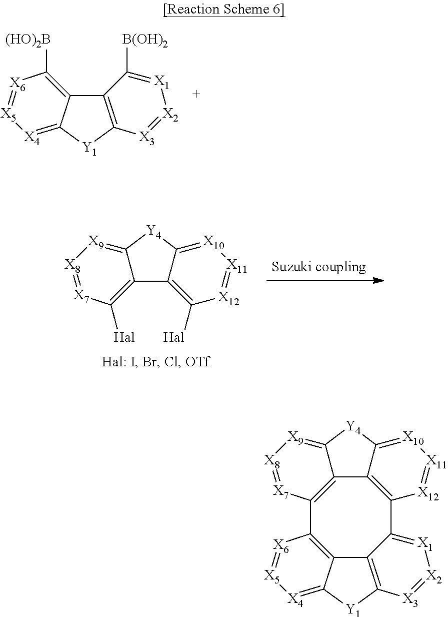

In the reaction schemes 3 to 6, the definition of the substituents is as defined in formulas 2-1 to 2-5.

As described above, exemplary synthesis examples of the compounds represented by formula 2 according to the present disclosure are described, but they are based on Buchwald-Hartwig cross coupling reaction, N-arylation reaction. H-mont-mediated etherification reaction, Miyaura borylation reaction, Suzuki cross-coupling reaction, Intramolecular acid-induced cyclization reaction, Pd(II)-catalyzed oxidative cyclization reaction, Grignard reaction, Heck reaction, Cyclic Dehydration reaction, SN₁ substitution reaction, SN₂ substitution reaction, and Phosphine-mediated reductive cyclization reaction, etc. It will be understood by one skilled in the art that the above reaction proceeds even if other substituents defined in formulas 2-1 to 2-5 other than the substituents described in the specific synthesis examples are bonded.

Hereinafter, an organic electroluminescent device to which the aforementioned plurality of host materials and/or the organic electroluminescent material comprising the same is applied, will be described.

The organic electroluminescent device according to one embodiment includes a first electrode; a second electrode; and at least one organic layer interposed between the first electrode and the second electrode. The organic layer may include a light-emitting layer, and the light-emitting layer may comprise a plurality of host materials comprising at least one first host material represented by the above formula 1 and at least one second host material represented by the above formula 2.

According to one embodiment, the organic electroluminescent material of the present disclosure comprises at least one compound(s) of compounds H1-1 to H1-124 as the first host material represented by the above formula 1 and at least one compound(s) of compounds C-1 to C-300 as the second host material represented by the above formula 2, and the plurality of host materials may be included in the same organic layer or may be included in different organic layers, respectively.

The light-emitting layer is a layer from which light is emitted, and can be a single layer or a multi-layer of which two or more layers are stacked. In the light-emitting layer, it is preferable that the doping concentration of the dopant compound based on the host compound may be less than 20 wt %, preferably, 17 wt %.

The organic layer may further comprise at least one layer(s) selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer, in addition to the light-emitting layer.

The organic layer may further comprise an amine-based compound and/or an azine-based compound, in addition to the light-emitting material of the present disclosure.

Specifically, the hole injection layer, the hole transport layer, the hole auxiliary layer, the light-emitting layer, the light-emitting auxiliary layer, or the electron blocking layer may comprise an amine-based compound, for example, arylamine-based compound, a styrylarylamine-based compound, etc., as a hole injection material, a hole transport material, a hole auxiliary material, a light-emitting material, a light-emitting auxiliary material, or an electron blocking material. In addition, the electron transport layer, the electron injection layer, the electron buffer layer, and the hole blocking layer may comprise an azine-based compound as an electron transport material, an electron injection material, an electron buffer material, and a hole blocking material.

In addition, the organic layer further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

An organic electroluminescent material according to one embodiment may be used as light-emitting materials for a white organic light-emitting device. The white organic light-emitting device has suggested various structures such as a parallel side-by-side arrangement method, a stacking arrangement method, or color conversion material (CCM) method, etc., according to the arrangement of R (Red), G (Green), YG (yellowish green), or B (Blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both electrode(s). Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

Further, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

The organic electroluminescent device according to one embodiment may further include at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), as necessary; more preferably an ortho-metallated complex compound(s) of a metal atom(s) selected from iridium (Ir), osmium (Os), copper (Cu), and platinum (Pt), as necessary; and even more preferably ortho-metallated iridium complex compound(s), as necessary.

The dopant comprised in the organic electroluminescent device of the present disclosure may use the compound represented by the following formula 101, but is not limited thereto:

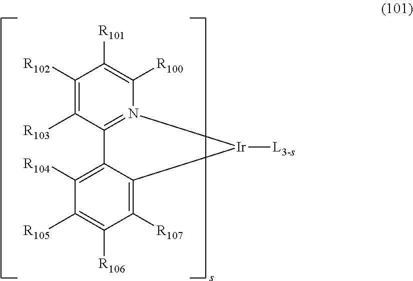

In formula 101,

L is selected from the following structure 1 or 2;

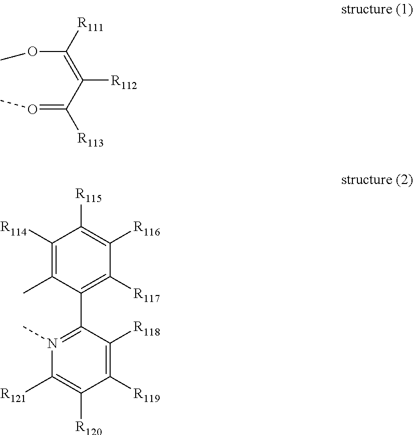

in structures 1 and 2,

R₁₀₀ to R₁₀₃ each independently represent hydrogen, deuterium, halogen, (C1-C30)alkyl unsubstituted or substituted with halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or R₁₀₀ to R₁₀₃ may be linked to the adjacent substituent to form a substituted or unsubstituted fused ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;

R₁₀₄ to R₁₀₇ each independently represent hydrogen, deuterium, halogen, substituted or unsubstituted (C1-C30)alkyl unsubstituted or substituted with halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or R₁₀₄ to R₁₀₇ may be linked to the adjacent substituent to form a substituted or unsubstituted fused ring(s), e.g., a substituted or unsubstituted naphthyl, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;

R₁₁₁ to R₁₂, each independently represent hydrogen, deuterium, halogen, (C1-C30)alkyl unsubstituted or substituted with halogen, a substituted or unsubstituted (C3-C30)cycloalkyl, or a substituted or unsubstituted (C6-C30)aryl; or may be linked to the adjacent substituent to form a substituted or unsubstituted fused ring(s); and

s represents an integer of 1 to 3.

Specifically, the specific examples of the dopant compound include the following, but are not limited thereto.

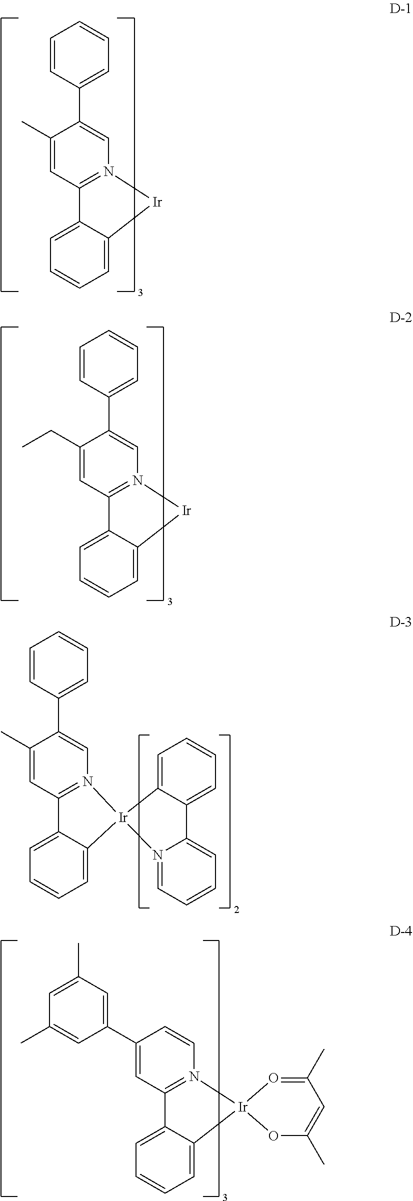

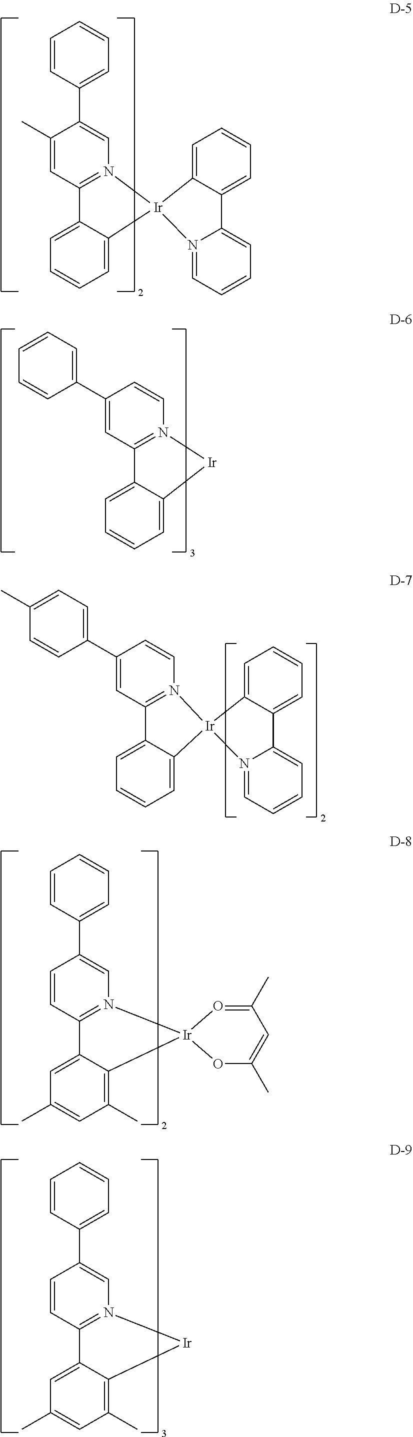

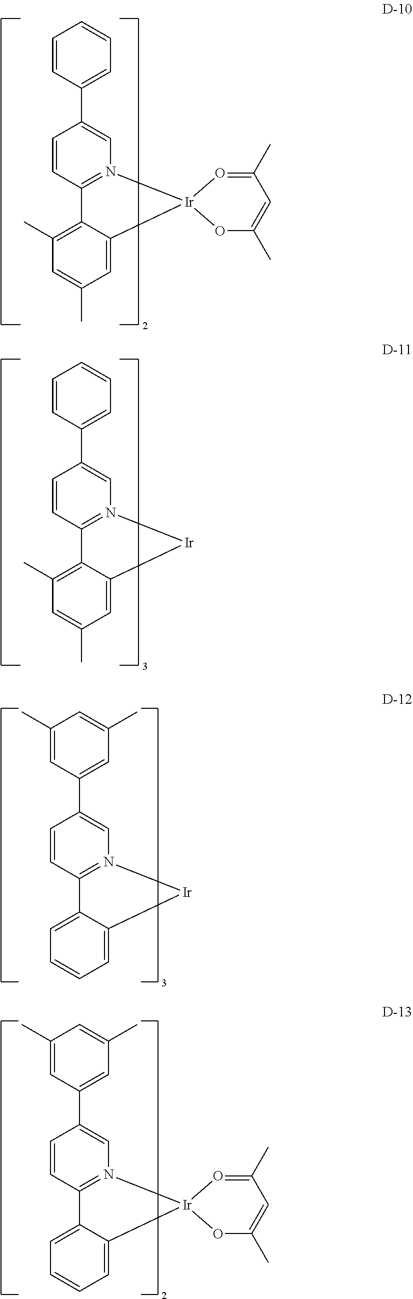

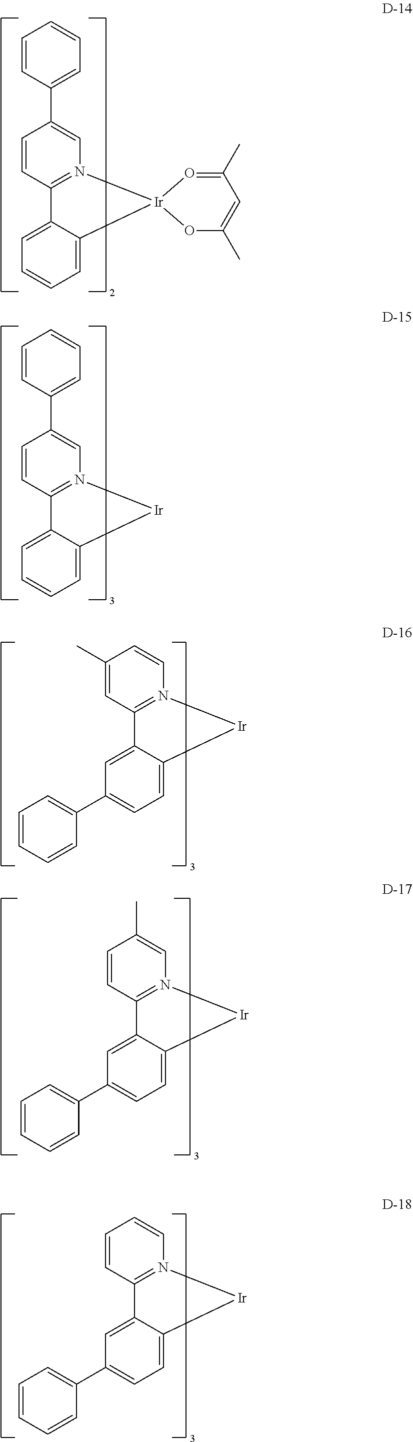

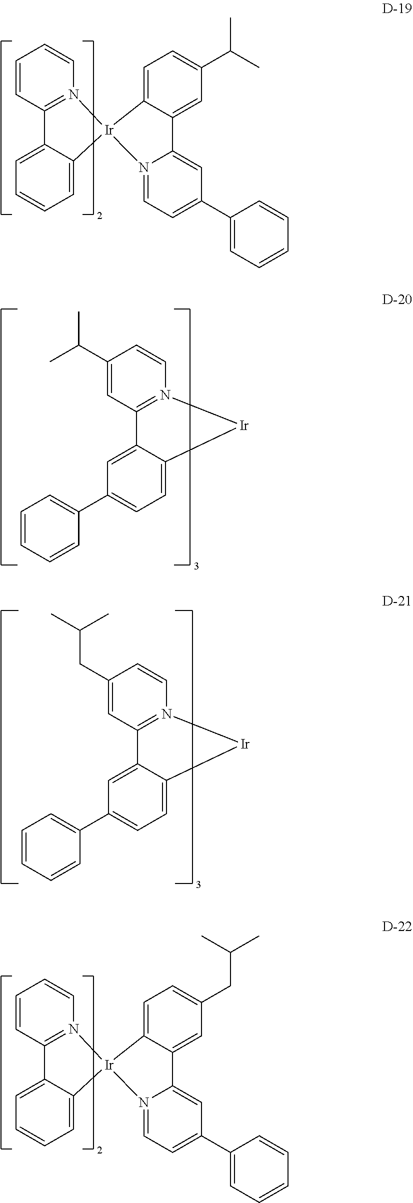

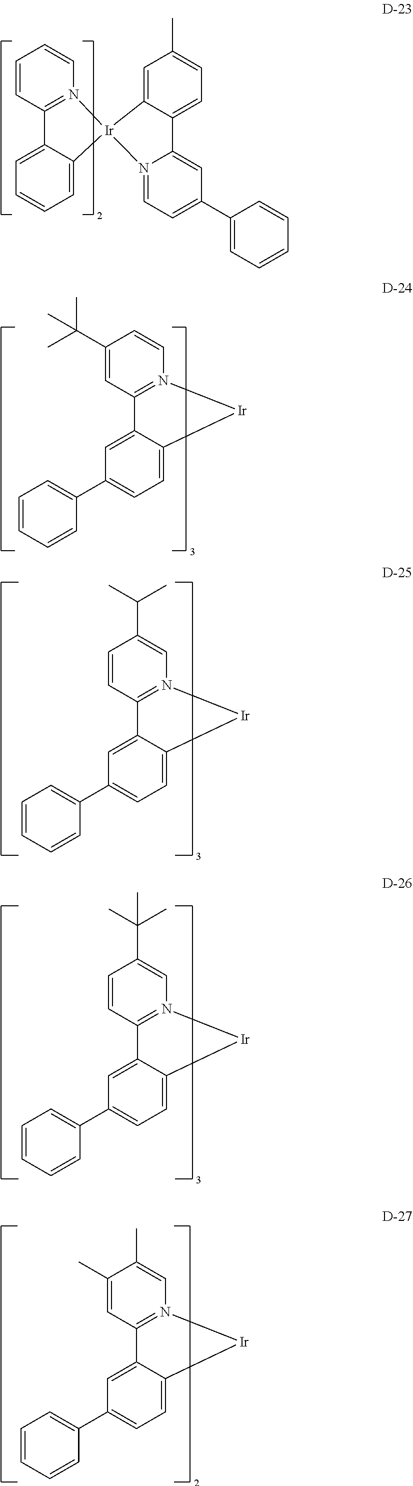

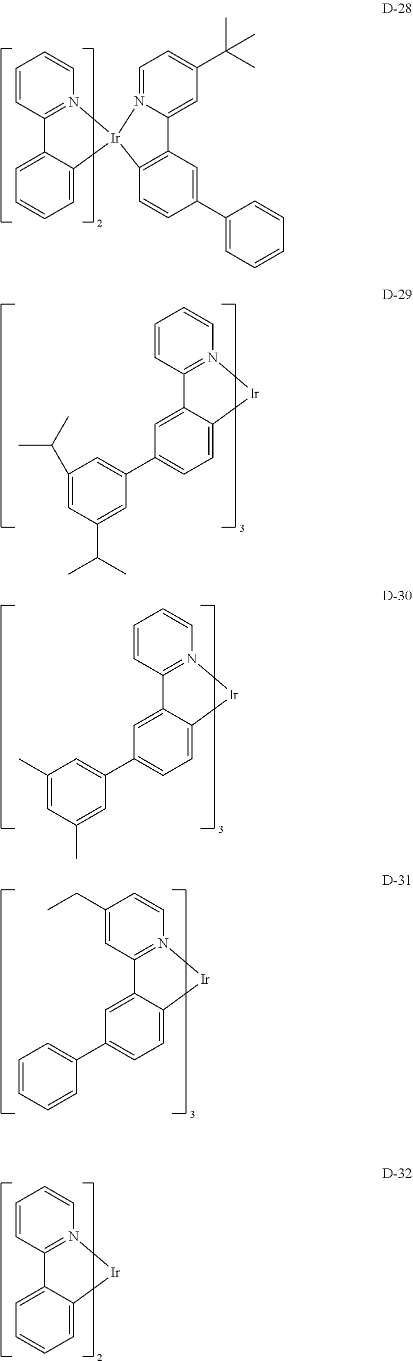

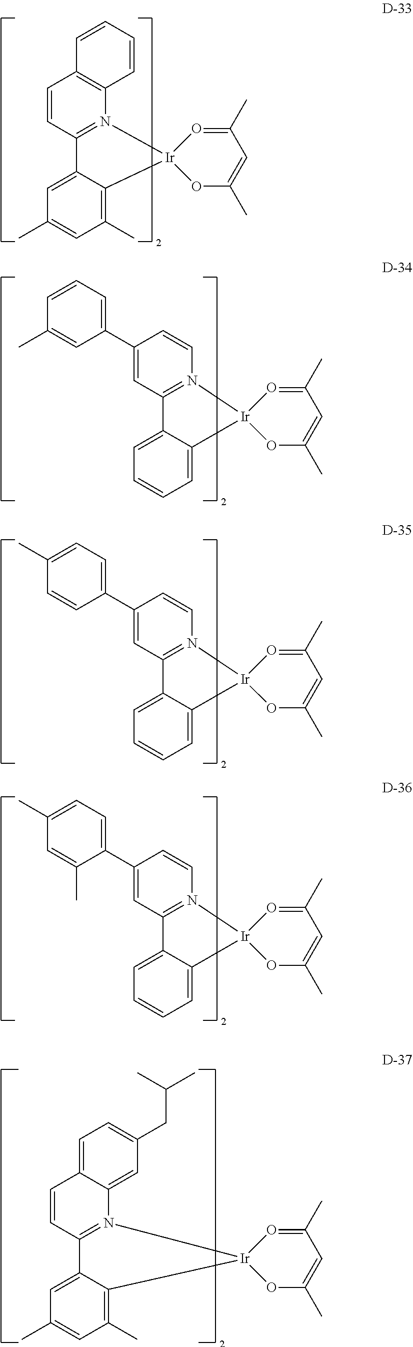

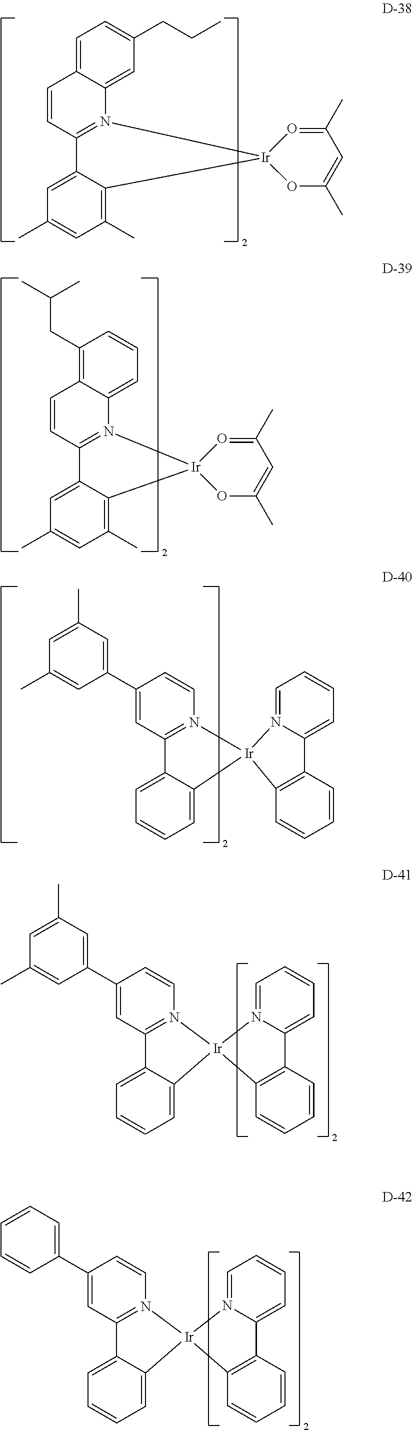

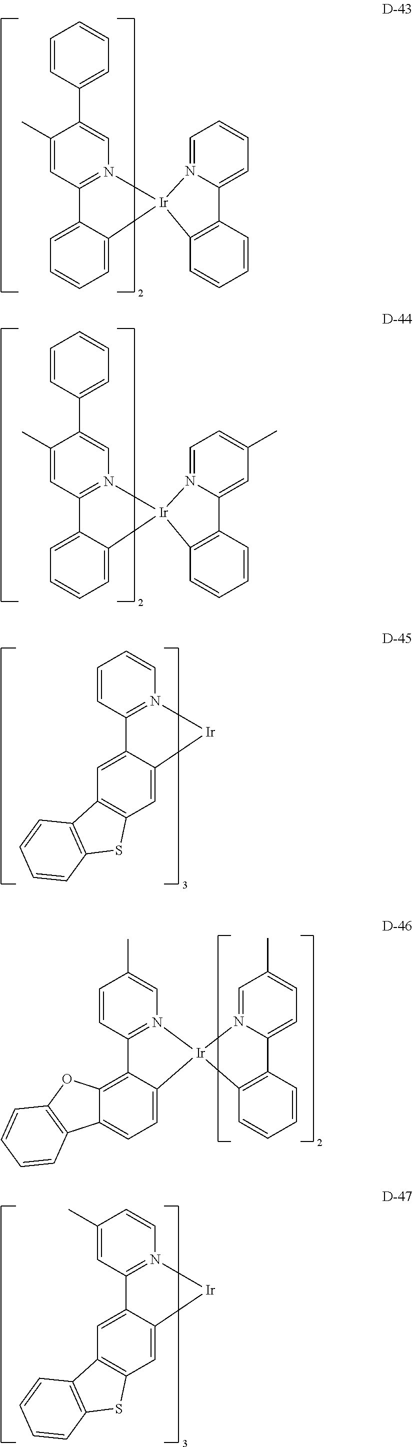

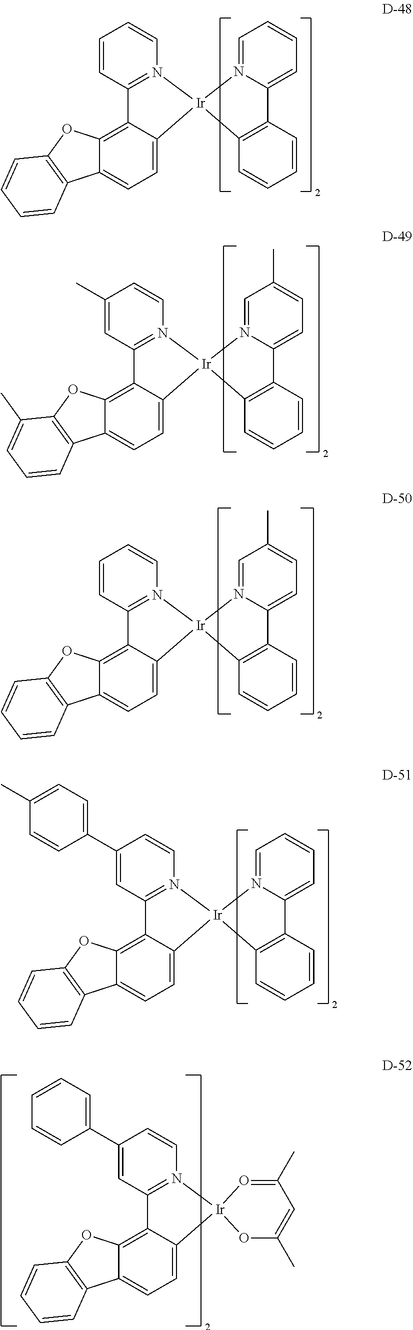

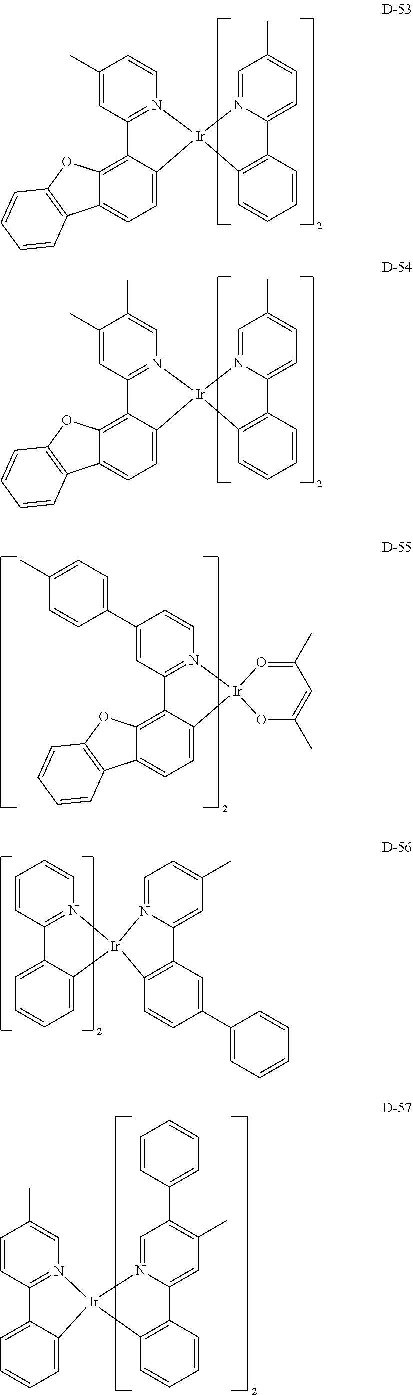

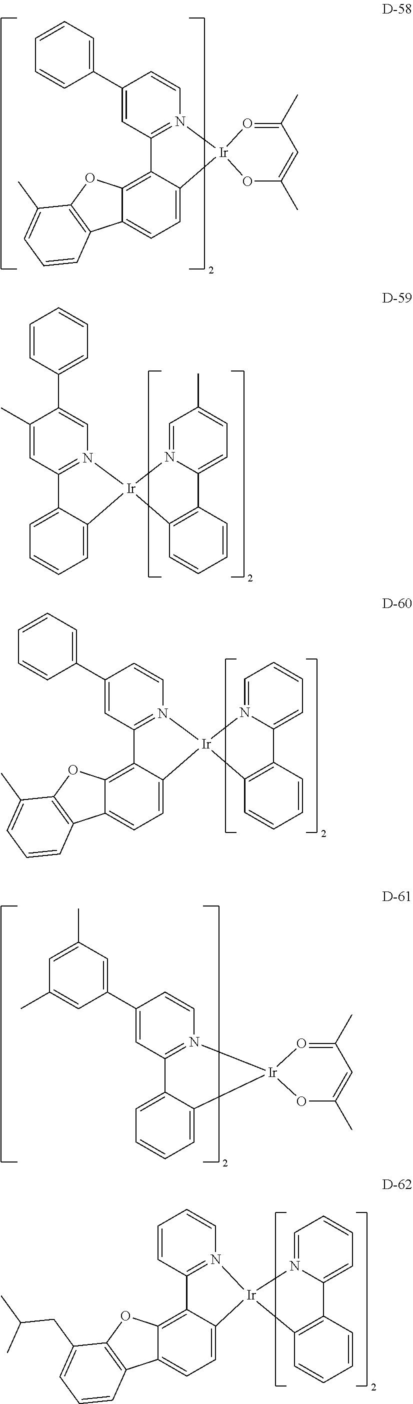

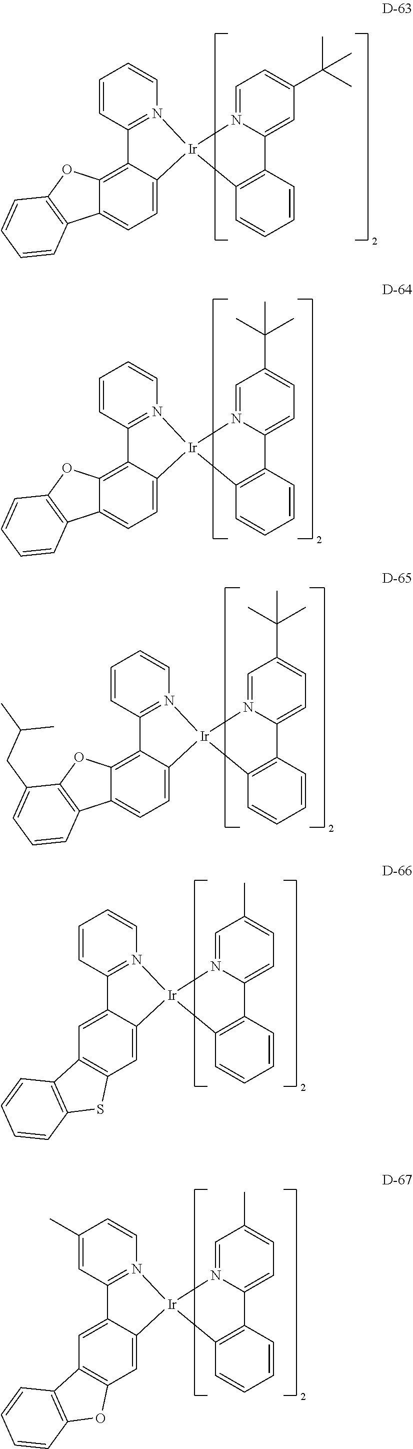

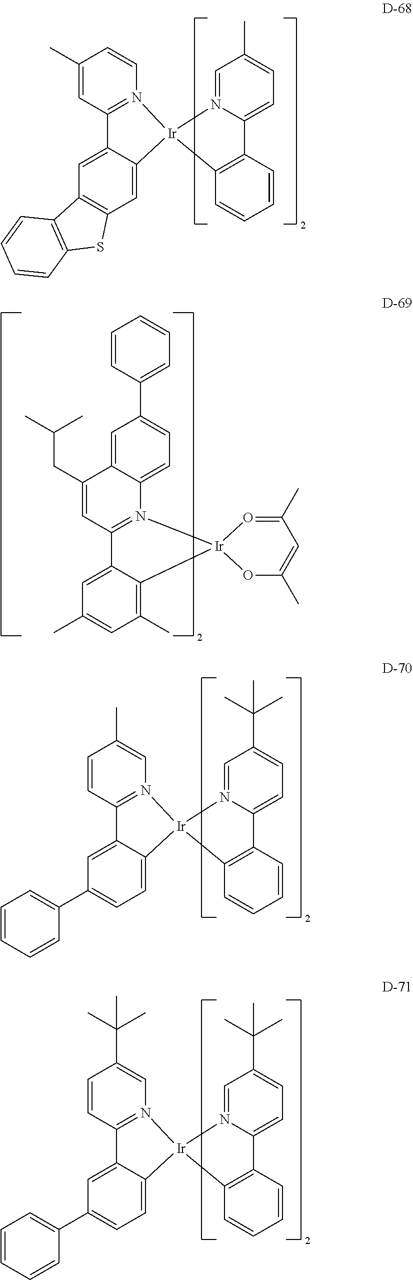

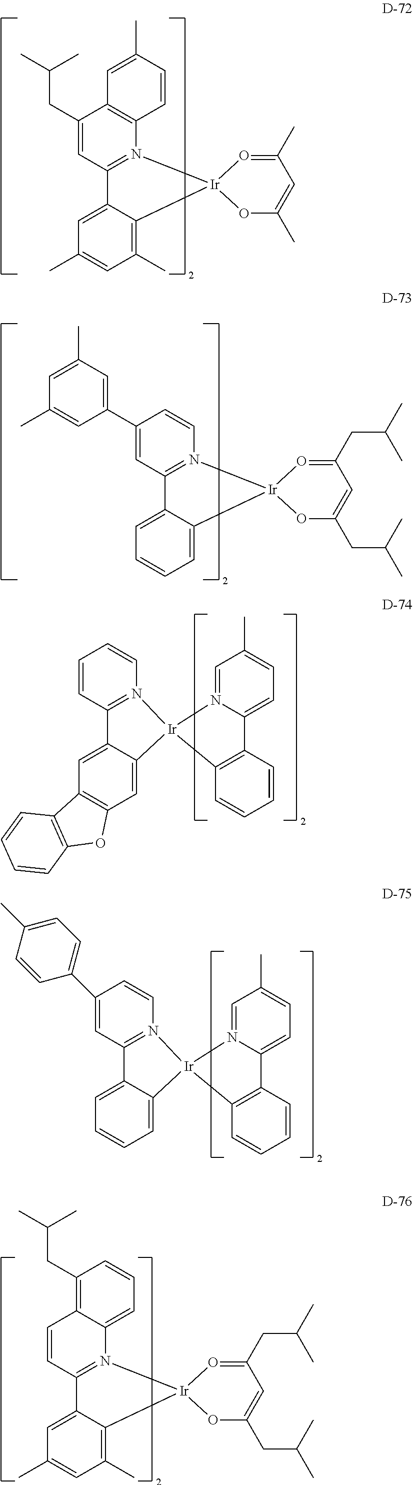

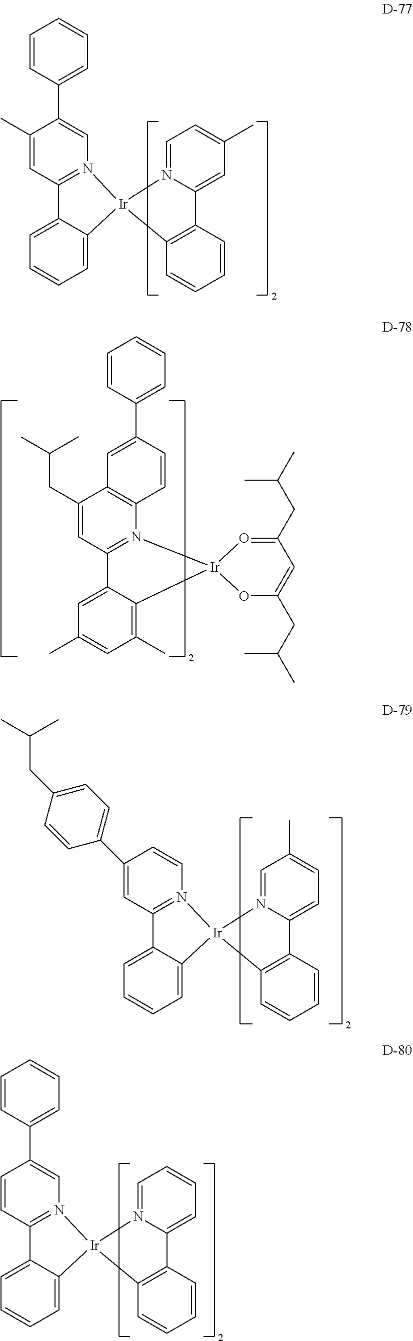

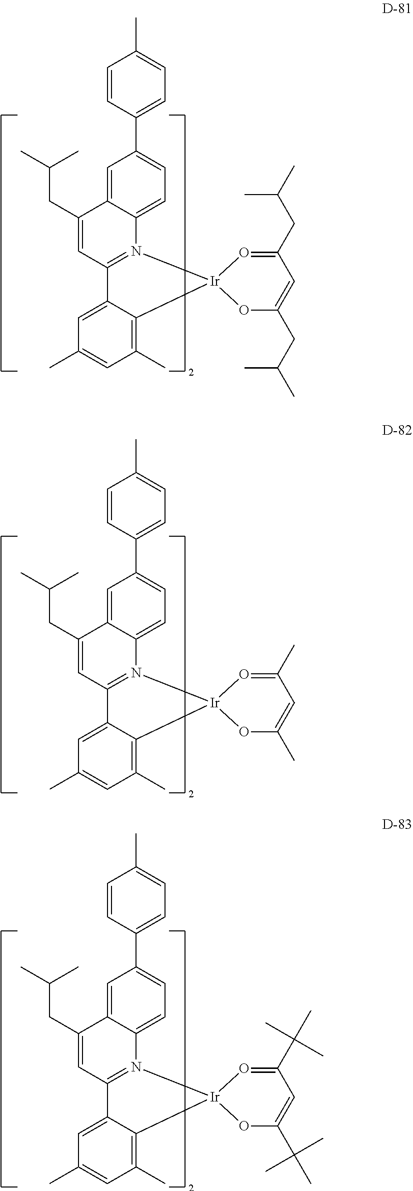

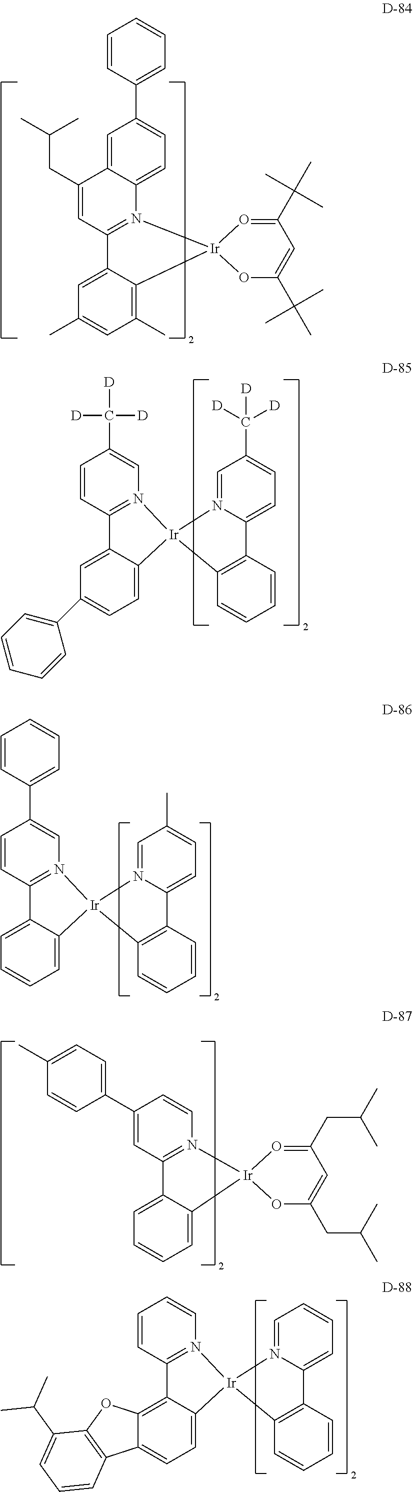

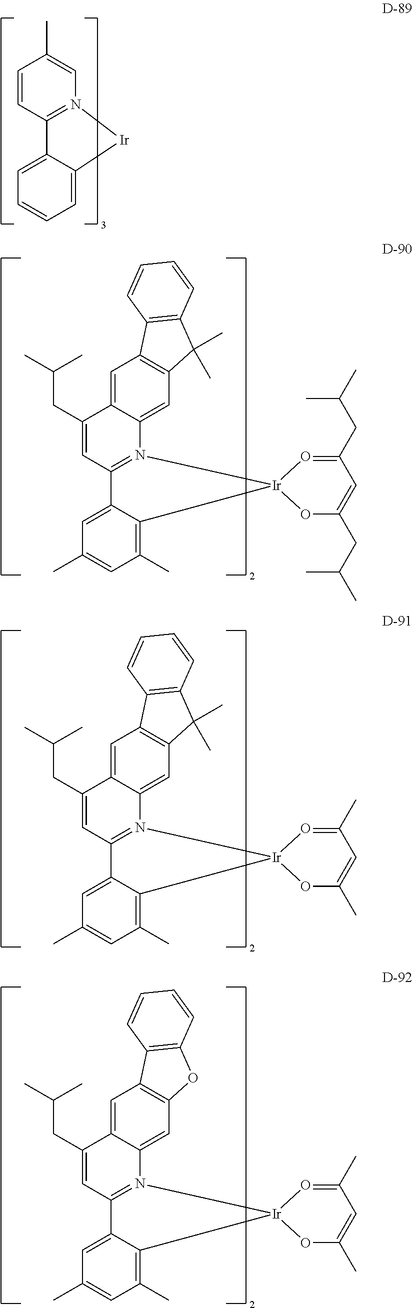

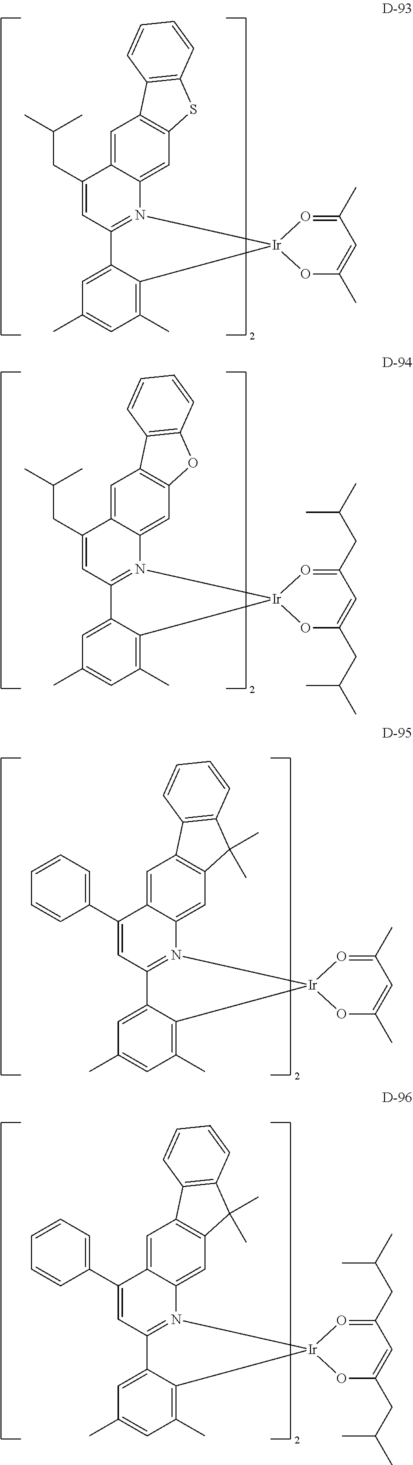

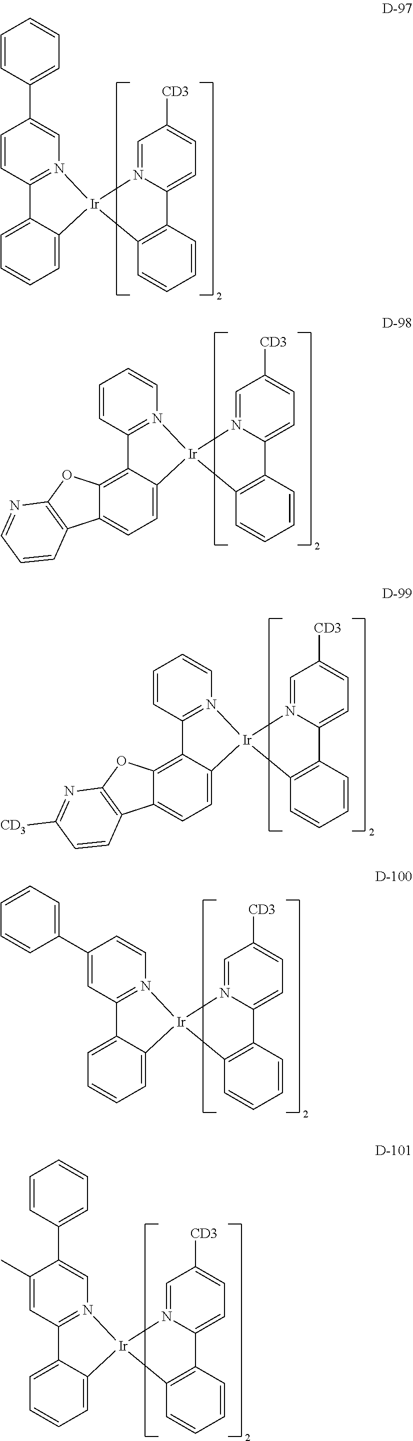

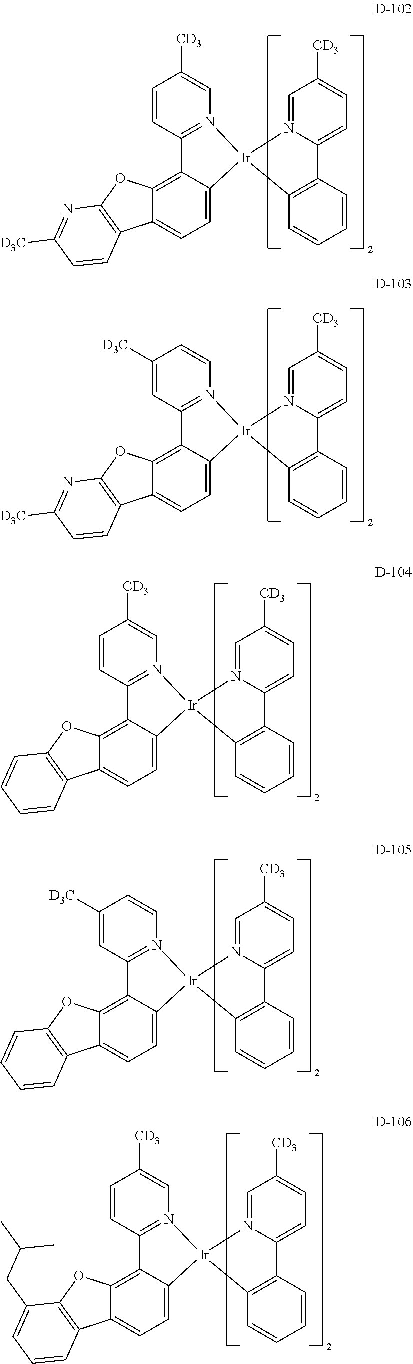

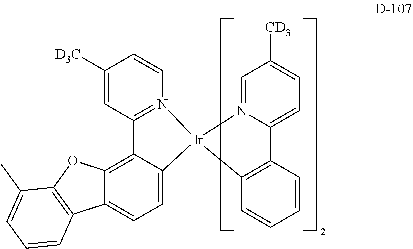

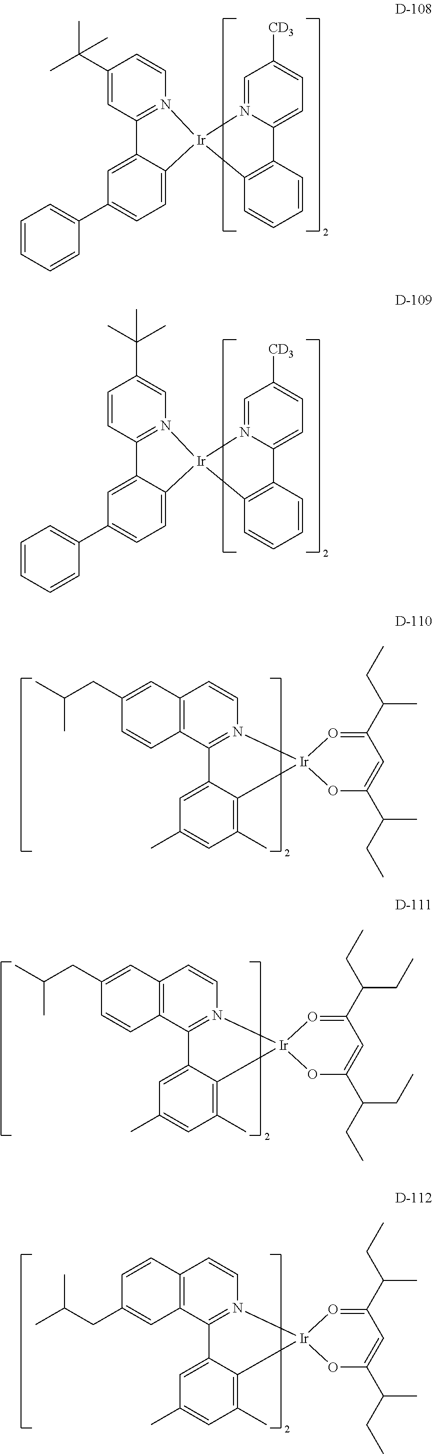

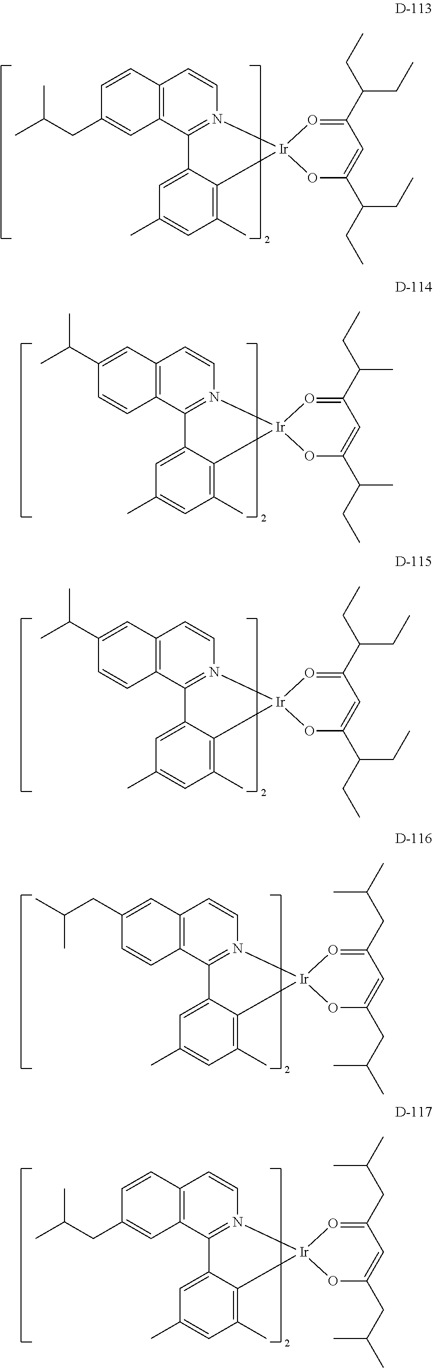

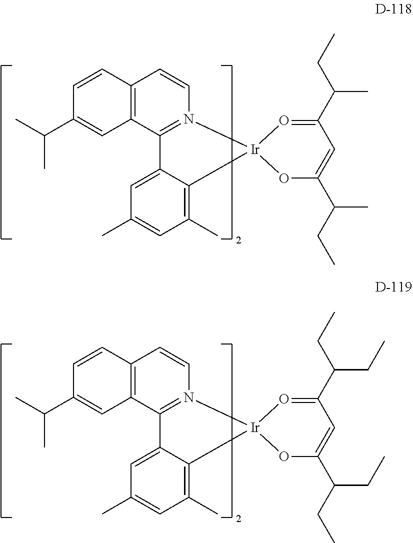

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host material and the second host material according to one embodiment, the layer can be formed by the above-listed methods, and can often be formed by co-deposition or mixture-deposition. The co-deposition is a mixed deposition method in which two or more materials are put into respective individual crucible sources and a current is applied to both cells simultaneously to evaporate the materials and to perform mixed deposition; and the mixed deposition is a mixed deposition method in which two or more materials are mixed in one crucible source before deposition, and then a current is applied to one cell to evaporate the materials.

According to one embodiment, when the first host material and the second host material exist in the same layer or different layers in the organic electroluminescent device, the layers by the two host compounds may be separately formed. For example, after depositing the first host material, a second host material may be deposited.

According to one embodiment, the present disclosure can provide display devices comprising a plurality of host materials including a first host material represented by formula 1 and a second host material represented by formula 2. In addition, it is possible to manufacture a display device or a lighting device using the organic electroluminescent device of the present disclosure. Specifically, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of compounds according to the present disclosure, and the properties thereof will be explained with reference to a representative compound in order to understand the present disclosure in detail.

### [Example 1] Preparation of Compound C-1

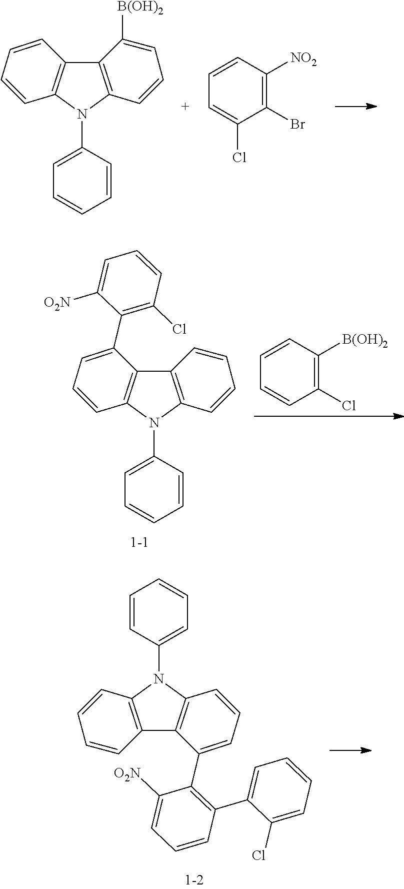

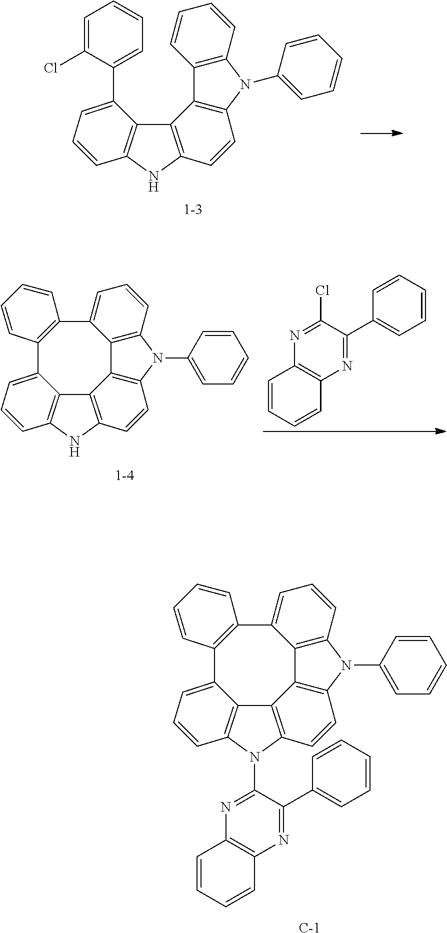

1) Synthesis of Compound 1-1

(9-phenyl-9H-carbazol-4-yl)boronic acid (96 g, 334.3 mmol), 2-bromo-1-chloro-3-nitorobenzene (71.8 g, 304 mmol), Pd₂(dba)₃ (15 g, 16.71 mmol), S-Phos (10.9 g, 26.76 mmol), and KsPO₄ (315 g, 1.64 mol) in a flask were dissolved in 1,500 mL of toluene, and then stirred at 130° C. for 4 hours. After completion of the reaction, the organic layer was extracted with ethyl acetate, and the residual water was removed with magnesium sulfate followed by drying, and then separated by column chromatography to obtain compound 1-1 (67 g, yield: 56.6%).

2) Synthesis of Compound 1-2

Compound 1-1 (23.5 g, 58.9 mmol), (2-chlorophenyl)boronic acid (18.4 g, 117.8 mmol), Pd₂(dba)₃ (2.7 g, 2.95 mmol), S-Phos (2.4 g, 5.89 mmol), and K₃PO₄ (63 g, 294.5 mmol) in a flask were dissolved in 300 mL of toluene, and then stirred at 130° C. for 12 hours. After completion of the reaction, the organic layer was extracted with ethyl acetate, and the residual water was removed with magnesium sulfate followed by drying, and then separated by column chromatography to obtain compound 1-2 (14 g, yield: 50%).

3) Synthesis of Compound 1-3

Compound 1-2 (13 g, 27.4 mmol), and triphenyl phosphine (21.5 g, 82.1 mmol) in a flask were dissolved in 140 mL of o-DCB, and then stirred at 220° C. for 7 hours. After completion of the reaction, the reactants were removed by distillation, and then separated by column chromatography to obtain compound 1-3 (4 g, yield: 32%).

4) Synthesis of Compound 1-4

Compound 1-3 (10 g, 22.5 mmol). Pd(OAc)₂ (505 mg, 2.25 mmol). Pcy₃-HBF₄ (1.63 g, 4.5 mmol), and Cs₂CO₃ (22 g, 67.5 mmol) in a flask were dissolved in 113 mL of o-xylene 113 mL, and then stirred at 160° C. for 4 hours. After completion of the reaction, the organic layer was extracted with ethyl acetate, and the residual water was removed with magnesium sulfate followed by drying, and then separated by column chromatography to obtain compound 1-4 (1 g, yield: 11%).

5) Synthesis of Compound C-1

Compound 1-4 (4.5 g, 11.06 mmol), 2-chloro-3-phenylquioxaline (4 g, 16.6 mmol), DMAP (67 mg, 0.553 mmol), and Cs₂CO₃ (10.8 g, 331.8 mmol) in a flask were dissolved in 60 mL of DMSO, and then refluxed at 140° C. for 4 hours. After completion of the reaction, the organic layer was extracted with ethyl acetate, and the residual water was removed with magnesium sulfate followed by drying, and then separated by column chromatography to obtain compound C-1 (2.5 g. yield: 37%).

| | MW | M.P. |
|---|---|---|
| C-1 | 610.22 | 246° C. |

### [Example 2] Preparation of Compound C-29

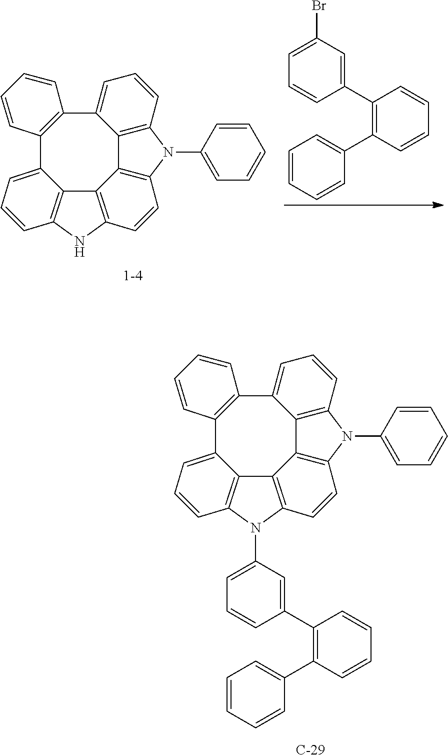

Compound 14 (4 g, 9.84 mmol), 3-bromo-1,1′:2′,1″-terphenyl (3.65 g, 11.8 mmol), Pd₂(dba)₃ (448 mg, 0.492 mmol), S-Phos (448 mg, 0.984 mmol), and NaOtBu (2.84 g, 29.52 mmol) in a flask were dissolved in 50 mL of o-xylene, and then stirred at 170° C. for 4 hours. After completion of the reaction, the organic layer was extracted with ethyl acetate, and the residual water was removed with magnesium sulfate followed by drying, and then separated by column chromatography to obtain compound C-29 (1.5 g, yield: 24%).

| | MW | M.P. |
|---|---|---|
| C-29 | 643.78 | 282° C. |

### [Example 3] Preparation of Compound C-196

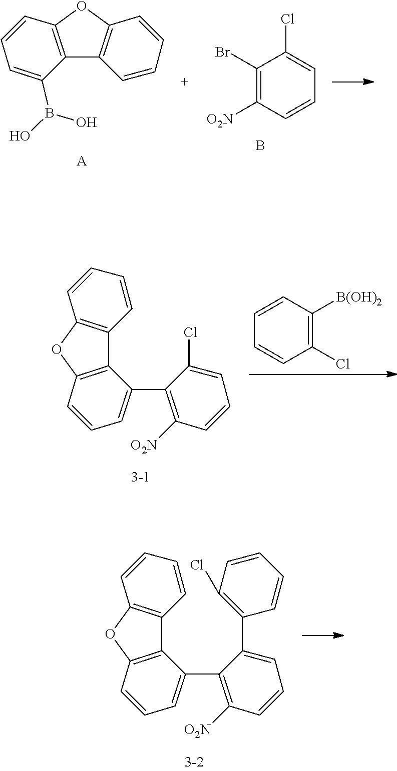

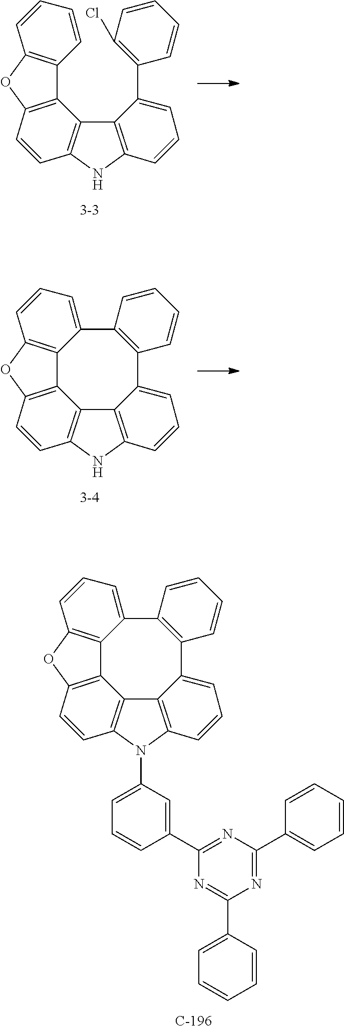

1) Synthesis of Compound 3-1

Compound A (60 g, 283 mmol), compound B (100 g, 424 mmol), tetrakis(triphenylphosphine)palladium (16.3 g, 14.1 mmol), cesium carbonate (276 g, 849 mmol), 1,400 mL of toluene, 350 mL of ethanol, and 350 mL of distilled water were added to a reaction vessel, and then stirred at 130° C. for 12 hours. After completion of the reaction, the reaction mixture was cooled to room temperature, and then the organic layer was extracted with ethyl acetate. The extracted organic layer was dried with magnesium sulfate, and then the solvent was removed with a rotary evaporator. Next, it was separated by column chromatography to obtain compound 3-1 (38 g, yield: 41%).

2) Synthesis of Compound 3-2

Compound 3-1 (38 g, 117 mmol), (2-chlorophenyl)boronic acid (35 g, 234 mmol), tris (dibenzylindeneacetone)dipalladium (5.3 g, 5.86 mmol), S-Phos (4.8 g, 11.7 mmol), potassium triphosphate (62 g, 293 mmol), and 600 mL of toluene were added to a reaction vessel, and stirred under reflux for 2 hours. After completion of the reaction, the reaction mixture was washed with distilled water, and then the organic layer was extracted with ethyl acetate. The organic layer was dried with magnesium sulfate, and then the solvent was removed with a rotary evaporator. Next, it was separated by column chromatography to obtain compound 3-2 (31 g, yield: 67%).

3) Synthesis of Compound 3-3

Compound 3-2 (21 g, 53.7 mmol), triphenyl phosphite (70 mL, 268 mmol), and 180 mL of DCB were added to a reaction vessel, and stirred at 200° C. for 12 hours. After completion of the reaction, DCB was removed by distilling under reduced pressure followed by washing with distilled water. Thereafter, the organic layer was extracted with ethyl acetate and was dried with magnesium sulfate, and then the solvent was removed with a rotary evaporator. Next, it was separated by column chromatography to obtain compound 3-3 (10 g, yield: 55%).

4) Synthesis of Compound 3-4

Compound 3-3 (6.6 g, 17.9 mmol), palladium(II) acetate (0.2 g, 0.89 mmol). PCy-BF₄ (1.3 g, 3.58 mmol), cesium carbonate (17 g, 53.7 mmol), and 90 mL of o-xylene were added to a reaction vessel, and then stirred under reflux at 160° C. for 4 hours. After completion of the reaction, the reaction mixture was washed with distilled water, and then the organic layer was extracted with ethyl acetate. Next, the organic layer was dried with magnesium sulfate, and then the solvent was removed with a rotary evaporator. Next, it was separated by column chromatography to obtain compound 3-4 (1.8 g, yield: 32%).

5) Synthesis of Compound C-196

Compound 3-4 (1.8 g, 5.43 mmol), 2-(3-bromophenyl)-4,6-diphenyl-1,3,5-triazine (2.3 g, 5.97 mmol), tris(dibenzylindeneacetone)dipalladium (0.2 g, 0.27 mmol), tri-tert-butylphosphine (0.3 mL, 0.54 mmol), sodium tert-butoxide (1.3 g, 13.5 mmol), and 30 mL of toluene were added to a reaction vessel, and then stirred under reflux for 3 hours. After completion of the reaction, the reaction mixture was washed with distilled water, and then the organic layer was extracted with ethyl acetate. Next, the organic layer was dried with magnesium sulfate, and then the solvent was removed with a rotary evaporator. Next, it was separated by column chromatography to obtain compound C-196 (3.3 g, yield: 95%).

| | MW | UV | PL | M.P. |
|---|---|---|---|---|
| C-196 | 638.21 | 410 nm | 522 nm | 240° C. |

### [Example 4] Preparation of Compound C-36

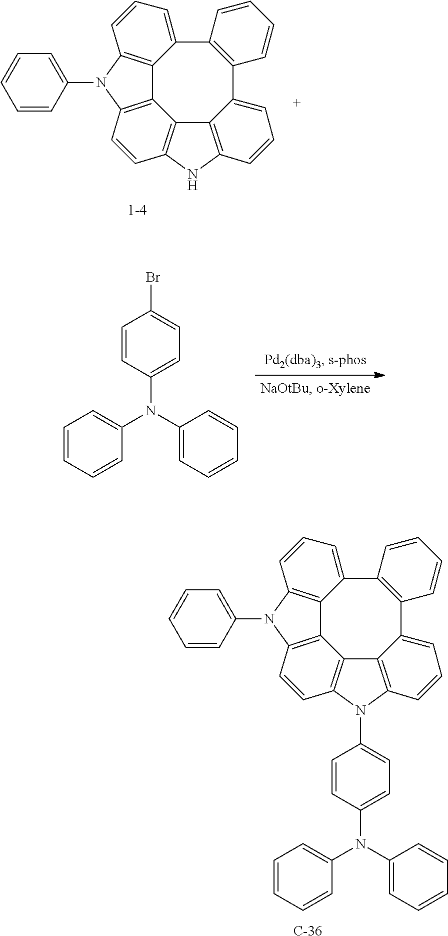

Compound 1-4 (4.0 g, 9.84 mmol), 4-bromo-N,N-diphenylaniline (3.2 g, 9.84 mmol), Pd₂(dba)₃ (0.45 g, 0.5 mmol), s-phos (0.4 g, 0.98 mmol), and NaOtBu (1.9 g, 19.7 mmol) in a flask were dissolved in 50 mL of o-xylene, and then stirred under reflux for 5 hours. After completion of the reaction, the organic layer was extracted with ethyl acetate, and then separated by column chromatography to obtain compound C-36 (2.67 g, yield: 42%).

| | MW | M.P. |
|---|---|---|
| C-36 | 649.78 | 312° C. |

### [Example 5] Preparation of Compound C-32

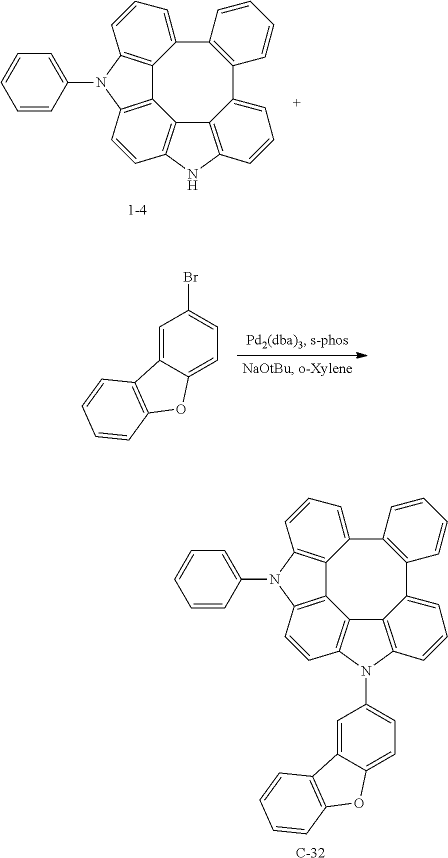

Compound 1-4 (4.0 g, 9.84 mmol), 2-bromodibenzo[b,d]furan (1.7 g, 9.84 mmol), Pd₂(dba)s (0.45 g, 0.5 mmol), s-phos (0.4 g, 0.98 mmol), and NaOtBu (1.9 g, 19.7 mmol) in a flask were dissolved in 50 mL of o-xylene, and then stirred under reflux for 5 hours. After completion of the reaction, the organic layer was extracted with ethyl acetate, and then separated by column chromatography to obtain Compound C-32 (1.68 g, yield: 30%).

| | MW | M.P. |
|---|---|---|
| C-32 | 572.65 | 291° C. |

Hereinafter, the preparation method of OLED according to the present disclosure and the property thereof will be explained in order to understand the present disclosure in detail. However, it is only to describe the characteristics of the OLED according to the present application, and is not limited to the following examples.

### [Device Examples 1 to 3] Preparation of OLEDs Comprising the Host Materials According to the Present Disclosure

OLEDs using the host materials according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and compound HT-1 as a first hole transport compound was introduced into another cell of the vacuum vapor deposition apparatus. The two materials were evaporated at different rates and the first hole injection compound was deposited in a doping amount of 3 wt % based on the total amount of the first hole injection compound and the first hole transport compound to form a first hole injection layer having a thickness of 10 nm. Next, compound HT-1 was deposited as a first hole transport layer having a thickness of 80 nm on the first hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The first host compound and second host compound shown in the following Table 1 were introduced into two cells of the vacuum vapor deposition apparatus as hosts, respectively, and compound D-39 was introduced into another cell as a dopant. The two host materials were evaporated at a rate of 1:1 and the dopant material was evaporated at a different rate, simultaneously, and was deposited in a doping amount of 3 wt % based on the total amount of the host and dopant to form a light-emitting layer having a thickness of 40 nm on the hole transport layer. Next, compound ETL-1 and compound EIL-1 as an electron transport material were deposited at a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials was purified by vacuum sublimation under 10⁻⁶ torr.

### [Device Comparative Examples 1 to 3] Preparation of OLEDs Comprising a Conventional Compound as a Host

OLEDs were produced in the same manner as in Device Example 1, except that the host compound of the following Table 1 alone was used as the host of the light-emitting layer.

The driving voltage, the luminous efficiency, and the light-emitting color at a luminance of 1,000 nits, and the time taken for luminance to decrease from 100% to 95% at a luminance of 5,000 nits (lifespan; T95) of the organic electroluminescent device according to Device Examples 1 to 3 and Device Comparative Examples 1 to 3 produced as described above, were measured, and the results thereof are shown in the following Table 1.

TABLE 1

| | | | Driving | Luminous | Light- | Lifespan |
|---|---|---|---|---|---|---|
| | First | Second | Voltage | Efficiency | Emitting | (T95) |
| | host | host | (V) | (cd/A) | Color | (hr) |
| Device | C-29 | H1-14 | 2.8 | 35.9 | Red | 306 |
| Example 1 | | | | | | |
| Device | C-32 | H1-14 | 2.9 | 35.8 | Red | 395 |
| Example 2 | | | | | | |
| Device | C-36 | H1-14 | 2.7 | 35.6 | Red | 205 |
| Example 3 | | | | | | |
| Device | C-29 | — | 4.7 | 5.8 | Red | 9.3 |
| Comparative | | | | | | |
| Example 1 | | | | | | |
| Device | C-36 | — | 3.9 | 5.8 | Red | 2.4 |
| Comparative | | | | | | |
| Example 2 | | | | | | |
| Devie | — | H1-14 | 4.0 | 29.0 | Red | 29.9 |
| Comparative | | | | | | |
| Example 3 | | | | | | |

From Table 1 above, it can be confirmed that by comprising the compounds of a specific combination according to the present disclosure as host materials, an organic electroluminescent device having long lifespan which exhibits low driving voltage and high luminous efficiency, and significantly improves lifespan characteristics can be provided.

The compounds used in Device Examples and Device Comparative Examples above are shown in the following Table 2.

TABLE 2

| | | | |
|---|---|---|---|
| Hole Injection Layer/ Hole Transport Layer | 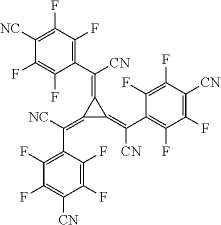 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] HI-1 | 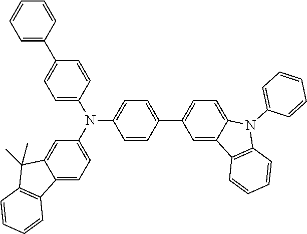 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] HT-1 | 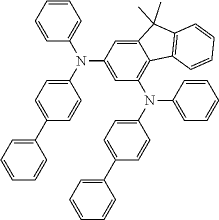 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] HT-2 |

| | | | |
|---|---|---|---|
| Light- Emitting Layer | 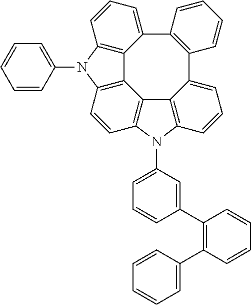 [SMILES: c1ccc(-c2ccccc2-c2cccc(-n3c4cccc5c6ccccc6c6cccc7c6c6c(c54)c3ccc6n7-c3ccccc3)c2)cc1] C-29 | 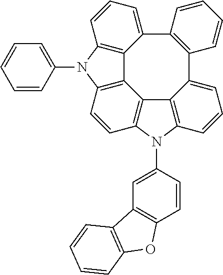 [SMILES: c1ccc(-n2c3cccc4c5ccccc5c5cccc6c5c5c(c43)c2ccc5n6-c2ccc3oc4ccccc4c3c2)cc1] C-32 | 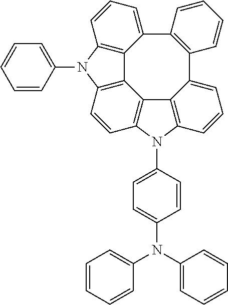 [SMILES: c1ccc(N(c2ccccc2)c2ccc(-n3c4cccc5c6ccccc6c6cccc7c6c6c(c54)c3ccc6n7-c3ccccc3)cc2)cc1] C-36 |

| | |
|---|---|
| 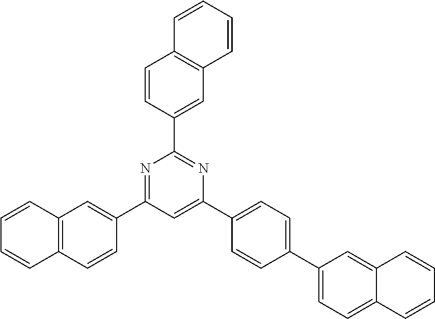 [SMILES: c1ccc2cc(-c3ccc(-c4cc(-c5ccc6ccccc6c5)nc(-c5ccc6ccccc6c5)n4)cc3)ccc2c1] H1-14 | 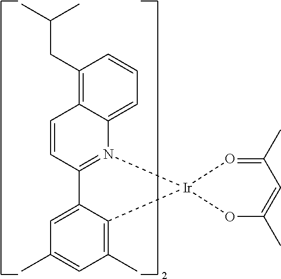 D-39 |

| | |
|---|---|
| Electron transport Layer/ Electron Injection Layer | 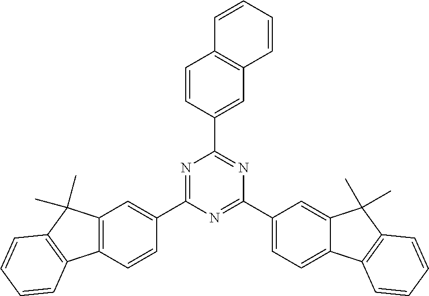 [SMILES: CC1(C)c2ccccc2-c2ccc(-c3nc(-c4ccc5c(c4)C(C)(C)c4ccccc4-5)nc(-c4ccc5ccccc5c4)n3)cc21] ETL-1 |
| | 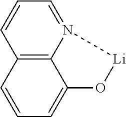 EIL-1 |

1. A plurality of host materials comprising a first host material and a second host material, wherein the first host material comprises a compound represented by the following formula 1 and the second host material comprises a compound represented by the following formula 2:
HAr-(L₁-Ar₁)ₐ (1)
wherein,
HAr represents a substituted or unsubstituted nitrogen-containing (3- to 10-membered)heteroaryl;
L₁ represents a single bond or a substituted or unsubstituted (C6-C30)arylene;
Ar₁ represents a substituted or unsubstituted (C6-C30)aryl; and
a represents an integer of 1 to 3, when a is an integer of 2 or more, each of (L₁-Ar₁) may be the same or different; 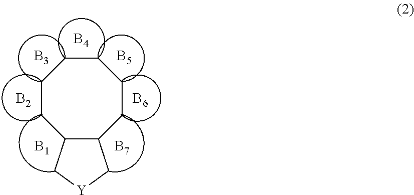
wherein,
B₁ to B₇ each independently are absent, or each independently represent a substituted or unsubstituted (C5-C20) ring, in which the carbon atom of the ring may be replaced with one or more heteroatom(s) selected from nitrogen, oxygen, and sulfur, with the proviso that at least five of B₁ to B₇ are present, and the adjacent rings of immediately adjacent rings in the B₁ to B₇ may be fused with each other:
Y represents —N-L₂-(Ar₂)ₙ, —O—, —S—, or —CR₁R₂;
L₂ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₂ represents a substituted or unsubstituted (C6-C30)aryl, a substituted or unsubstituted (3- to 30-membered)heteroaryl, or —NR₃R₄;
R₁ to R₄ each independently represent hydrogen, deuterium, halogen, cyano, a substituted or unsubstituted (C1-C30)alkyl, a substituted or unsubstituted (C2-C30)alkenyl, a substituted or unsubstituted fused ring of (C3-C30)aliphatic ring and (C6-C30)aromatic ring, a substituted or unsubstituted (C6-C30)aryl, a substituted or unsubstituted (3- to 30-membered)heteroaryl, or a substituted or unsubstituted (C3-C30)cycloalkyl; or may be linked to the adjacent substituent to form a ring(s); and
n represents an integer of 1 or 2, when n is 2, each of Ar₂ may be the same or different.

2. The plurality of host materials according to claim 1, wherein the compound represented by formula 1 is represented by the following formula 1-1 or 1-2: 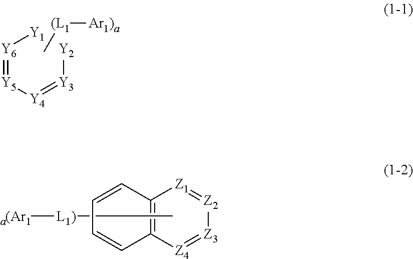
wherein,
Y₁ to Y₆ and Z₁ to Z₄ each independently represent CR₈ or N, provided that at least one of Y₁ to Y₆ represents N, and at least one of Z₁ to Z₄ represents N;
Rₐ each independently represent hydrogen, a substituted or unsubstituted (C1-C30)alkyl, a substituted or unsubstituted (C2-C30)alkenyl, or a substituted or unsubstituted (C6-C30)aryl; or may be linked to the adjacent substituent to form a ring(s); and
L₁, Ar₁, and a are as defined in claim 1.

3. The plurality of host materials according to claim 1, wherein HAr in formula 1 represents a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthiridinyl, a substituted or unsubstituted triazanaphthyl, or a substituted or unsubstituted benzothienopyrimidinyl.

4. The plurality of host materials according to claim 1, wherein B₁ to B₇ in formula 2 each independently are absent, or each independently represent a substituted or unsubstituted benzene ring, a substituted or unsubstituted naphthalene ring, a substituted or unsubstituted pyrrole ring, a substituted or unsubstituted furan ring, a substituted or unsubstituted thiophene ring, a substituted or unsubstituted cyclopentadiene ring, a substituted or unsubstituted fluorene ring, a substituted or unsubstituted pyridine ring, or a substituted or unsubstituted dibenzofuran ring.

5. The plurality of host materials according to claim 1, wherein the compound represented by formula 2 is represented by any one of the following formulas 2-1 to 2-5: 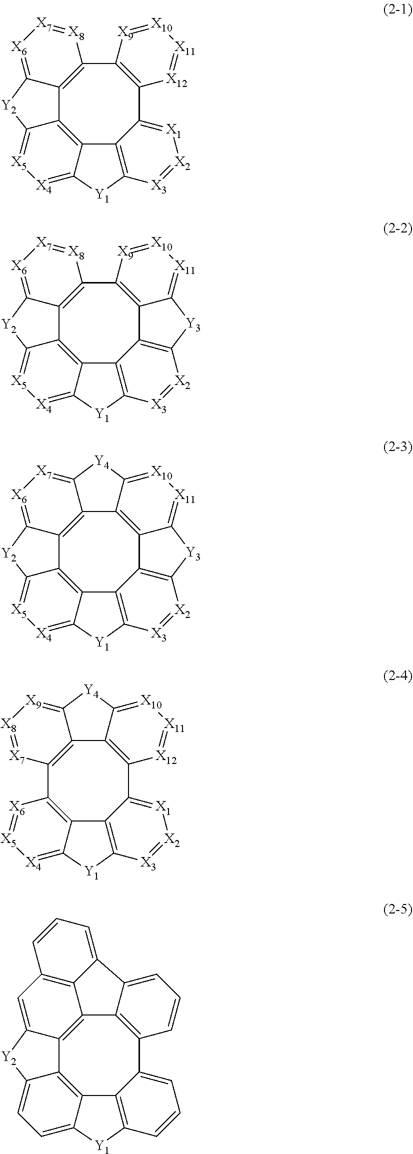
wherein,
Y₁, Y₂, Y₃, and Y₄ each independently are as defined as Y in claim 1, and where if a plurality of Ar₂ is present, each of Ar₂ may be the same or different;
X₁ to X₁₂ each independently represent —N═ or —C(R_{b})═; and
R_{b} represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or the adjacent R_{b}'s may be linked to each other to form a ring(s), and where if a plurality of R_{b} is present, each of R_{b} may be the same or different.

6. The plurality of host materials according to claim 5, wherein the Ar₂ and R_{b} each independently are selected from any one of the substituents listed in the following Group 1: 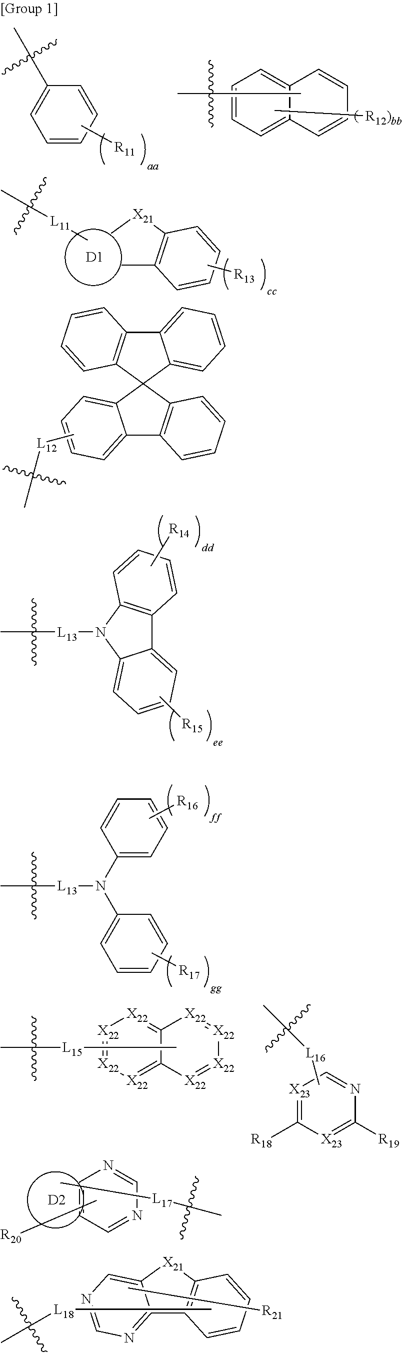
wherein,
D1 and D2 each independently represent a benzene ring or a naphthalene ring;
X₂₁ represents O, S, NR₅, or CR₆NR₇;
X₂₂ each independently represent CR or N, provided that at least one of X₂₂ represents N;
X₂₃ each independently represent CR₉ or N;
L₁₁ to L₁₈ each independently represent a single bond, a substituted or unsubstituted (C6-C30)arylene, or a substituted or unsubstituted (3- to 30-membered)heteroarylene;
R₁₁ to R₂₁ and R₅ to R₉ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to the adjacent substituent to form a ring(s); and
aa, ff, and gg each independently represent an integer of 1 to 5, bb represents an integer of 1 to 7, and cc, dd, and ee each independently represent an integer of 1 to 4.

7. The plurality of host materials according to claim 5, wherein the Ar₂ and R_{b} each independently are selected from any one of the substituents listed in the following Groups 2 and 3: 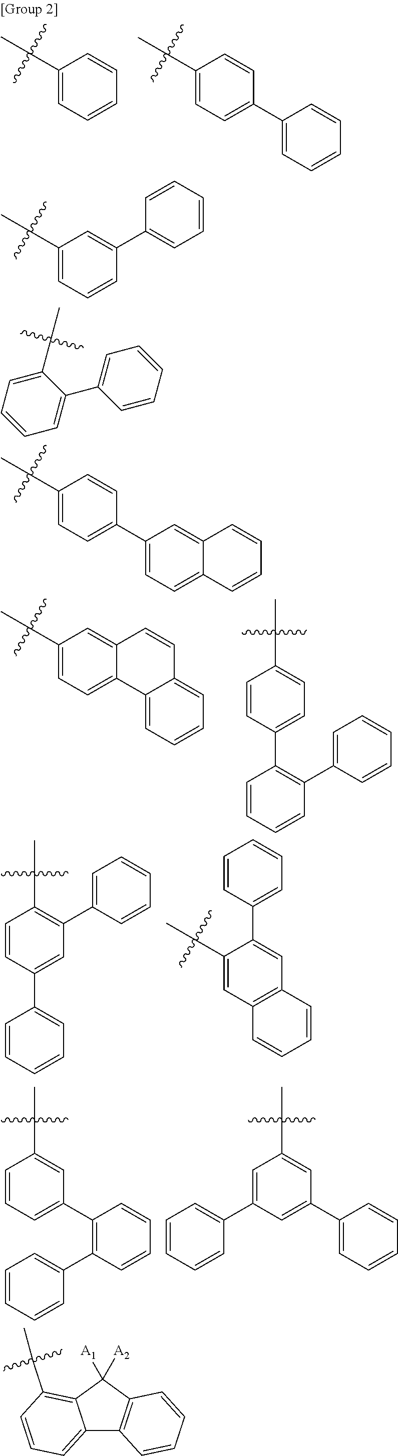 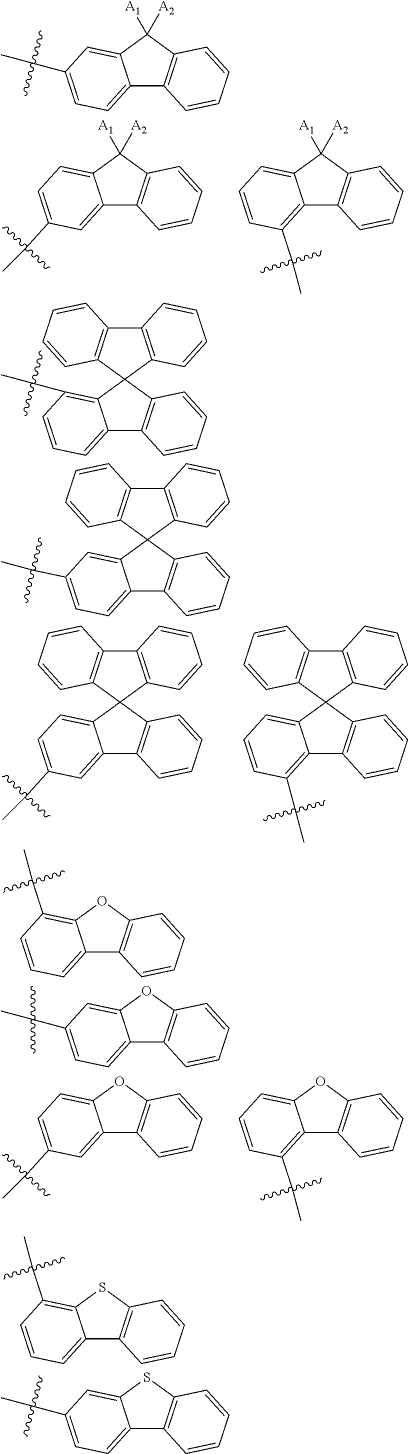 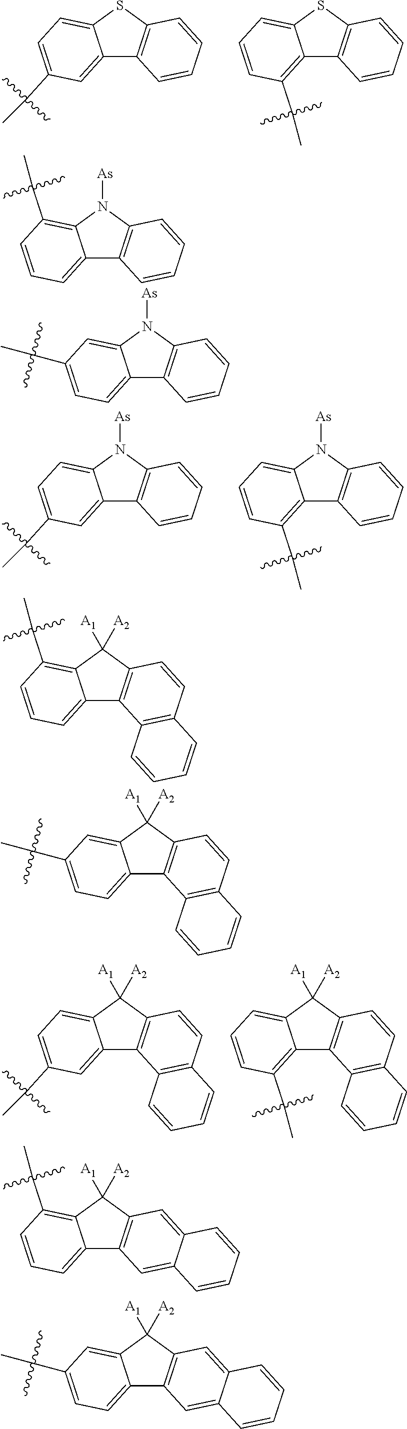 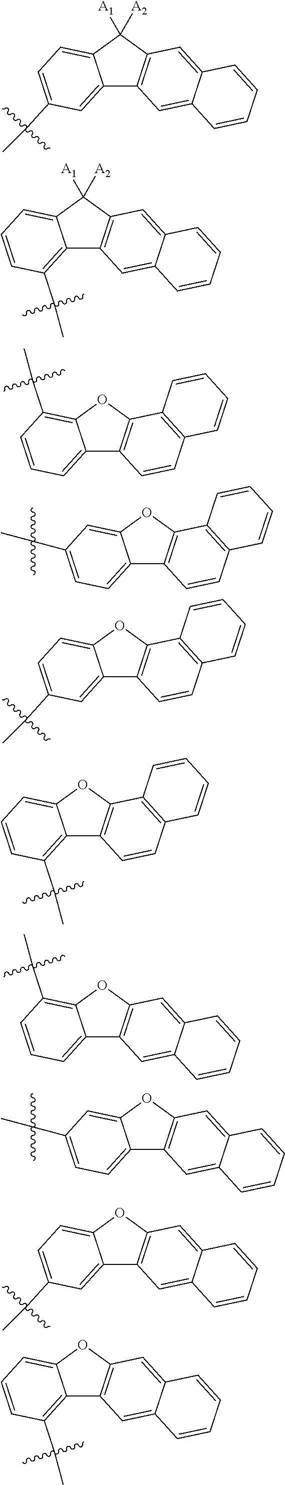 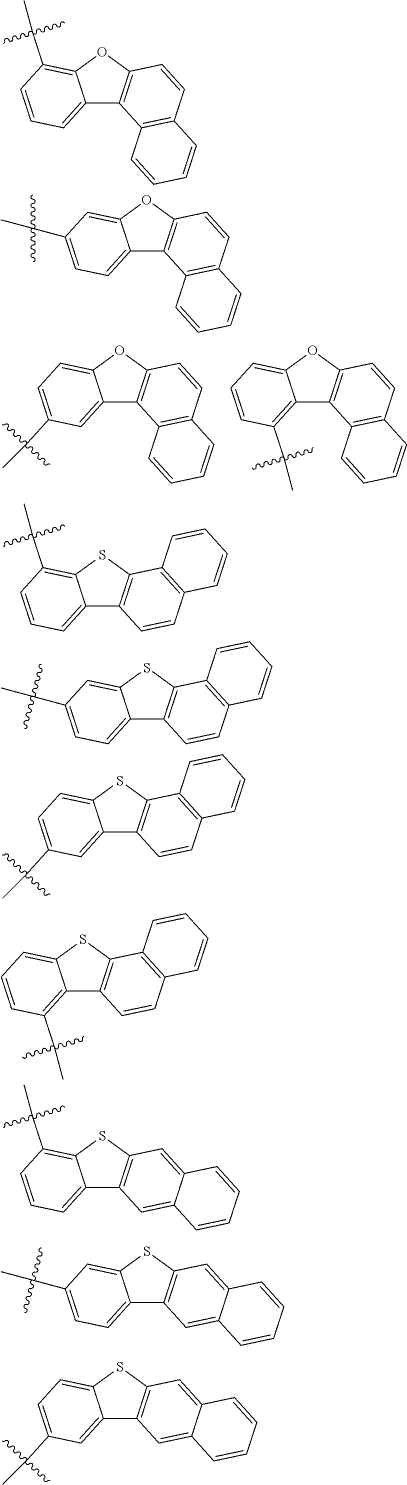 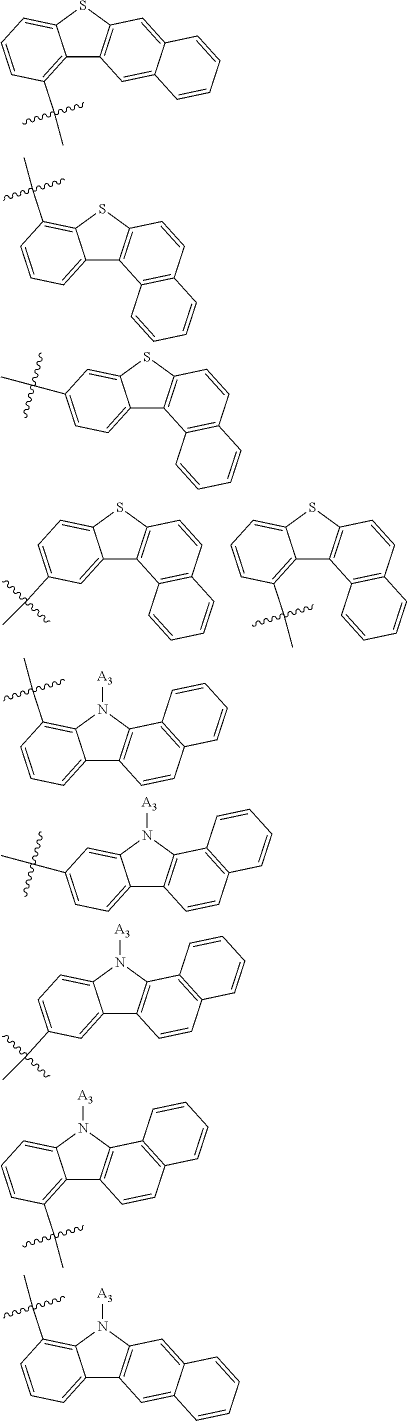 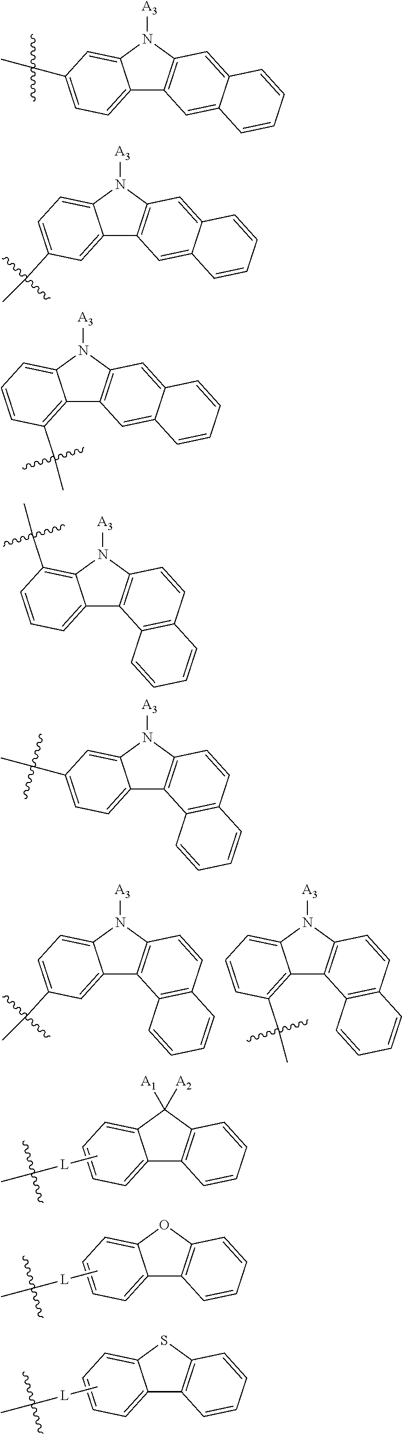 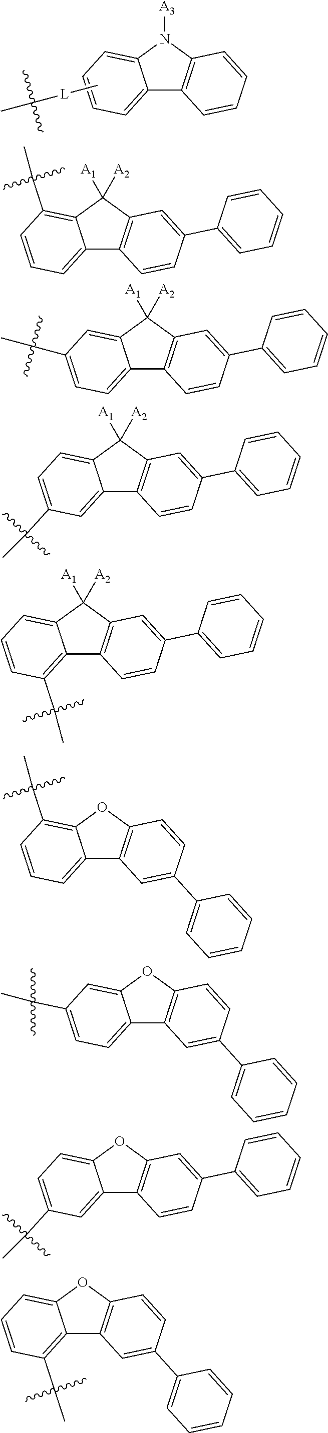 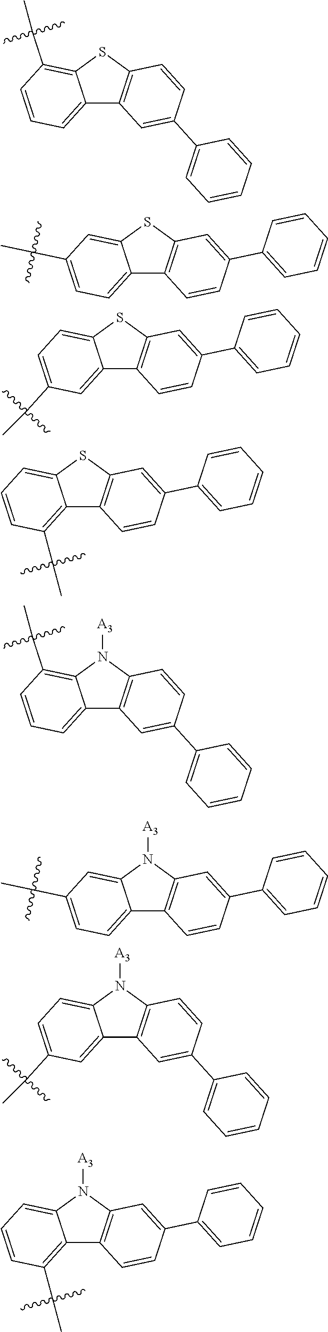 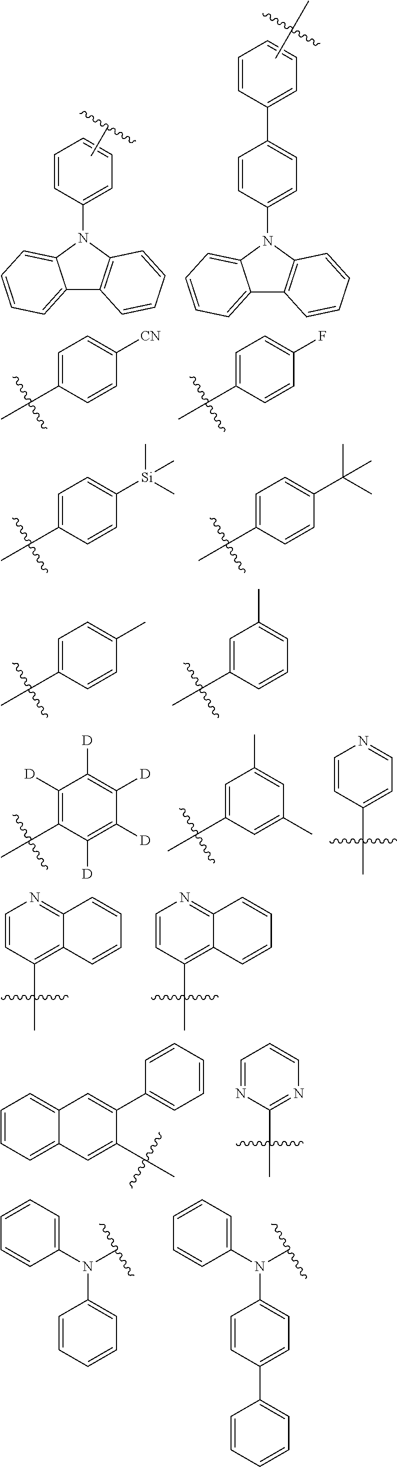 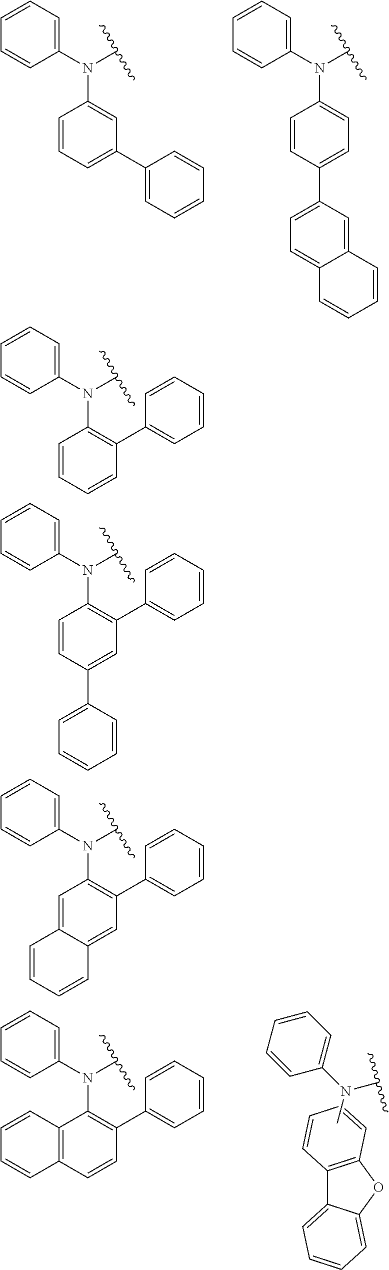 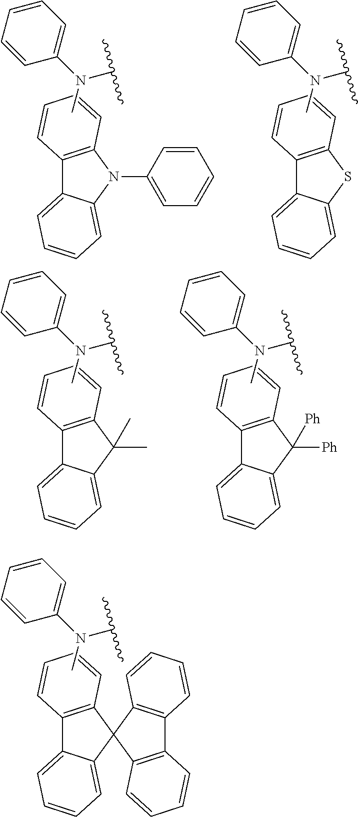 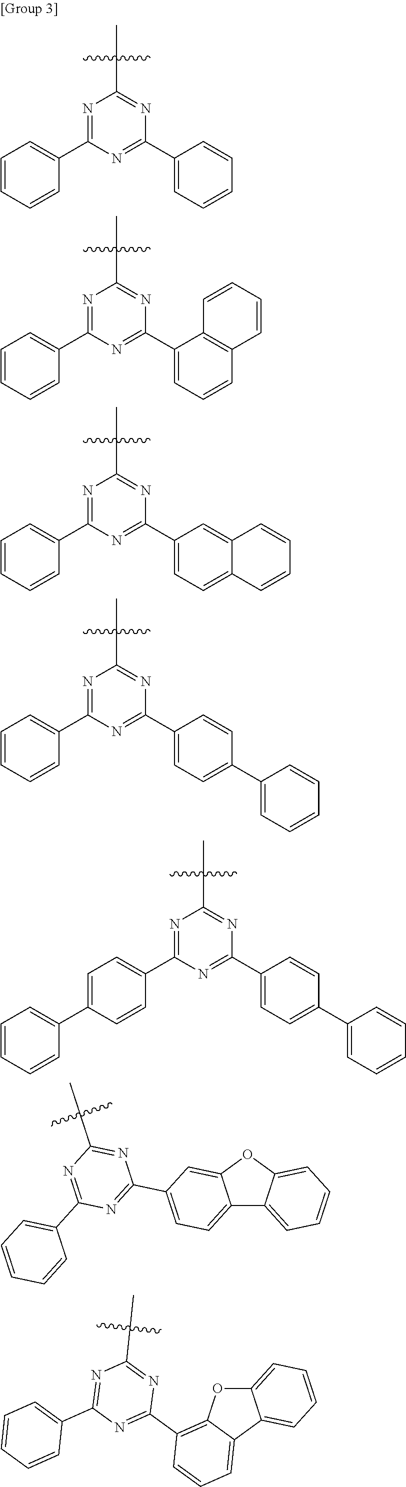 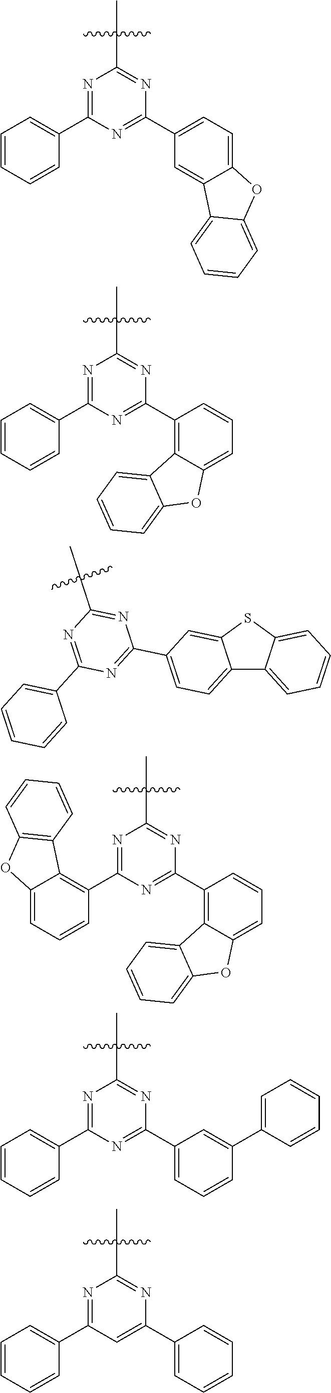 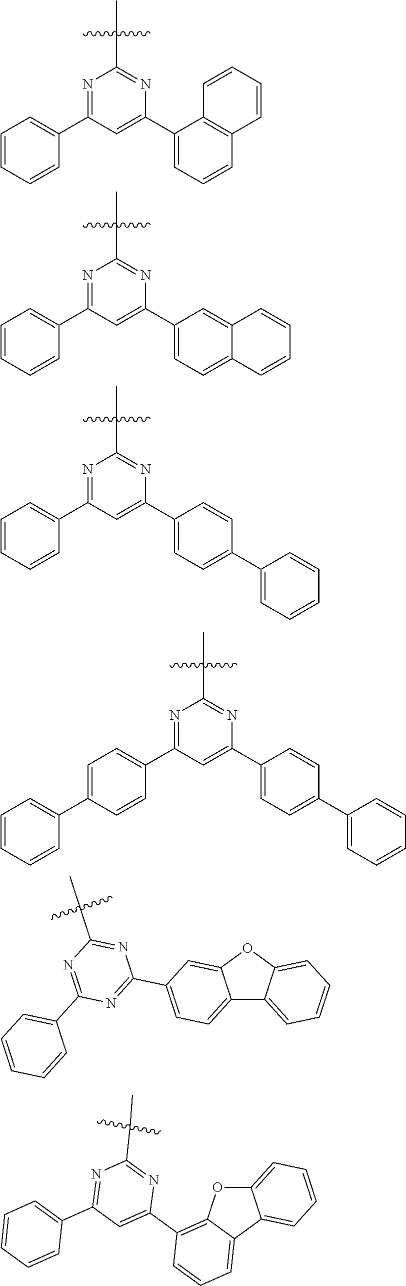 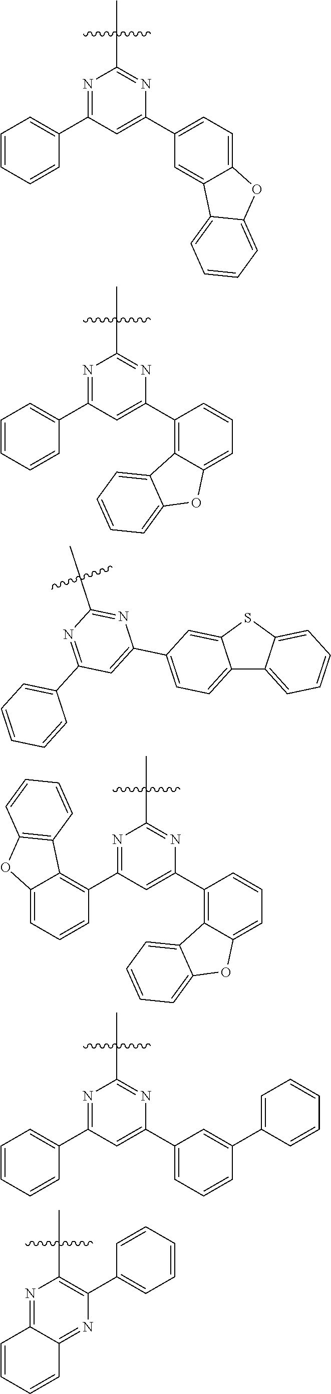 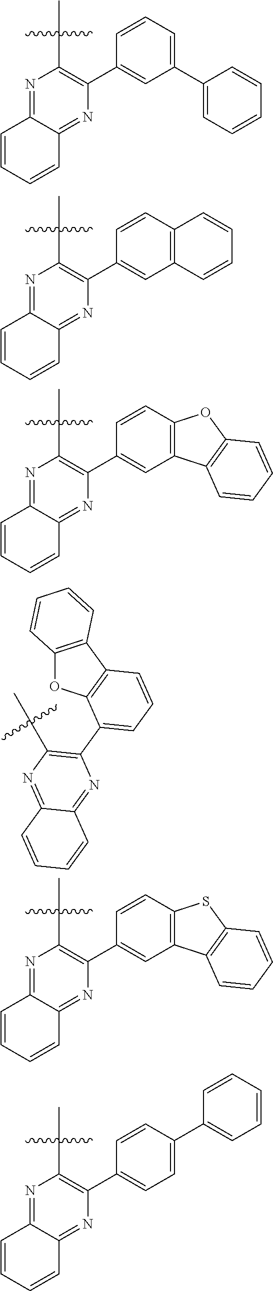 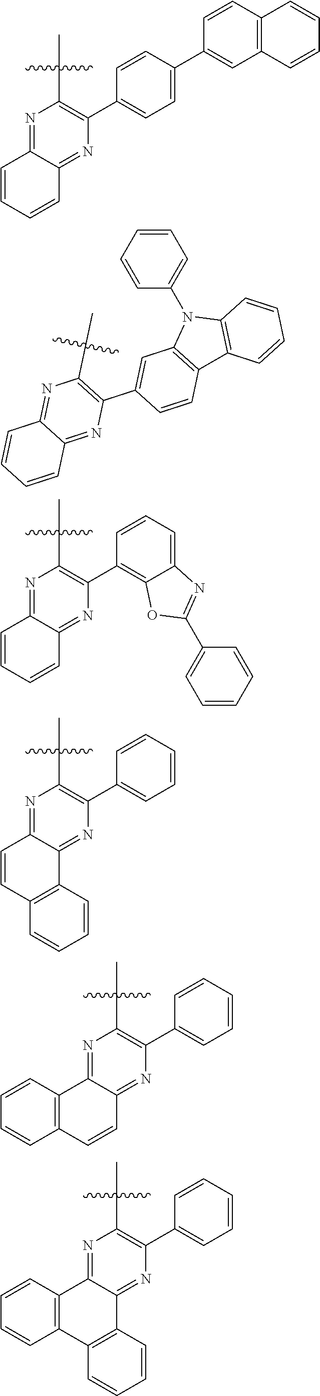 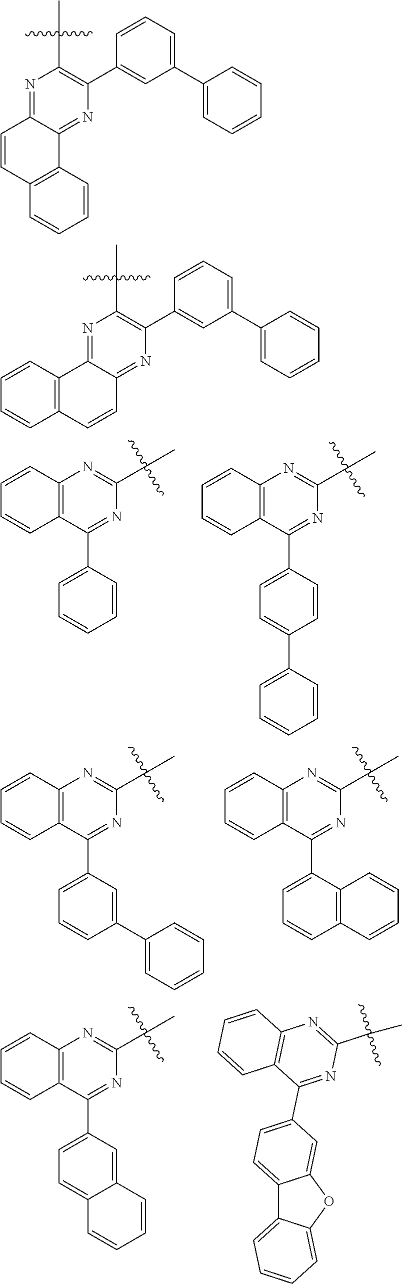 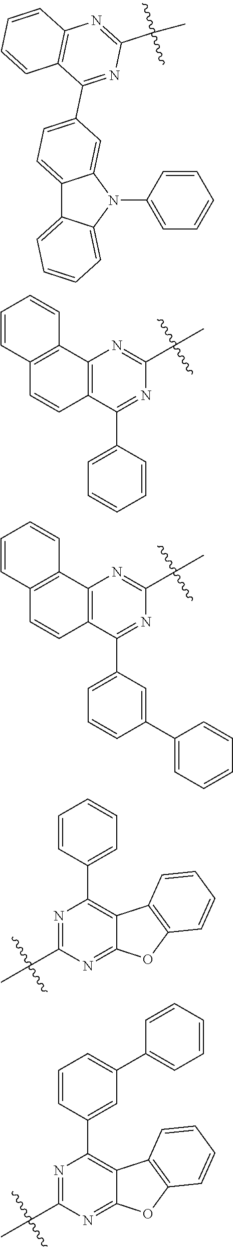 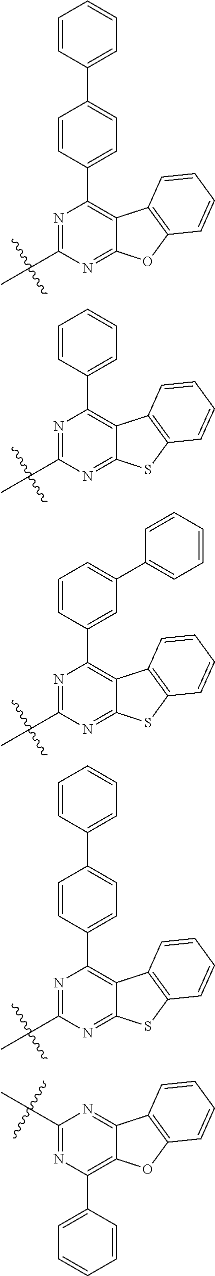 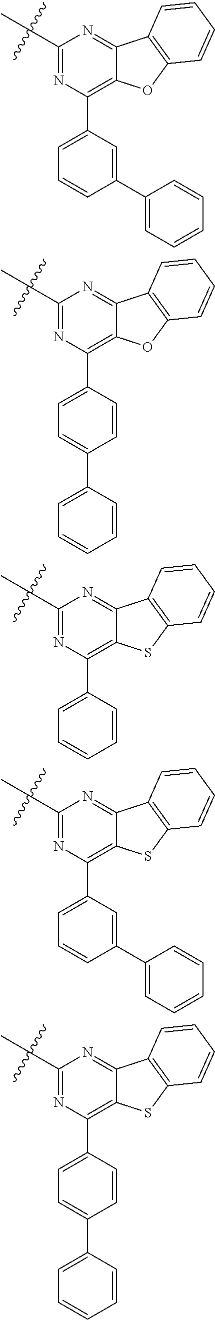 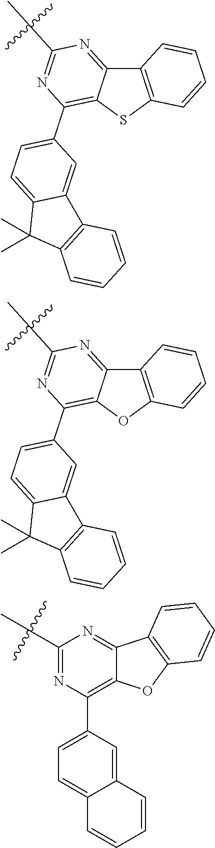
wherein,
L represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene; and
A₁ to A₃ each independently represent a substituted or unsubstituted (C1-C30)alkyl or a substituted or unsubstituted (C6-C30)aryl.

8. The plurality of host materials according to claim 1, wherein the compound represented by formula 1 is selected from the following compounds:

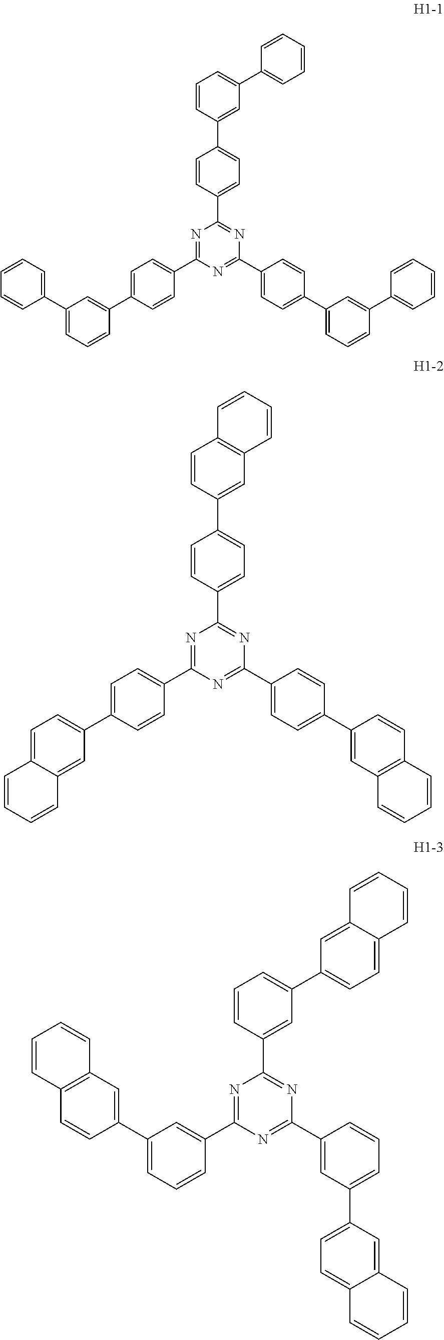 [SMILES: c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2nc(-c3cccc(-c4ccc5ccccc5c4)c3)nc(-c3cccc(-c4ccc5ccccc5c4)c3)n2)c1]

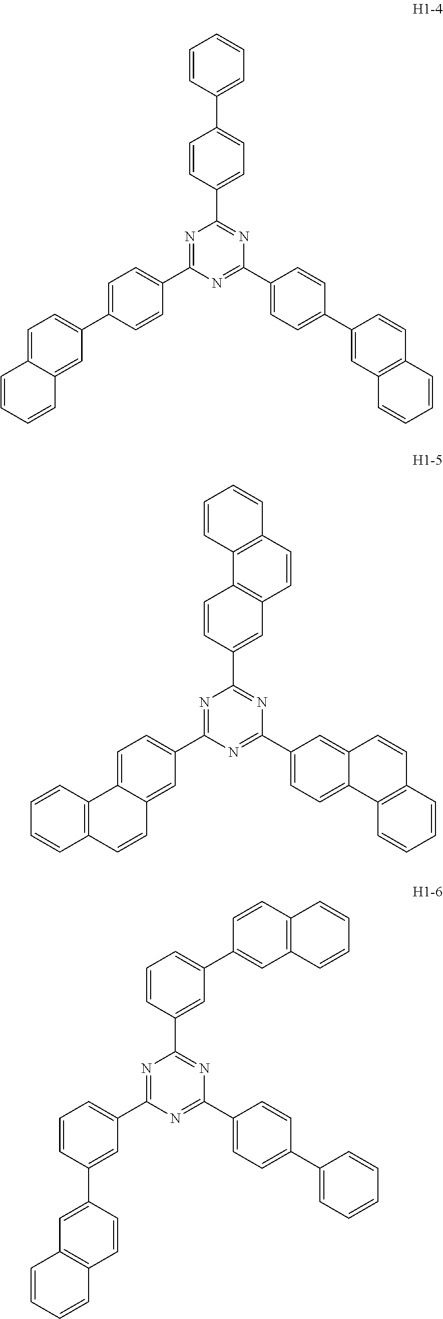 [SMILES: c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

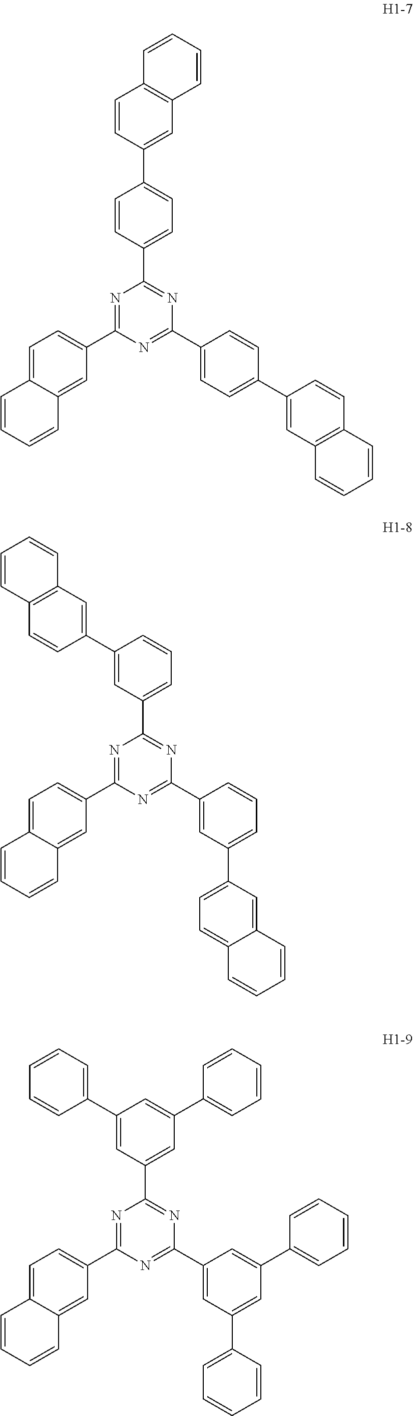 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1 ; c1ccc(-c2cc(-c3ccccc3)cc(-c3nc(-c4cc(-c5ccccc5)cc(-c5ccccc5)c4)nc(-c4ccc5ccccc5c4)n3)c2)cc1]

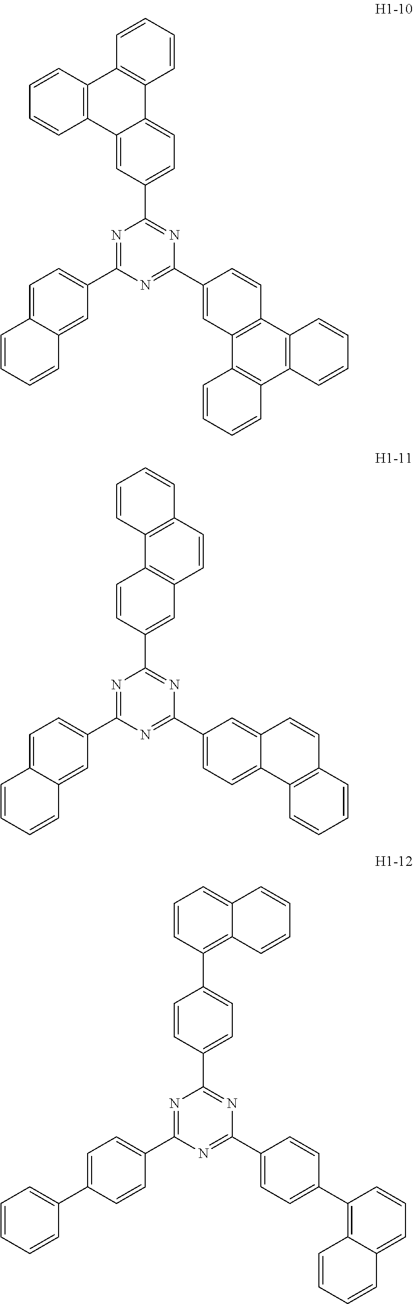 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1 ; c1ccc(-c2ccc(-c3nc(-c4ccc(-c5cccc6ccccc56)cc4)nc(-c4ccc(-c5cccc6ccccc56)cc4)n3)cc2)cc1]

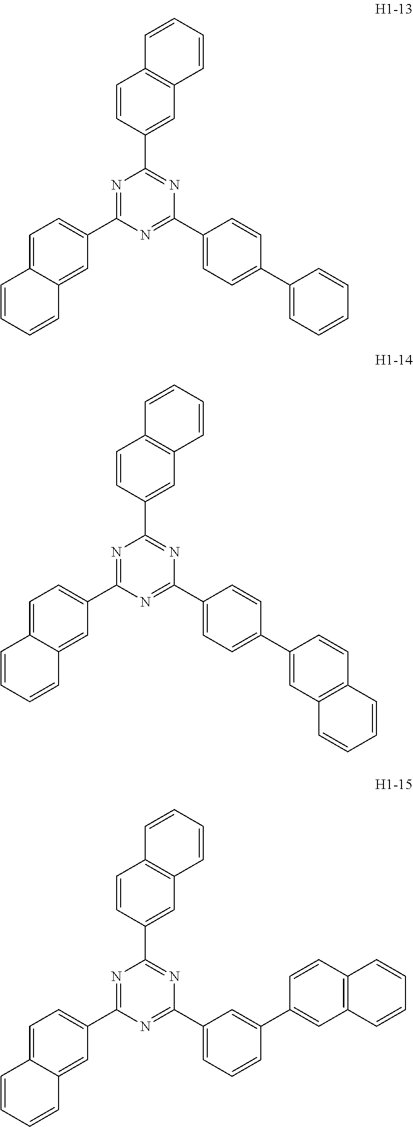 [SMILES: c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1 ; c1cc(-c2ccc3ccccc3c2)cc(-c2nc(-c3ccc4ccccc4c3)nc(-c3ccc4ccccc4c3)n2)c1]

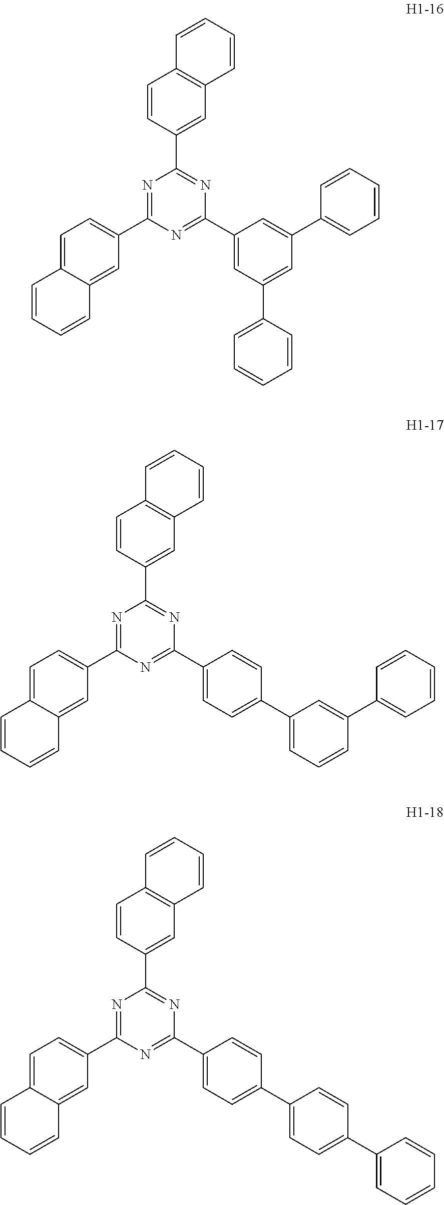 [SMILES: c1ccc(-c2cc(-c3ccccc3)cc(-c3nc(-c4ccc5ccccc5c4)nc(-c4ccc5ccccc5c4)n3)c2)cc1 ; c1ccc(-c2cccc(-c3ccc(-c4nc(-c5ccc6ccccc6c5)nc(-c5ccc6ccccc6c5)n4)cc3)c2)cc1 ; c1ccc(-c2ccc(-c3ccc(-c4nc(-c5ccc6ccccc6c5)nc(-c5ccc6ccccc6c5)n4)cc3)cc2)cc1]

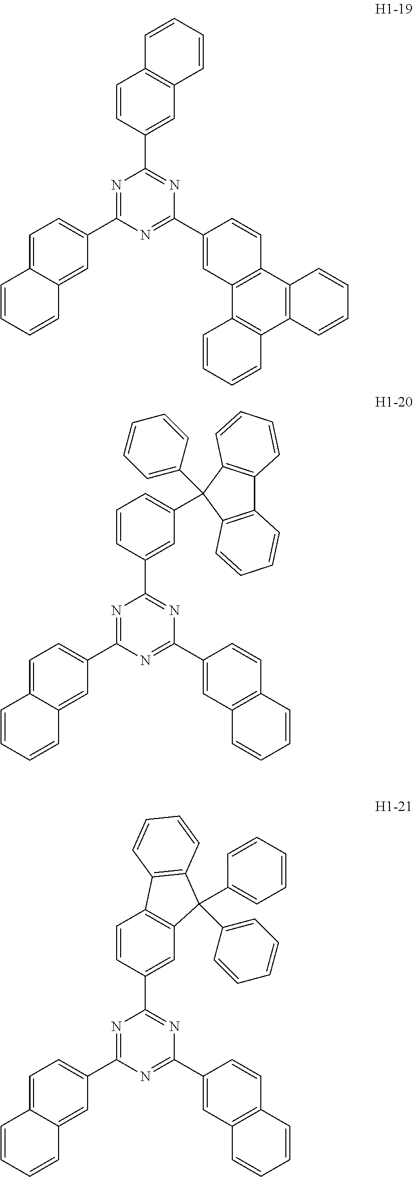

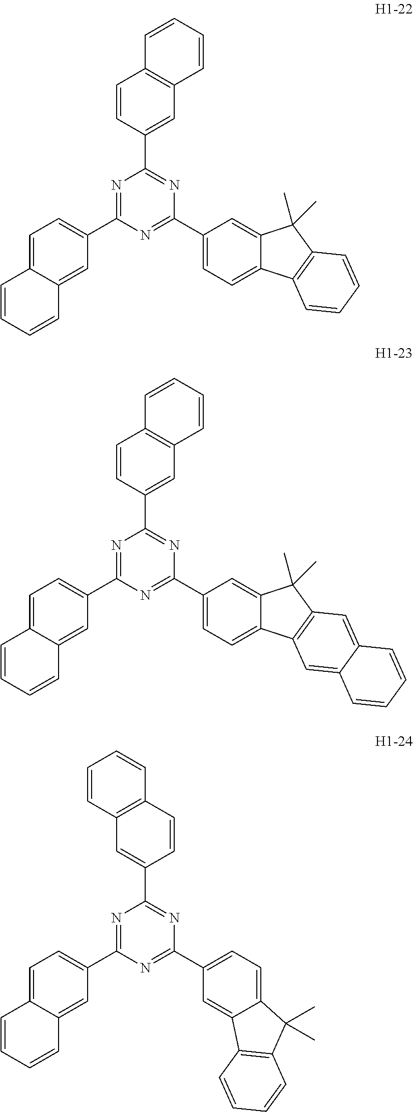 [SMILES: CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21 ; CC1(C)c2ccccc2-c2cc(-c3nc(-c4ccc5ccccc5c4)nc(-c4ccc5ccccc5c4)n3)ccc21]

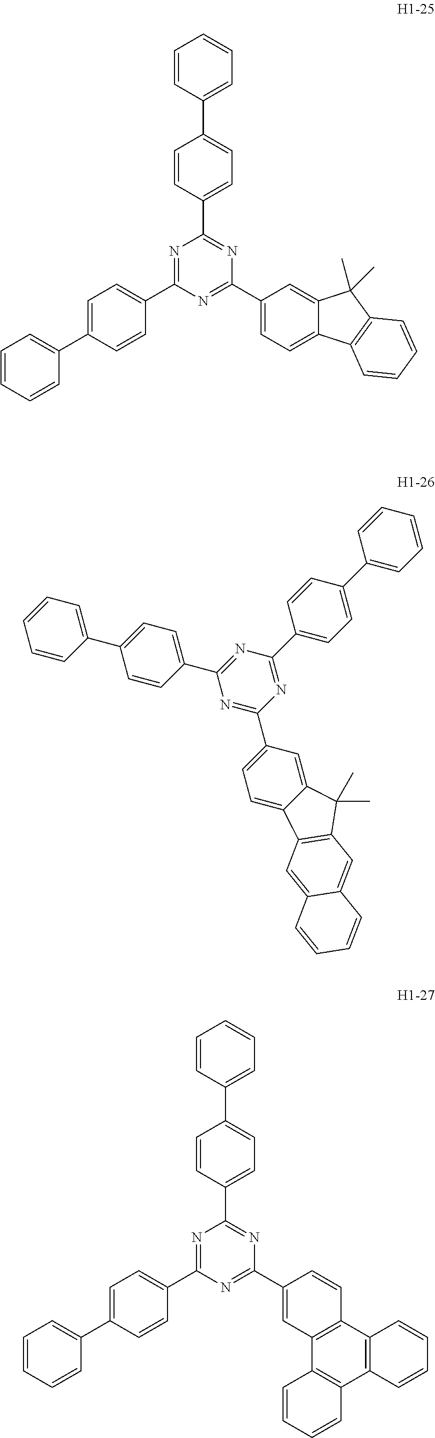 [SMILES: CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1]

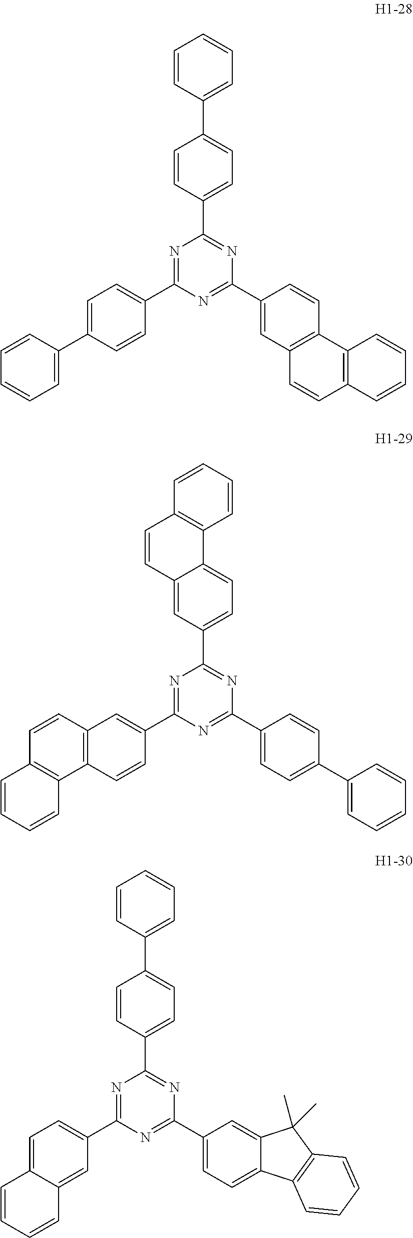 [SMILES: c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1 ; CC1(C)c2ccccc2-c2ccc(-c3nc(-c4ccc(-c5ccccc5)cc4)nc(-c4ccc5ccccc5c4)n3)cc21]

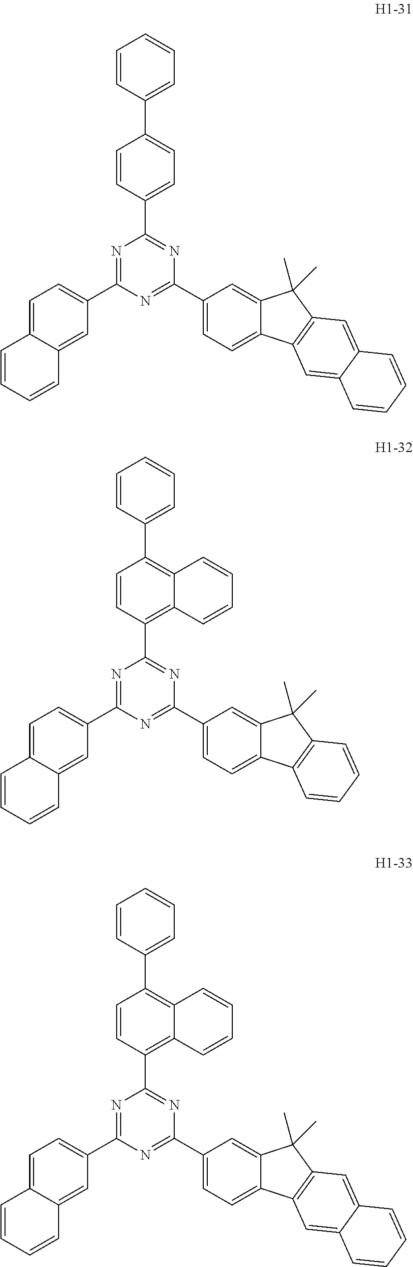 [SMILES: CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21]

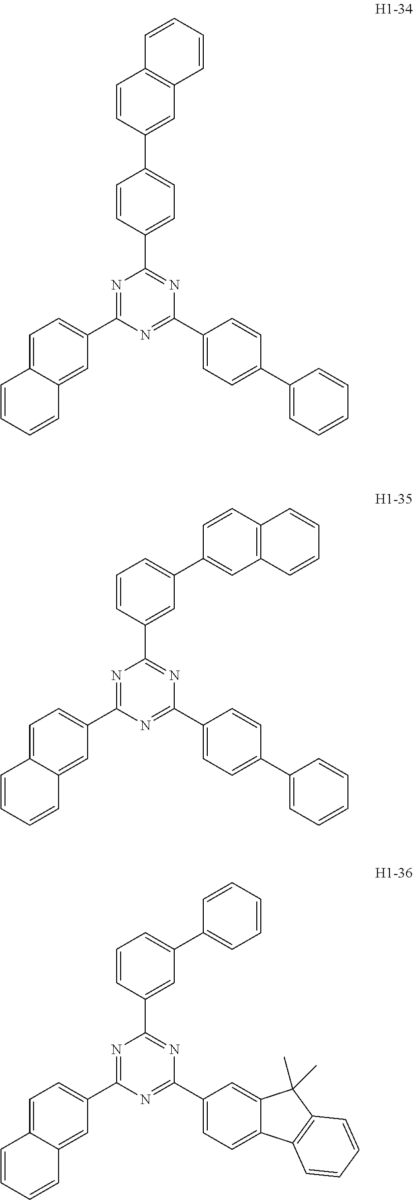 [SMILES: c1ccc(-c2ccc(-c3nc(-c4ccc(-c5ccc6ccccc6c5)cc4)nc(-c4ccc5ccccc5c4)n3)cc2)cc1 ; c1ccc(-c2ccc(-c3nc(-c4cccc(-c5ccc6ccccc6c5)c4)nc(-c4ccc5ccccc5c4)n3)cc2)cc1 ; CC1(C)c2ccccc2-c2ccc(-c3nc(-c4cccc(-c5ccccc5)c4)nc(-c4ccc5ccccc5c4)n3)cc21]

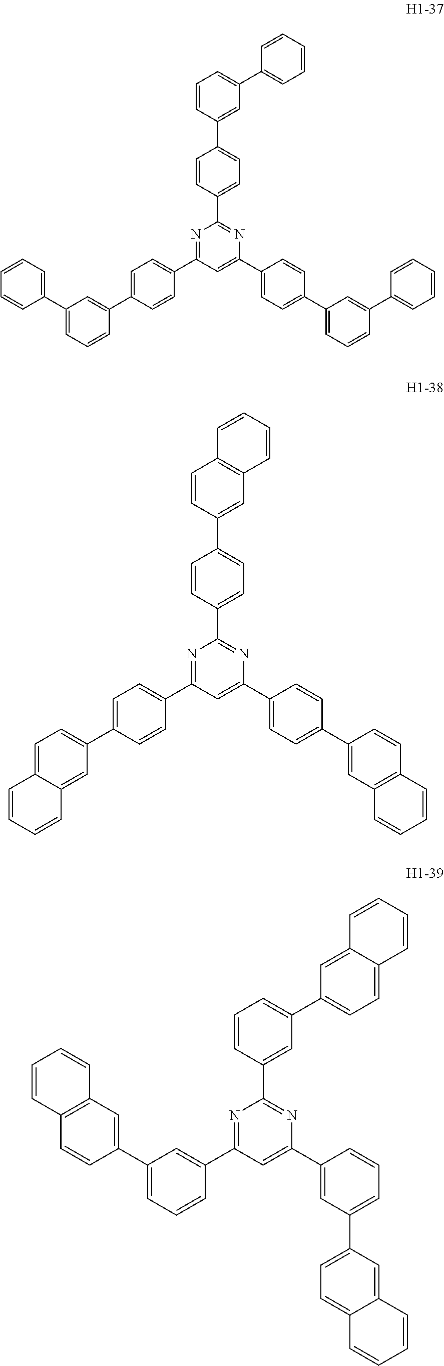 [SMILES: c1ccc(-c2cccc(-c3ccc(-c4cc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4cc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2cc(-c3cccc(-c4ccc5ccccc5c4)c3)nc(-c3cccc(-c4ccc5ccccc5c4)c3)n2)c1]

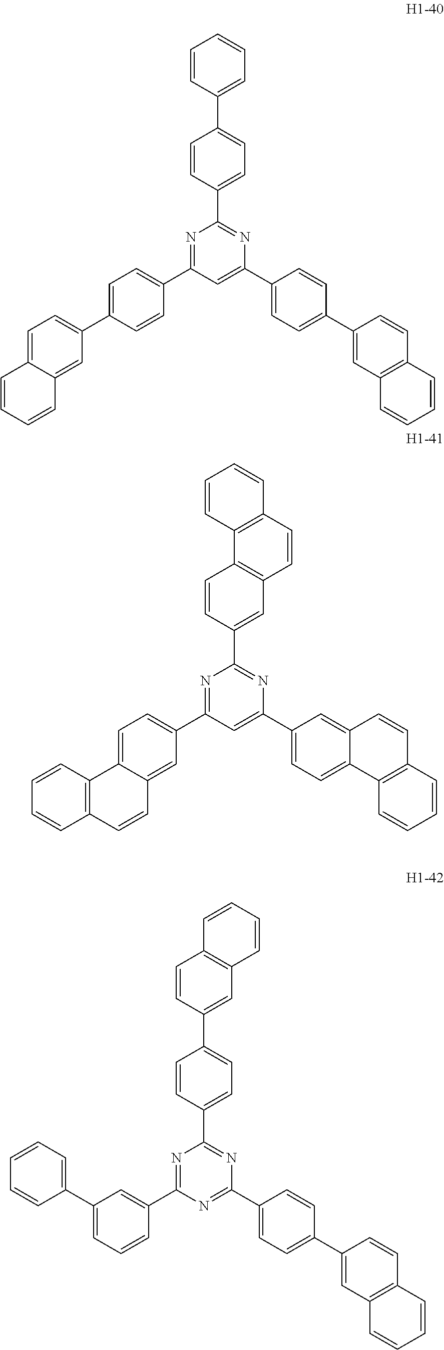 [SMILES: c1ccc(-c2ccc(-c3nc(-c4ccc(-c5ccc6ccccc6c5)cc4)cc(-c4ccc(-c5ccc6ccccc6c5)cc4)n3)cc2)cc1 ; c1ccc2c(c1)ccc1cc(-c3cc(-c4ccc5c(ccc6ccccc65)c4)nc(-c4ccc5c(ccc6ccccc65)c4)n3)ccc12 ; c1ccc(-c2cccc(-c3nc(-c4ccc(-c5ccc6ccccc6c5)cc4)nc(-c4ccc(-c5ccc6ccccc6c5)cc4)n3)c2)cc1]

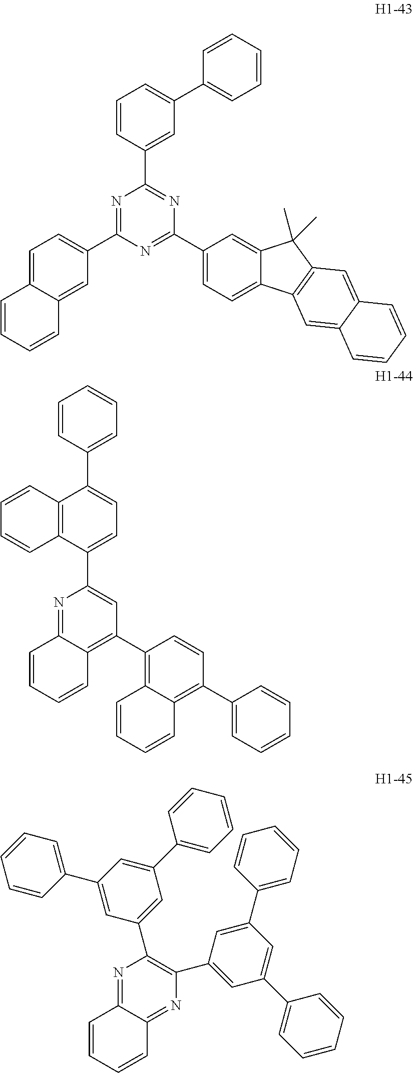 [SMILES: CC1(C)c2cc(-c3nc(-c4cccc(-c5ccccc5)c4)nc(-c4ccc5ccccc5c4)n3)ccc2-c2cc3ccccc3cc21 ; c1ccc(-c2ccc(-c3cc(-c4ccc(-c5ccccc5)c5ccccc45)c4ccccc4n3)c3ccccc23)cc1 ; c1ccc(-c2cc(-c3ccccc3)cc(-c3nc4ccccc4nc3-c3cc(-c4ccccc4)cc(-c4ccccc4)c3)c2)cc1]

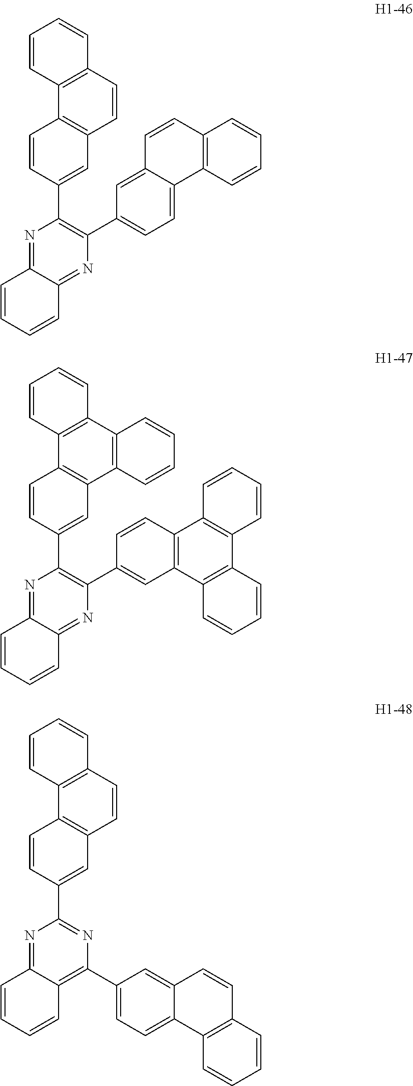

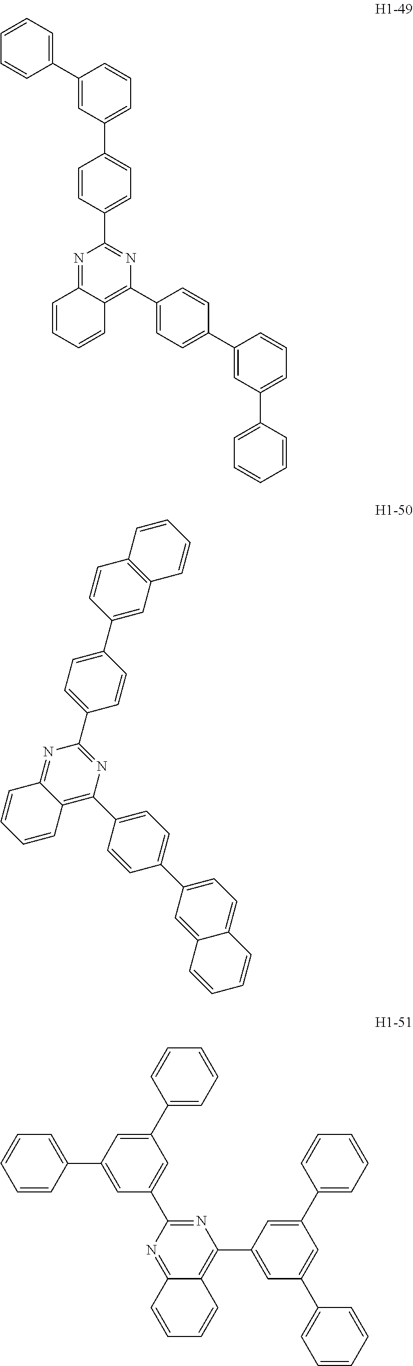 [SMILES: c1ccc(-c2cccc(-c3ccc(-c4nc(-c5ccc(-c6cccc(-c7ccccc7)c6)cc5)c5ccccc5n4)cc3)c2)cc1 ; c1ccc2cc(-c3ccc(-c4nc(-c5ccc(-c6ccc7ccccc7c6)cc5)c5ccccc5n4)cc3)ccc2c1 ; c1ccc(-c2cc(-c3ccccc3)cc(-c3nc(-c4cc(-c5ccccc5)cc(-c5ccccc5)c4)c4ccccc4n3)c2)cc1]

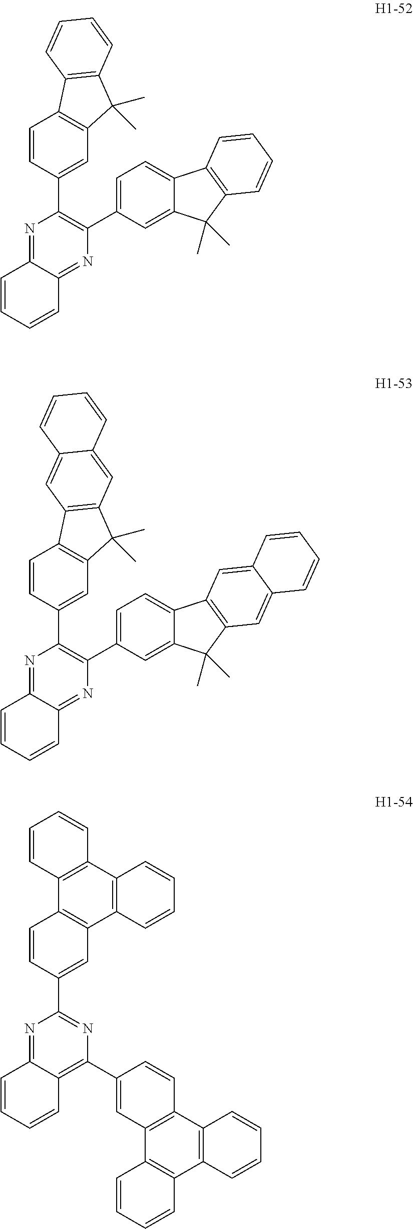 [SMILES: CC1(C)c2ccccc2-c2ccc(-c3nc4ccccc4nc3-c3ccc4c(c3)C(C)(C)c3ccccc3-4)cc21 ; CC1(C)c2cc(-c3nc4ccccc4nc3-c3ccc4c(c3)C(C)(C)c3cc5ccccc5cc3-4)ccc2-c2cc3ccccc3cc21 ; c1ccc2c(-c3ccc4c5ccccc5c5ccccc5c4c3)nc(-c3ccc4c5ccccc5c5ccccc5c4c3)nc2c1]

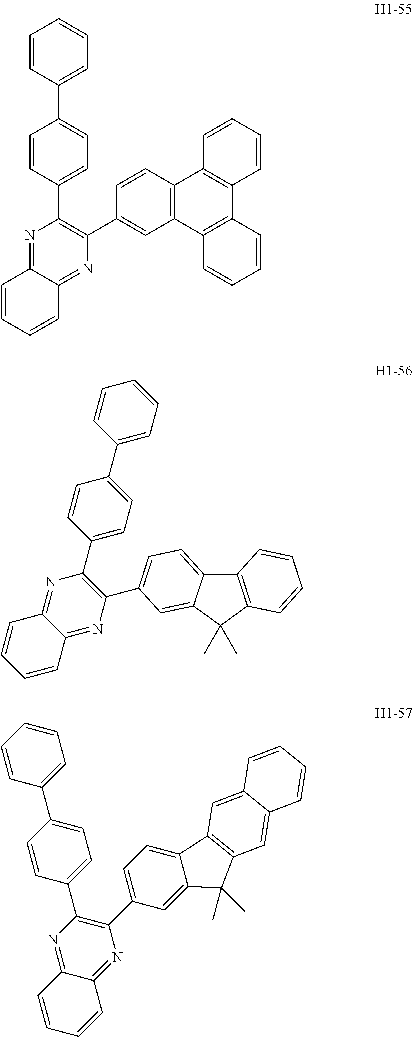

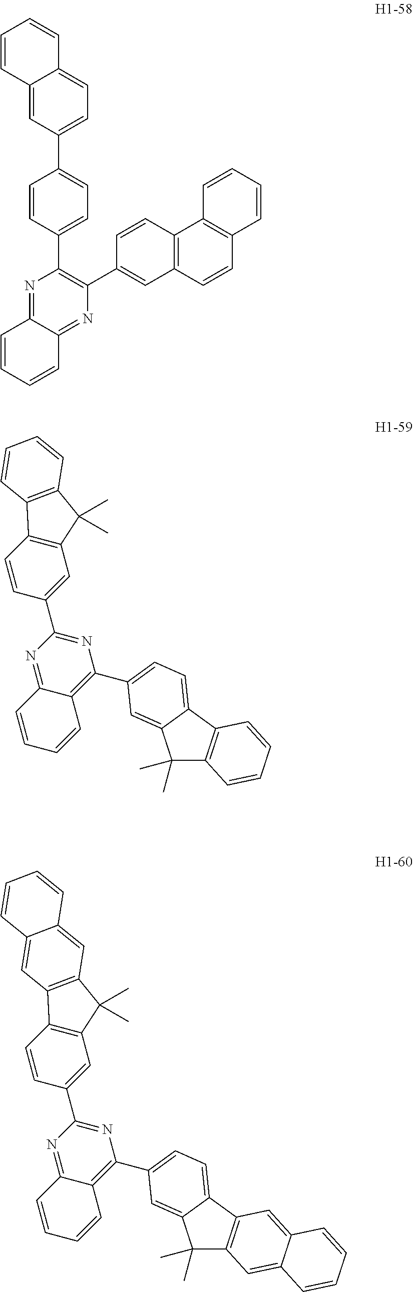 [SMILES: c1ccc2cc(-c3ccc(-c4nc5ccccc5nc4-c4ccc5c(ccc6ccccc65)c4)cc3)ccc2c1 ; CC1(C)c2ccccc2-c2ccc(-c3nc(-c4ccc5c(c4)C(C)(C)c4ccccc4-5)c4ccccc4n3)cc21 ; CC1(C)c2cc(-c3nc(-c4ccc5c(c4)C(C)(C)c4cc6ccccc6cc4-5)c4ccccc4n3)ccc2-c2cc3ccccc3cc21]

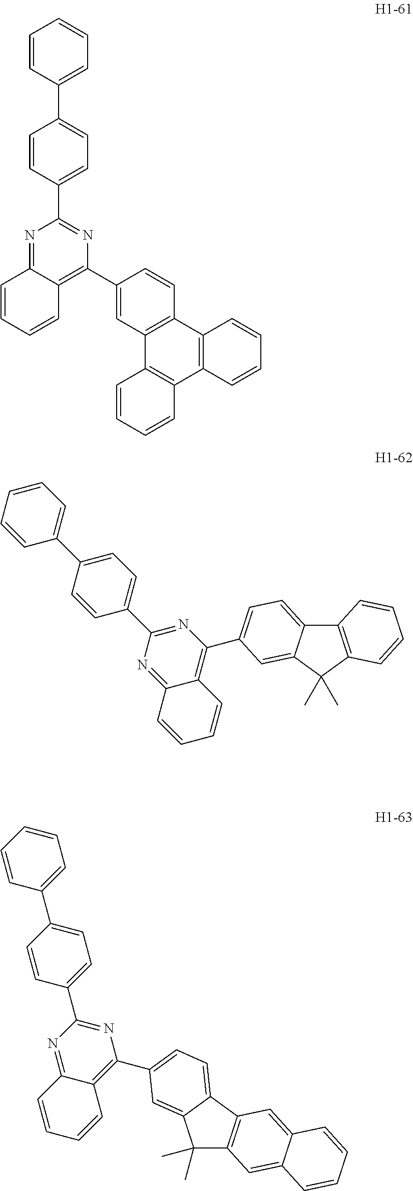 [SMILES: c1ccc(-c2ccc(-c3nc(-c4ccc5c6ccccc6c6ccccc6c5c4)c4ccccc4n3)cc2)cc1 ; CC1(C)c2ccccc2-c2ccc(-c3nc(-c4ccc(-c5ccccc5)cc4)nc4ccccc34)cc21 ; CC1(C)c2cc(-c3nc(-c4ccc(-c5ccccc5)cc4)nc4ccccc34)ccc2-c2cc3ccccc3cc21]

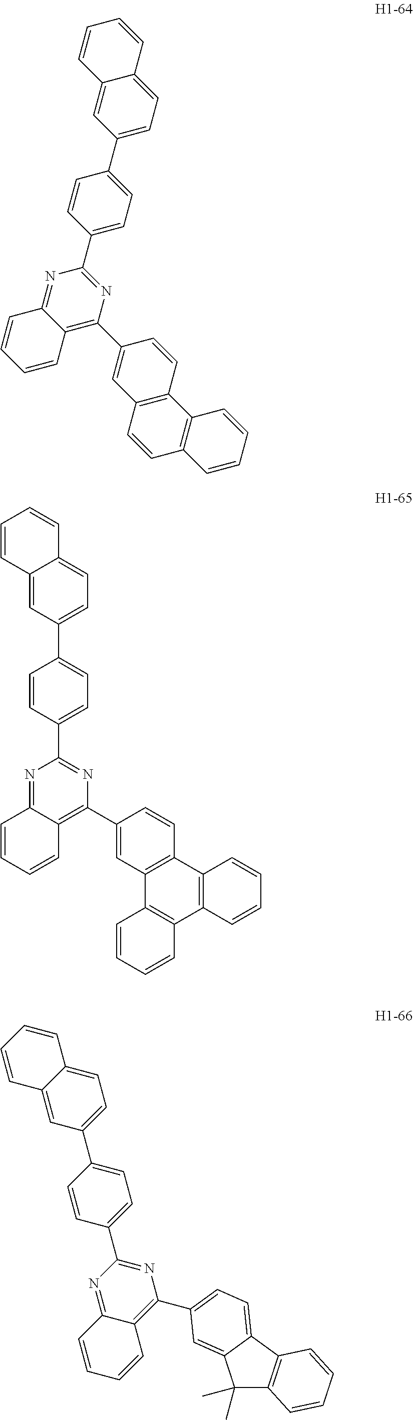 [SMILES: c1ccc2cc(-c3ccc(-c4nc(-c5ccc6c(ccc7ccccc76)c5)c5ccccc5n4)cc3)ccc2c1 ; c1ccc2cc(-c3ccc(-c4nc(-c5ccc6c7ccccc7c7ccccc7c6c5)c5ccccc5n4)cc3)ccc2c1 ; CC1(C)c2ccccc2-c2ccc(-c3nc(-c4ccc(-c5ccc6ccccc6c5)cc4)nc4ccccc34)cc21]

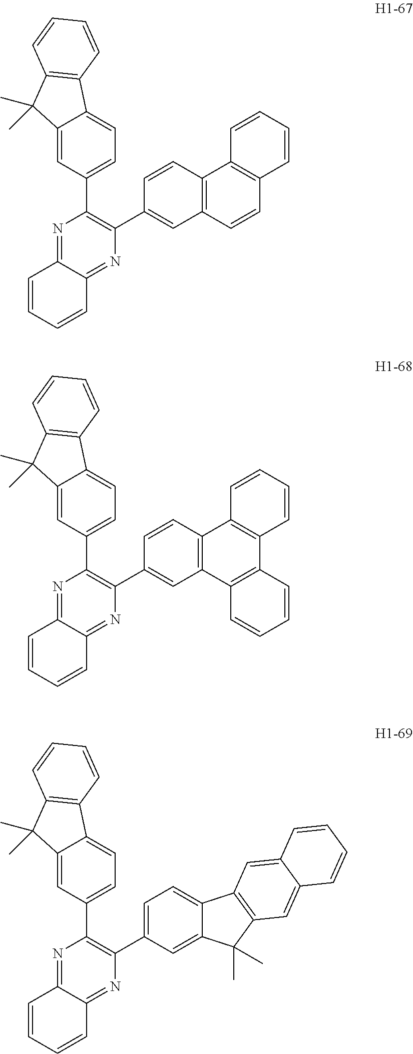 [SMILES: CC1(C)c2ccccc2-c2ccc(-c3nc4ccccc4nc3-c3ccc4c(ccc5ccccc54)c3)cc21 ; CC1(C)c2ccccc2-c2ccc(-c3nc4ccccc4nc3-c3ccc4c5ccccc5c5ccccc5c4c3)cc21 ; CC1(C)c2ccccc2-c2ccc(-c3nc4ccccc4nc3-c3ccc4c(c3)C(C)(C)c3cc5ccccc5cc3-4)cc21]

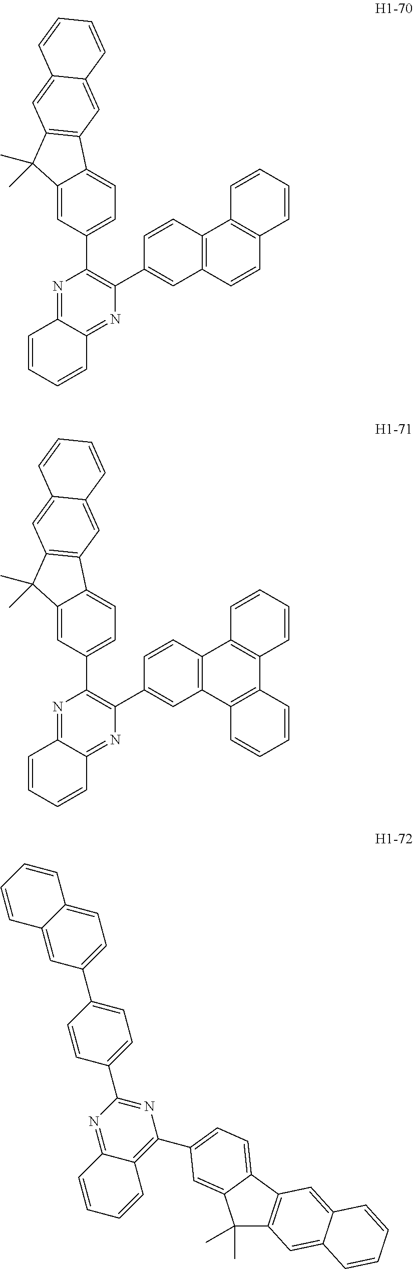 [SMILES: CC1(C)c2cc(-c3nc4ccccc4nc3-c3ccc4c(ccc5ccccc54)c3)ccc2-c2cc3ccccc3cc21 ; CC1(C)c2cc(-c3nc4ccccc4nc3-c3ccc4c5ccccc5c5ccccc5c4c3)ccc2-c2cc3ccccc3cc21 ; CC1(C)c2cc(-c3nc(-c4ccc(-c5ccc6ccccc6c5)cc4)nc4ccccc34)ccc2-c2cc3ccccc3cc21]

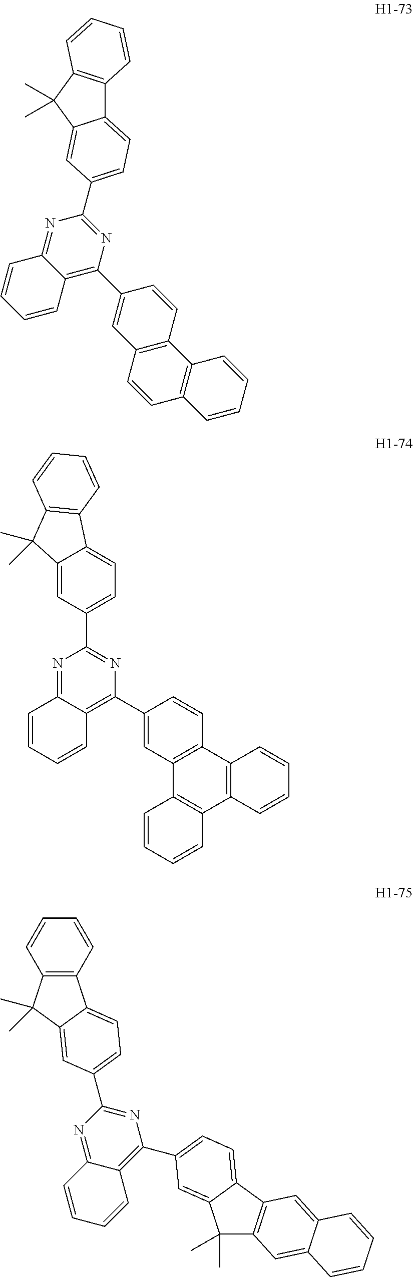 [SMILES: CC1(C)c2ccccc2-c2ccc(-c3nc(-c4ccc5c(ccc6ccccc65)c4)c4ccccc4n3)cc21 ; CC1(C)c2ccccc2-c2ccc(-c3nc(-c4ccc5c6ccccc6c6ccccc6c5c4)c4ccccc4n3)cc21 ; CC1(C)c2ccccc2-c2ccc(-c3nc(-c4ccc5c(c4)C(C)(C)c4cc6ccccc6cc4-5)c4ccccc4n3)cc21]

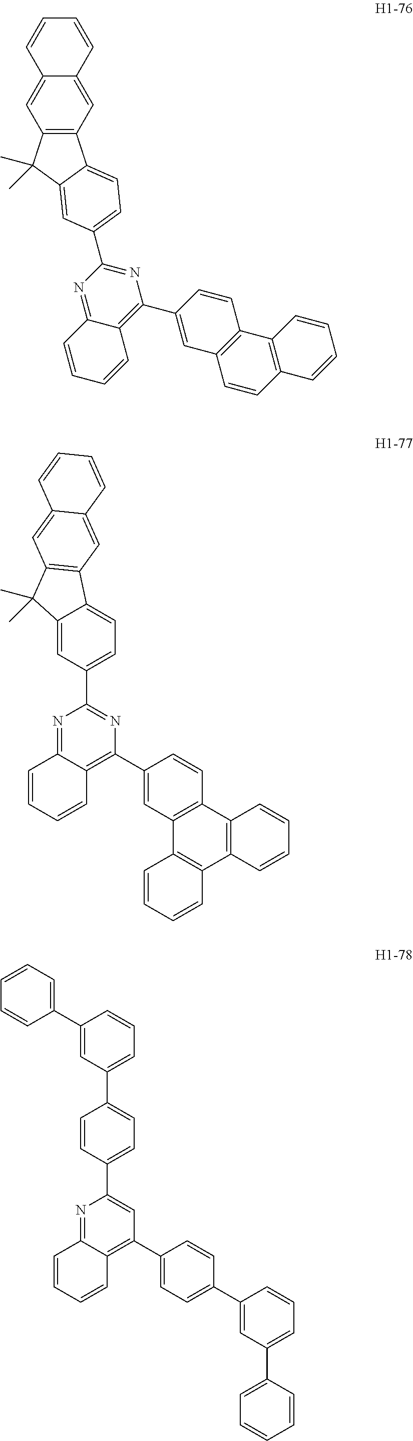 [SMILES: CC1(C)c2cc(-c3nc(-c4ccc5c(ccc6ccccc65)c4)c4ccccc4n3)ccc2-c2cc3ccccc3cc21 ; CC1(C)c2cc(-c3nc(-c4ccc5c6ccccc6c6ccccc6c5c4)c4ccccc4n3)ccc2-c2cc3ccccc3cc21 ; c1ccc(-c2cccc(-c3ccc(-c4cc(-c5ccc(-c6cccc(-c7ccccc7)c6)cc5)c5ccccc5n4)cc3)c2)cc1]

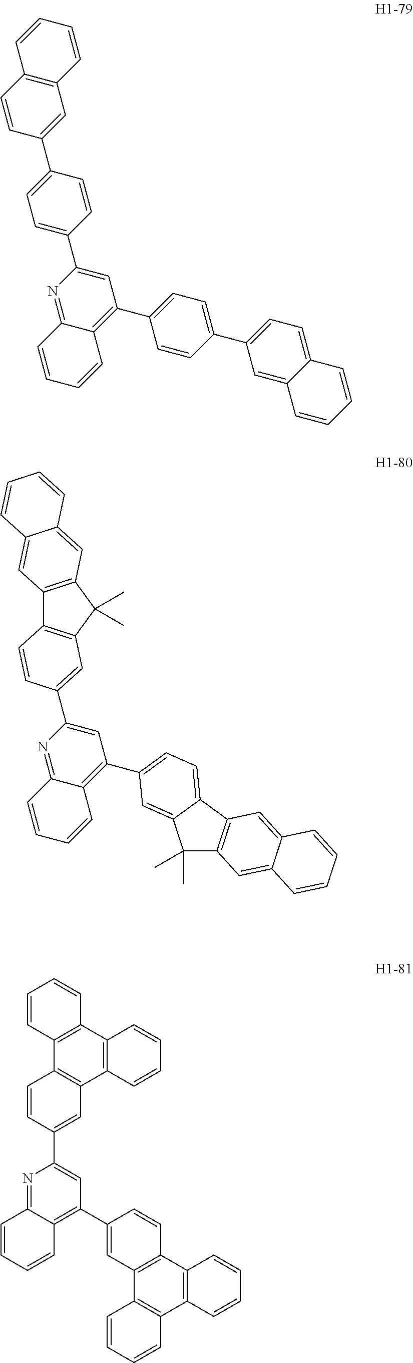 [SMILES: c1ccc2cc(-c3ccc(-c4cc(-c5ccc(-c6ccc7ccccc7c6)cc5)c5ccccc5n4)cc3)ccc2c1 ; CC1(C)c2cc(-c3cc(-c4ccc5c(c4)C(C)(C)c4cc6ccccc6cc4-5)c4ccccc4n3)ccc2-c2cc3ccccc3cc21 ; c1ccc2c(-c3ccc4c5ccccc5c5ccccc5c4c3)cc(-c3ccc4c5ccccc5c5ccccc5c4c3)nc2c1]

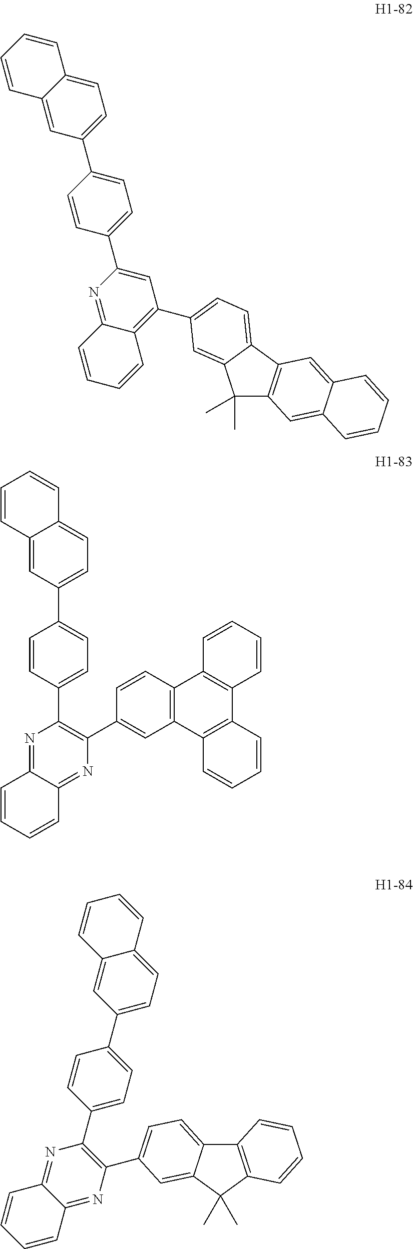 [SMILES: CC1(C)c2cc(-c3cc(-c4ccc(-c5ccc6ccccc6c5)cc4)nc4ccccc34)ccc2-c2cc3ccccc3cc21 ; c1ccc2cc(-c3ccc(-c4nc5ccccc5nc4-c4ccc5c6ccccc6c6ccccc6c5c4)cc3)ccc2c1 ; CC1(C)c2ccccc2-c2ccc(-c3nc4ccccc4nc3-c3ccc(-c4ccc5ccccc5c4)cc3)cc21]

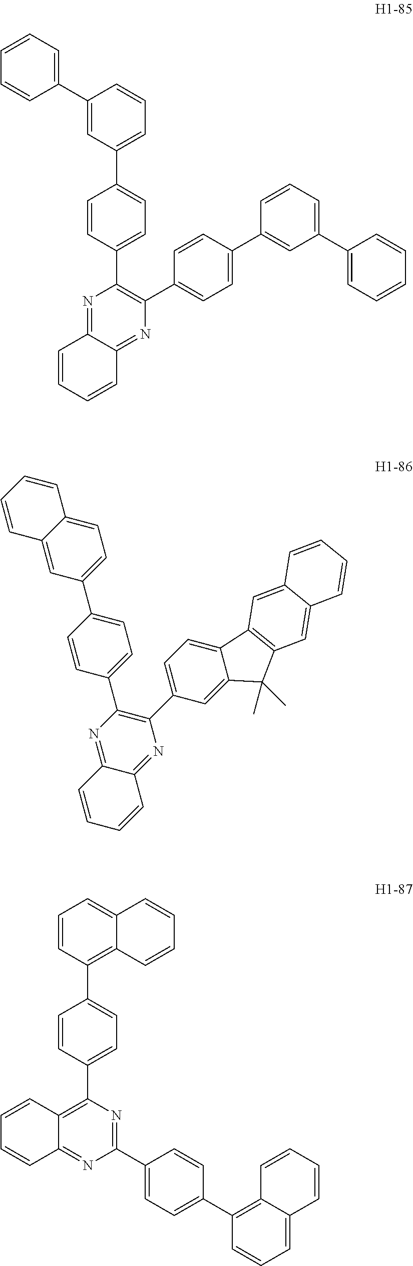 [SMILES: c1ccc(-c2cccc(-c3ccc(-c4nc5ccccc5nc4-c4ccc(-c5cccc(-c6ccccc6)c5)cc4)cc3)c2)cc1 ; CC1(C)c2cc(-c3nc4ccccc4nc3-c3ccc(-c4ccc5ccccc5c4)cc3)ccc2-c2cc3ccccc3cc21 ; c1ccc2c(-c3ccc(-c4nc(-c5ccc(-c6cccc7ccccc67)cc5)c5ccccc5n4)cc3)cccc2c1]

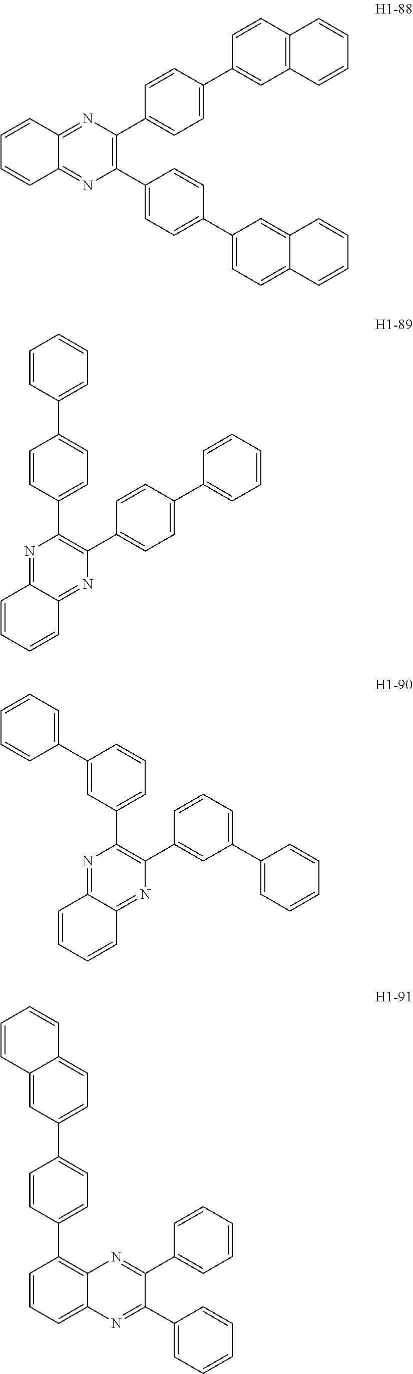

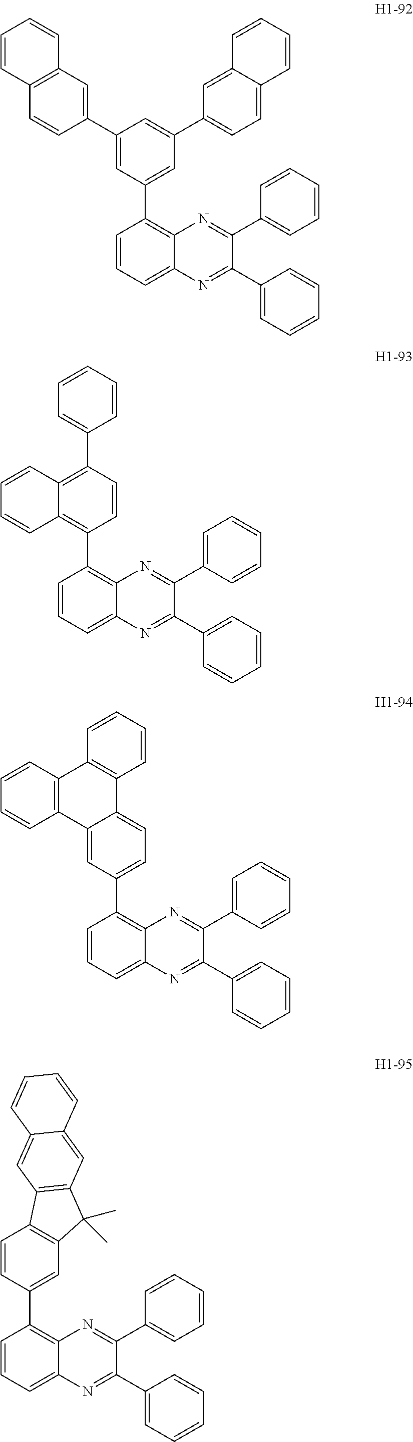 [SMILES: c1ccc(-c2nc3cccc(-c4cc(-c5ccc6ccccc6c5)cc(-c5ccc6ccccc6c5)c4)c3nc2-c2ccccc2)cc1 ; c1ccc(-c2nc3cccc(-c4ccc(-c5ccccc5)c5ccccc45)c3nc2-c2ccccc2)cc1 ; c1ccc(-c2nc3cccc(-c4ccc5c6ccccc6c6ccccc6c5c4)c3nc2-c2ccccc2)cc1 ; CC1(C)c2cc(-c3cccc4nc(-c5ccccc5)c(-c5ccccc5)nc34)ccc2-c2cc3ccccc3cc21]

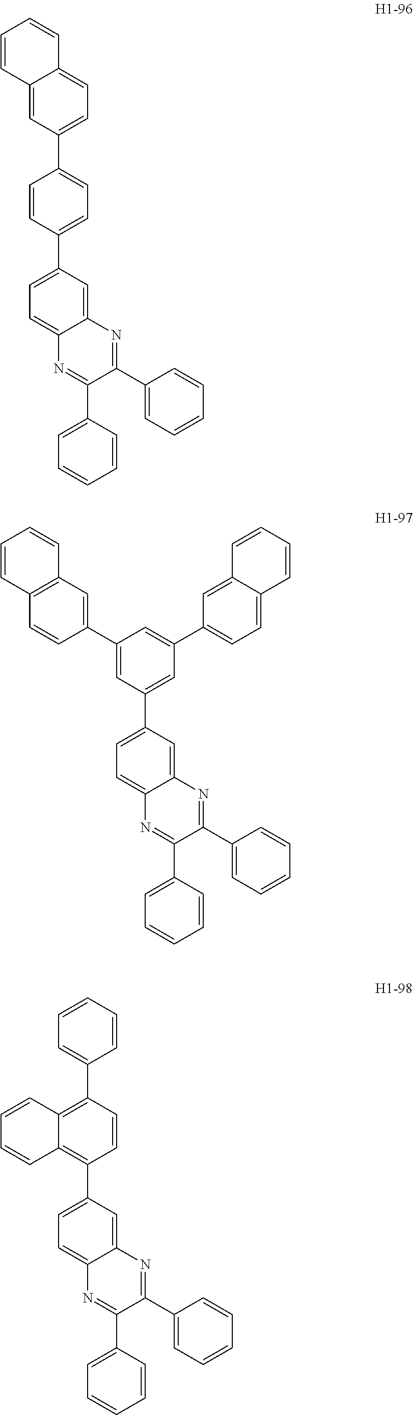 [SMILES: c1ccc(-c2nc3ccc(-c4ccc(-c5ccc6ccccc6c5)cc4)cc3nc2-c2ccccc2)cc1 ; c1ccc(-c2nc3ccc(-c4cc(-c5ccc6ccccc6c5)cc(-c5ccc6ccccc6c5)c4)cc3nc2-c2ccccc2)cc1 ; c1ccc(-c2nc3ccc(-c4ccc(-c5ccccc5)c5ccccc45)cc3nc2-c2ccccc2)cc1]

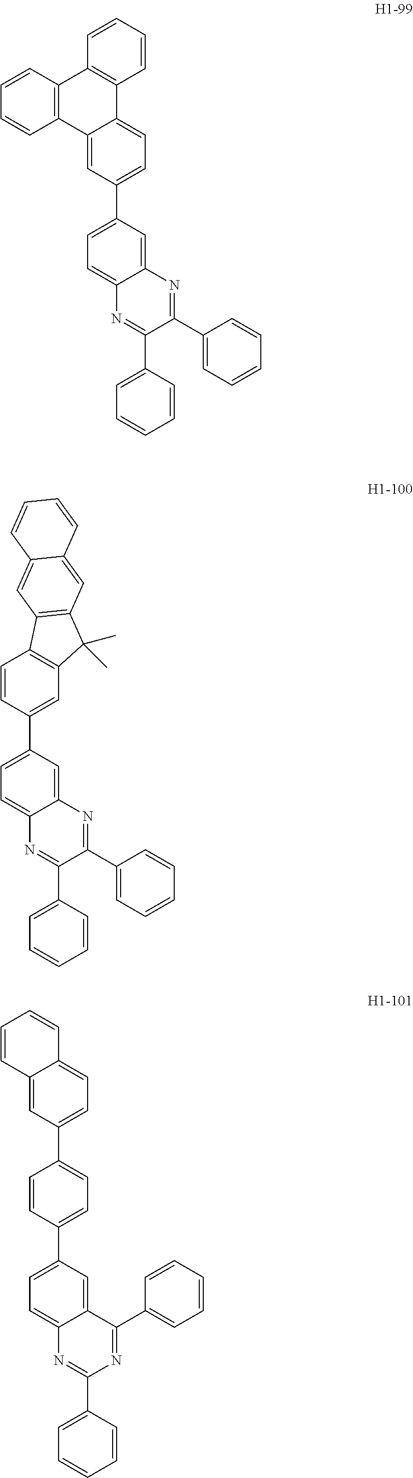 [SMILES: c1ccc(-c2nc3ccc(-c4ccc5c6ccccc6c6ccccc6c5c4)cc3nc2-c2ccccc2)cc1 ; CC1(C)c2cc(-c3ccc4nc(-c5ccccc5)c(-c5ccccc5)nc4c3)ccc2-c2cc3ccccc3cc21 ; c1ccc(-c2nc(-c3ccccc3)c3cc(-c4ccc(-c5ccc6ccccc6c5)cc4)ccc3n2)cc1]

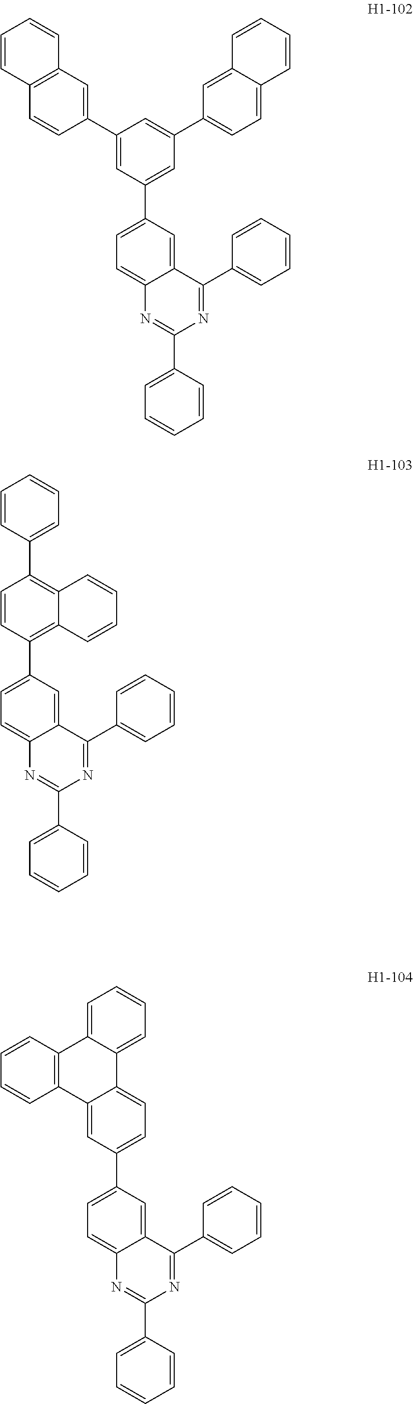 [SMILES: c1ccc(-c2nc(-c3ccccc3)c3cc(-c4cc(-c5ccc6ccccc6c5)cc(-c5ccc6ccccc6c5)c4)ccc3n2)cc1 ; c1ccc(-c2nc(-c3ccccc3)c3cc(-c4ccc(-c5ccccc5)c5ccccc45)ccc3n2)cc1 ; c1ccc(-c2nc(-c3ccccc3)c3cc(-c4ccc5c6ccccc6c6ccccc6c5c4)ccc3n2)cc1]

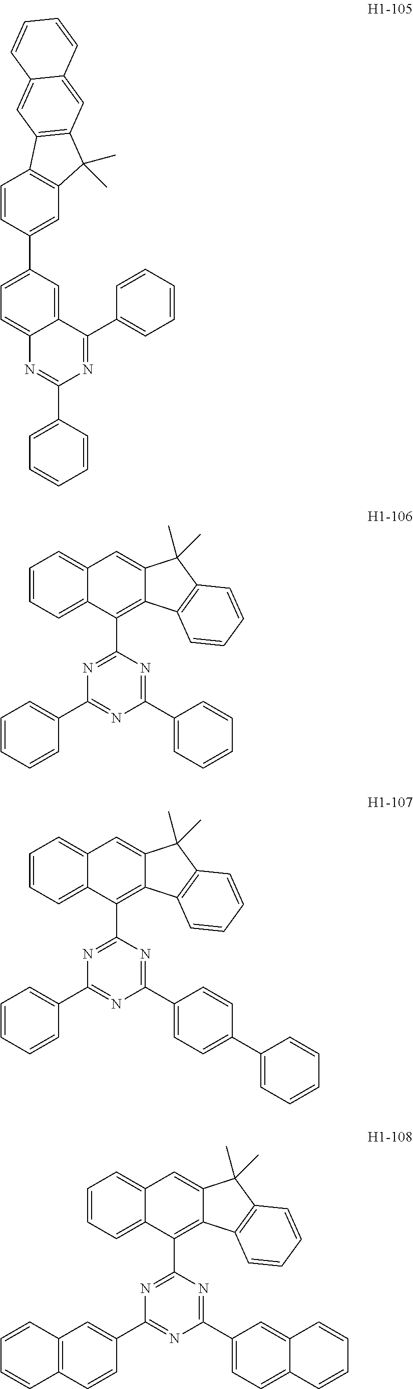 [SMILES: CC1(C)c2cc(-c3ccc4nc(-c5ccccc5)nc(-c5ccccc5)c4c3)ccc2-c2cc3ccccc3cc21 ; CC1(C)c2ccccc2-c2c1cc1ccccc1c2-c1nc(-c2ccccc2)nc(-c2ccccc2)n1 ; CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc1ccccc1c2-c1nc(-c2ccc3ccccc3c2)nc(-c2ccc3ccccc3c2)n1]

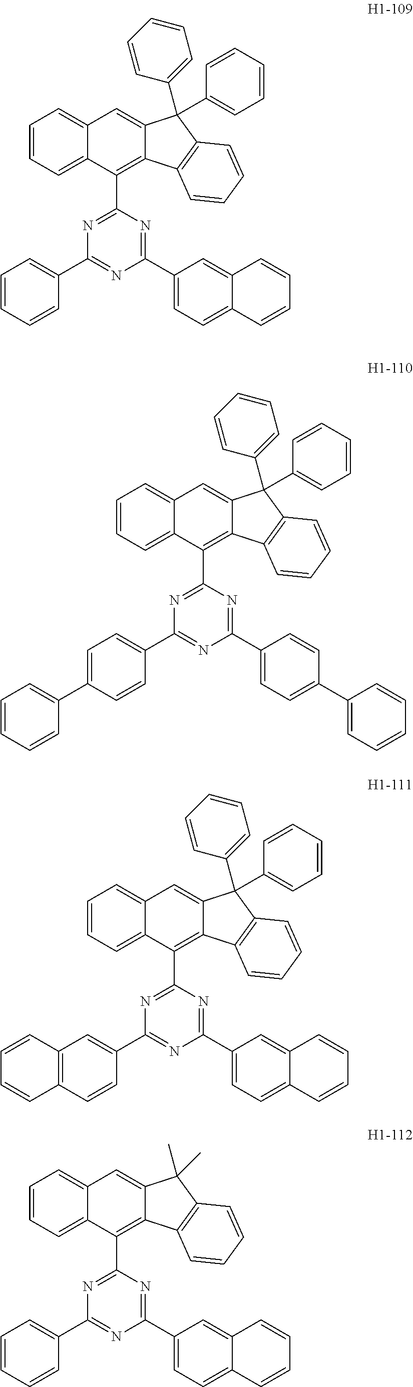 [SMILES: c1ccc(-c2nc(-c3ccc4ccccc4c3)nc(-c3c4c(cc5ccccc35)C(c3ccccc3)(c3ccccc3)c3ccccc3-4)n2)cc1 ; c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1 ; CC1(C)c2ccccc2-c2c1cc1ccccc1c2-c1nc(-c2ccccc2)nc(-c2ccc3ccccc3c2)n1]

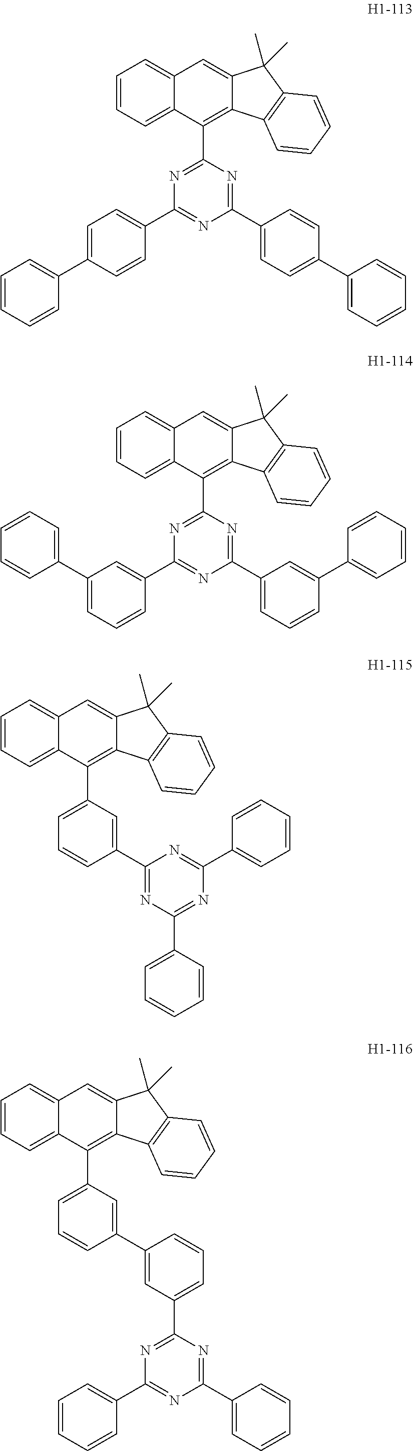 [SMILES: CC1(C)c2ccccc2-c2c1cc1ccccc1c2-c1nc(-c2ccc(-c3ccccc3)cc2)nc(-c2ccc(-c3ccccc3)cc2)n1 ; CC1(C)c2ccccc2-c2c1cc1ccccc1c2-c1nc(-c2cccc(-c3ccccc3)c2)nc(-c2cccc(-c3ccccc3)c2)n1 ; CC1(C)c2ccccc2-c2c1cc1ccccc1c2-c1cccc(-c2nc(-c3ccccc3)nc(-c3ccccc3)n2)c1 ; CC1(C)c2ccccc2-c2c1cc1ccccc1c2-c1cccc(-c2cccc(-c3nc(-c4ccccc4)nc(-c4ccccc4)n3)c2)c1]

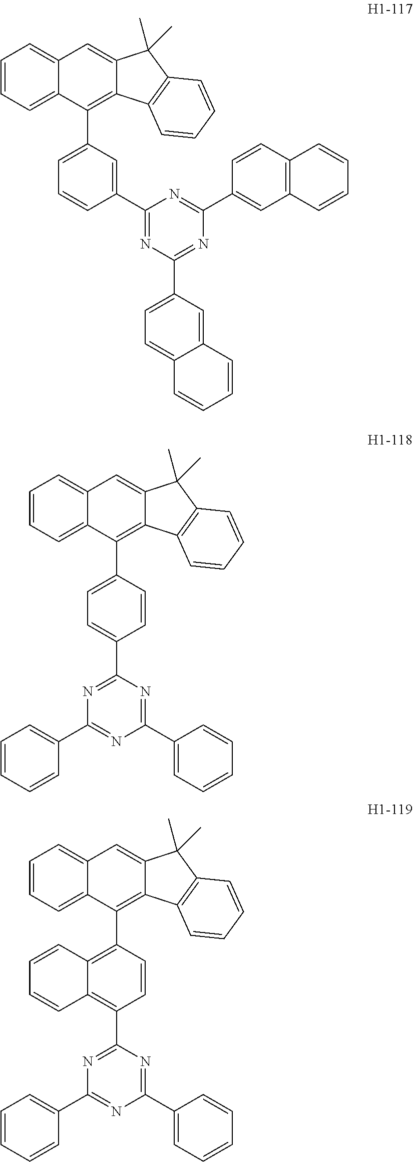

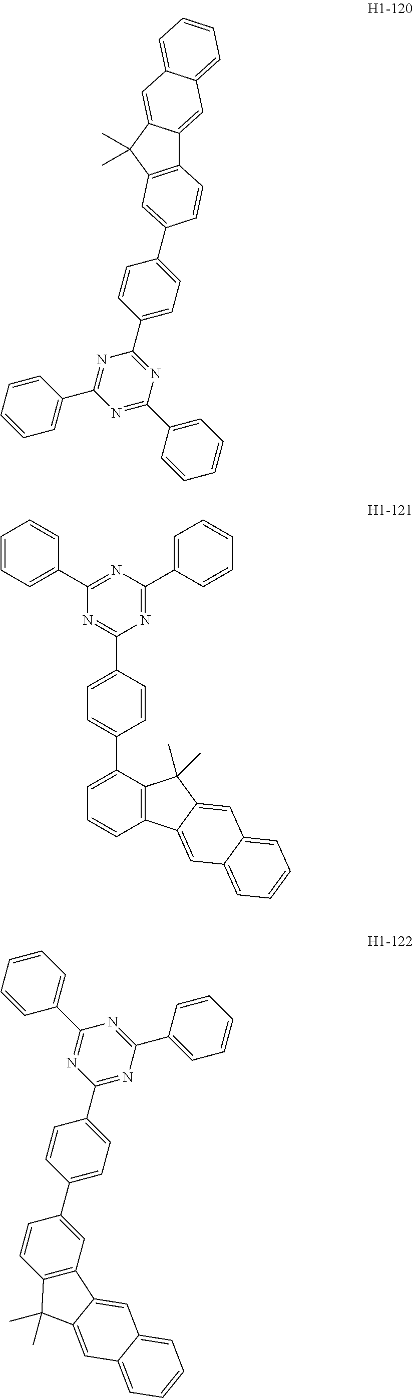

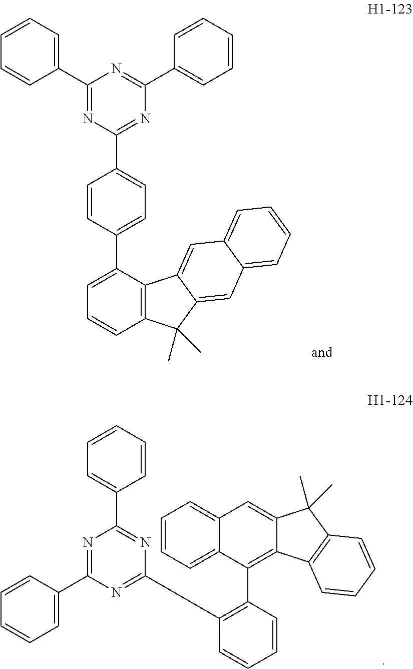 [SMILES: CC1(C)c2cc3ccccc3cc2-c2c(-c3ccc(-c4nc(-c5ccccc5)nc(-c5ccccc5)n4)cc3)cccc21 ; CC1(C)c2ccccc2-c2c1cc1ccccc1c2-c1ccccc1-c1nc(-c2ccccc2)nc(-c2ccccc2)n1]

9. The plurality of host materials according to claim 1, wherein the compound represented by formula 2 is selected from the following compounds:

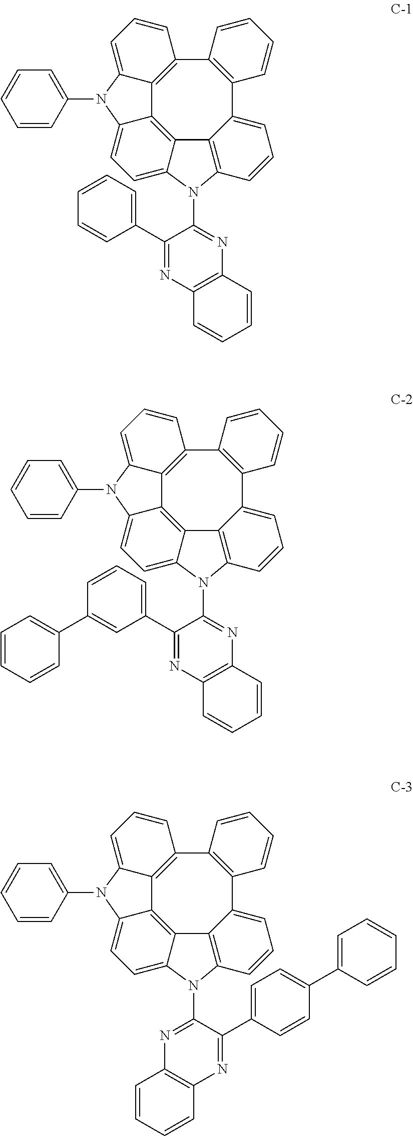 [SMILES: c1ccc(-c2nc3ccccc3nc2-n2c3cccc4c5ccccc5c5cccc6c5c5c(c43)c2ccc5n6-c2ccccc2)cc1 ; c1ccc(-c2cccc(-c3nc4ccccc4nc3-n3c4cccc5c6ccccc6c6cccc7c6c6c(c54)c3ccc6n7-c3ccccc3)c2)cc1 ; c1ccc(-c2ccc(-c3nc4ccccc4nc3-n3c4cccc5c6ccccc6c6cccc7c6c6c(c54)c3ccc6n7-c3ccccc3)cc2)cc1]

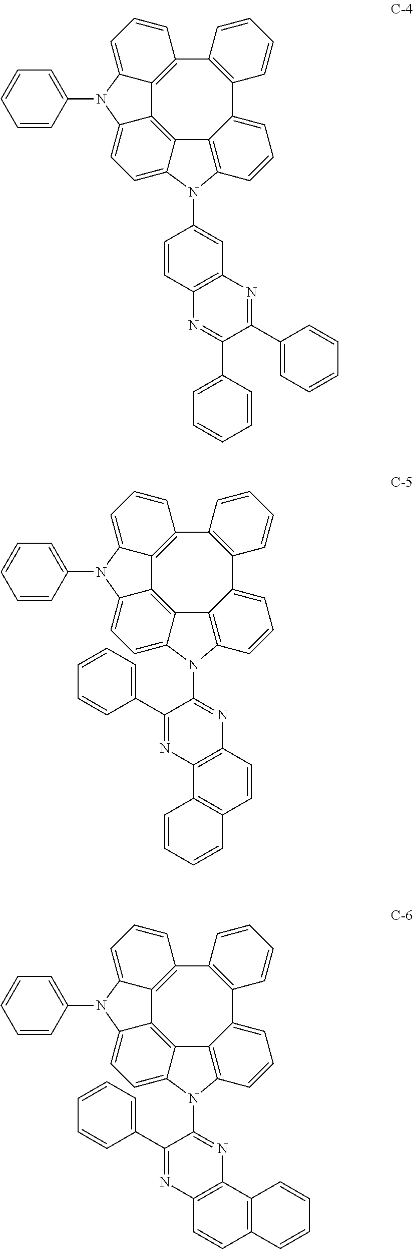 [SMILES: c1ccc(-c2nc3ccc(-n4c5cccc6c7ccccc7c7cccc8c7c7c(c65)c4ccc7n8-c4ccccc4)cc3nc2-c2ccccc2)cc1 ; c1ccc(-c2nc3c(ccc4ccccc43)nc2-n2c3cccc4c5ccccc5c5cccc6c5c5c(c43)c2ccc5n6-c2ccccc2)cc1 ; c1ccc(-c2nc3ccc4ccccc4c3nc2-n2c3cccc4c5ccccc5c5cccc6c5c5c(c43)c2ccc5n6-c2ccccc2)cc1]

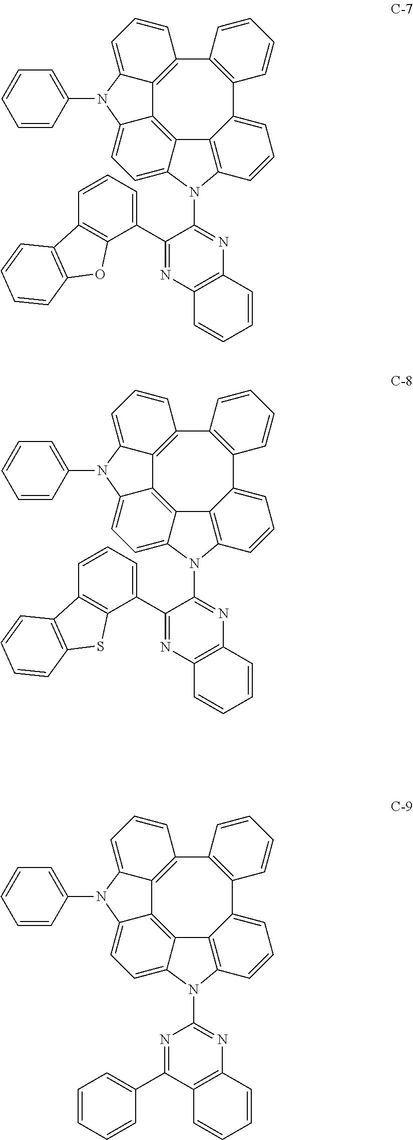 [SMILES: c1ccc(-n2c3cccc4c5ccccc5c5cccc6c5c5c(c43)c2ccc5n6-c2nc3ccccc3nc2-c2cccc3c2oc2ccccc23)cc1 ; c1ccc(-n2c3cccc4c5ccccc5c5cccc6c5c5c(c43)c2ccc5n6-c2nc3ccccc3nc2-c2cccc3c2sc2ccccc23)cc1 ; c1ccc(-c2nc(-n3c4cccc5c6ccccc6c6cccc7c6c6c(c54)c3ccc6n7-c3ccccc3)nc3ccccc23)cc1]

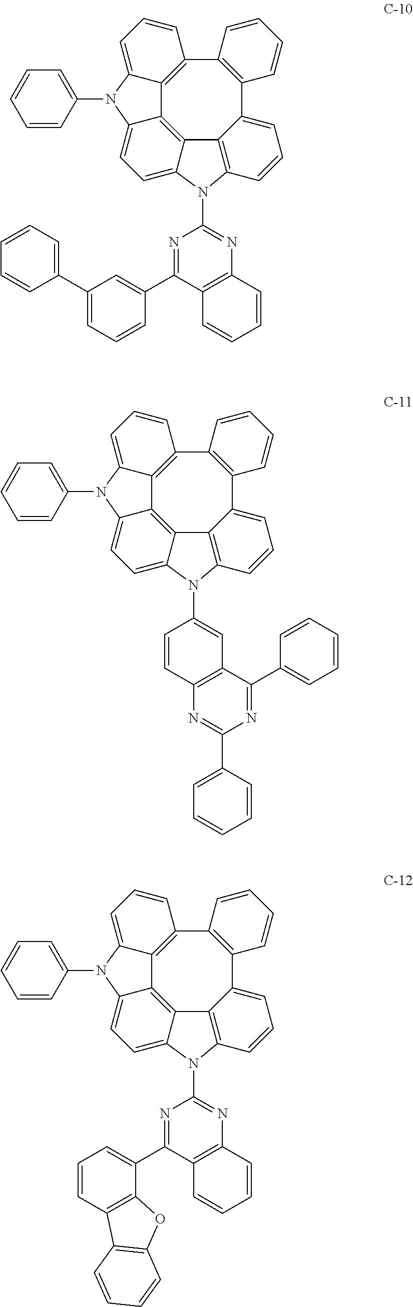 [SMILES: c1ccc(-c2cccc(-c3nc(-n4c5cccc6c7ccccc7c7cccc8c7c7c(c65)c4ccc7n8-c4ccccc4)nc4ccccc34)c2)cc1 ; c1ccc(-c2nc(-c3ccccc3)c3cc(-n4c5cccc6c7ccccc7c7cccc8c7c7c(c65)c4ccc7n8-c4ccccc4)ccc3n2)cc1 ; c1ccc(-n2c3cccc4c5ccccc5c5cccc6c5c5c(c43)c2ccc5n6-c2nc(-c3cccc4c3oc3ccccc34)c3ccccc3n2)cc1]

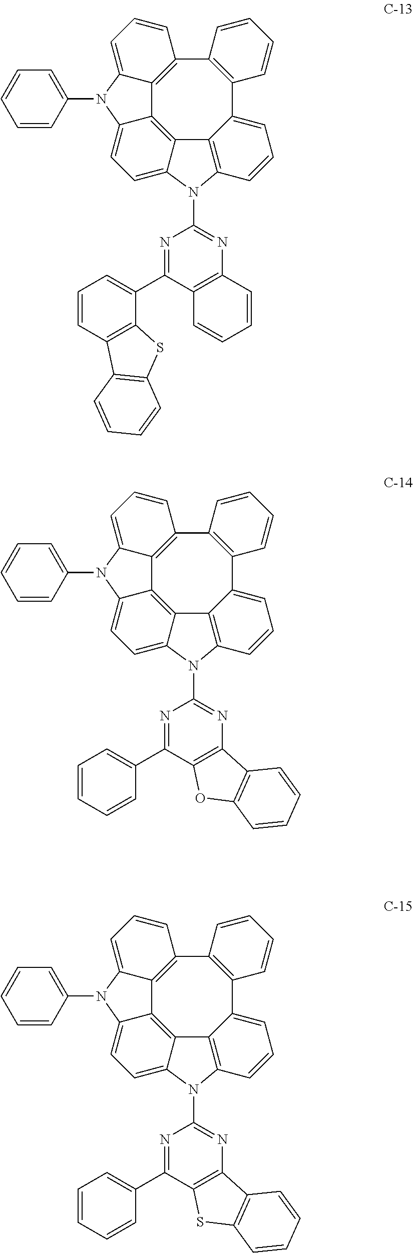 [SMILES: c1ccc(-n2c3cccc4c5ccccc5c5cccc6c5c5c(c43)c2ccc5n6-c2nc(-c3cccc4c3sc3ccccc34)c3ccccc3n2)cc1 ; c1ccc(-c2nc(-n3c4cccc5c6ccccc6c6cccc7c6c6c(c54)c3ccc6n7-c3ccccc3)nc3c2oc2ccccc23)cc1 ; c1ccc(-c2nc(-n3c4cccc5c6ccccc6c6cccc7c6c6c(c54)c3ccc6n7-c3ccccc3)nc3c2sc2ccccc23)cc1]

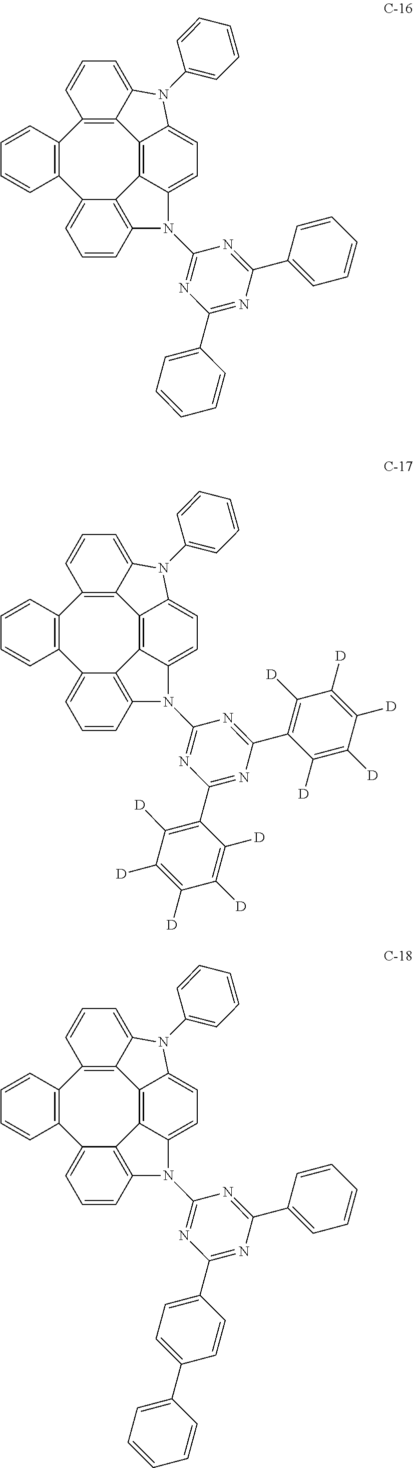 [SMILES: c1ccc(-c2nc(-c3ccccc3)nc(-n3c4cccc5c6ccccc6c6cccc7c6c6c(c54)c3ccc6n7-c3ccccc3)n2)cc1 ; [2H]c1c([2H])c([2H])c(-c2nc(-c3c([2H])c([2H])c([2H])c([2H])c3[2H])nc(-n3c4cccc5c6ccccc6c6cccc7c6c6c(c54)c3ccc6n7-c3ccccc3)n2)c([2H])c1[2H] ; c1ccc(-c2ccc(-c3nc(-c4ccccc4)nc(-n4c5cccc6c7ccccc7c7cccc8c7c7c(c65)c4ccc7n8-c4ccccc4)n3)cc2)cc1]

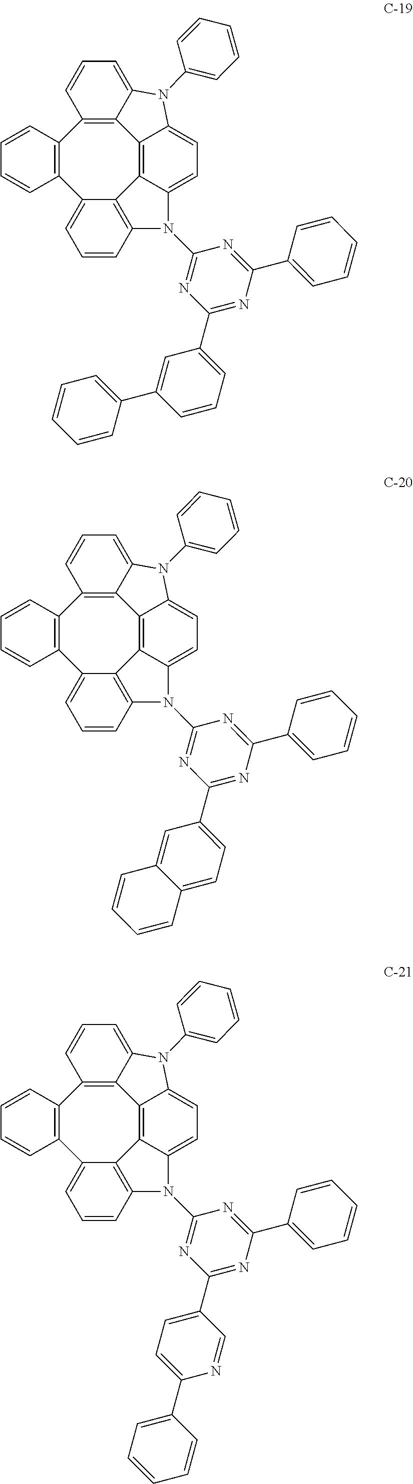 [SMILES: c1ccc(-c2cccc(-c3nc(-c4ccccc4)nc(-n4c5cccc6c7ccccc7c7cccc8c7c7c(c65)c4ccc7n8-c4ccccc4)n3)c2)cc1 ; c1ccc(-c2nc(-c3ccc4ccccc4c3)nc(-n3c4cccc5c6ccccc6c6cccc7c6c6c(c54)c3ccc6n7-c3ccccc3)n2)cc1 ; c1ccc(-c2ccc(-c3nc(-c4ccccc4)nc(-n4c5cccc6c7ccccc7c7cccc8c7c7c(c65)c4ccc7n8-c4ccccc4)n3)cn2)cc1]

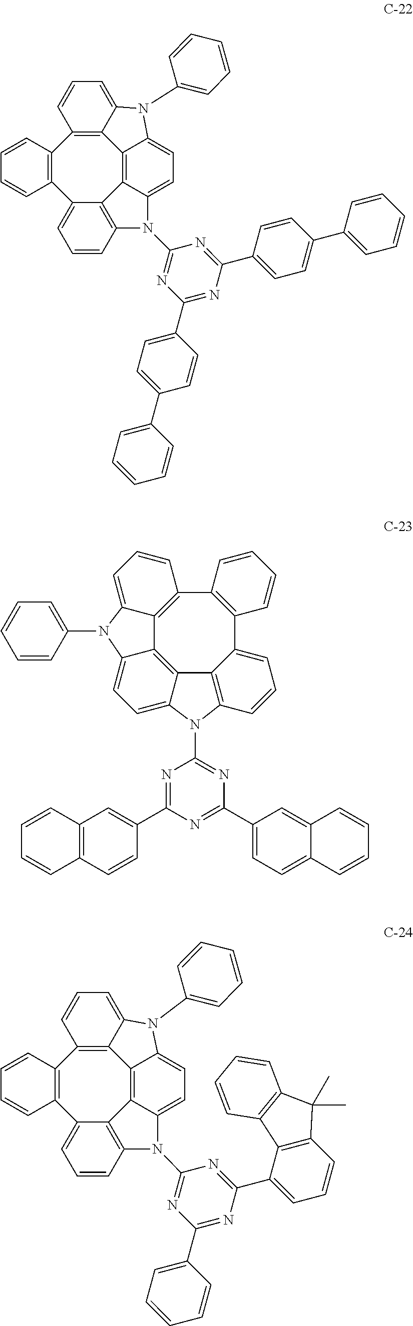 [SMILES: c1ccc(-c2ccc(-c3nc(-c4ccc(-c5ccccc5)cc4)nc(-n4c5cccc6c7ccccc7c7cccc8c7c7c(c65)c4ccc7n8-c4ccccc4)n3)cc2)cc1 ; c1ccc(-n2c3cccc4c5ccccc5c5cccc6c5c5c(c43)c2ccc5n6-c2nc(-c3ccc4ccccc4c3)nc(-c3ccc4ccccc4c3)n2)cc1 ; CC1(C)c2ccccc2-c2c(-c3nc(-c4ccccc4)nc(-n4c5cccc6c7ccccc7c7cccc8c7c7c(c65)c4ccc7n8-c4ccccc4)n3)cccc21]

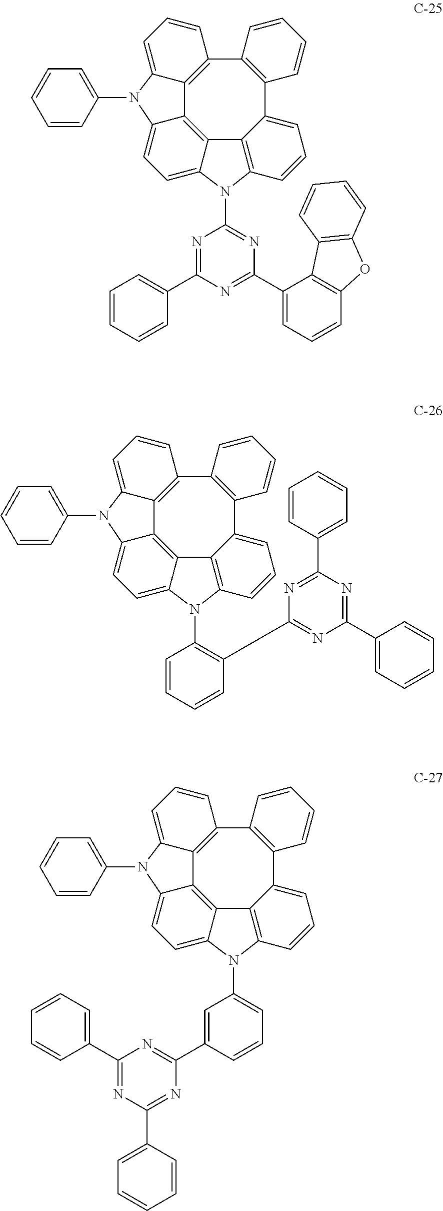 [SMILES: c1ccc(-c2nc(-c3cccc4oc5ccccc5c34)nc(-n3c4cccc5c6ccccc6c6cccc7c6c6c(c54)c3ccc6n7-c3ccccc3)n2)cc1 ; c1ccc(-c2nc(-c3ccccc3)nc(-c3ccccc3-n3c4cccc5c6ccccc6c6cccc7c6c6c(c54)c3ccc6n7-c3ccccc3)n2)cc1 ; c1ccc(-c2nc(-c3ccccc3)nc(-c3cccc(-n4c5cccc6c7ccccc7c7cccc8c7c7c(c65)c4ccc7n8-c4ccccc4)c3)n2)cc1]

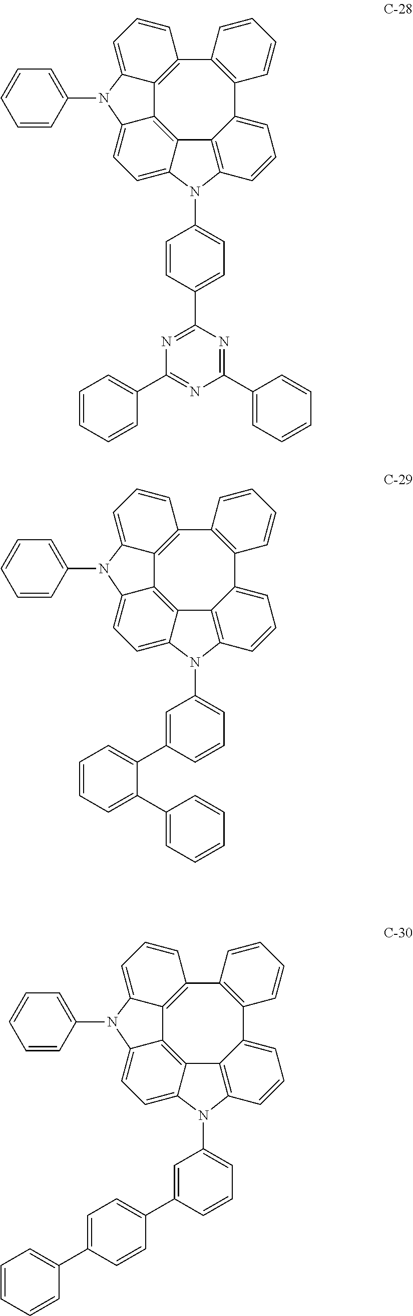 [SMILES: c1ccc(-c2nc(-c3ccccc3)nc(-c3ccc(-n4c5cccc6c7ccccc7c7cccc8c7c7c(c65)c4ccc7n8-c4ccccc4)cc3)n2)cc1 ; c1ccc(-c2ccccc2-c2cccc(-n3c4cccc5c6ccccc6c6cccc7c6c6c(c54)c3ccc6n7-c3ccccc3)c2)cc1 ; c1ccc(-c2ccc(-c3cccc(-n4c5cccc6c7ccccc7c7cccc8c7c7c(c65)c4ccc7n8-c4ccccc4)c3)cc2)cc1]

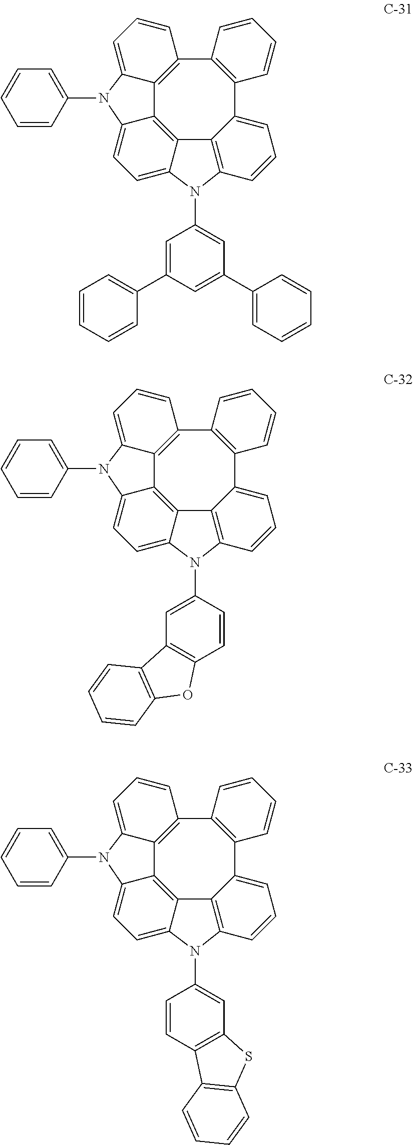 [SMILES: c1ccc(-c2cc(-c3ccccc3)cc(-n3c4cccc5c6ccccc6c6cccc7c6c6c(c54)c3ccc6n7-c3ccccc3)c2)cc1 ; c1ccc(-n2c3cccc4c5ccccc5c5cccc6c5c5c(c43)c2ccc5n6-c2ccc3oc4ccccc4c3c2)cc1 ; c1ccc(-n2c3cccc4c5ccccc5c5cccc6c5c5c(c43)c2ccc5n6-c2ccc3c(c2)sc2ccccc23)cc1]

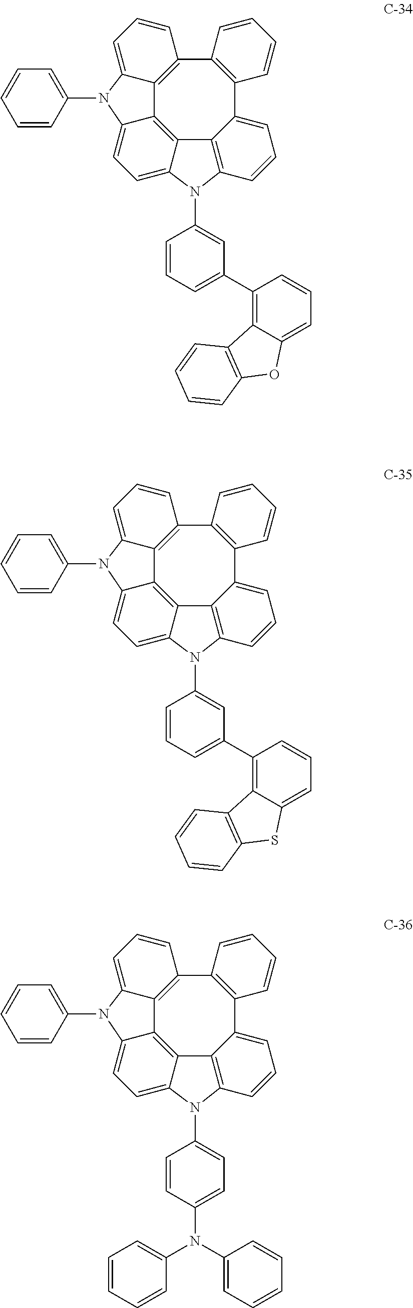 [SMILES: c1ccc(-n2c3cccc4c5ccccc5c5cccc6c5c5c(c43)c2ccc5n6-c2cccc(-c3cccc4oc5ccccc5c34)c2)cc1 ; c1ccc(-n2c3cccc4c5ccccc5c5cccc6c5c5c(c43)c2ccc5n6-c2cccc(-c3cccc4sc5ccccc5c34)c2)cc1 ; c1ccc(N(c2ccccc2)c2ccc(-n3c4cccc5c6ccccc6c6cccc7c6c6c(c54)c3ccc6n7-c3ccccc3)cc2)cc1]

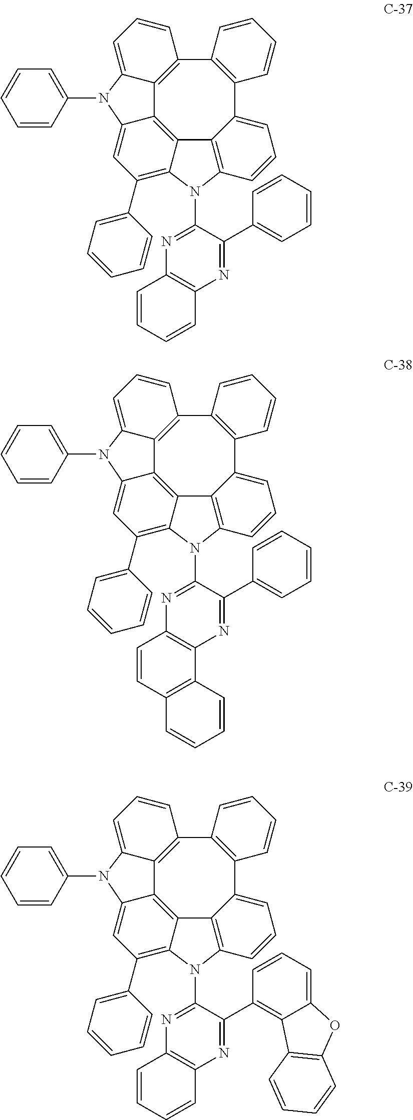

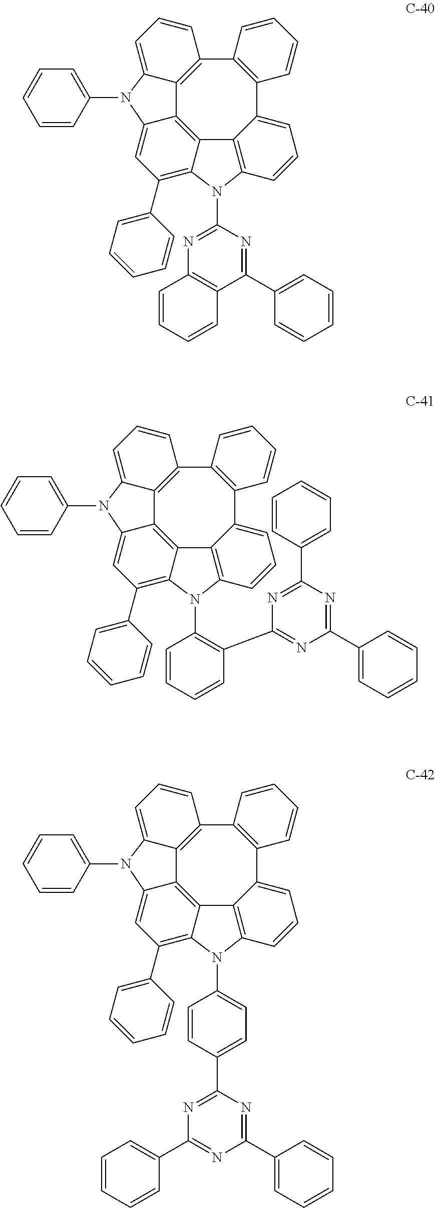 [SMILES: c1ccc(-c2nc(-n3c4cccc5c6ccccc6c6cccc7c6c6c(c54)c3c(-c3ccccc3)cc6n7-c3ccccc3)nc3ccccc23)cc1 ; c1ccc(-c2nc(-c3ccccc3)nc(-c3ccccc3-n3c4cccc5c6ccccc6c6cccc7c6c6c(c54)c3c(-c3ccccc3)cc6n7-c3ccccc3)n2)cc1 ; c1ccc(-c2nc(-c3ccccc3)nc(-c3ccc(-n4c5cccc6c7ccccc7c7cccc8c7c7c(c65)c4c(-c4ccccc4)cc7n8-c4ccccc4)cc3)n2)cc1]

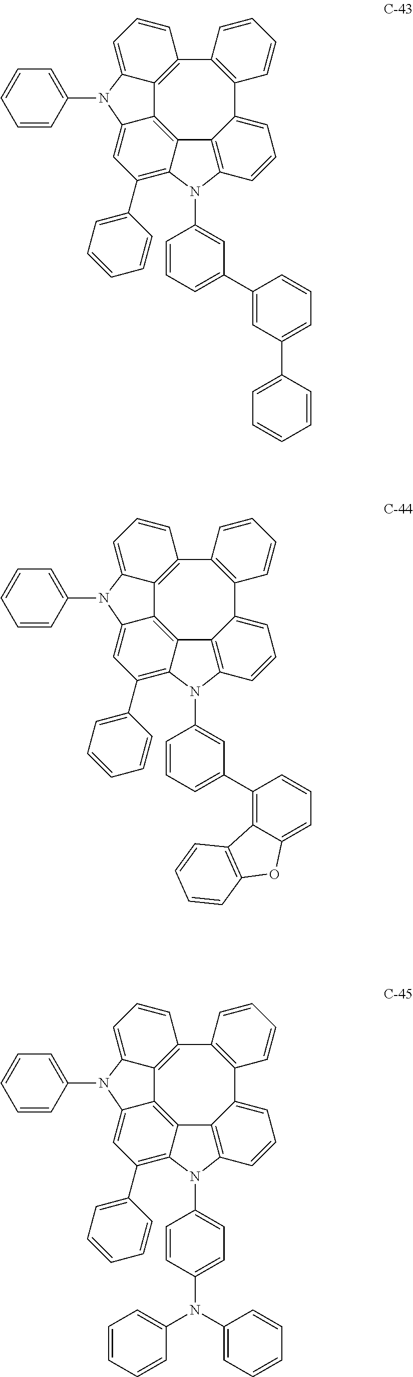 [SMILES: c1ccc(-c2cccc(-c3cccc(-n4c5cccc6c7ccccc7c7cccc8c7c7c(c65)c4c(-c4ccccc4)cc7n8-c4ccccc4)c3)c2)cc1 ; c1ccc(-c2cc3c4c5c(cccc5n3-c3ccccc3)c3ccccc3c3cccc5c3c4c2n5-c2cccc(-c3cccc4oc5ccccc5c34)c2)cc1 ; c1ccc(-c2cc3c4c5c(cccc5n3-c3ccccc3)c3ccccc3c3cccc5c3c4c2n5-c2ccc(N(c3ccccc3)c3ccccc3)cc2)cc1]

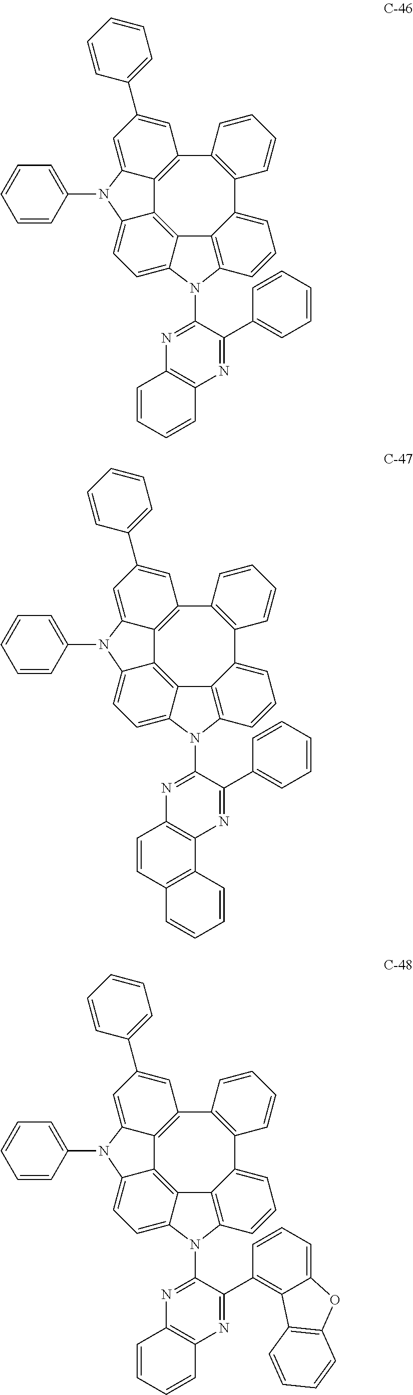

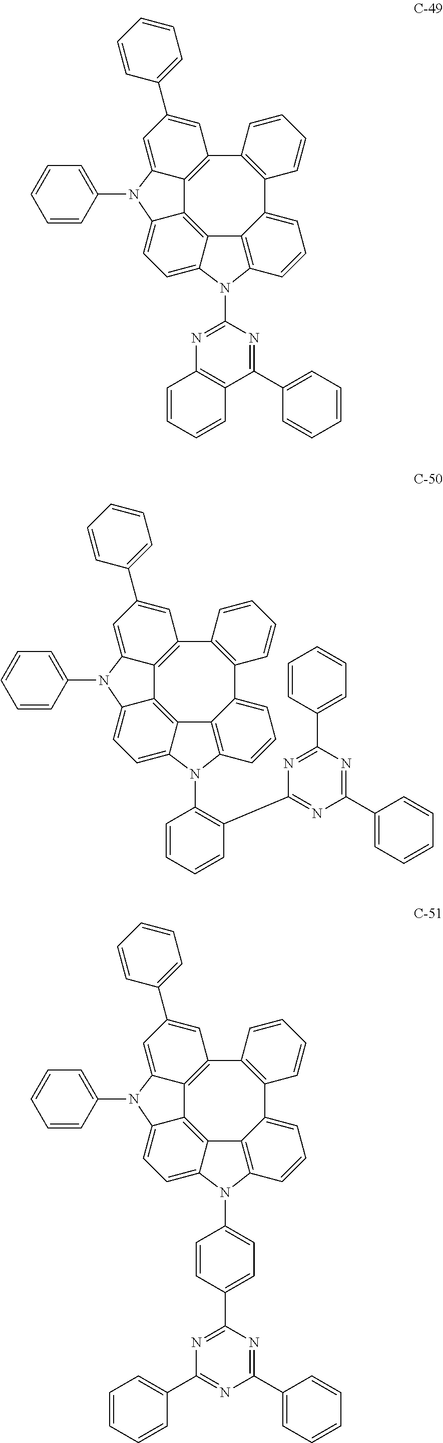 [SMILES: c1ccc(-c2cc3c4ccccc4c4cccc5c4c4c6c3c(c2)n(-c2ccccc2)c6ccc4n5-c2nc(-c3ccccc3)c3ccccc3n2)cc1 ; c1ccc(-c2cc3c4ccccc4c4cccc5c4c4c6c3c(c2)n(-c2ccccc2)c6ccc4n5-c2ccccc2-c2nc(-c3ccccc3)nc(-c3ccccc3)n2)cc1 ; c1ccc(-c2cc3c4ccccc4c4cccc5c4c4c6c3c(c2)n(-c2ccccc2)c6ccc4n5-c2ccc(-c3nc(-c4ccccc4)nc(-c4ccccc4)n3)cc2)cc1]

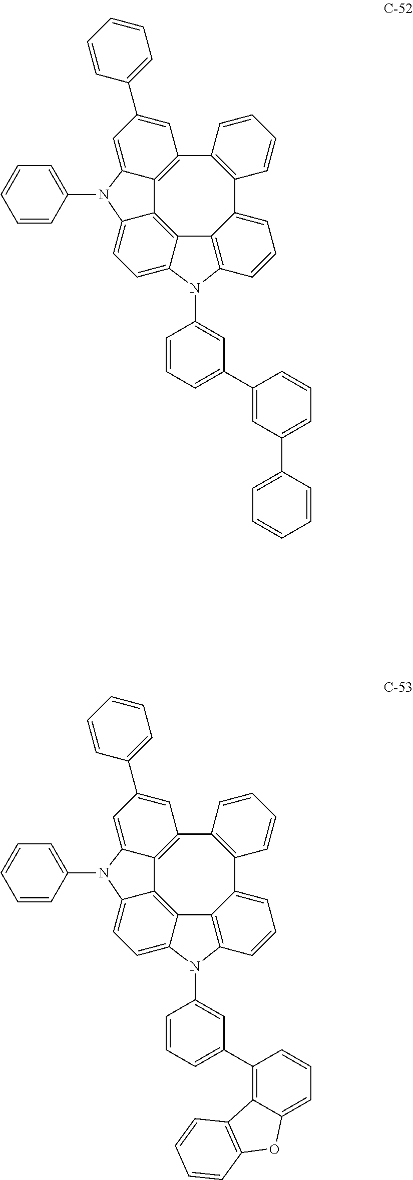 [SMILES: c1ccc(-c2cccc(-c3cccc(-n4c5cccc6c7ccccc7c7cc(-c8ccccc8)cc8c7c7c(c65)c4ccc7n8-c4ccccc4)c3)c2)cc1 ; c1ccc(-c2cc3c4ccccc4c4cccc5c4c4c6c3c(c2)n(-c2ccccc2)c6ccc4n5-c2cccc(-c3cccc4oc5ccccc5c34)c2)cc1]

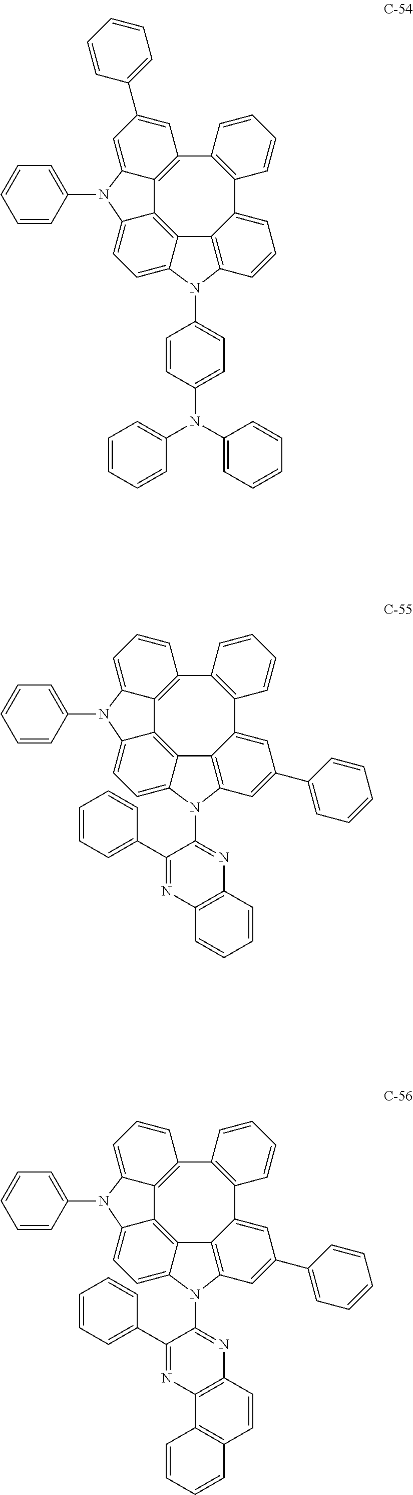 [SMILES: c1ccc(-c2cc3c4ccccc4c4cccc5c4c4c6c3c(c2)n(-c2ccccc2)c6ccc4n5-c2ccc(N(c3ccccc3)c3ccccc3)cc2)cc1 ; c1ccc(-c2cc3c4ccccc4c4cccc5c4c4c6c3c(c2)n(-c2nc3ccccc3nc2-c2ccccc2)c6ccc4n5-c2ccccc2)cc1 ; c1ccc(-c2cc3c4ccccc4c4cccc5c4c4c6c3c(c2)n(-c2nc3ccc7ccccc7c3nc2-c2ccccc2)c6ccc4n5-c2ccccc2)cc1]

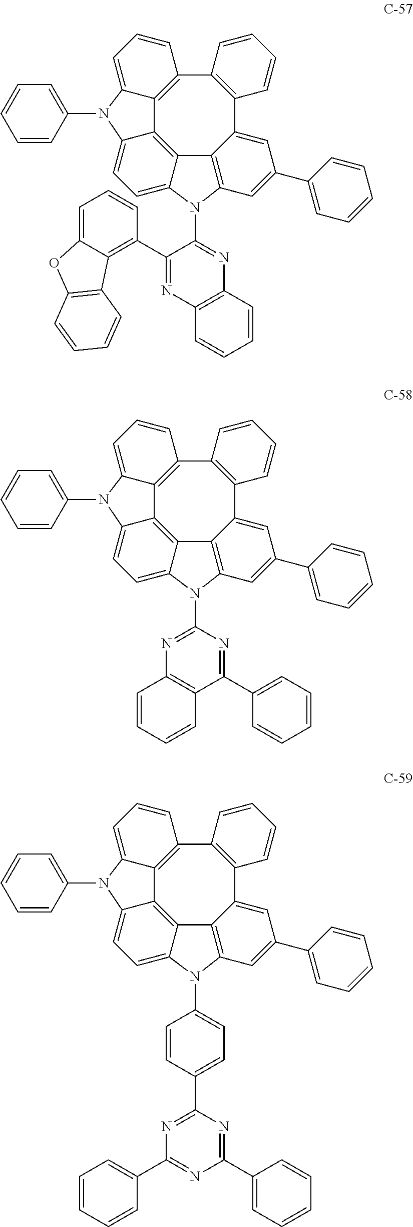 [SMILES: c1ccc(-c2cc3c4ccccc4c4cccc5c4c4c6c3c(c2)n(-c2nc3ccccc3nc2-c2cccc3oc7ccccc7c23)c6ccc4n5-c2ccccc2)cc1 ; c1ccc(-c2cc3c4ccccc4c4cccc5c4c4c6c3c(c2)n(-c2nc(-c3ccccc3)c3ccccc3n2)c6ccc4n5-c2ccccc2)cc1 ; c1ccc(-c2cc3c4ccccc4c4cccc5c4c4c6c3c(c2)n(-c2ccc(-c3nc(-c7ccccc7)nc(-c7ccccc7)n3)cc2)c6ccc4n5-c2ccccc2)cc1]

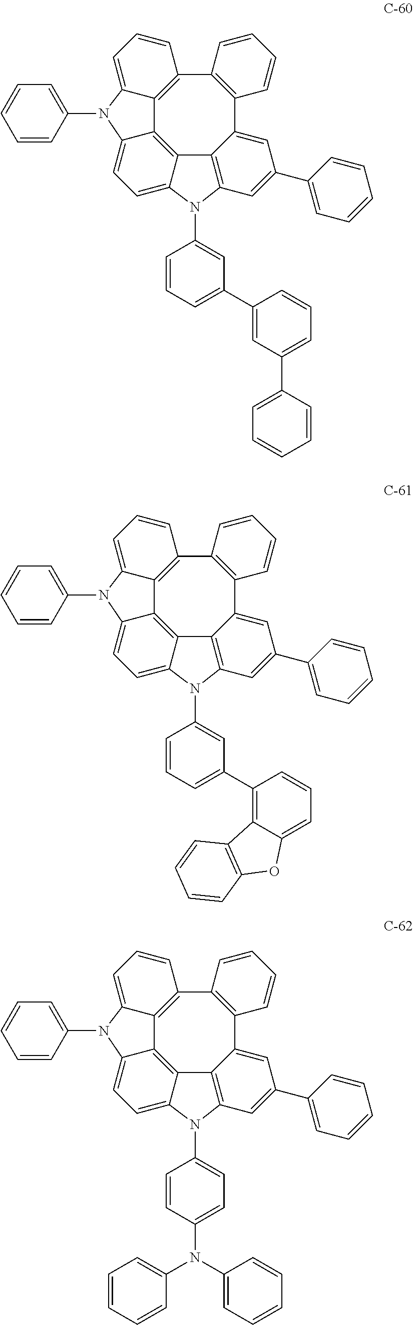 [SMILES: c1ccc(-c2cccc(-c3cccc(-n4c5cc(-c6ccccc6)cc6c7ccccc7c7cccc8c7c7c(c65)c4ccc7n8-c4ccccc4)c3)c2)cc1 ; c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1]

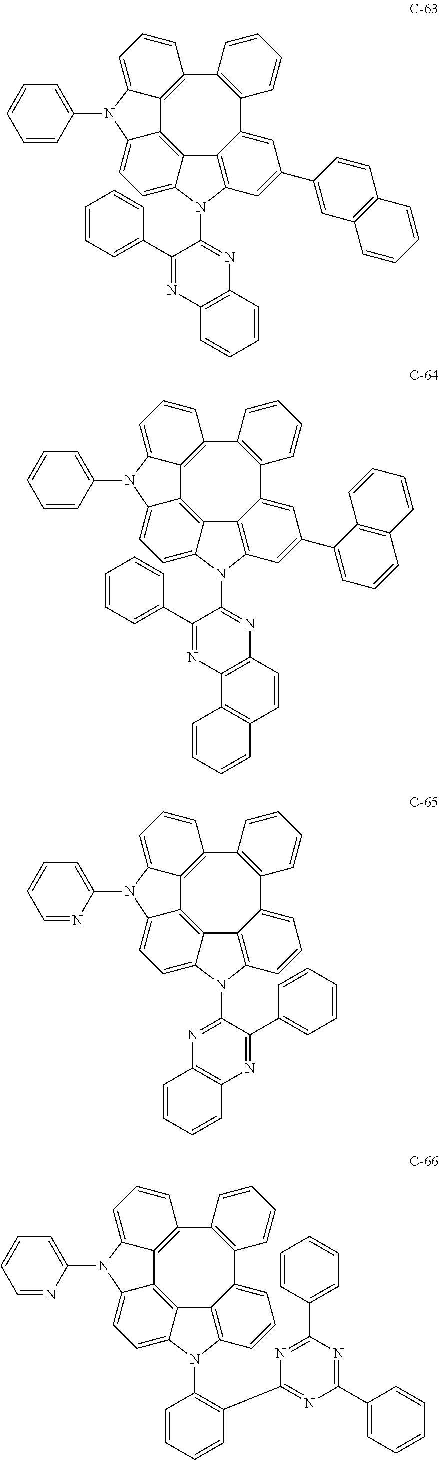 [SMILES: c1ccc(-c2nc3ccccc3nc2-n2c3cc(-c4ccc5ccccc5c4)cc4c5ccccc5c5cccc6c5c5c(c43)c2ccc5n6-c2ccccc2)cc1 ; c1ccc(-c2nc3c(ccc4ccccc43)nc2-n2c3cc(-c4cccc5ccccc45)cc4c5ccccc5c5cccc6c5c5c(c43)c2ccc5n6-c2ccccc2)cc1 ; c1ccc(-c2nc3ccccc3nc2-n2c3cccc4c5ccccc5c5cccc6c5c5c(c43)c2ccc5n6-c2ccccn2)cc1 ; c1ccc(-c2nc(-c3ccccc3)nc(-c3ccccc3-n3c4cccc5c6ccccc6c6cccc7c6c6c(c54)c3ccc6n7-c3ccccn3)n2)cc1]

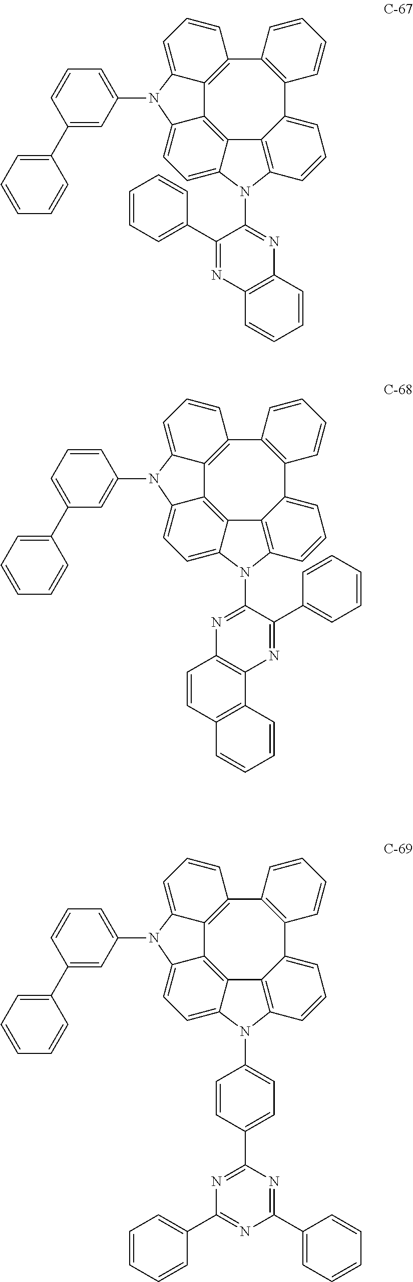 [SMILES: c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3nc4ccc5ccccc5c4nc3-c3ccccc3)c2)cc1 ; c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1]

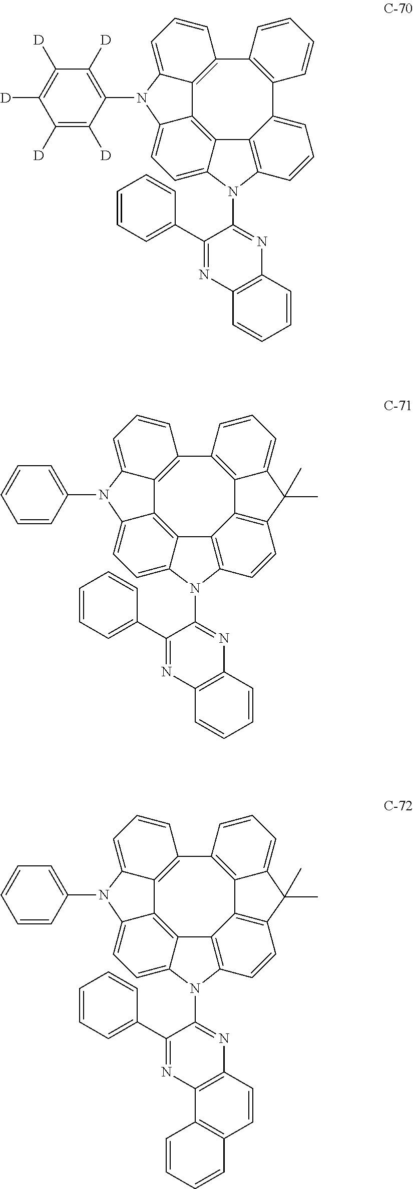 [SMILES: [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H] ; CC1(C)c2cccc3c4cccc5c4c4c6c7c(c1ccc7n(-c1nc7ccccc7nc1-c1ccccc1)c6ccc4n5-c1ccccc1)c23 ; CC1(C)c2cccc3c4cccc5c4c4c6c7c(c1ccc7n(-c1nc7ccc8ccccc8c7nc1-c1ccccc1)c6ccc4n5-c1ccccc1)c23]

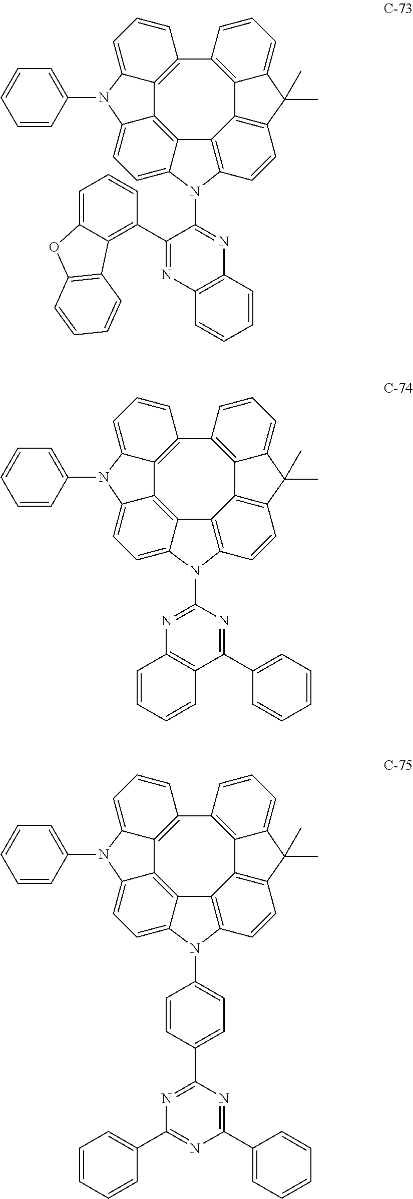 [SMILES: CC1(C)c2cccc3c4cccc5c4c4c6c7c(c1ccc7n(-c1nc7ccccc7nc1-c1cccc7oc8ccccc8c17)c6ccc4n5-c1ccccc1)c23 ; CC1(C)c2cccc3c4cccc5c4c4c6c7c(c1ccc7n(-c1nc(-c7ccccc7)c7ccccc7n1)c6ccc4n5-c1ccccc1)c23 ; CC1(C)c2cccc3c4cccc5c4c4c6c7c(c1ccc7n(-c1ccc(-c7nc(-c8ccccc8)nc(-c8ccccc8)n7)cc1)c6ccc4n5-c1ccccc1)c23]

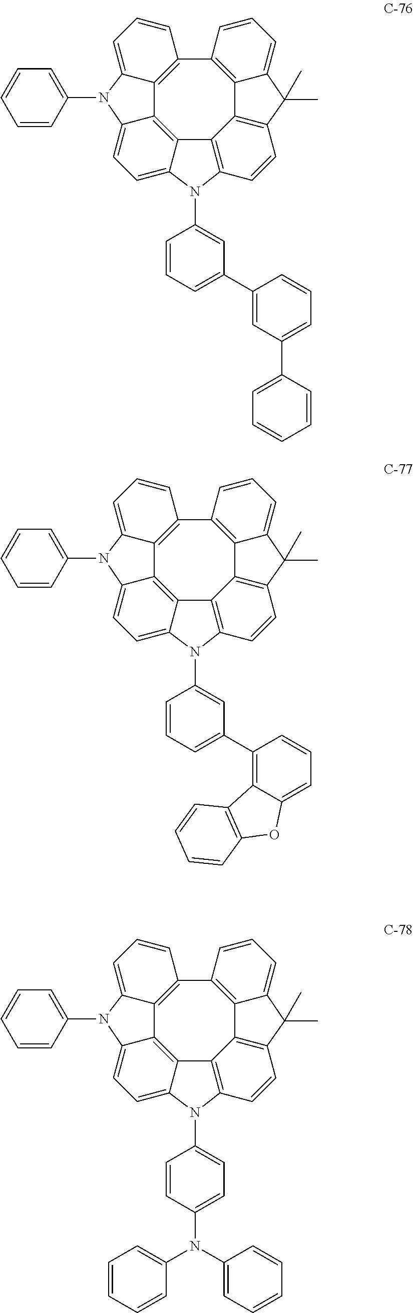 [SMILES: CC1(C)c2cccc3c4cccc5c4c4c6c7c(c1ccc7n(-c1cccc(-c7cccc(-c8ccccc8)c7)c1)c6ccc4n5-c1ccccc1)c23 ; CC1(C)c2cccc3c4cccc5c4c4c6c7c(c1ccc7n(-c1cccc(-c7cccc8oc9ccccc9c78)c1)c6ccc4n5-c1ccccc1)c23 ; CC1(C)c2cccc3c4cccc5c4c4c6c7c(c1ccc7n(-c1ccc(N(c7ccccc7)c7ccccc7)cc1)c6ccc4n5-c1ccccc1)c23]

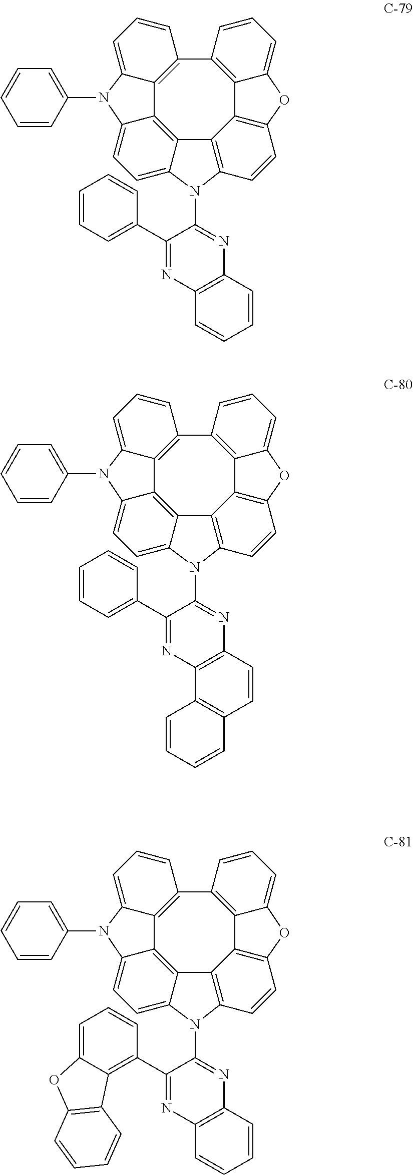 [SMILES: c1ccc(-c2nc3ccccc3nc2-n2c3ccc4oc5cccc6c7cccc8c7c7c(c3c4c56)c2ccc7n8-c2ccccc2)cc1 ; c1ccc(-c2nc3c(ccc4ccccc43)nc2-n2c3ccc4oc5cccc6c7cccc8c7c7c(c3c4c56)c2ccc7n8-c2ccccc2)cc1 ; c1ccc(-n2c3cccc4c5cccc6oc7ccc8c(c7c65)c5c(c43)c2ccc5n8-c2nc3ccccc3nc2-c2cccc3oc4ccccc4c23)cc1]

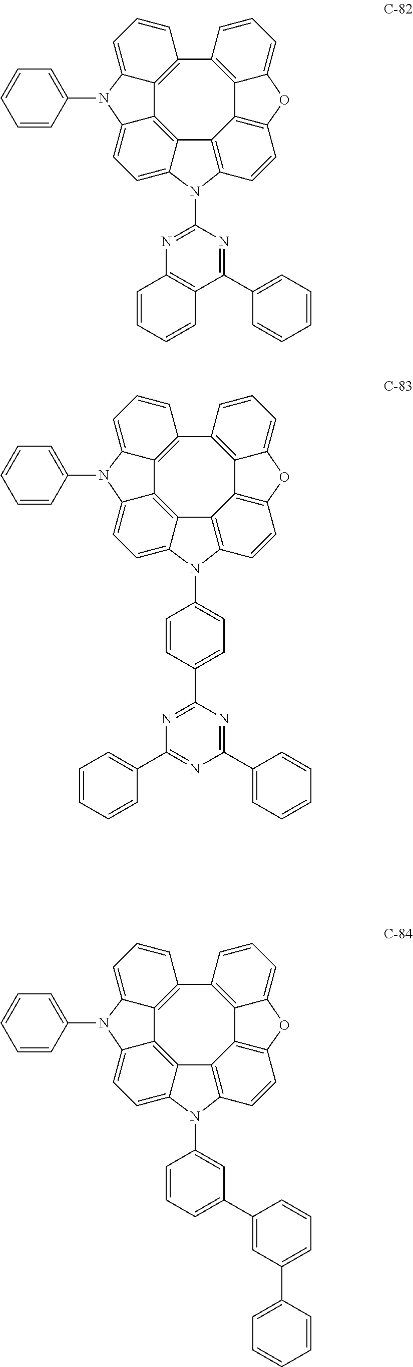 [SMILES: c1ccc(-c2nc(-n3c4ccc5oc6cccc7c8cccc9c8c8c(c4c5c67)c3ccc8n9-c3ccccc3)nc3ccccc23)cc1 ; c1ccc(-c2nc(-c3ccccc3)nc(-c3ccc(-n4c5ccc6oc7cccc8c9cccc%10c9c9c(c5c6c78)c4ccc9n%10-c4ccccc4)cc3)n2)cc1 ; c1ccc(-c2cccc(-c3cccc(-n4c5ccc6oc7cccc8c9cccc%10c9c9c(c5c6c78)c4ccc9n%10-c4ccccc4)c3)c2)cc1]

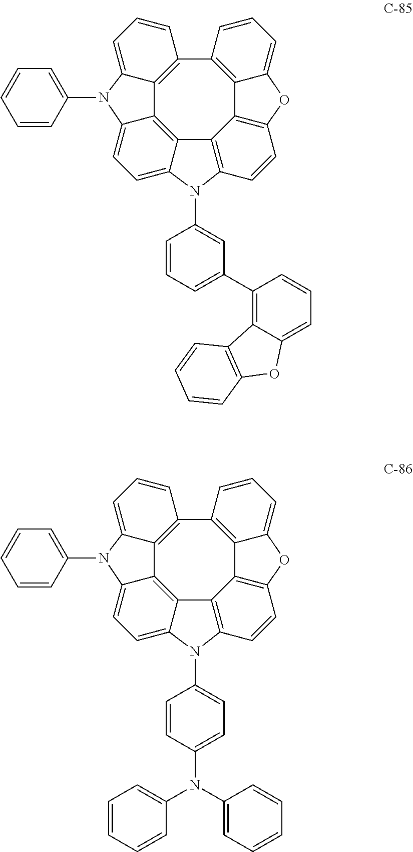 [SMILES: c1ccc(-n2c3cccc4c5cccc6oc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5oc6cccc7c8cccc9c8c8c(c4c5c67)c3ccc8n9-c3ccccc3)cc2)cc1]

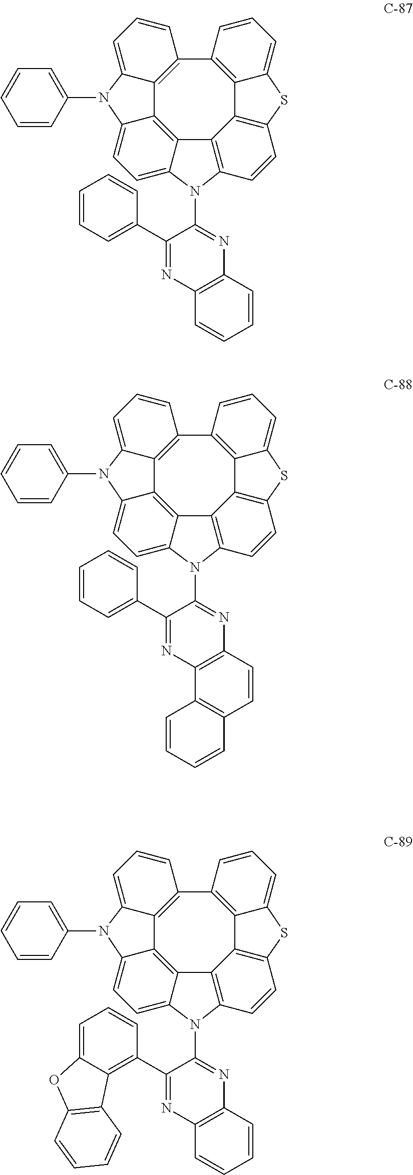 [SMILES: c1ccc(-c2nc3ccccc3nc2-n2c3ccc4sc5cccc6c7cccc8c7c7c(c3c4c56)c2ccc7n8-c2ccccc2)cc1 ; c1ccc(-c2nc3c(ccc4ccccc43)nc2-n2c3ccc4sc5cccc6c7cccc8c7c7c(c3c4c56)c2ccc7n8-c2ccccc2)cc1 ; c1ccc(-n2c3cccc4c5cccc6sc7ccc8c(c7c65)c5c(c43)c2ccc5n8-c2nc3ccccc3nc2-c2cccc3oc4ccccc4c23)cc1]

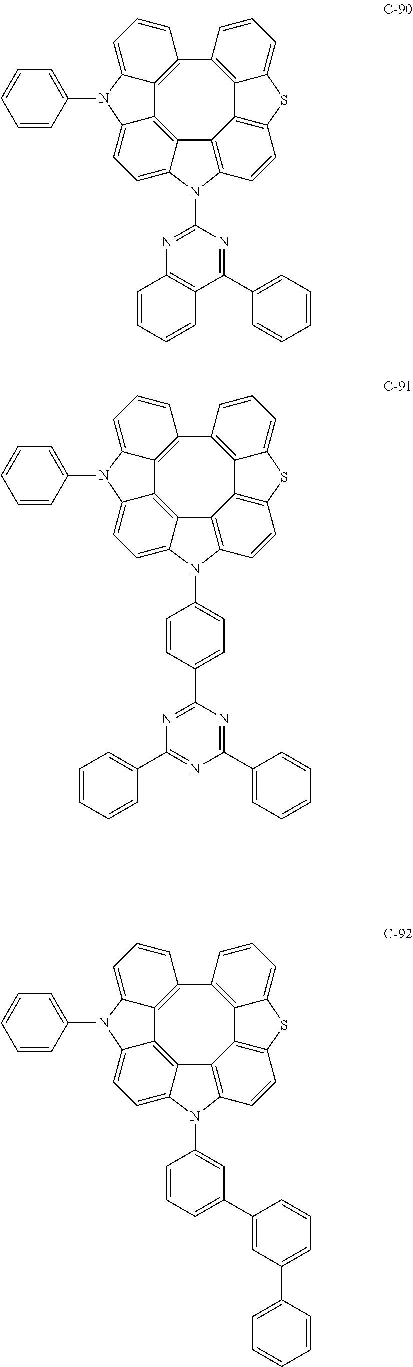 [SMILES: c1ccc(-c2nc(-n3c4ccc5sc6cccc7c8cccc9c8c8c(c4c5c67)c3ccc8n9-c3ccccc3)nc3ccccc23)cc1 ; c1ccc(-c2nc(-c3ccccc3)nc(-c3ccc(-n4c5ccc6sc7cccc8c9cccc%10c9c9c(c5c6c78)c4ccc9n%10-c4ccccc4)cc3)n2)cc1 ; c1ccc(-c2cccc(-c3cccc(-n4c5ccc6sc7cccc8c9cccc%10c9c9c(c5c6c78)c4ccc9n%10-c4ccccc4)c3)c2)cc1]

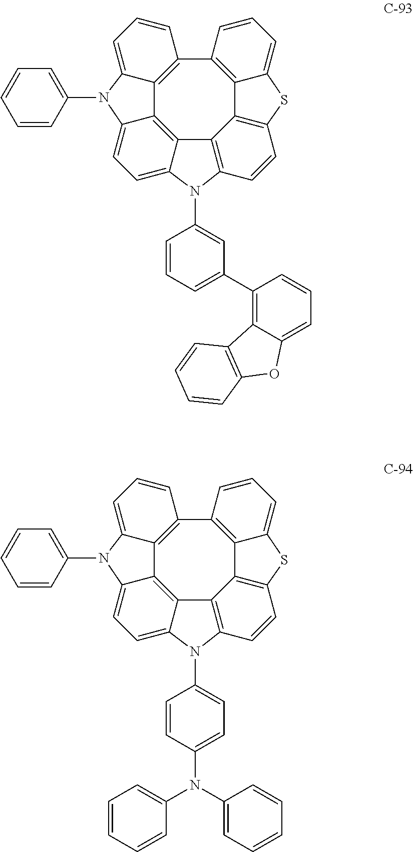 [SMILES: c1ccc(-n2c3cccc4c5cccc6sc7ccc8c(c7c65)c5c(c43)c2ccc5n8-c2cccc(-c3cccc4oc5ccccc5c34)c2)cc1 ; c1ccc(N(c2ccccc2)c2ccc(-n3c4ccc5sc6cccc7c8cccc9c8c8c(c4c5c67)c3ccc8n9-c3ccccc3)cc2)cc1]

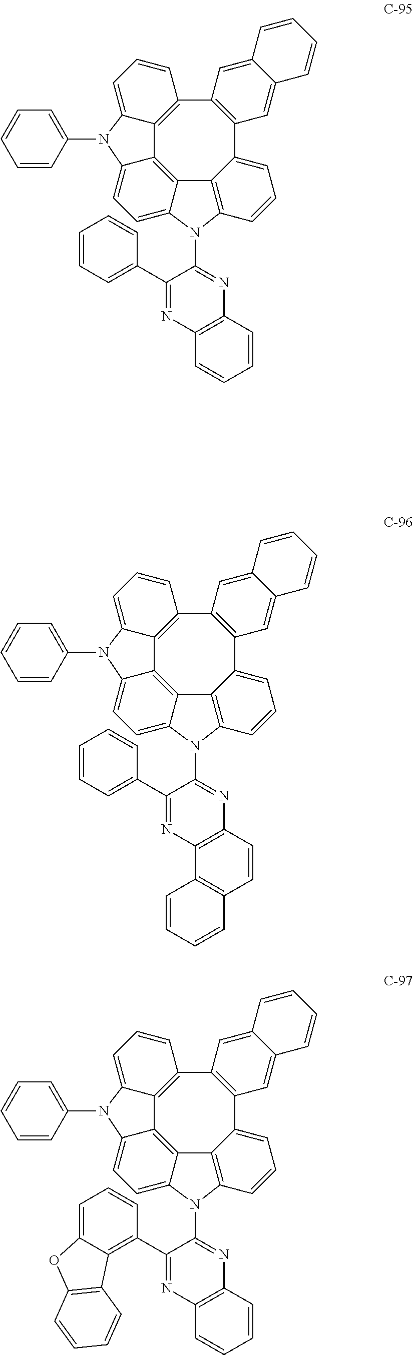 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

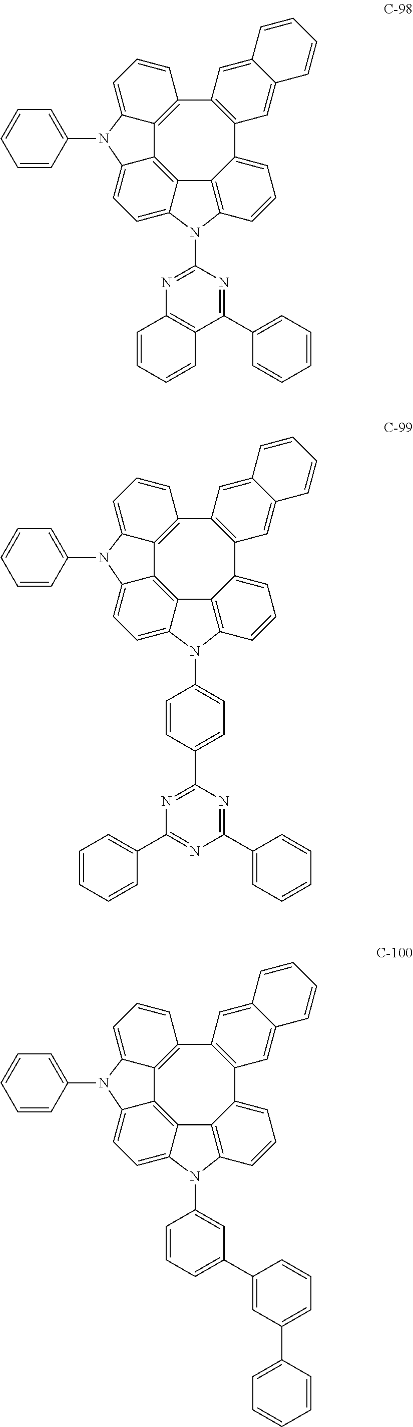 [SMILES: c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1 ; c1ccc(-c2cccc(-c3cccc(-n4c5cccc6c7cc8ccccc8cc7c7cccc8c7c7c(c65)c4ccc7n8-c4ccccc4)c3)c2)cc1]

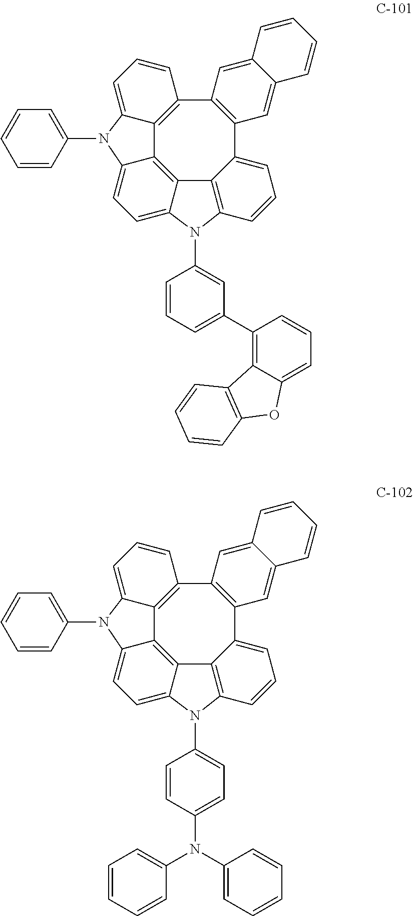 [SMILES: c1ccc(-n2c3cccc4c5cc6ccccc6cc5c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cc7ccccc7cc6c6cccc7c6c6c(c54)c3ccc6n7-c3ccccc3)cc2)cc1]

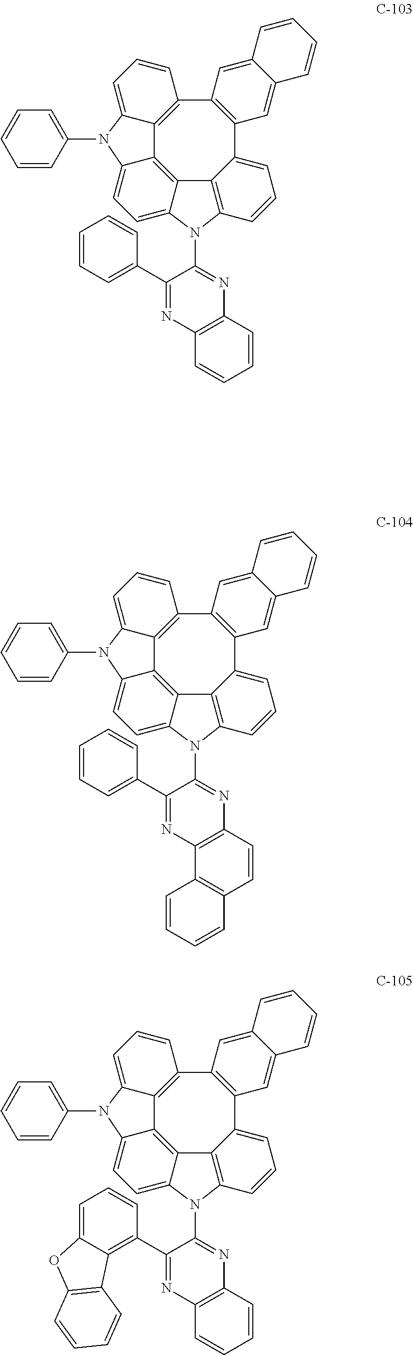 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

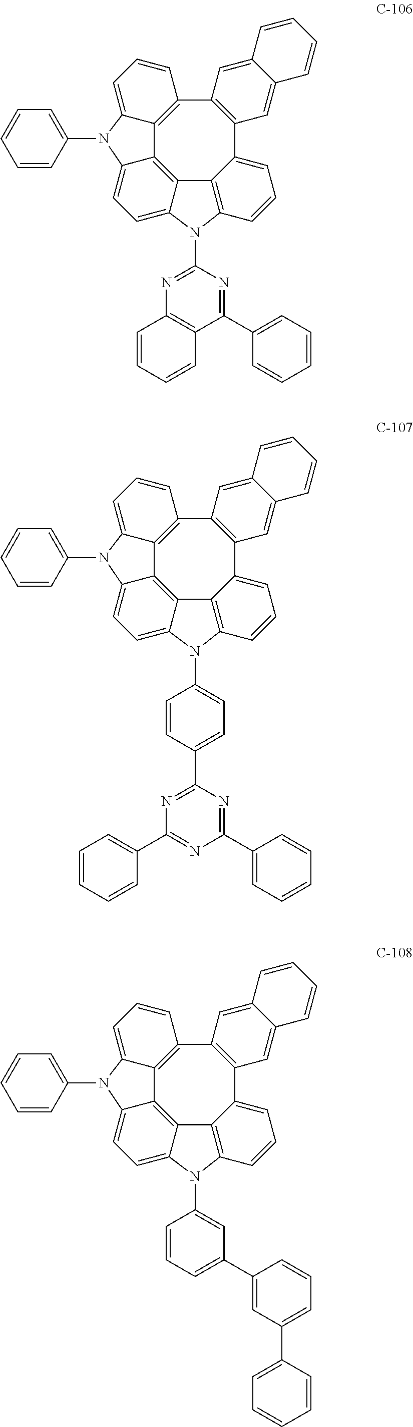 [SMILES: c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1 ; c1ccc(-c2cccc(-c3cccc(-n4c5cccc6c7cc8ccccc8cc7c7cccc8c7c7c(c65)c4ccc7n8-c4ccccc4)c3)c2)cc1]

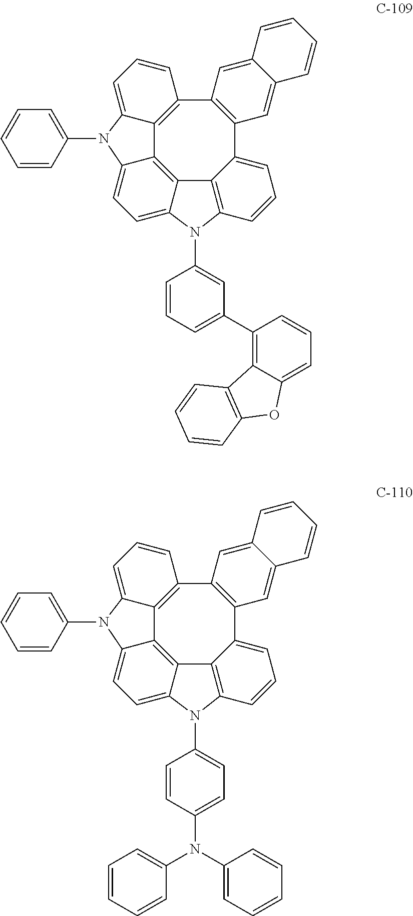 [SMILES: c1ccc(-n2c3cccc4c5cc6ccccc6cc5c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cc7ccccc7cc6c6cccc7c6c6c(c54)c3ccc6n7-c3ccccc3)cc2)cc1]

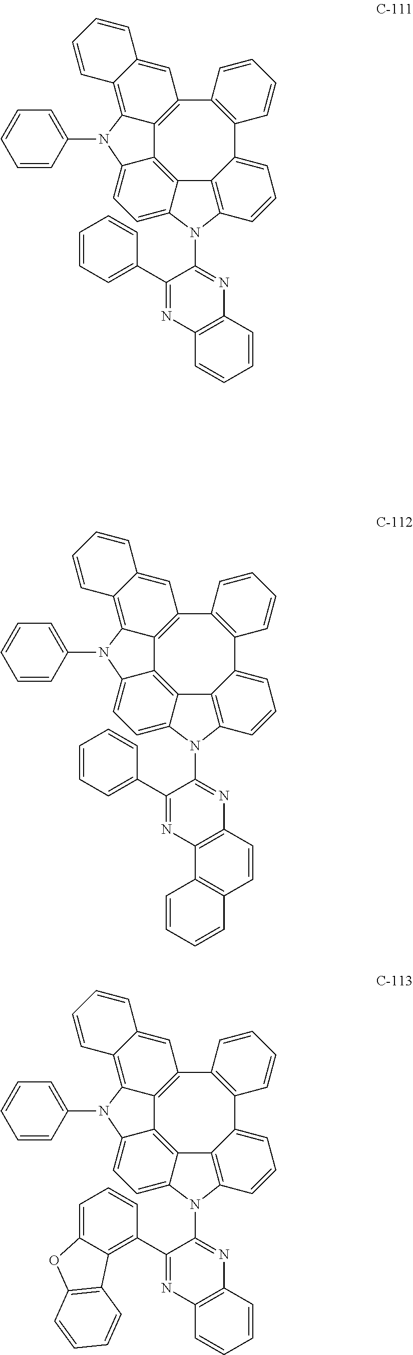 [SMILES: c1ccc(-c2nc3ccccc3nc2-n2c3cccc4c5ccccc5c5cc6ccccc6c6c5c5c(c43)c2ccc5n6-c2ccccc2)cc1 ; c1ccc(-c2nc3c(ccc4ccccc43)nc2-n2c3cccc4c5ccccc5c5cc6ccccc6c6c5c5c(c43)c2ccc5n6-c2ccccc2)cc1 ; c1ccc(-n2c3ccc4c5c6c(cccc6n4-c4nc6ccccc6nc4-c4cccc6oc7ccccc7c46)c4ccccc4c4cc6ccccc6c2c4c53)cc1]

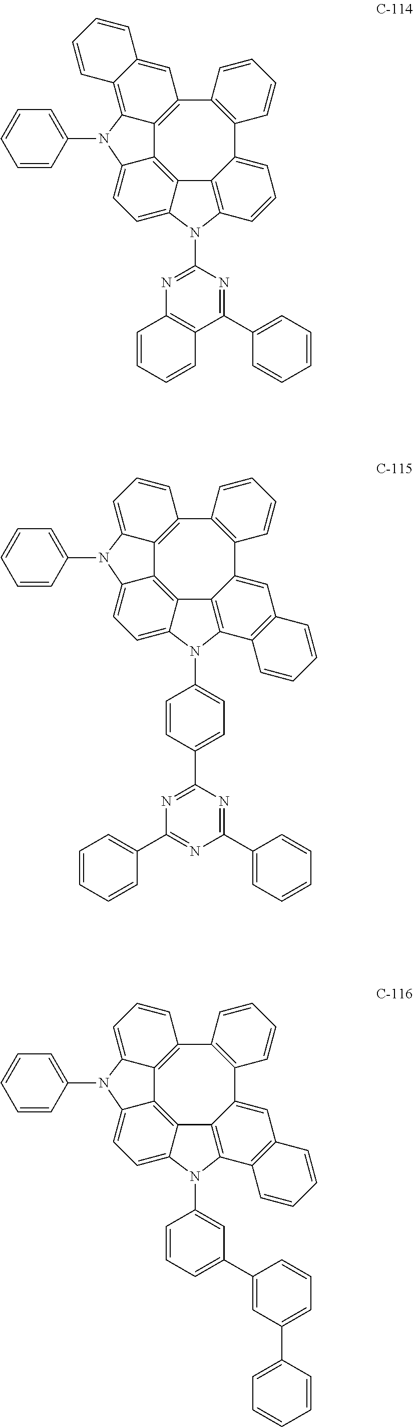 [SMILES: c1ccc(-c2nc(-n3c4cccc5c6ccccc6c6cc7ccccc7c7c6c6c(c54)c3ccc6n7-c3ccccc3)nc3ccccc23)cc1 ; c1ccc(-c2nc(-c3ccccc3)nc(-c3ccc(-n4c5ccc6c7c8c(cccc8n6-c6ccccc6)c6ccccc6c6cc8ccccc8c4c6c75)cc3)n2)cc1 ; c1ccc(-c2cccc(-c3cccc(-n4c5ccc6c7c8c(cccc8n6-c6ccccc6)c6ccccc6c6cc8ccccc8c4c6c75)c3)c2)cc1]

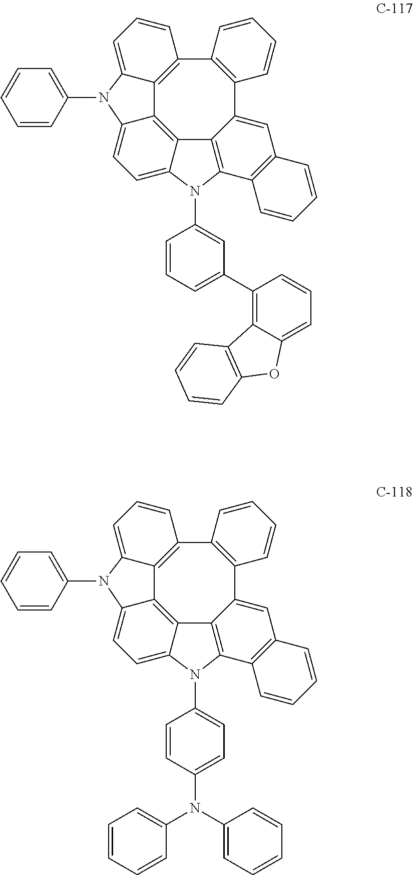 [SMILES: c1ccc(-n2c3cccc4c5ccccc5c5cc6ccccc6c6c5c5c(c43)c2ccc5n6-c2cccc(-c3cccc4oc5ccccc5c34)c2)cc1 ; c1ccc(N(c2ccccc2)c2ccc(-n3c4ccc5c6c7c(cccc7n5-c5ccccc5)c5ccccc5c5cc7ccccc7c3c5c64)cc2)cc1]

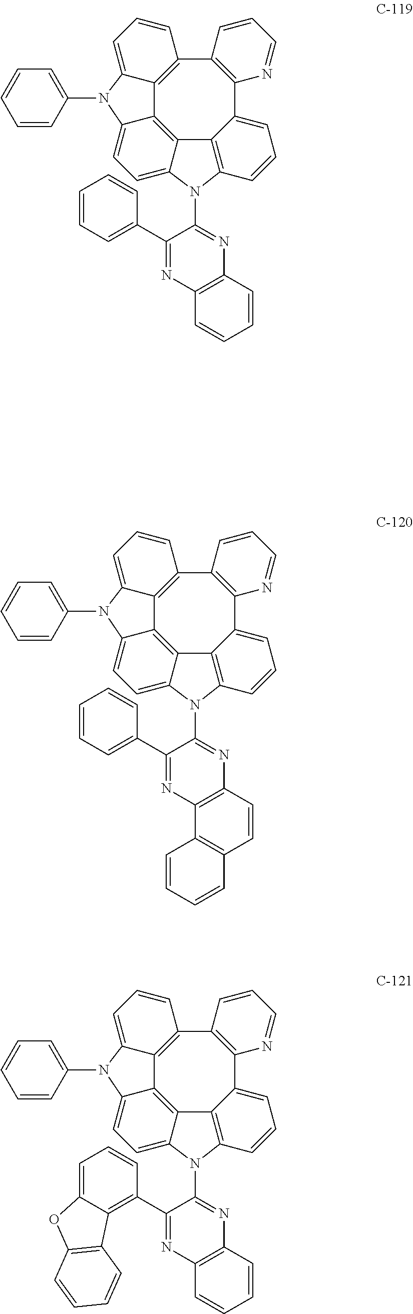 [SMILES: c1ccc(-c2nc3ccccc3nc2-n2c3cccc4c5ncccc5c5cccc6c5c5c(c43)c2ccc5n6-c2ccccc2)cc1 ; c1ccc(-c2nc3c(ccc4ccccc43)nc2-n2c3cccc4c5ncccc5c5cccc6c5c5c(c43)c2ccc5n6-c2ccccc2)cc1 ; c1ccc(-n2c3cccc4c5cccnc5c5cccc6c5c5c(c43)c2ccc5n6-c2nc3ccccc3nc2-c2cccc3oc4ccccc4c23)cc1]

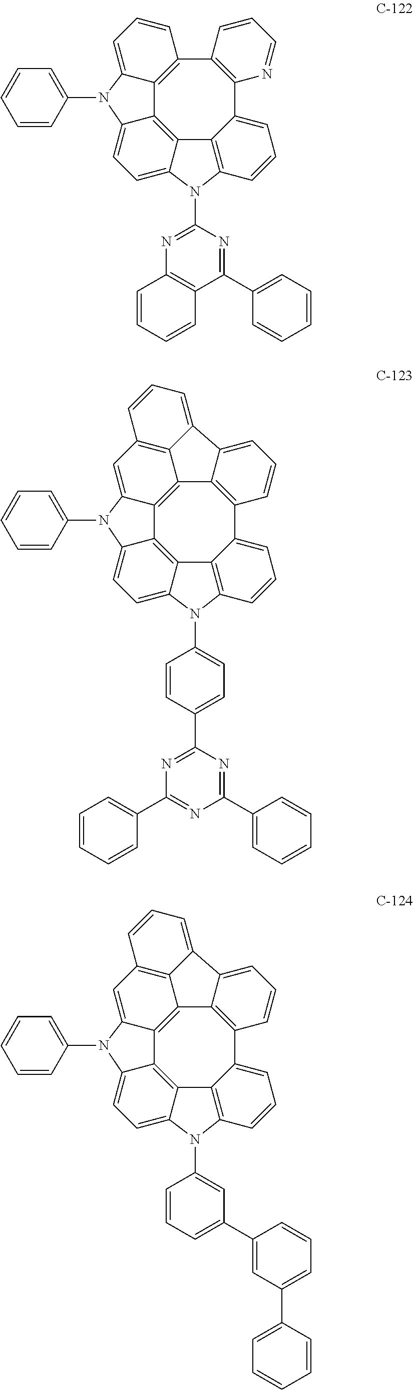

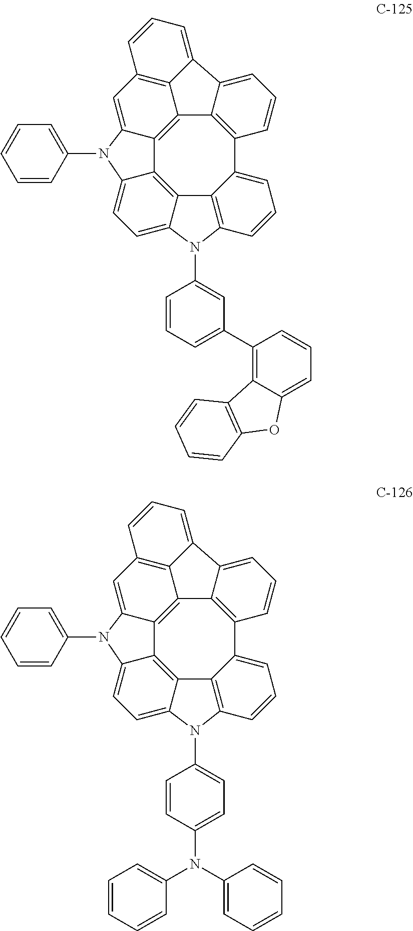 [SMILES: c1ccc(-n2c3cc4cccc5c4c4c6c-5cccc6c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cccc7c6c6c8c-7cccc8cc7c6c6c(c54)c3ccc6n7-c3ccccc3)cc2)cc1]

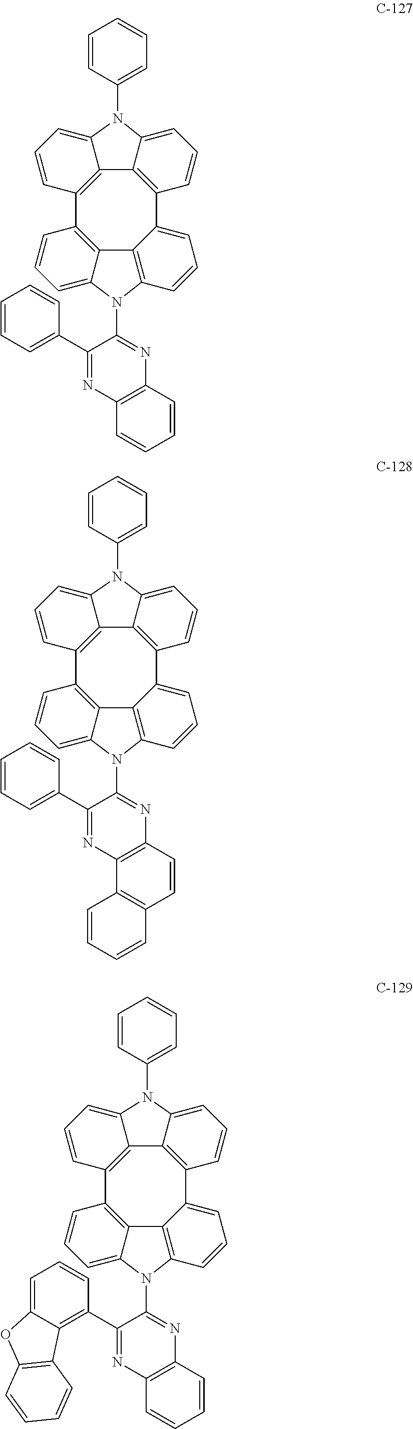

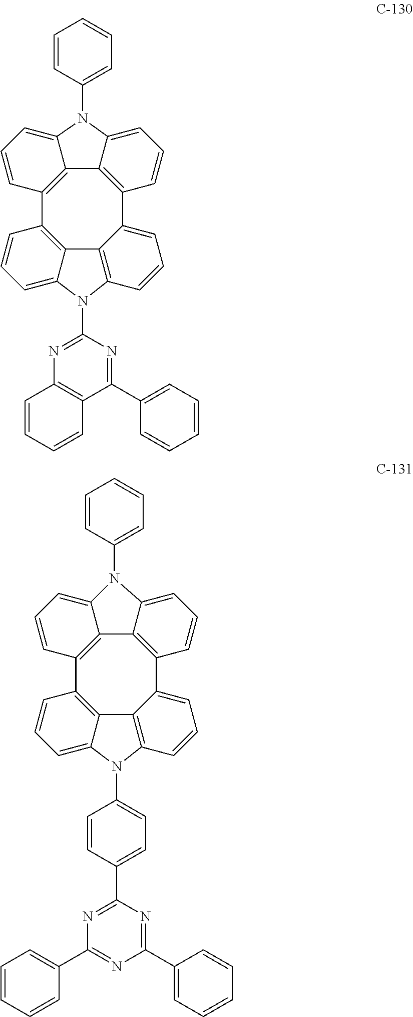 [SMILES: c1ccc(-c2nc(-n3c4cccc5c6cccc7c6c6c(cccc6n7-c6ccccc6)c6cccc3c6c54)nc3ccccc23)cc1 ; c1ccc(-c2nc(-c3ccccc3)nc(-c3ccc(-n4c5cccc6c7cccc8c7c7c(cccc7n8-c7ccccc7)c7cccc4c7c65)cc3)n2)cc1]

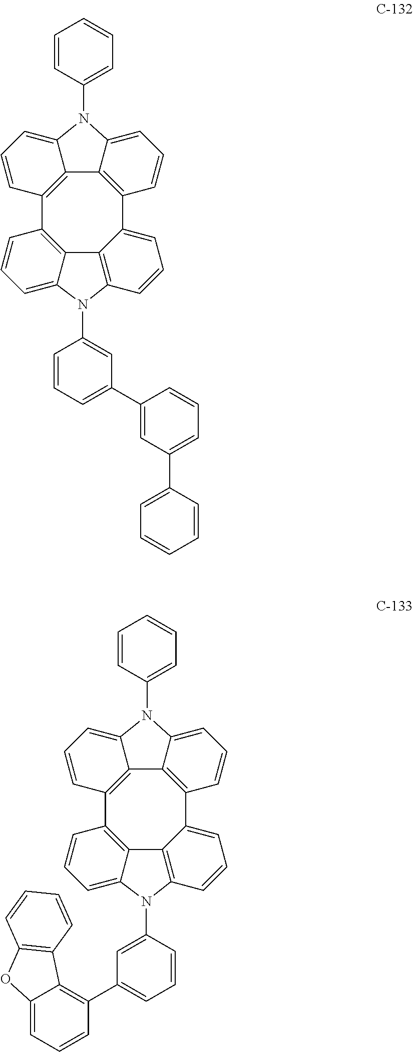 [SMILES: c1ccc(-c2cccc(-c3cccc(-n4c5cccc6c7cccc8c7c7c(cccc7n8-c7ccccc7)c7cccc4c7c65)c3)c2)cc1 ; c1ccc(-n2c3cccc4c5cccc6c5c5c(cccc5n6-c5cccc(-c6cccc7oc8ccccc8c67)c5)c5cccc2c5c43)cc1]

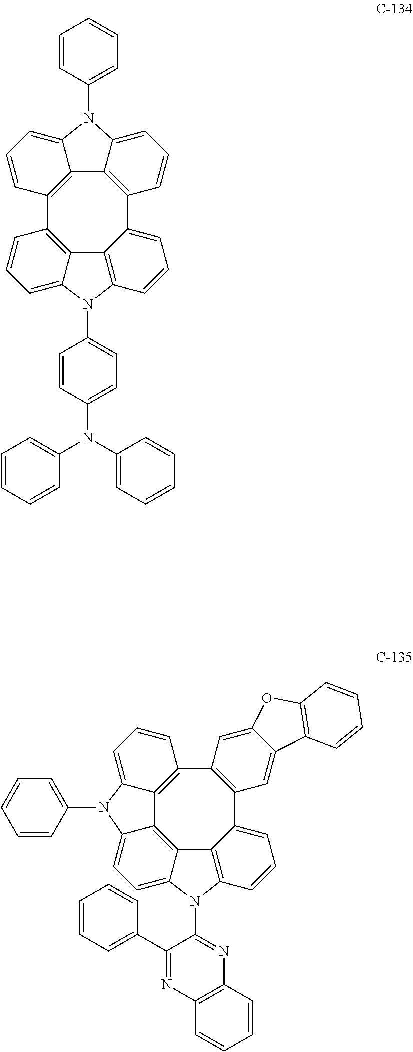 [SMILES: c1ccc(N(c2ccccc2)c2ccc(-n3c4cccc5c6cccc7c6c6c(cccc6n7-c6ccccc6)c6cccc3c6c54)cc2)cc1 ; c1ccc(-c2nc3ccccc3nc2-n2c3cccc4c5cc6c(cc5c5cccc7c5c5c(c43)c2ccc5n7-c2ccccc2)oc2ccccc26)cc1]

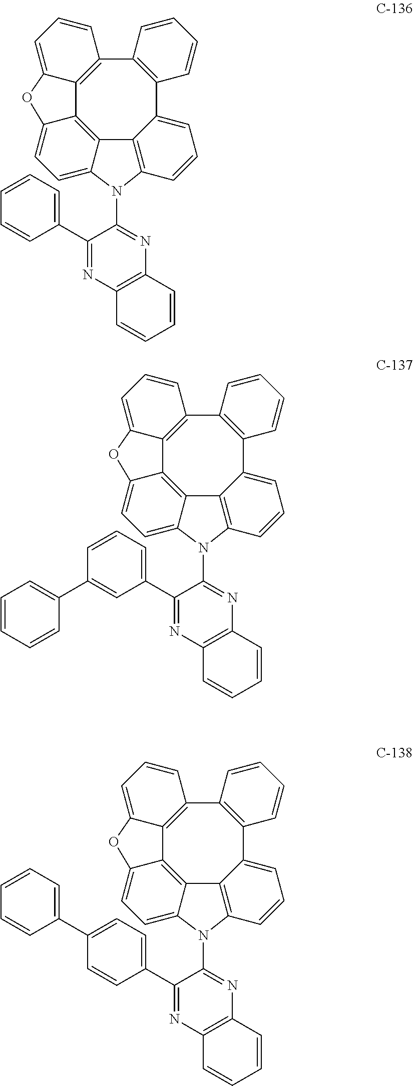

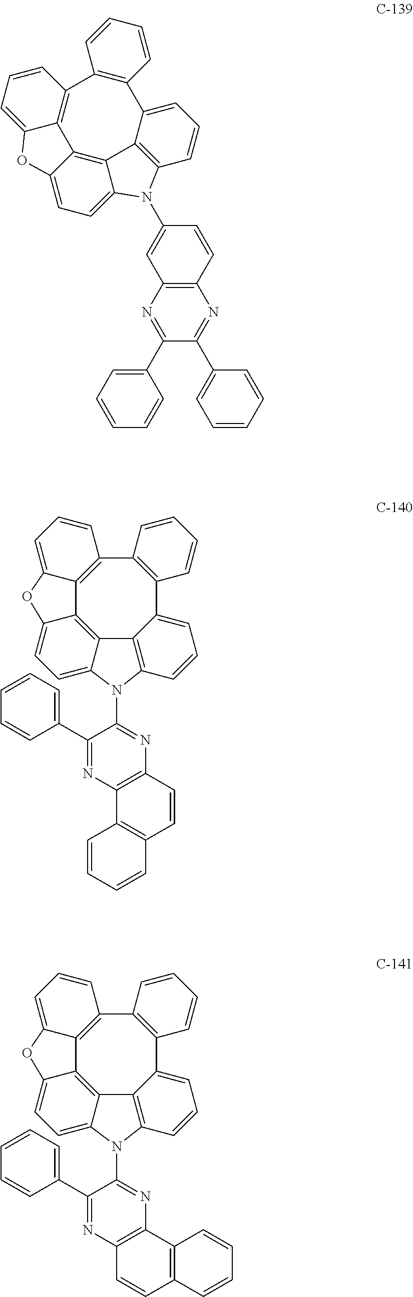 [SMILES: c1ccc(-c2nc3ccc(-n4c5cccc6c7ccccc7c7cccc8oc9ccc4c(c9c87)c65)cc3nc2-c2ccccc2)cc1 ; c1ccc(-c2nc3c(ccc4ccccc43)nc2-n2c3cccc4c5ccccc5c5cccc6oc7ccc2c(c7c65)c43)cc1 ; c1ccc(-c2nc3ccc4ccccc4c3nc2-n2c3cccc4c5ccccc5c5cccc6oc7ccc2c(c7c65)c43)cc1]

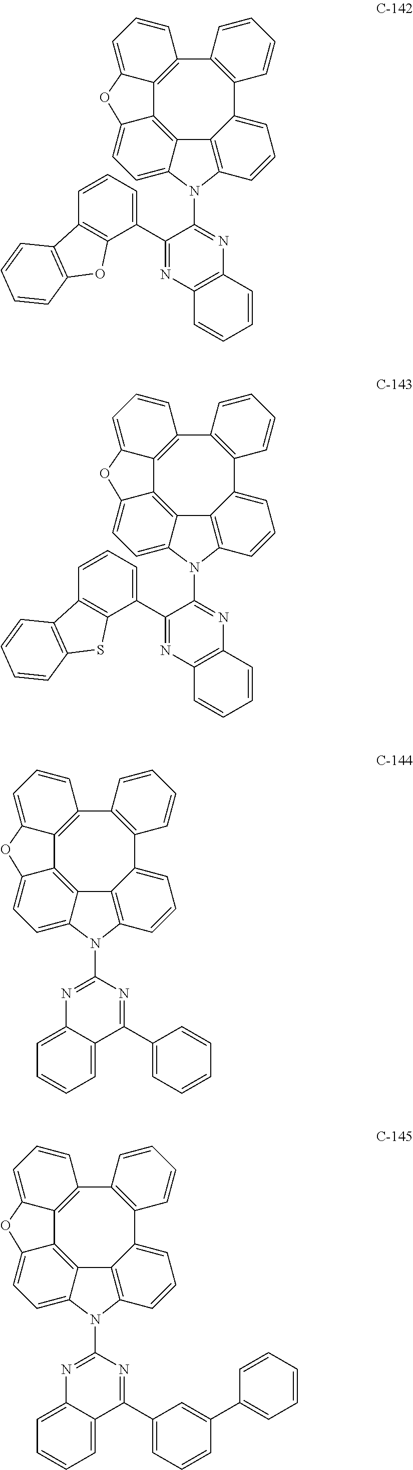 [SMILES: c1ccc2nc(-n3c4cccc5c6ccccc6c6cccc7oc8ccc3c(c8c76)c54)c(-c3cccc4c3oc3ccccc34)nc2c1 ; c1ccc2nc(-n3c4cccc5c6ccccc6c6cccc7oc8ccc3c(c8c76)c54)c(-c3cccc4c3sc3ccccc34)nc2c1 ; c1ccc(-c2nc(-n3c4cccc5c6ccccc6c6cccc7oc8ccc3c(c8c76)c54)nc3ccccc23)cc1 ; c1ccc(-c2cccc(-c3nc(-n4c5cccc6c7ccccc7c7cccc8oc9ccc4c(c9c87)c65)nc4ccccc34)c2)cc1]

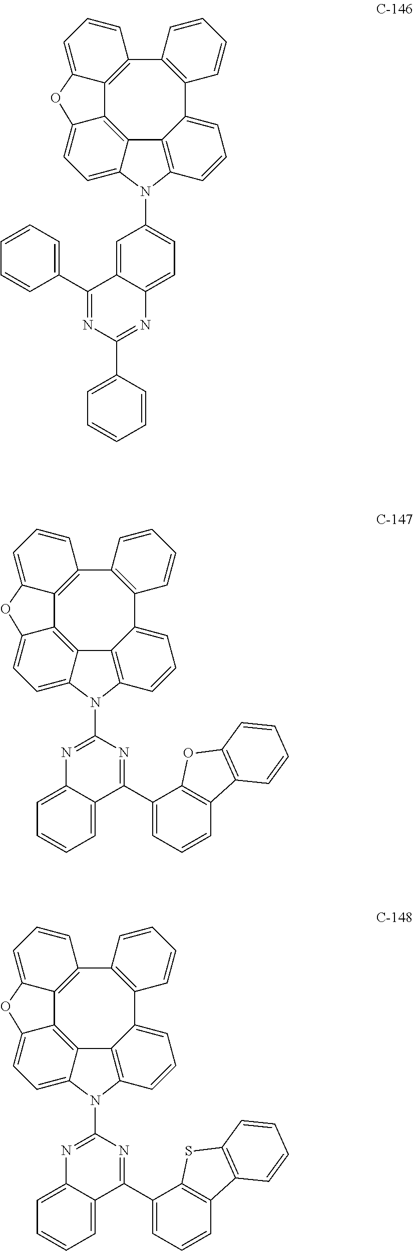 [SMILES: c1ccc(-c2nc(-c3ccccc3)c3cc(-n4c5cccc6c7ccccc7c7cccc8oc9ccc4c(c9c87)c65)ccc3n2)cc1 ; c1ccc2c(-c3cccc4c3oc3ccccc34)nc(-n3c4cccc5c6ccccc6c6cccc7oc8ccc3c(c8c76)c54)nc2c1 ; c1ccc2c(-c3cccc4c3sc3ccccc34)nc(-n3c4cccc5c6ccccc6c6cccc7oc8ccc3c(c8c76)c54)nc2c1]

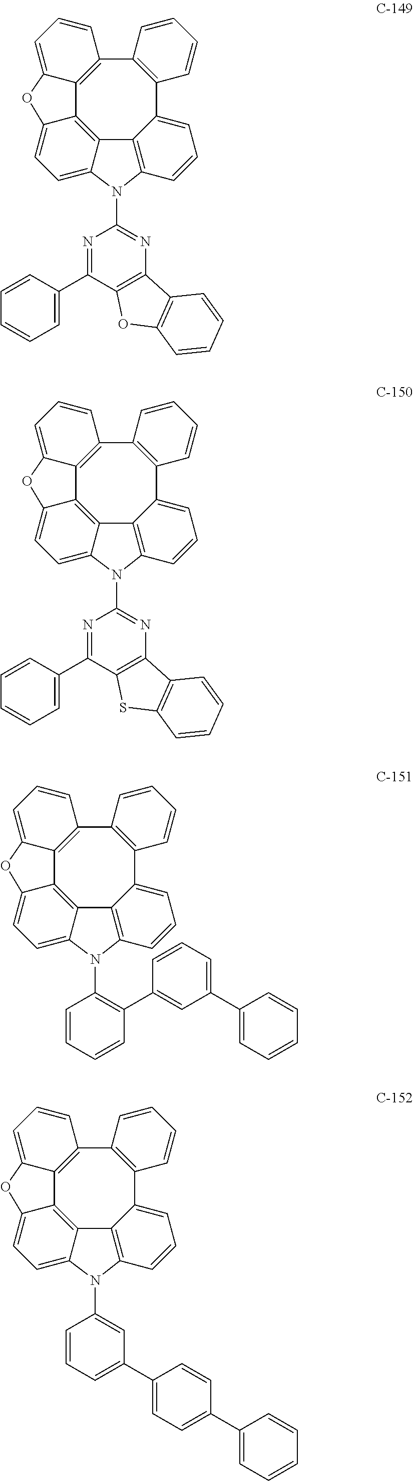 [SMILES: c1ccc(-c2nc(-n3c4cccc5c6ccccc6c6cccc7oc8ccc3c(c8c76)c54)nc3c2oc2ccccc23)cc1 ; c1ccc(-c2nc(-n3c4cccc5c6ccccc6c6cccc7oc8ccc3c(c8c76)c54)nc3c2sc2ccccc23)cc1 ; c1ccc(-c2cccc(-c3ccccc3-n3c4cccc5c6ccccc6c6cccc7oc8ccc3c(c8c76)c54)c2)cc1 ; c1ccc(-c2ccc(-c3cccc(-n4c5cccc6c7ccccc7c7cccc8oc9ccc4c(c9c87)c65)c3)cc2)cc1]

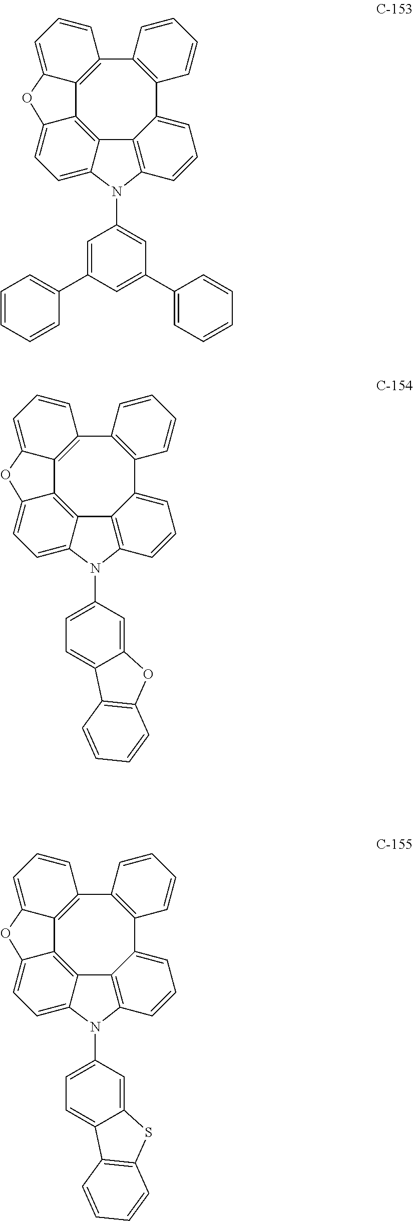 [SMILES: c1ccc(-c2cc(-c3ccccc3)cc(-n3c4cccc5c6ccccc6c6cccc7oc8ccc3c(c8c76)c54)c2)cc1 ; c1ccc2c(c1)oc1cc(-n3c4cccc5c6ccccc6c6cccc7oc8ccc3c(c8c76)c54)ccc12 ; c1ccc2c(c1)sc1cc(-n3c4cccc5c6ccccc6c6cccc7oc8ccc3c(c8c76)c54)ccc12]

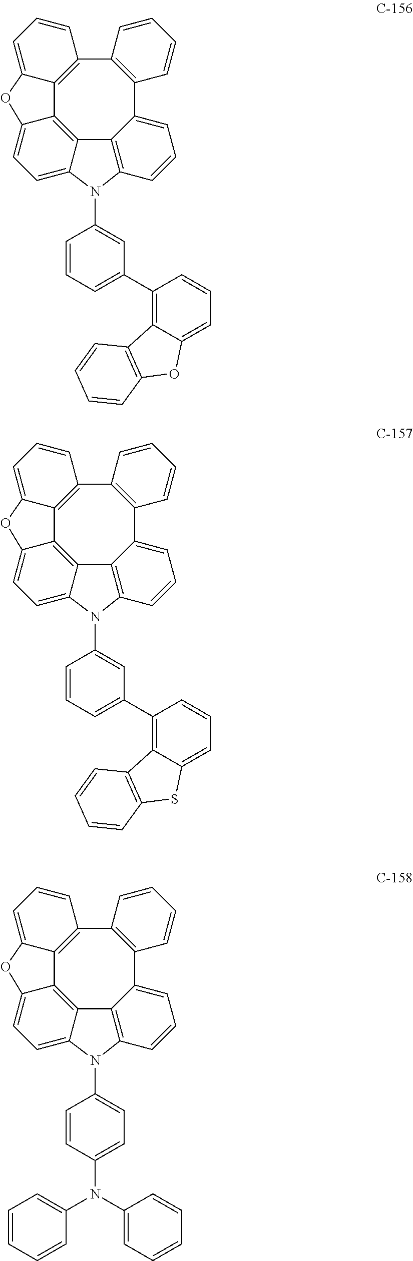 [SMILES: c1cc(-c2cccc3oc4ccccc4c23)cc(-n2c3cccc4c5ccccc5c5cccc6oc7ccc2c(c7c65)c43)c1 ; c1cc(-c2cccc3sc4ccccc4c23)cc(-n2c3cccc4c5ccccc5c5cccc6oc7ccc2c(c7c65)c43)c1 ; c1ccc(N(c2ccccc2)c2ccc(-n3c4cccc5c6ccccc6c6cccc7oc8ccc3c(c8c76)c54)cc2)cc1]

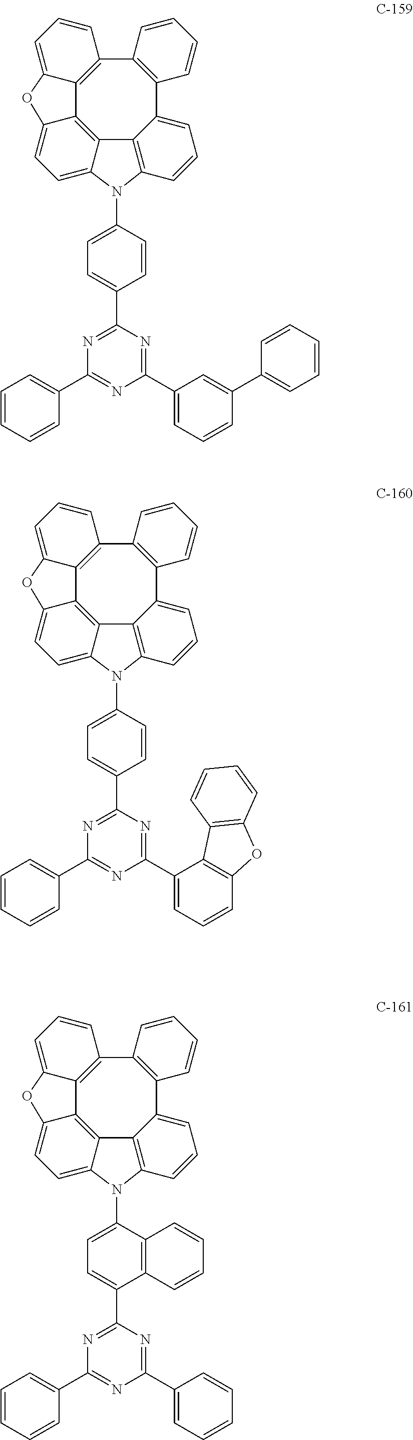 [SMILES: c1ccc(-c2cccc(-c3nc(-c4ccccc4)nc(-c4ccc(-n5c6cccc7c8ccccc8c8cccc9oc%10ccc5c(c%10c98)c76)cc4)n3)c2)cc1 ; c1ccc(-c2nc(-c3ccc(-n4c5cccc6c7ccccc7c7cccc8oc9ccc4c(c9c87)c65)cc3)nc(-c3cccc4oc5ccccc5c34)n2)cc1 ; c1ccc(-c2nc(-c3ccccc3)nc(-c3ccc(-n4c5cccc6c7ccccc7c7cccc8oc9ccc4c(c9c87)c65)c4ccccc34)n2)cc1]

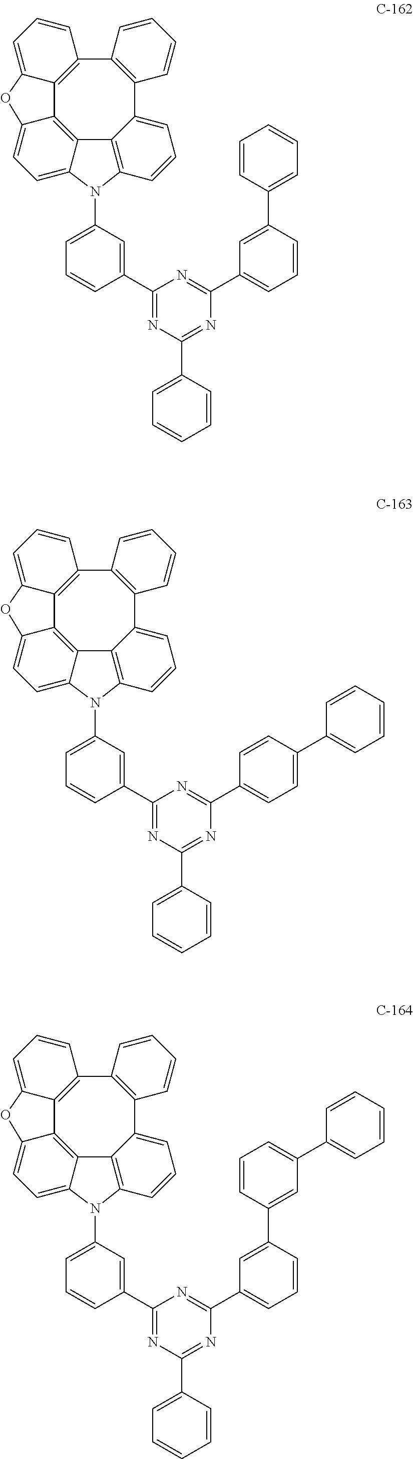 [SMILES: c1ccc(-c2cccc(-c3nc(-c4ccccc4)nc(-c4cccc(-n5c6cccc7c8ccccc8c8cccc9oc%10ccc5c(c%10c98)c76)c4)n3)c2)cc1 ; c1ccc(-c2ccc(-c3nc(-c4ccccc4)nc(-c4cccc(-n5c6cccc7c8ccccc8c8cccc9oc%10ccc5c(c%10c98)c76)c4)n3)cc2)cc1 ; c1ccc(-c2cccc(-c3cccc(-c4nc(-c5ccccc5)nc(-c5cccc(-n6c7cccc8c9ccccc9c9cccc%10oc%11ccc6c(c%11c%109)c87)c5)n4)c3)c2)cc1]

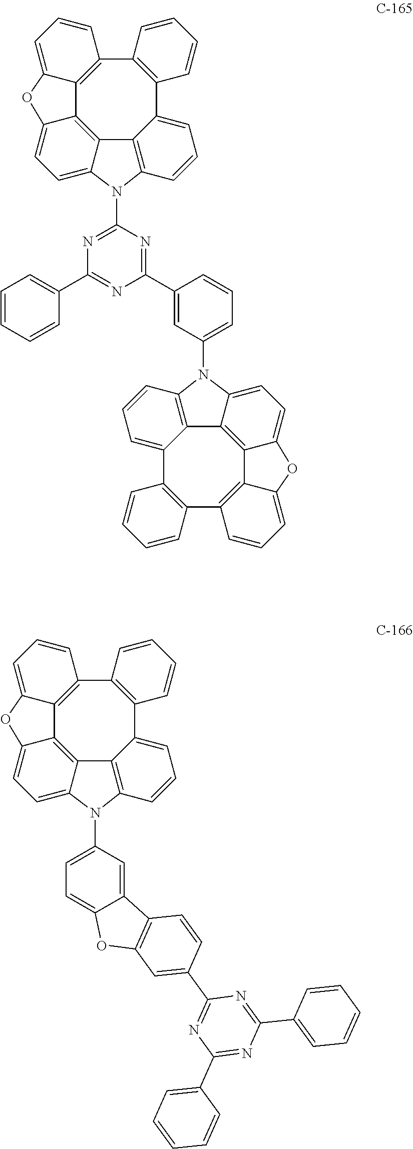 [SMILES: c1ccc(-c2nc(-c3cccc(-n4c5cccc6c7ccccc7c7cccc8oc9ccc4c(c9c87)c65)c3)nc(-n3c4cccc5c6ccccc6c6cccc7oc8ccc3c(c8c76)c54)n2)cc1 ; c1ccc(-c2nc(-c3ccccc3)nc(-c3ccc4c(c3)oc3ccc(-n5c6cccc7c8ccccc8c8cccc9oc%10ccc5c(c%10c98)c76)cc34)n2)cc1]

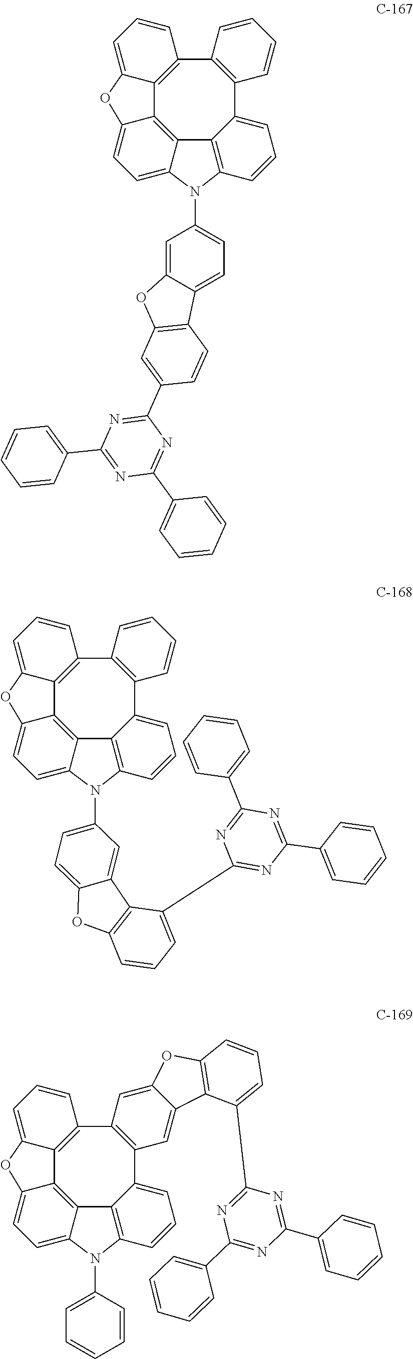 [SMILES: c1ccc(-c2nc(-c3ccccc3)nc(-c3ccc4c(c3)oc3cc(-n5c6cccc7c8ccccc8c8cccc9oc%10ccc5c(c%10c98)c76)ccc34)n2)cc1 ; c1ccc(-c2nc(-c3ccccc3)nc(-c3cccc4oc5ccc(-n6c7cccc8c9ccccc9c9cccc%10oc%11ccc6c(c%11c%109)c87)cc5c34)n2)cc1 ; c1ccc(-c2nc(-c3ccccc3)nc(-c3cccc4oc5cc6c(cc5c34)c3cccc4c3c3c5c(ccc3n4-c3ccccc3)oc3cccc6c35)n2)cc1]

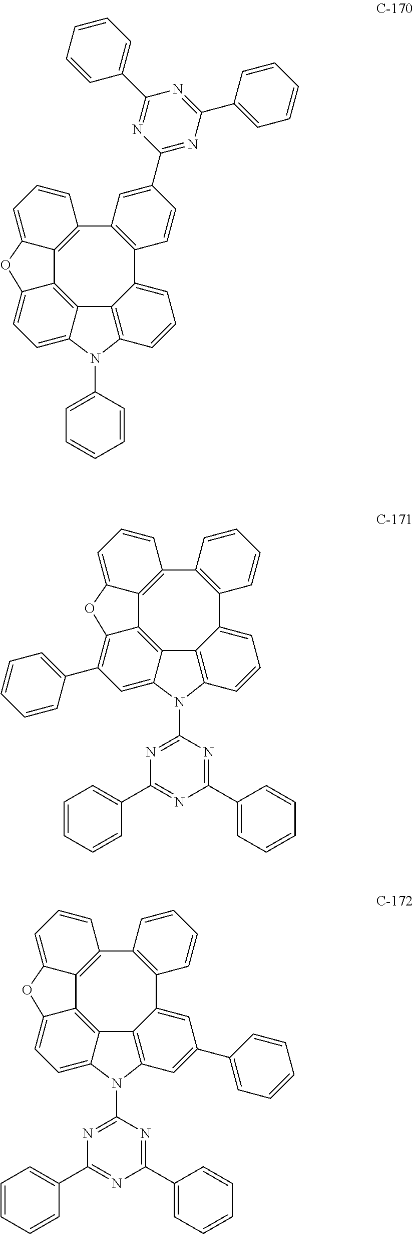 [SMILES: c1ccc(-c2nc(-c3ccccc3)nc(-c3ccc4c(c3)c3cccc5oc6ccc7c(c6c53)c3c4cccc3n7-c3ccccc3)n2)cc1 ; c1ccc(-c2nc(-c3ccccc3)nc(-n3c4cccc5c6ccccc6c6cccc7oc8c(-c9ccccc9)cc3c(c8c76)c54)n2)cc1 ; c1ccc(-c2cc3c4ccccc4c4cccc5oc6ccc7c(c6c54)c3c(c2)n7-c2nc(-c3ccccc3)nc(-c3ccccc3)n2)cc1]

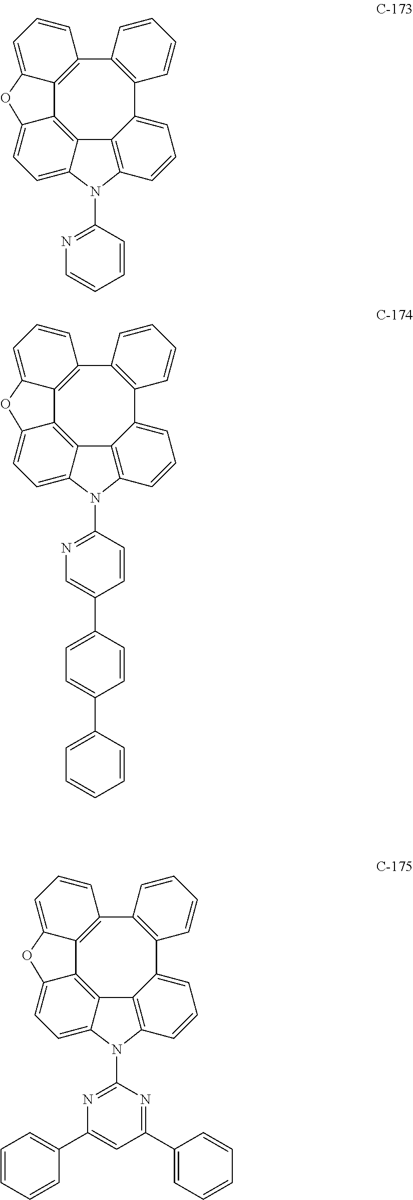 [SMILES: c1ccc(-n2c3cccc4c5ccccc5c5cccc6oc7ccc2c(c7c65)c43)nc1 ; c1ccc(-c2ccc(-c3ccc(-n4c5cccc6c7ccccc7c7cccc8oc9ccc4c(c9c87)c65)nc3)cc2)cc1 ; c1ccc(-c2cc(-c3ccccc3)nc(-n3c4cccc5c6ccccc6c6cccc7oc8ccc3c(c8c76)c54)n2)cc1]

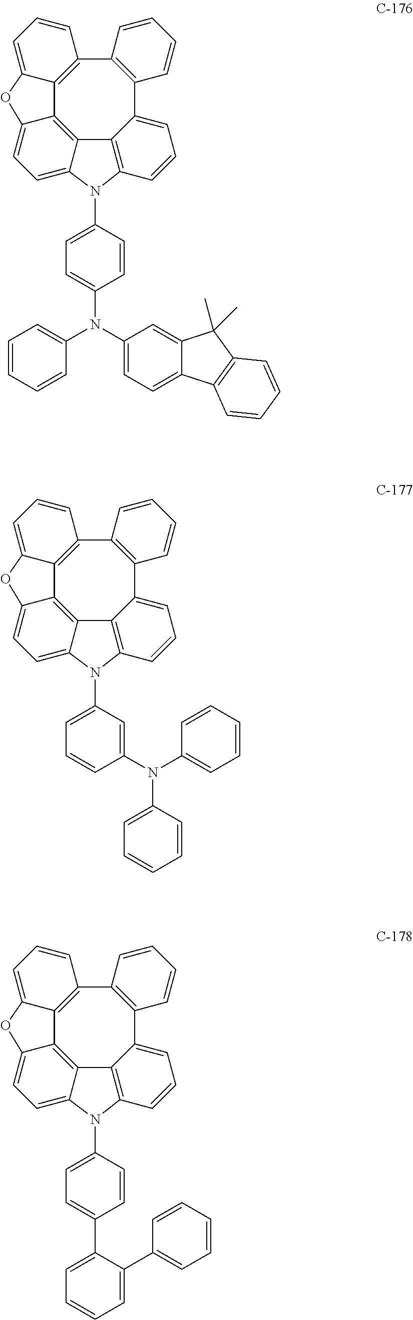 [SMILES: CC1(C)c2ccccc2-c2ccc(N(c3ccccc3)c3ccc(-n4c5cccc6c7ccccc7c7cccc8oc9ccc4c(c9c87)c65)cc3)cc21 ; c1ccc(N(c2ccccc2)c2cccc(-n3c4cccc5c6ccccc6c6cccc7oc8ccc3c(c8c76)c54)c2)cc1 ; c1ccc(-c2ccccc2-c2ccc(-n3c4cccc5c6ccccc6c6cccc7oc8ccc3c(c8c76)c54)cc2)cc1]

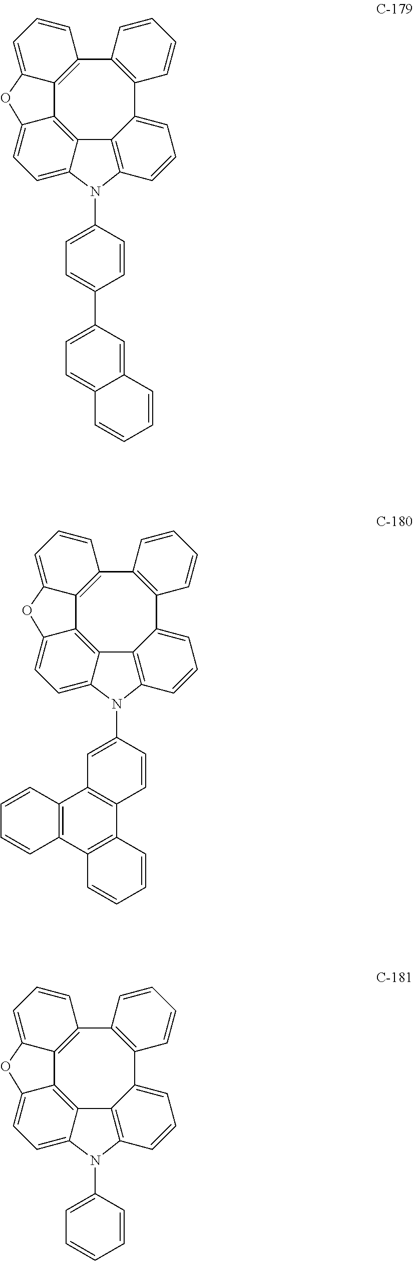 [SMILES: c1ccc2cc(-c3ccc(-n4c5cccc6c7ccccc7c7cccc8oc9ccc4c(c9c87)c65)cc3)ccc2c1 ; c1ccc2c(c1)c1ccccc1c1cc(-n3c4cccc5c6ccccc6c6cccc7oc8ccc3c(c8c76)c54)ccc21 ; c1ccc(-n2c3cccc4c5ccccc5c5cccc6oc7ccc2c(c7c65)c43)cc1]

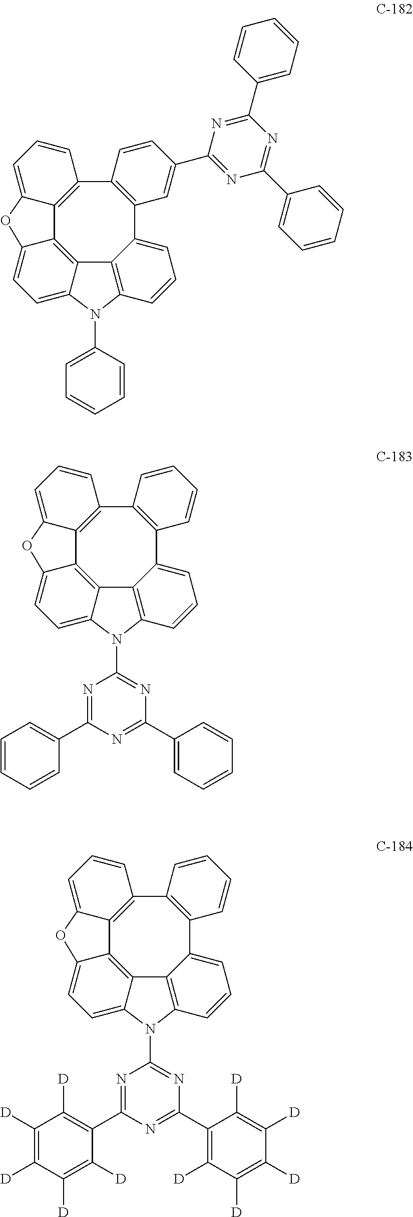 [SMILES: c1ccc(-c2nc(-c3ccccc3)nc(-c3ccc4c(c3)c3cccc5c3c3c6c(ccc3n5-c3ccccc3)oc3cccc4c36)n2)cc1 ; c1ccc(-c2nc(-c3ccccc3)nc(-n3c4cccc5c6ccccc6c6cccc7oc8ccc3c(c8c76)c54)n2)cc1 ; [2H]c1c([2H])c([2H])c(-c2nc(-c3c([2H])c([2H])c([2H])c([2H])c3[2H])nc(-n3c4cccc5c6ccccc6c6cccc7oc8ccc3c(c8c76)c54)n2)c([2H])c1[2H]]

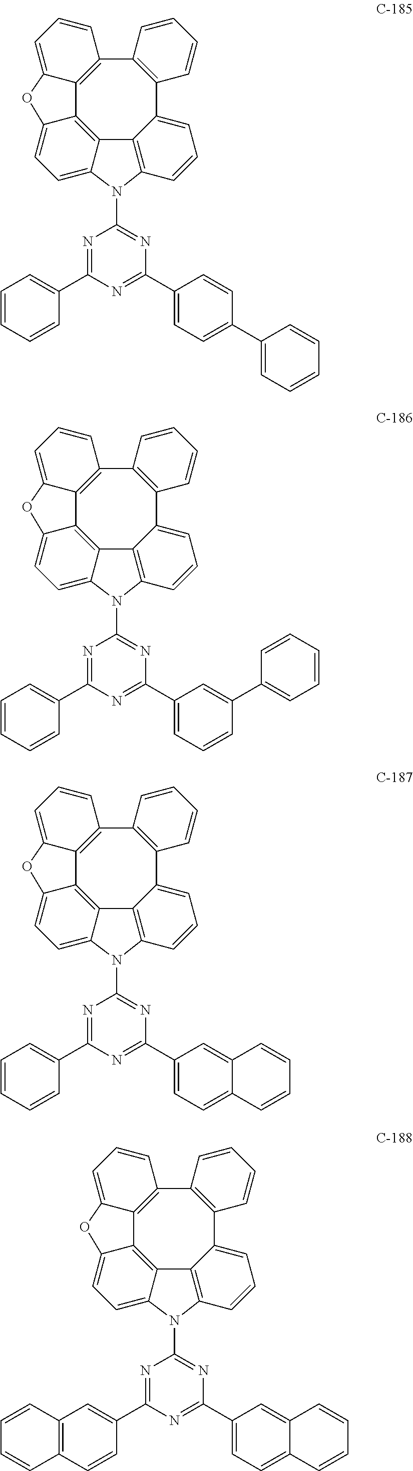

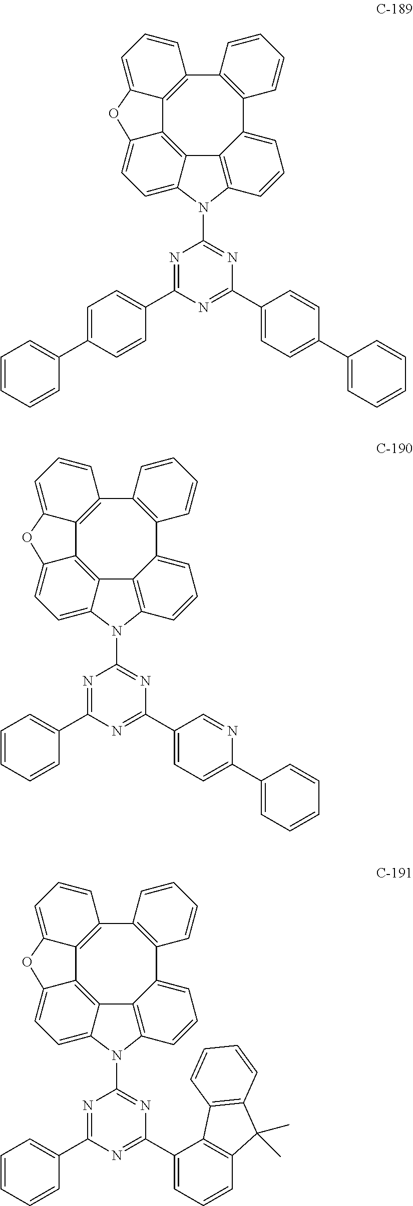 [SMILES: c1ccc(-c2ccc(-c3nc(-c4ccc(-c5ccccc5)cc4)nc(-n4c5cccc6c7ccccc7c7cccc8oc9ccc4c(c9c87)c65)n3)cc2)cc1 ; c1ccc(-c2ccc(-c3nc(-c4ccccc4)nc(-n4c5cccc6c7ccccc7c7cccc8oc9ccc4c(c9c87)c65)n3)cn2)cc1 ; CC1(C)c2ccccc2-c2c(-c3nc(-c4ccccc4)nc(-n4c5cccc6c7ccccc7c7cccc8oc9ccc4c(c9c87)c65)n3)cccc21]

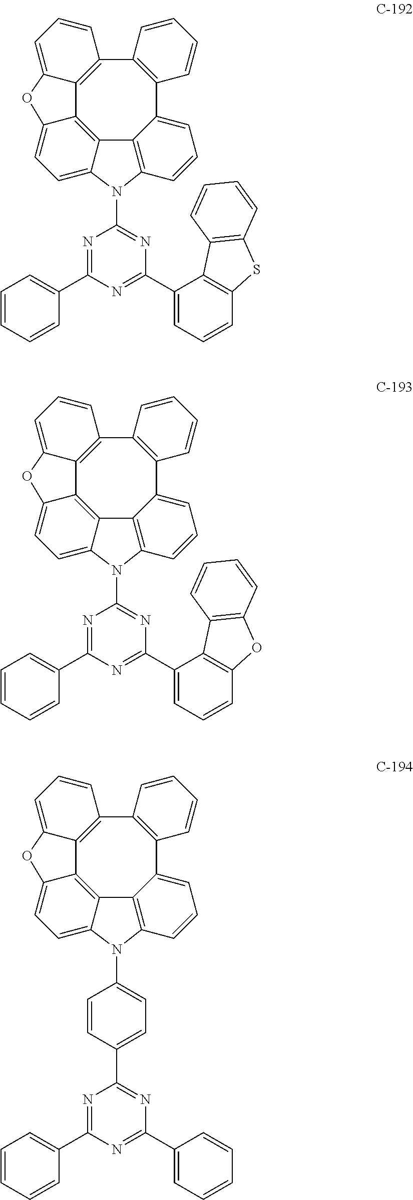 [SMILES: c1ccc(-c2nc(-c3cccc4sc5ccccc5c34)nc(-n3c4cccc5c6ccccc6c6cccc7oc8ccc3c(c8c76)c54)n2)cc1 ; c1ccc(-c2nc(-c3cccc4oc5ccccc5c34)nc(-n3c4cccc5c6ccccc6c6cccc7oc8ccc3c(c8c76)c54)n2)cc1 ; c1ccc(-c2nc(-c3ccccc3)nc(-c3ccc(-n4c5cccc6c7ccccc7c7cccc8oc9ccc4c(c9c87)c65)cc3)n2)cc1]

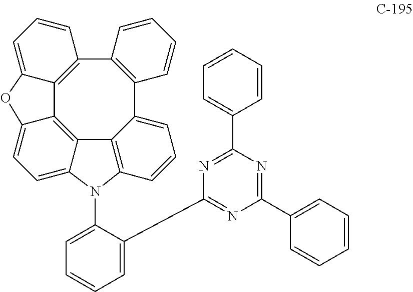 [SMILES: c1ccc(-c2nc(-c3ccccc3)nc(-c3ccccc3-n3c4cccc5c6ccccc6c6cccc7oc8ccc3c(c8c76)c54)n2)cc1]

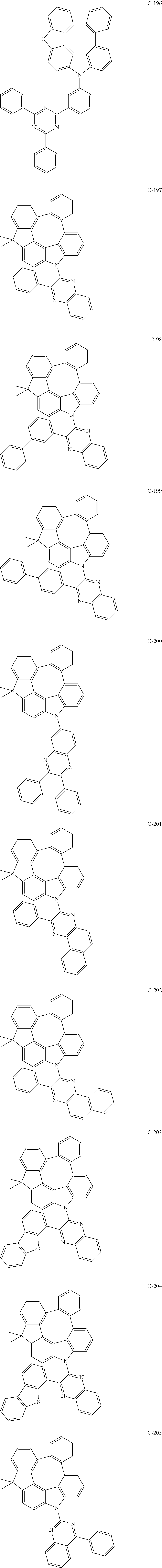

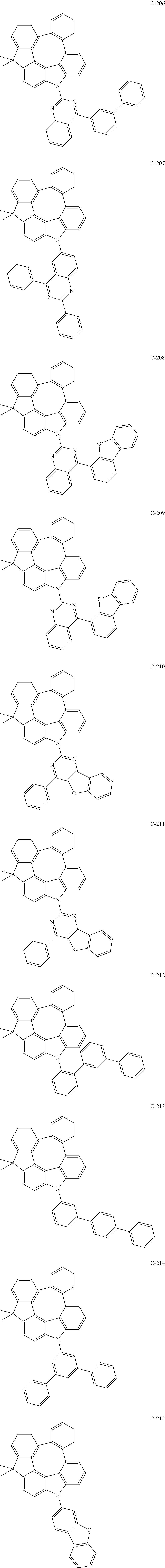

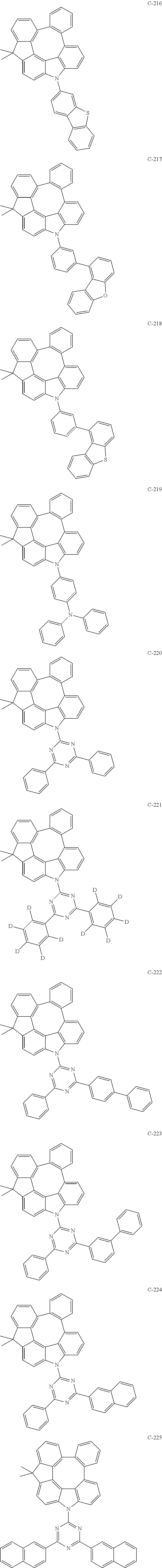

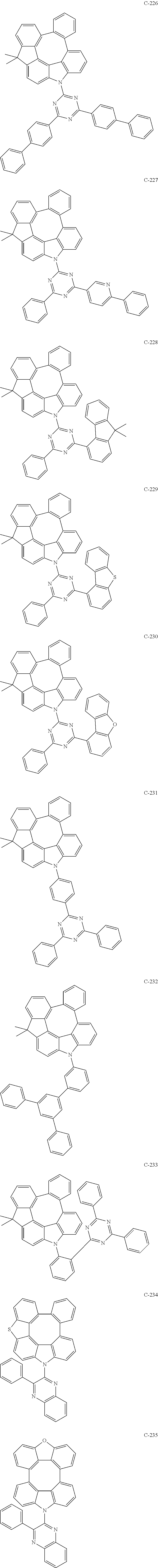

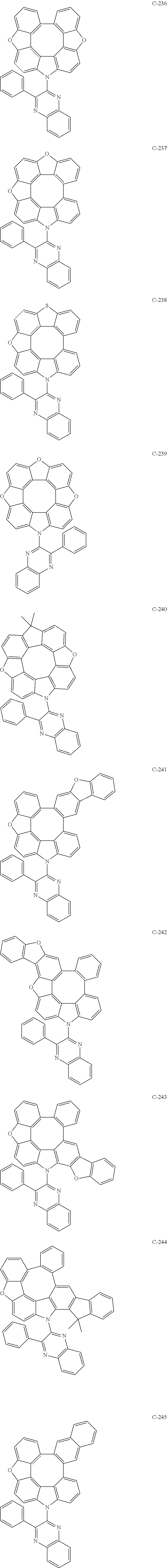

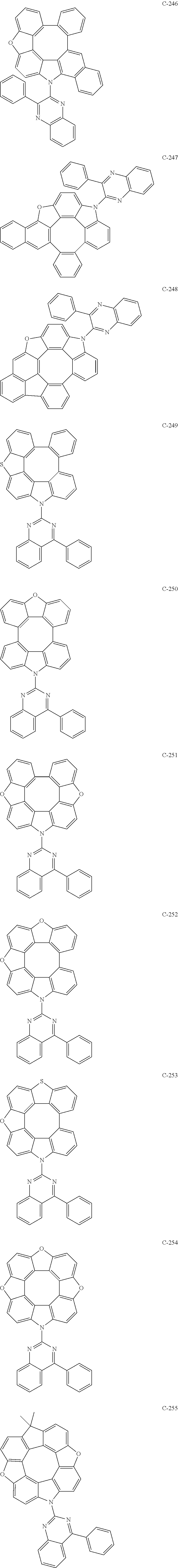

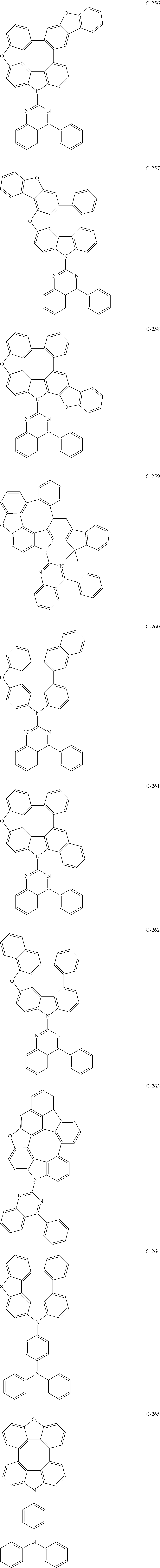

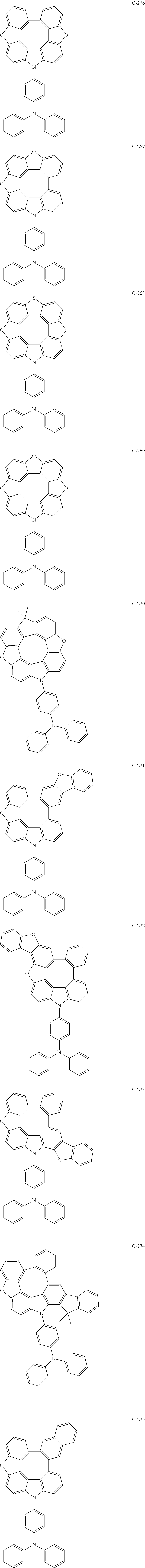

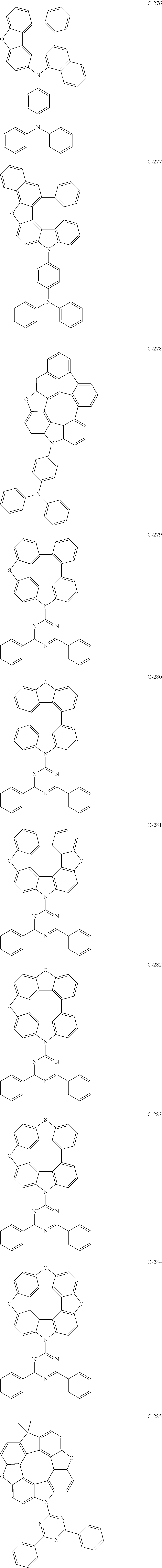

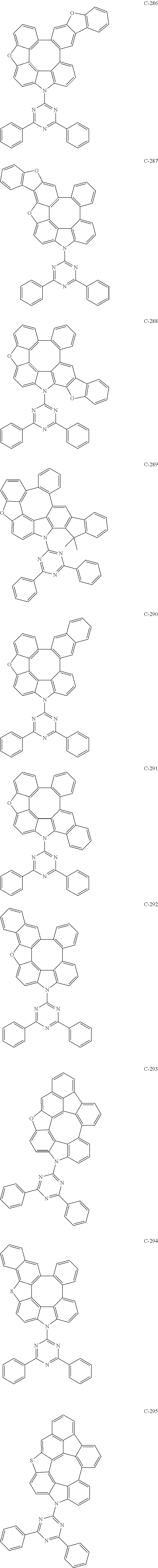

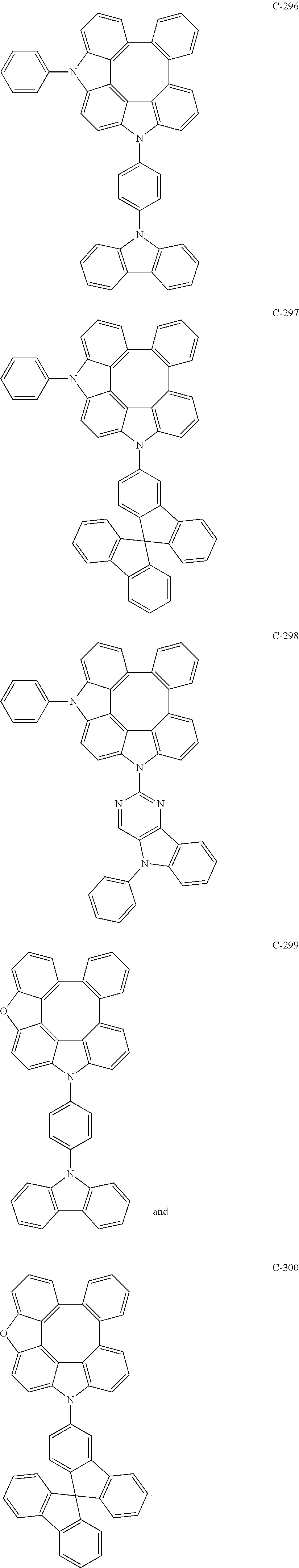

10. An organic electroluminescent device comprising an anode; a cathode; and at least one light-emitting layer between the anode and the cathode, wherein the at least one light-emitting layer comprise a plurality of host materials according to claim 1.
